(19) United States
(12) Patent Application Publication
(10) Pub. No.: US 2009/0036448 A1
(43) Pub. Date: Feb. 5, 2009

(54) PYRIMIDINE DERIVATIVES AND METHODS OF TREATMENT RELATED TO THE USE THEREOF

(75) Inventors: Yoshinori Sekiguchi, Tokyo (JP); Kosuke Kanuma, Tokyo (JP); Katsunori Omodera, Tokyo (JP); Thuy-Anh Tran, San Diego, CA (US); Graeme Semple, San Diego, CA (US); Bryan A. Kramer, San Diego, CA (US)

Correspondence Address: SUGHRUE MION, PLLC, 2100 PENNSYLVANIA AVENUE, N.W., SUITE 800, WASHINGTON, DC, 20037, US

(73) Assignees: TAISHO PHARMECUTICAL CO., LTD., Tokyo (JP); ARENA PHARMACEUTICALS, INC., San Diego, CA (US)

(21) Appl. No.: 10/599,505

(22) PCT Filed: Mar. 29, 2005
(86) PCT No.: PCT/JP2005/006582

§ 371 Date: Dec. 13, 2006

Related U.S. Application Data
(60) Provisional application No. 60/557,406, filed on Mar. 30, 2004.

(51) Int. Cl. A61K 31/506; C07D 239/48; A61K 31/5377; A61P 3/04; C07D 413/02

(52) U.S. Cl. 514/235.8; 544/326; 544/122; 514/256

(57) ABSTRACT

The present invention encompasses novel substituted pyrimidine compounds of Formula (I):

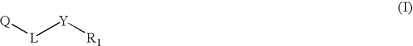

which act as MCH receptor antagonists. These compounds are useful in pharmaceutical compositions whose use includes prophylaxis or treatment of improving memory function, sleeping and arousal, anxiety, depression, mood disorders, seizure, obesity, diabetes, appetite and eating disorders, cardiovascular disease, hypertension, dyslipidemia, myocardial infarction, binge eating disorders including bulimia, anorexia, mental disorders including manic depression, schizophrenia, delirium, dementia, stress, cognitive disorders, attention deficit disorder, substance abuse disorders and dyskinesias including Parkinson's disease, epilepsy, and addiction.

### FIELD OF THE INVENTION

The present invention relates to compounds which act as antagonists for MCH receptors and to the use of these compounds in pharmaceutical compositions.

### BACKGROUND OF THE INVENTION

Melanin Concentrating Hormone (MCH), a cyclic peptide, has been identified as the endogenous ligand of the orphan G-protein coupled receptor SLC-1. See, for example, Shimomura et al., Biochem. Biophys. Res. Commun. 261, 622-26 (1999). Studies have indicated that MCH acts as a neurotransmitter/neuromodulator to alter a number of behavioral responses such as feeding habits. For example, injection of MCH into rats has been reported to increase their consumption of food. Reports indicate that genetically engineered mice which lack MCH show lower body weight and increased metabolism. See Saito et al., TEM, vol. 11, 299 (2000). As such, the literature suggests that discovery of MCH antagonists that interact with SCL-1 expressing cells will be useful in developing obesity treatments. See Shimomura et al., Biochem. Biophys. Res. Commun. 261, 622-26 (1999).

G protein-coupled receptors (GPCRs) share a common structural motif. All these receptors have seven sequences of between 22 to 24 hydrophobic amino acids that form seven alpha helices, each of which spans the membrane. The fourth and fifth transmembrane helices are joined on the extracellular side of the membrane by a strand of amino acids that forms a relatively large loop. Another larger loop, composed primarily of hydrophilic amino acids, joins transmembrane helices five and six on the intracellular side of the membrane. The carboxy terminus of the receptor lies intracellularly, and the amino terminus lies in the extracellular space. It is thought that the loop joining helices five and six, as well as the carboxy terminus, interact with the G protein. Currently, Gq, Gs, Gi, and Go are G proteins that have been identified as possible proteins that interact with the receptor.

Under physiological conditions, GPCRs exist in the cell membrane in equilibrium between two different states or conformations: an “inactive” state and an “active” state. A receptor in an inactive state is unable to link to the intracellular transduction pathway to produce a biological response. Changing the receptor conformation to the active state allows linkage to the transduction pathway and produces a biological response.

A receptor may be stabilized in an active state by an endogenous ligand or an exogenous agonist ligand. Recent discoveries, including but not exclusively limited to, modifications to the amino acid sequence of the receptor, provide alternative mechanisms other than ligands to stabilize the active state conformation. These approaches effectively stabilize the receptor in an active state by simulating the effect of a ligand binding to the receptor. Stabilization by such ligand-independent approaches is termed “constitutive receptor activation.” In contrast, antagonists can competitively bind to the receptor at the same site as agonists, but do not activate the intracellular response initiated by the active form of the receptor, and therefore inhibit the intracellular responses by agonists.

Certain 2-aminoquinazoline derivatives have been reported to be NPY antagonists which are said to be effective in the treatment of disorders and diseases associated with the NPY receptor subtype Y5. See PCT Patent Application 97/20823. Quinazoline derivatives have also been found to be useful by enhancing antitumor activity. See PCT Patent Application 92/07844. And also the quinoline derivatives which have an antagonist activity for MCH receptor are known in these patents, WO03/070244, WO03/105850, WO03/45313, WO03/045920, and WO04/04726.

Recently, our current knowledge of human obesity has advanced dramatically. Previously, obesity was viewed as an oppugnant behavior of inappropriate eating in the setting of appealing foods. Studies of animal models of obesity, biochemical alterations in both humans and animals, and the complex interactions of psychosocial and cultural factors that create receptiveness to human obesity indicate that this disease in humans is multifaceted and deeply entrenched in biologic systems. Thus, it is almost certain that obesity has multiple causes and that there are different types of obesity. Not only does MCHR1 antagonist have potent and durable anti-obesity effects in rodents, it has surprising antidepressant and anxiolytic properties as well (Borowsky et al., Nature Medicine, 8, 825-830, 2002). MCHR1 antagonists have been reported to show antidepressant and anxiolytic activities in rodent models such as social interaction, forced swimming test and ultrasonic vocalization. These findings indicate that MCHR1 antagonists could be useful for treatment of obesity patients with multiple causes. Moreover, MCHR1 antagonists could be used to treat subjects not only with obesity, but also those with depression and anxiety. These advantages make it different from NPY receptor antagonists, with which anxiogenic-like activity can be expected, as NPY itself has anxiolytic-like effect.

Obesity is also regarded as a chronic disease and the possibly of long-term treatment is a concept that is receiving more attention. In this context, it is noteworthy that the depletion of MCH leads to hypophagia as well as leanness (Shimada et al., Nature, 396, 670-674, 1998). By contrast, NPY (Erickson et al., Nature, 381, 415-418, 1996), as well as the Y1 (Pedrazzini et al., Nature Medicine, 4, 722-726, 1998) and Y5 receptors (Marsh et al., Nature Medicine, 4, 718-721, 1998), disrupted mice maintained a stable body weight or rather became obese. Considering the above reports, MCHR1 antagonists can be more attractive than Y1 or Y5 receptor antagonists in terms of long-term treatment of obese patients.

Obesity, which is the result of an imbalance between caloric intake and energy expenditure, is highly correlated with insulin resistance and diabetes in experimental animals and human. However, the molecular mechanisms that are involved in obesity-diabetes syndromes are not clear. During early development of obesity, increase insulin secretion balances insulin resistance and protects patients from hyperglycemia (Le Stunff, et al. Diabetes 43, 696-702 (1989)). However, after several decades, β cell function deteriorates and non-insulin-dependent diabetes develops in about 20% of the obese population (Pederson, P. Diab. Metab. Rev. 5, 505-509 (1989)) and (Brancati, F. L., et al., Arch. Intern. Med. 159, 957-963 (1999)). Given its high prevalence in modern societies, obesity has thus become the leading risk factor for NIDDM (Hill, J. O., et al., Science 280, 1371-1374 (1998)). However, the factors which predispose a fraction of patients to alteration of insulin secretion in response to fat accumulation remain unknown.

Whether someone is classified as overweight or obese is generally determined on the basis of their body mass index (BMI) which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m² (see TABLE below). There are problems with this definition in that it does not take into account the proportion of body mass that is muscle in relation to fat (adipose tissue). To account for this, obesity can also be defined on the basis of body fat content: greater than 25% and 30% in males and females, respectively.

CLASSIFICATION OF WEIGHT BY
BODY MASS INDEX (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases there is an increased risk of death from a variety of causes that is independent of other risk factors. The most common diseases with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease.

Compounds marketed as anti-obesity agents include Orlistat (XENICAL™) and Sibutramine. Orlistat (a lipase inhibitor) inhibits fat absorption directly and tends to produce a high incidence of unpleasant (though relatively harmless) side-effects such as diarrhea. Sibutramine (a mixed 5-HT/noradrenaline reuptake inhibitor) can increase blood pressure and heart rate in some patients. The serotonin releaser/reuptake inhibitors fenfluramine (Pondimin™) and dexfenfluramine (Redux™) have been reported to decrease food intake and body weight over a prolonged period (greater than 6 months). However, both products were withdrawn after reports of preliminary evidence of heart valve abnormalities associated with their use. Accordingly, there is a need for the development of a safer anti-obesity agent.

Obesity considerably increases the risk of developing cardiovascular diseases as well. Coronary insufficiency, atheromatous disease, and cardiac insufficiency are at the forefront of the cardiovascular complication induced by obesity. It is estimated that if the entire population had an ideal weight, the risk of coronary insufficiency would decrease by 25% and the risk of cardiac insufficiency and of cerebral vascular accidents by 35%. The incidence of coronary diseases is doubled in subjects less than 50 years of age who are 30% overweight. The diabetes patient faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions based on the prevention of these conditions based on the prevention of obesity (Perry, I. J., et al., BMJ 310, 560-564 (1995)).

An increasing number of children and adolescents are overweight. Although not all overweight children will necessarily become overweight adults, the growing occurrence of obesity in childhood is likely to be reflected in increasing obesity in adult years. The high prevalence of obesity in our adult population and the likelihood that the nation of the future will be even more obese demands a re-examination of the health implications of this disease. See, Health Implications of Obesity. NIH Consens. Statement Online 1985 Feb. 11-13; 5 (9):1-7.

“Clinical obesity” is a measurement of the excess body fat relative to lean body mass and is defined as a body weight more than 20% above the ideal body weight. Recent estimates suggest that 1 in 2 adults in the United States is clinically obese, an increase of more than 25% over the past decades. Flegal M. D. et al., 22 Int. J. Obes. Relat. Metab. Disor. 39 (1998). Both overweight conditions and clinical obesity are a major health concerns worldwide, in particular because clinical obesity is often accompanied by numerous complications, i.e., hypertension and Type II diabetes, which in turn can cause coronary artery disease, stroke, late-stage complications of diabetes and premature death. (See, e.g., Nishina P. M. et al., 43 Metab. 554 (1994)).

Although the etiologic mechanisms underlying obesity require further clarification, the net effect of such mechanisms leads to an imbalance between energy intake and expenditure. Both genetic and environmental factors are likely to be involved in the pathogenesis of obesity. These include excess caloric intake, decreased physical activity, and metabolic and endocrine abnormalities.

Treatment of overweight conditions and clinical obesity via pharmaceutical agents are not only of importance with respect to the conditions themselves, but also with respect to the possibility of preventing other diseases that are associated with, e.g., clinical obesity, as well as enhancement of the positive feeling of “self” that often accompanies those who are overweight or clinically obese and who encounter a significant reduction in body weight. Given the foregoing discussion, it is apparent that compounds which help in the treatment of such disorders would be useful and would provide an advance in both research and clinical medicine. The present invention is directed to these, as well as other, important ends.

### SUMMARY OF THE INVENTION

The present invention is drawn to compounds, which bind to and modulate the activity of a GPCR referred to herein as MCH, and uses thereof. The term MCH, as used herein, includes the human sequences found in GeneBank accession number NM_{—}005297, naturally-occurring allelic variants, mammalian orthologs, biologically active fragments and recombinant mutants thereof.

One aspect of the present invention relates to certain substituted pyrimidine compounds represented by Formula (I):

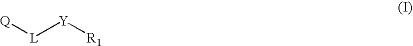

wherein Q is:

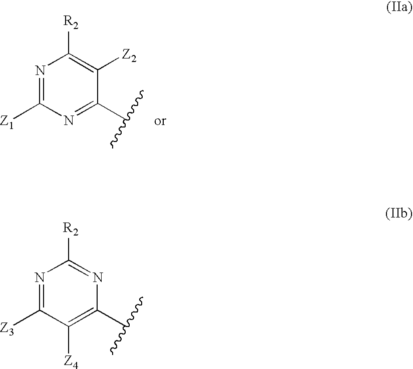

R₁ is selected from the group consisting of:
(i) C₁₋₁₆ alkyl, and
   C₁₋₁₆ alkyl substituted by substituent(s) independently selected from the group consisting of:
      halogen,
      hydroxy,
      oxo,
      C₁₋₅ alkoxy,
      C₁₋₅ alkoxy substituted by substituent(s) independently selected from the group consisting of:
         carbocyclic aryl,
         heterocyclyl, and
         heterocyclyl substituted by C₁₋₅ alkyl,
      C₁₋₅ alkylcarbonyloxy,
      carbocyclyloxy,
      carbocyclic aryloxy,
      carbocyclic aryloxy substituted by substituent(s) independently selected from the group consisting of:
         halogen,
         hydroxy,
         carboxy,
         carbamoyl,
         nitro,
         cyano,
         amino,
         carbocyclic aryl,
         carbocyclic aryl substituted by C₁₋₅ alkoxy,
         C₁₋₅ alkoxy,
         C₁₋₅ alkoxy substituted by halogen,
         C₁₋₅ alkyl, and
         C₁₋₅ alkyl substituted by substituent(s) independently selected from the group consisting of
            halogen,
            hydroxy,
            carboxy,
            oxo,
            mono-C₁₋₅ alkylamino,
            di-C₁₋₅ alkylamino,
            mono-C₁₋₅ alkylamino substituted by carbocyclic aryl,
            di-C₁₋₅ alkylamino substituted by carbocyclic aryl,
            mono-C₁₋₅ alkylamino substituted by halogenated carbocyclic aryl,
            di-C₁₋₅ alkylamino substituted by halogenated carbocyclic aryl,
            carbocyclic arylcarbonylamino, and
            carbocyclic arylcarbonylamino substituted by halogen,
      heterocyclyloxy,
      heterocyclyloxy substituted by substituent(s) independently selected from the group consisting of:
         halogen,
         hydroxy,
         carboxy,
         carbamoyl,
         nitro,
         cyano,
         amino,
         carbocyclic aryl,
         carbocyclic aryl substituted by C₁₋₅ alkoxy,
         C₁₋₅ alkoxy,
         C₁₋₅ alkoxy substituted by substituent(s) independently selected from the group consisting of:
            halogen,
            hydroxy, and
            carboxy,
         C₁₋₅ alkyl, and
         C₁₋₅ alkyl substituted by substituent(s) independently selected from the group consisting of:
            halogen,
            hydroxy, and
            carboxy,
      substituted heterocyclyl-ethylideneaminooxy,
      C₁₋₅ alkoxycarbonyl,
      C₁₋₅ alkoxycarbonyl substituted by carbocyclic aryl,
      mono-C₁₋₅ alkylaminocarbonyl,
      di-C₁₋₅ alkylaminocarbonyl,
      mono-C₁₋₅ alkylamino,
      mono-C₁₋₅ alkylamino substituted by substituent(s) independently selected from the group consisting of:
         cyano,
         carbocyclic aryl, and
         heterocyclyl,
      di-C₁₋₅ alkylamino,
      di-C₁₋₅ alkylamino substituted by substituent(s) independently selected from the group consisting of:
         cyano,
         carbocyclic aryl, and
         heterocyclyl,
      mono-carbocyclic arylamino,
      mono-carbocyclic arylamino substituted by substituent(s) independently selected from the group consisting of:
         halogen,
         hydroxy,
         carboxy,
         carbamoyl,
         nitro,
         cyano,
         amino,
         carbocyclic aryl,
         carbocyclic aryl substituted by C₁₋₅ alkoxy,
         C₁₋₅ alkoxy,
         C₁₋₅ alkoxy substituted by substituent(s) independently selected from the group consisting of:
            halogen,
            hydroxy, and
            carboxy,
         C₁₋₅ alkyl, and
         C₁₋₅ alkyl substituted by substituent(s) independently selected from the group consisting of:
            halogen,
            hydroxy, and
            carboxy,
      di-carbocyclic arylamino,
      di-carbocyclic arylamino substituted by substituent(s) independently selected from the group consisting of:
         halogen,
         hydroxy,
         carboxy,
         carbamoyl,
         nitro,
         cyano,
         amino,
         carbocyclic aryl,
         carbocyclic aryl substituted by C₁₋₅ alkoxy,
         C₁₋₅ alkoxy,
         C₁₋₅ alkoxy substituted by substituent(s) independently selected from the group consisting of:
            halogen,
            hydroxy, and
            carboxy,
         C₁₋₅ alkyl, and
         C₁₋₅ alkyl substituted by substituent(s) independently selected from the group consisting of:
            halogen,
            hydroxy, and
            carboxy,
      mono-heterocyclylamino,
      mono-heterocyclylamino substituted by substituent(s) independently selected from the group consisting of:
         halogen,
         hydroxy,
         carboxy,
         carbamoyl,
         nitro,
         cyano,
         amino,
         carbocyclic aryl,
         carbocyclic aryl substituted by C₁₋₅ alkoxy,
         C₁₋₅ alkoxy,
         C₁₋₅ alkoxy substituted by substituent(s) independently selected from the group consisting of:
            halogen,
            hydroxy, and
            carboxy,
         C₁₋₅ alkyl, and
         C₁₋₅ alkyl substituted by substituent(s) independently selected from the group consisting of:
            halogen,
            hydroxy, and
            carboxy,
      di-heterocyclylamino,
      di-heterocyclylamino substituted by substituent(s) independently selected from the group consisting of:
         halogen,
         hydroxy,
         carboxy,
         carbamoyl,
         nitro,
         cyano,
         amino,
         carbocyclic aryl,
         carbocyclic aryl substituted by C₁₋₅ alkoxy,
         C₁₋₅ alkoxy,
         C₁₋₅ alkoxy substituted by substituent(s) independently selected from the group consisting of:
            halogen,
            hydroxy, and
            carboxy,
         C₁₋₅ alkyl, and
         C₁₋₅ alkyl substituted by substituent(s) independently selected from the group consisting of:
            halogen,
            hydroxy, and
            carboxy,
      C₁₋₅ alkylcarbonylamino,
      C₁₋₅ alkylcarbonylamino substituted by substituent(s) independently selected from the group consisting of:
         C₁₋₅ alkylcarbonylamino,
         carbocyclic arylcarbonylamino, and
         heterocyclyl,
      C₁₋₅ alkoxycarbonylamino,
      carbocyclic arylcarbonylamino,
      heterocyclyl carbonylamino,
      carbocyclic arylsulfonylamino,
      carbocyclic arylsulfonylamino substituted by substituent(s) independently selected from the group consisting of:
         nitro,
         C₁₋₅ alkyl,
         mono-C₁₋₅ alkylamino, and
         di-C₁₋₅ alkylamino,
      C₁₋₅ alkylthio,
      C₁₋₅ alkylthio substituted by substituent(s) independently selected from the group consisting of:
         mono-carbocyclic arylaminocarbonyl,
         mono-carbocyclic arylaminocarbonyl substituted by halogen,
         di-carbocyclic arylaminocarbonyl,
         di-carbocyclic arylaminocarbonyl substituted by halogen,
         mono-carbocyclic arylamino,
         mono-carbocyclic arylamino substituted by halogen,
         di-carbocyclic arylamino,
         di-carbocyclic arylamino substituted by halogen,
         carbocyclic aryl, and
         carbocyclic aryl substituted by substituent(s) independently selected from the group consisting of:
            halogen, and
            C₁₋₅ alkoxy,
      carbocyclic arylthio,
      carbocyclic arylthio substituted by substituent(s) independently selected from the group consisting of:
         halogen,
         C₁₋₅ alkyl, and
         C₁₋₅ alkyl substituted by halogen,
      carbocyclic arylsulfinyl,
      carbocyclic arylsulfinyl substituted by substituent(s) independently selected from the group consisting of:
         halogen,
         C₁₋₅ alkyl, and
         C₁₋₅ alkyl substituted by halogen,
      carbocyclic arylsulfonyl,
      carbocyclic arylsulfonyl substituted by substituent(s) independently selected from the group consisting of:
         halogen,
         C₁₋₅ alkyl, and
         C₁₋₅ alkyl substituted by halogen,
      heterocyclylthio,
      heterocyclylthio substituted by substituent(s) independently selected from the group consisting of:
         nitro, and
         C₁₋₅ alkyl,
      C₃₋₆ cycloalkyl,
      C₃₋₆ cycloalkyl substituted by C₁₋₅ alkyl,
      C₃₋₆ cycloalkyl substituted by carbocyclic aryl,
      C₃₋₆ cycloalkenyl,
      carbocyclyl,
      carbocyclyl substituted by substituent(s) independently selected from the group consisting of:
         halogen,
         C₁₋₅ alkyl,
         C₁₋₅ alkoxy,
         C₂₋₅ alkenyl, and
         C₂₋₅ alkenyl substituted by substituent(s) independently selected from the group consisting of:
            carbocyclic aryl, and
            carbocyclic aryl substituted by C₁₋₅ alkylsulfinyl,
      carbocyclic aryl,
      carbocyclic aryl substituted by substituent(s) independently selected from the group consisting of:
         halogen,
         hydroxy,
         carboxy,
         carbamoyl,
         cyano,
         nitro,
         amino,
         C₁₋₅ alkylcarbonylamino,
         C₃₋₆ cycloalkylcarbonylamino,
         C₁₋₅ alkyl,
         C₁₋₅ alkyl substituted by substituent(s) independently selected from the group consisting of:
            halogen,
            hydroxy,
            carboxy,
            carbamoyl,
            oxo,
            carbocyclic aryl,
            heterocyclyl,
            mono-carbocyclic arylamino,
            di-carbocyclic arylamino,
            mono-carbocyclic arylamino substituted by substituent(s) independently selected from the group consisting of:
            halogen,
            nitro,
            C₁₋₅ alkyl,
            C₁₋₅ alkoxy, and
            C₁₋₅ alkoxy substituted by halogen,
            di-carbocyclic arylamino substituted by substituent(s) independently selected from the group consisting of:
            halogen,
            nitro,
            C₁₋₅ alkyl,
            C₁₋₅ alkoxy, and
            C₁₋₅ alkoxy substituted by halogen,
         C₂₋₅ alkenyl,
         C₁₋₅ alkoxy,
         C₁₋₅ alkoxy substituted by substituent(s) independently selected from the group consisting of:
            halogen, and
            carbocyclic aryl,
         carbocyclic aryloxy,
         C₁₋₅ alkoxycarbonyl,
         C₁₋₅ alkylcarbonyloxy,
         mono-C₁₋₅ alkylamino,
         di-C₁₋₅ alkylamino,
         mono-carbocyclic arylamino,
         mono-carbocyclic arylamino substituted by halogen,
         di-carbocyclic arylamino,
         di-carbocyclic arylamino substituted by halogen,
         mono-carbocyclic arylaminocarbonyl,
         mono-carbocyclic arylaminocarbonyl substituted by substituent(s) selected from the group consisting of:
            halogen,
            nitro,
            C₁₋₅ alkyl,
            C₁₋₅ alkoxy, and
            C₁₋₅ alkoxy substituted by halogen,
         di-carbocyclic arylaminocarbonyl,
         di-carbocyclic arylaminocarbonyl substituted by substituent(s) selected from the group consisting of:
            halogen,
            nitro,
            C₁₋₅ alkyl,
            C₁₋₅ alkoxy, and
            C₁₋₅ alkoxy substituted by halogen,
         mercapto,
         C₁₋₅ alkylthio,
         C₁₋₅ alkylthio substituted by halogen,
         C₁₋₅ alkylsulfonyl,
         C₃₋₆ cycloalkyl,
         carbocyclic aryl, and
         heterocyclyl,
      heterocyclyl, and
      heterocyclyl substituted by substituent(s) independently selected from the group consisting of:
         halogen,
         hydroxy,
         carboxy,
         carbamoyl,
         cyano,
         nitro,
         amino,
         C₁₋₅ alkyl,
         C₁₋₅ alkyl substituted by substituent(s) independently selected from the group consisting of:
            halogen,
            hydroxy,
            carboxy, and
            carbamoyl,
         C₁₋₅ alkyl substituted by carbocyclic aryl,
         C₁₋₅ alkoxy,
         C₁₋₅ alkoxy substituted by halogen,
         C₁₋₅ alkoxy substituted by carbocyclic aryl,
         carbocyclic aryl, and
         carbocyclic aryl substituted by halogen,
(ii) C₂₋₈ alkenyl, and
   C₂₋₈ alkenyl substituted by substituent(s) independently selected from the group consisting of:
      halogen,
      oxo,
      C₁₋₅ alkoxy,
      C₁₋₅ alkoxy substituted by carbocyclic aryl,
      carbocyclic aryl,
      carbocyclic aryl substituted by substituent(s) independently selected from the group consisting of:
         halogen,
         hydroxy,
         nitro,
         C₁₋₅ alkyl,
         C₁₋₅ alkyl substituted by halogen,
         C₁₋₅ alkoxy, and
         C₁₋₅ alkoxy substituted by halogen,
      heterocyclyl, and
      heterocyclyl substituted by substituent(s) independently selected from the group consisting of:
         hydroxy,
         nitro,
         C₁₋₅ alkyl, and
         C₁₋₅ alkoxy,
(iii) C₂₋₅ alkynyl, and
   C₂₋₅ alkynyl substituted by carbocyclic aryl,
(iv) C₃₋₁₂ cycloalkyl, and
   C₃₋₁₂ cycloalkyl substituted by substituent(s) independently selected from the group consisting of:
      C₁₋₅ alkyl,
      C₁₋₅ alkyl substituted by substituent(s) independently selected from the group consisting of:
         hydroxy,
         oxo, and
         carbocyclic aryl,
      mono-C₁₋₅ alkylamino,
      mono-C₁₋₅ alkylamino substituted by carbocyclic aryl,
      di-C₁₋₅ alkylamino,
      di-C₁₋₅ alkylamino substituted by carbocyclic aryl,
      carbocyclic arylcarbonylamino,
      carbocyclic aryl, and
      carbocyclic aryl substituted by halogen,
(v) C₃₋₆ cycloalkenyl, and
   C₃₋₆ cycloalkenyl substituted by C₁₋₅ alkyl,
(vi) carbocyclyl, and
   carbocyclyl substituted by substitutent(s) independently selected from the group consisting of:
      hydroxy, and
      nitro,
(vii) carbocyclic aryl, and
   carbocyclic aryl substituted by substituent(s) independently selected from the group consisting of:
      halogen,
      hydroxy,
      cyano,
      nitro,
      C₁₋₁₀ alkyl,
      C₁₋₁₀ alkyl substituted by substituent(s) independently selected from the group consisting of:
         halogen,
         hydroxy,
         carboxy,
         carbamoyl,
         oxo,
         C₁₋₅ alkoxy,
         carbocyclic aryloxy,
         mono-C₁₋₅ alkylamino-N-oxy,
         di-C₁₋₅ alkylamino-N-oxy,
         mono-C₁₋₅ alkylamino,
         di-C₁₋₅ alkylamino,
         mono-C₁₋₅ alkylamino substituted by carbocyclic aryl,
         di-C₁₋₅ alkylamino substituted by carbocyclic aryl,
         mono-carbocyclic arylamino,
         di-carbocyclic arylamino,
         carbocyclylimino,
         carbocyclylimino substituted by carbocyclic aryl,
         mono-carbocyclic arylamino,
         di-carbocyclic arylamino,
         mono-carbocyclic arylamino substituted by C₁₋₅ alkoxy,
         di-carbocyclic arylamino substituted by C₁₋₅ alkoxy,
         mono-carbocyclic arylaminocarbonyl,
         di-carbocyclic arylaminocarbonyl,
         mono-carbocyclic arylaminocarbonyl substituted by C₁₋₅ alkoxy,
         di-carbocyclic arylaminocarbonyl substituted by C₁₋₅ alkoxy,
         carbocyclic aryl,
         carbocyclic aryl substituted by substituent(s) independently selected from the group consisting of:
            halogen,
            C₁₋₅ alkyl, and
            C₁₋₅ alkyl substituted by halogen,
         heterocyclyl, and
         heterocyclyl substituted by C₁₋₅ alkyl,
      C₂₋₅ alkenyl,
      C₂₋₅ alkenyl substituted by carbocyclic aryl,
      C₁₋₉ alkoxy,
      C₁₋₉ alkoxy substituted by substituent(s) independently selected from the group consisting of:
         hydroxy,
         halogen,
         carboxy,
         mono-C₁₋₅ alkylamino,
         di-C₁₋₅ alkylamino,
         carbocyclic aryl,
         halogenated carbocyclic aryl,
         heterocyclyl,
         heterocyclyl substituted by substituent(s) independently selected from the group consisting of:
            halogen,
            heterocyclyl, and
            heterocyclyl substituted by substituent(s) independently selected from the group consisting of:
            halogen,
            C₁₋₅ alkyl, and
            C₁₋₅ alkyl substituted by halogen,
      C₂₋₅ alkenyloxy,
      C₃₋₆ cycloalkoxy,
      C₁₋₅ alkylcarbonyloxy,
      carbocyclic aryloxy,
      carbocyclic aryloxy substituted by substituent(s) independently selected from the group consisting of:
         halogen,
         hydroxy,
         carboxy,
         carbamoyl,
         cyano,
         nitro,
         amino,
         C₁₋₅ alkyl,
         C₁₋₅ alkyl substituted by substituent(s) independently selected from the group consisting of:
            halogen,
            hydroxy,
            carboxy, and
            carbamoyl,
         C₁₋₅ alkoxy, and
         C₁₋₅ alkoxy substituted by halogen,
      heterocyclyloxy,
      heterocyclyloxy substituted by substituent(s) independently selected from the group consisting of:
         halogen,
         hydroxy,
         carboxy,
         carbamoyl,
         cyano,
         nitro,
         amino,
         C₁₋₅ alkyl,
         C₁₋₅ alkyl substituted by substituent(s) independently selected from the group consisting of:
            halogen,
            hydroxy,
            carboxy, and
            carbamoyl,
         C₁₋₅ alkoxy, and
         C₁₋₅ alkoxy substituted by halogen,
      (carbocyclic aryl)S(O)₂O,
      carboxy,
      carbamoyl,
      C₁₋₅ alkoxycarbonyl,
      mono-C₁₋₅ alkylaminocarbonyl,
      di-C₁₋₅ alkylaminocarbonyl,
      mono-C₁₋₅ alkylaminocarbonyl substituted by carbocyclic aryl,
      di-C₁₋₅ alkylaminocarbonyl substituted by carbocyclic aryl,
      mono-carbocyclic arylaminocarbonyl,
      di-carbocyclic arylaminocarbonyl,
      mono-carbocyclic arylaminocarbonyl substituted by C₁₋₅ alkyl,
      di-carbocyclic arylaminocarbonyl substituted by C₁₋₅ alkyl,
      amino,
      mono-C₁₋₅ alkylamino,
      di-C₁₋₅ alkylamino,
      mono-C₁₋₅ alkylamino substituted by cyano,
      di-C₁₋₅ alkylamino substituted by cyano,
      mono-carbocyclic arylamino,
      di-carbocyclic arylamino,
      C₁₋₅ alkylcarbonylamino,
      C₃₋₆ cycloalkylcarbonylamino,
      C₂₋₅ alkynylcarbonylamino,
      C₂₋₅ alkynylcarbonylamino substituted by carbocyclic aryl,
      C₁₋₅ alkoxycarbonylamino,
      carbocyclic arylsulfonylamino,
      carbocyclic arylsulfonylamino substituted by C₁₋₅ alkyl,
      (carbocyclic aryl)NHC(O)NH,
      (carbocyclic aryl)NHC(O)NH substituted by C₁₋₅ alkoxy,
      (carbocyclic aryl)NHC(O)NH substituted by halogenated C₁₋₅ alkoxy,
      carbocyclic aryl azo,
      carbocyclic aryl azo substituted by mono-C₁₋₅ alkylamino,
      carbocyclic aryl azo substituted by di-C₁₋₅ alkylamino,
      C₁₋₅ alkylthio,
      C₁₋₅ alkylthio substituted by halogen,
      carbocyclic arylthio,
      carbocyclic arylthio substituted by substituent(s) independently selected from the group consisting of:
         halogen,
         nitro,
         cyano, and
         C₁₋₅ alkyl,
      aminosulfonyl,
      heterocyclylthio,
      C₁₋₅ alkylsulfonyl,
      mono-C₁₋₅ alkylaminosulfonyl,
      di-C₁₋₅ alkylaminosulfonyl,
      heterocyclylsulfonyl,
      C₃₋₆ cycloalkyl,
      C₃₋₆ cycloalkyl substituted by C₁₋₅ alkyl,
      carbocyclic aryl,
      carbocyclic aryl substituted by substituent(s) independently selected from the group consisting of:
         C₁₋₇ alkyl, and
         C₁₋₇ alkyl substituted by halogen,
      heterocyclyl, and
      heterocyclyl substituted by substituent(s) independently selected from the group consisting of:
         C₁₋₅ alkyl,
         carbocyclic aryl, and
         halogenated carbocyclic aryl,
      C₁₋₅ alkoxycarbonyl substituted by carbocyclic aryl, and
(viii) heterocyclyl, and
   heterocyclyl substituted by substituent(s) independently selected from the group consisting of:
      halogen,
      hydroxy,
      carboxy,
      carbamoyl,
      cyano,
      nitro,
      amino,
      C₁₋₅ alkyl,
      C₁₋₅ alkyl substituted by substituent(s) independently selected from the group consisting of:
         halogen,
         hydroxy,
         carboxy,
         carbamoyl,
         oxo,
         C₁₋₅ alkylcarbonyloxy,
         carbocyclic arylcarbonylamino,
         carbocyclic arylcarbonylamino substituted by halogen,
         C₁₋₅ alkoxycarbonyl,
         C₁₋₅ alkylthio,
         C₁₋₅ alkylthio substituted by carbocyclic aryl,
         C₁₋₅ alkylthio substituted by halogenated carbocyclic
         aryl,
         carbocyclic aryl,
         carbocyclic aryl substituted by substituent(s) independently selected from the group consisting of:
            halogen, and
            nitro,
         heterocyclyl, and
         heterocyclyl substituted by substituent(s) independently selected from the group consisting of:
            halogen,
            C₁₋₅ alkyl, and
            C₁₋₅ alkyl substituted by halogen,
      C₁₋₅ alkoxy,
      C₁₋₅ alkoxy substituted by halogen,
      C₁₋₅ alkoxy substituted by carbocyclic aryl,
      carbocyclic aryloxy,
      carbocyclic aryloxy substituted by substituent(s) independently selected from the group consisting of:
         halogen,
         nitro,
         cyano,
         hydroxy,
         carboxy,
         carbamoyl,
         amino,
         C₁₋₅ alkyl,
         C₁₋₅ alkyl substituted by substituent(s) independently selected from the group consisting of:
            halogen,
            hydroxy,
            carboxy, and
            carbamoyl,
         mono-C₁₋₅ alkylamino,
         di-C₁₋₅ alkylamino,
         C₁₋₅ alkylcarbonylamino,
         C₃₋₆ cycloalkycarbonylamino,
         C₁₋₅ alkoxy,
         C₁₋₅ alkoxy substituted by halogen,
         C₃₋₆ cycloalkyl,
         C₂₋₅ alkenyl,
         C₂₋₅ alkynyl,
         carboxy,
         C₁₋₅ alkoxycarbonyl,
         mono-C₁₋₅ alkylaminocarbonyl,
         di-C₁₋₅ alkylaminocarbonyl,
         mono-C₃₋₆ cycloalkylaminocarbonyl,
         di-C₃₋₆ cycloalkylaminocarbonyl,
         mono-C₁₋₅ alkylaminocarbonylamino,
         di-C₁₋₅ alkylaminocarbonylamino,
         mono-C₃₋₆ cycloalkylaminocarbonylamino,
         di-C₃₋₆ cycloalkylaminocarbonylamino,
         C₁₋₅ alkylthio,
         C₁₋₅ alkylthio substituted by halogen,
         C₁₋₅ alkylsulfinyl,
         C₁₋₅ alkylsulfinyl substituted by halogen,
         C₁₋₅ alkylsulfonyl, and
         C₁₋₅ alkylsulfonyl substituted by halogen,
      heterocyclyloxy,
      heterocyclyloxy substituted by substituent(s) independently selected from the group consisting of:
         halogen,
         nitro,
         hydroxy,
         carboxy,
         carbamoyl,
         cyano,
         amino,
         C₁₋₅ alkyl,
         C₁₋₅ alkyl substituted by substituent(s) independently selected from the group consisting of:
            halogen,
            hydroxy,
            carboxy, and
            carbamoyl,
         C₁₋₅ alkoxy, and
         C₁₋₅ alkoxy substituted by halogen,
      mono-C₁₋₅ alkylamino,
      di-C₁₋₅ alkylamino,
      C₁₋₅ alkylcarbonylamino,
      C₁₋₅ alkylthio,
      C₂₋₅ alkenylthio,
      carbocyclic arylthio,
      carbocyclic arylthio substituted by halogen,
      carbocyclic arylthio substituted by C₁₋₅ alkoxycarbonyl,
      heterocyclylthio,
      heterocyclylthio substituted by C₁₋₅ alkyl,
      C₁₋₅ alkylsulfinyl,
      C₁₋₅ alkylsulfonyl,
      carbocyclic arylsulfinyl,
      carbocyclic arylsulfinyl substituted by halogen,
      carbocyclic arylsulfonyl,
      carbocyclic arylsulfonyl substituted by halogen,
      carbocyclic arylsulfonyl substituted by C₁₋₅ alkyl,
      C₁₋₅ alkoxycarbonyl,
      C₁₋₅ alkoxycarbonyl substituted by carbocyclic aryl,
      carbocyclic aryl,
      carbocyclic aryl substituted by substituent(s) independently selected from the group consisting of:
         halogen,
         nitro,
         C₁₋₅ alkyl,
         C₁₋₅ alkyl substituted by halogen,
         C₁₋₅ alkoxy, and
         C₁₋₅ alkoxy substituted by halogen,
      heterocyclyl, and
      heterocyclyl substituted by substituent(s) independently selected from the group consisting of:
         halogen,
         C₁₋₅ alkyl,
         C₁₋₅ alkyl substituted by halogen,
         C₁₋₅ alkoxy, and
         C₁₋₅ alkoxycarbonyl;
   R₂ is halogen, C₁₋₅ alkyl, C₁₋₅ alkyl substituted by halogen, C₁₋₅ alkyl substituted by hydroxy, C₁₋₅ alkyl substituted by carbocyclic aryl, C₁₋₅ alkyl substituted by halogenated carbocyclic aryl, C₁₋₅ alkyl substituted by heterocyclyl, C₁₋₅ alkyl substituted by halogenated heterocyclyl, C₂₋₅ alkenyl, C₂₋₅ alkynyl, C₁₋₅ alkoxy, C₁₋₅ alkoxy substituted by halogen, C₁₋₅ alkylthio, —N(R₂ₐ)(R_{2b}); wherein R₂ₐ and R_{2b} are each independently hydrogen, C₁₋₅ alkyl, or C₁₋₅ alkyl substituted by substituent(s) independently selected from the group consisting of:
      halogen,
      hydroxy,
      carboxy,
      carbamoyl,
      C₁₋₅ alkoxy,
      amino,
      C₃₋₆ cycloalkyl,
      carbocyclic aryl,
      carbocyclic aryl substituted by substituent(s) independently selected from the group consisting of:
         halogen,
         C₁₋₅ alkyl,
         C₁₋₅ alkoxy,
         C₁₋₅ alkyl substituted by halogen,
         C₁₋₅ alkoxy substituted by halogen, and
         SO₂NH₂,
      heterocyclyl, and
      heterocyclyl substituted by substituent(s) independently selected from the group consisting of:
         halogen,
         C₁₋₅ alkyl,
         C₁₋₅ alkoxy,
         C₁₋₅ alkyl substituted by halogen, and
         C₁₋₅ alkoxy substituted by halogen,
   C₃₋₆ cycloalkyl, carbocyclic aryl, carbocyclic aryl substituted by substituent(s) independently selected from the group consisting of:
      halogen,
      C₁₋₅ alkyl,
      C₁₋₅ alkoxy,
      C₁₋₅ alkyl substituted by halogen, and
      C₁₋₅ alkoxy substituted by halogen,
   heterocyclyl, or heterocyclyl substituted by substituent(s) independently selected from the group consisting of:
      halogen,
      C₁₋₅ alkyl,
      C₁₋₅ alkoxy,
      C₁₋₅ alkyl substituted by halogen, and
      C₁₋₅ alkoxy substituted by halogen;

L is selected from the group consisting of Formulae (III), (IIIa), (IIIb), (IV), (IVa), and (IVb);

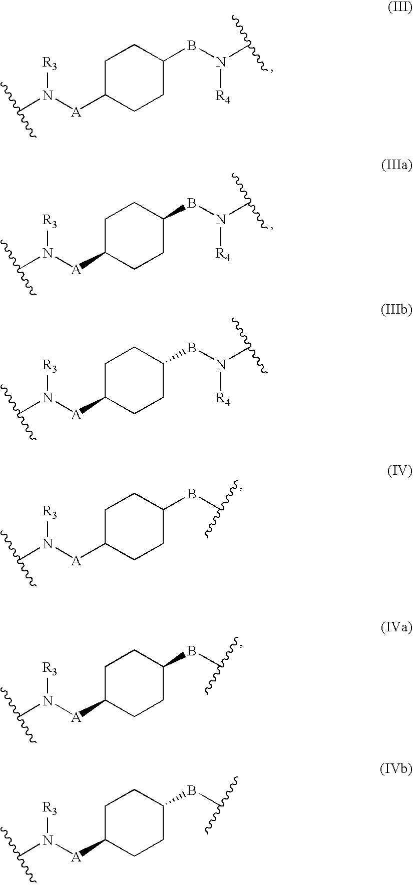

wherein R₃ and R₄ are each independently hydrogen or C₁₋₅ alkyl; and A and B are each independently a single bond, —CH₂—, or —(CH₂)₂—; Z₁, Z₂, Z₃, and Z₄ are each independently hydrogen, halogen, C₁₋₅ alkyl, C₁₋₅ alkyl substituted by halogen, C₁₋₅ alkyl substituted by hydroxy, C₁₋₅ alkyl substituted by carbocyclic aryl, C₁₋₅ alkyl substituted by halogenated carbocyclic aryl, C₁₋₅ alkyl substituted by heterocyclyl, C₁₋₅ alkyl substituted by halogenated heterocyclyl, C₂₋₅ alkenyl, C₂₋₅ alkynyl, C₃₋₆ cycloalkyl, C₁₋₅ alkoxy, C₁₋₅ alkoxy substituted by halogen, mono-C₁₋₅ alkyl amino, di-C₁₋₅ alkyl amino, C₁₋₅ alkylthio, carbocyclic aryl, substituted carbocyclic aryl, heterocyclyl, or substituted heterocyclyl; or R₂ and Z₂ are bonded to each other to form a ring and —R₂-Z₂- is —(CH₂)n- or —(CH₂)o-CH═CH—(CH₂)p-; wherein one —CH₂— group of —R₂-Z₂- can optionally be replaced by C(O), NR₆, O, S, S(O), or S(O)₂; wherein n is 2, 3, 4, 5, or 6; o and p are each independently 0, 1, 2, 3, or 4 provided that o+p=0, 1, 2, 3, or 4; and R₆ is hydrogen, C₁₋₅ alkyl, or substituted C₁₋₅ alkyl;
   and
Y represents:
(i) —C(O)NR₅—, —C(S)NR₅—, —C(O)O—, —S(O)₂—, —C(O)—, —C(S)—, or —(CH₂)ₘ— when L is selected from the group consisting of Formulae (III), (IIIa), and (IIIb); or
(ii) —C(O)NR₅—, —C(S)NR₅—, —C(O)O—, or —OC(O)— when L is selected from the group consisting of Formulae (IV), (IVa), and (IVb);
wherein R₅ is hydrogen or C₁₋₅ alkyl; and m is 0, 1, 2, 3, 4, or 5;
   wherein carbocyclic aryl is phenyl, naphthyl, anthranyl, phenanthryl, or biphenyl;
   carbocyclyl is 10,11-dihydro-5-oxo-dibenzo[a,d]cycloheptyl, 1-oxo-indanyl, 7,7-dimethyl-2-oxo-bicyclo[2.2.1]heptyl, 9H-fluorenyl, 9-oxo-fluorenyl, acenaphthyl, anthraquinonyl, C-fluoren-9-ylidene, indanyl, indenyl, menthyl, 1,2,3,4-tetrahydro-naphthyl, or bicyclo[2.2.1]heptenyl;
   heterocyclyl is 1,2,3,4-tetrahydro-isoquinolyl, 1,2,3-thiadiazolyl, 1,2,3-triazolyl, 1,2-dihydro-3-oxo-pyrazolyl, 1,3,4-thiadiazolyl, 1,3-dioxo-isoindolyl, 1,3-dioxolanyl, 1H-indolyl, 1H-pyrrolo[2,3-c]pyridyl, 1H-pyrrolyl, 1-oxo-3H-isobenzofuranyl, 2,2′,5′,2″-terthiophenyl, 2,2′-bithiophenyl, 2,3-dihydro-1-oxo-isoindolyl, 2,3-dihydro-benzo[1,4]dioxinyl, 2,3-dihydro-benzofuryl, 2,4-dihydro-3-oxo-pyrazolyl, 2H-benzopyranyl, 2-oxo-benzopyranyl, 2-oxo-pyrrolidinyl, 3,4-dihydro-2H-benzo[1,4]oxazinyl, 3,4-dihydro-2H-benzo[b][1,4]dioxepinyl, 4H-benzo[1,3]dioxinyl, 4H-benzopyranyl, 4-oxo-1,5,6,7-tetrahydro-indolyl, 4-oxo-3,4-dihydro-phthalazinyl, 4-oxo-benzopyranyl, 9,10,10-trioxo-thioxanthenyl, 9H-carbazolyl, 9H-xanthenyl, azetidinyl, benzimidazolyl, benzo[1,3]dioxolyl, benzo[2,1,3]oxadiazolyl, benzo[1,2,5]oxadiazolyl, benzo[b]thienyl, benzofuryl, benzothiazolyl, cinnolyl, furyl, imidazo[2,1-b]thiazolyl, imidazolyl, isoxazolyl, morpholino, morpholinyl, oxazolyl, oxolanyl, piperazyl, piperidyl, piridyl, pyrazolo[5,1-b]thiazolyl, pyrazolyl, pyrazinyl, pyridyl, pyrimidyl, pyrrolidyl, quinolyl, quinoxalyl, thiazolidyl, thiazolyl, thienyl, thiolanyl, 2,3-dihydro-benzofuryl, tetrahydro-thienyl, or benzofuranyl;
   halogen is fluoro, chloro, bromo, or iodo;
or a pharmaceutically acceptable salt, hydrate, or solvate thereof.

One aspect of the present invention pertains to pharmaceutical compositions comprising a therapeutically effective amount of at least one compound, as described herein, in combination with a pharmaceutically acceptable carrier.

One aspect of the present invention pertains to methods for the prophylaxis or treatment of improving memory function, sleeping and arousal, anxiety, depression, mood disorders, seizure, obesity, diabetes, appetite and eating disorders, cardiovascular disease, hypertension, dyslipidemia, myocardial infarction, binge eating disorders including bulimia, anorexia, mental disorders including manic depression, schizophrenia, delirium, dementia, stress, cognitive disorders, attention deficit disorder, substance abuse disorders and dyskinesias including Parkinson's disease, epilepsy, and addiction comprising administering to an individual suffering from said condition a therapeutically effective amount of a compound, as described herein, or a pharmaceutical composition thereof.

One aspect of the present invention pertains to methods for the prophylaxis or treatment of an eating disorder, obesity or an obesity related disorder comprising administering to an individual suffering from the condition a therapeutically effective amount of a compound, as described herein, or a pharmaceutical composition thereof.

One aspect of the present invention pertains to methods for the prophylaxis or treatment of anxiety, depression, schizophrenia, addiction, or epilepsy comprising administering to an individual suffering from the condition a therapeutically effective amount of a compound, as described herein, or a pharmaceutical composition.

One aspect of the present invention pertains to compounds of the present invention, as described herein, or a pharmaceutical composition thereof, for use in a method of treatment of the human or animal body by therapy.

One aspect of the present invention pertains to compounds of the present invention, as described herein, or a pharmaceutical composition thereof, for use in a method of prophylaxis or treatment of an eating disorder, obesity or an obesity related disorder of the human or animal body by therapy.

One aspect of the present invention pertains to compounds of the present invention, as described herein, or a pharmaceutical composition thereof, for use in a method of prophylaxis or treatment of anxiety, depression, schizophrenia, addiction, or epilepsy of the human or animal body by therapy.

One aspect of the present invention pertains to compounds of the present invention, as described herein, for the manufacture of a medicament for use in the prophylaxis or treatment of an eating disorder, obesity or obesity related disorders.

One aspect of the present invention pertains to compounds of the present invention, as described herein, for the manufacture of a medicament for use in the prophylaxis or treatment of anxiety, depression, schizophrenia, addiction, or epilepsy.

One aspect of the present invention pertains to methods of decreasing food intake of an individual comprising administering to the individual a therapeutically effective amount of a compound, as described herein, or a pharmaceutical composition thereof.

One aspect of the present invention pertains to methods of inducing satiety in an individual comprising administering to said individual a therapeutically effective amount of a compound, as described herein, or a pharmaceutical composition thereof.

One aspect of the present invention pertains to methods of controlling or reducing weight gain in an individual comprising administering to said individual a therapeutically effective amount of a compound, as described herein, or a pharmaceutical composition thereof.

One aspect of the present invention pertains to methods of modulating a MCH receptor in an individual comprising contacting the receptor with a compound, as described herein. In some embodiments, the compound is an antagonist. In some embodiments, the modulation of the MCH receptor is for the prophylaxis or treatment of an eating disorder, obesity or obesity related disorder. In some embodiments, the modulation of the MCH receptor reduces food intake of the individual. In some embodiments, the modulation of the MCH receptor induces satiety in the individual. In some embodiments, the modulation of the MCH receptor controls or reduces weight gain of the individual. In some embodiments, the modulation of the MCH receptor is for prophylaxis or treatment of anxiety, depression, schizophrenia, addiction, or epilepsy.

In some embodiments, the individual is a mammal.

In some embodiments, the mammal is a human.

In some embodiments, the human has a body mass index of about 18.5 to about 45. In some embodiments, the human has a body mass index of about 25 to about 45. In some embodiments, the human has a body mass index of about 30 to about 45. In some embodiments, the human has a body mass index of about 35 to about 45.

One aspect of the present invention pertains to methods of producing a pharmaceutical composition comprising admixing a compound, as described herein, and a pharmaceutically acceptable carrier.

### DETAILED DESCRIPTION OF THE INVENTION

One aspect of the present invention relates to certain substituted pyrimidine compounds represented by Formula (I):

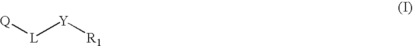

or a pharmaceutically acceptable salt, hydrate or solvate thereof, wherein Q, L, Y, and R₁ are as described herein, supra and infra.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

In some embodiments, compounds of the present invention are of Formula (I) wherein Q is Formula (IIa); Z₁ is hydrogen, halogen, C₁₋₅ alkyl, C₁₋₅ alkyl substituted by halogen, C₃₋₆ cycloalkyl, C₁₋₅ alkoxy, C₁₋₅ alkoxy substituted by halogen, or C₁₋₅ alkylthio or a pharmaceutically acceptable salt, hydrate, or solvate thereof.

In some embodiments, compounds of the present invention are of Formula (I) wherein R₁ is selected from the group consisting of:
(i) C₁₋₁₀ alkyl, and
   C₁₋₁₀ alkyl substituted by substituent(s) independently selected from the group consisting of:
      halogen,
      oxo,
      C₁₋₅ alkoxy,
      C₁₋₅ alkoxy substituted by carbocyclic aryl,
      C₁₋₅ alkylcarbonyloxy,
      C₁₋₅ alkoxycarbonyl,
      C₁₋₅ alkoxycarbonyl substituted by carbocyclic aryl,
      carbocyclic aryloxy, and
      carbocyclic aryloxy substituted by substituent(s) independently selected from the group consisting of:
         halogen,
         nitro,
         C₁₋₅ alkyl, and
         C₁₋₅ alkyl substituted by oxo,
      heterocyclyloxy,
      heterocyclyloxy substituted by C₁₋₅ alkyl,
      mono-carbocyclic arylamino,
      di-carbocyclic arylamino,
      carbocyclic arylsulfonylamino,
      carbocyclic arylsulfonylamino substituted by C₁₋₅ alkyl,
      C₁₋₅ alkylthio,
      C₁₋₅ alkylthio substituted by carbocyclic aryl,
      carbocyclic arylthio,
      carbocyclic arylthio substituted by halogen,
      carbocyclic arylthio substituted by C₁₋₅ alkyl,
      carbocyclic arylsulfonyl,
      carbocyclic arylsulfonyl substituted by halogen,
      heterocyclylthio,
      heterocyclylthio substituted by C₁₋₅ alkyl,
      C₃₋₆ cycloalkyl,
      C₃₋₆ cycloalkenyl,
      carbocyclyl,
      carbocyclyl substituted by C₁₋₅ alkoxy,
      carbocyclic aryl, and
      carbocyclic aryl substituted by substituent(s) independently selected from the group consisting of:
         halogen,
         nitro,
         C₁₋₅ alkyl, and
         C₁₋₅ alkyl substituted by substituent(s) independently selected from the group consisting of:
            halogen,
            carbocyclic aryl, and
            heterocyclyl,
         C₁₋₅ alkoxy,
         C₁₋₅ alkoxy substituted by halogen,
         C₁₋₅ alkoxy substituted by carbocyclic aryl,
         carbocyclic aryloxy,
         mono-carbocyclic arylaminocarbonyl, and
         mono-carbocyclic arylaminocarbonyl substituted by substituent(s) selected from the group consisting of:
            halogen,
            C₁₋₅ alkyl,
            C₁₋₅ alkoxy, and
            C₁₋₅ alkoxy substituted by halogen,
         di-carbocyclic arylaminocarbonyl, and
         di-carbocyclic arylaminocarbonyl substituted by substituent(s) selected from the group consisting of:
            halogen,
            C₁₋₅ alkyl,
            C₁₋₅ alkoxy, and
            C₁₋₅ alkoxy substituted by halogen,
         C₁₋₅ alkylthio,
         C₁₋₅ alkylthio substituted by halogen,
         C₁₋₅ alkylsulfonyl,
         carbocyclic aryl, and
         heterocyclyl,
      heterocyclyl, and
      heterocyclyl substituted by substituent(s) independently selected from the group consisting of:
         C₁₋₅ alkyl,
         C₁₋₅ alkoxy,
         C₁₋₅ alkoxy substituted by carbocyclic aryl,
         carbocyclic aryl, and
         carbocyclic aryl substituted by halogen,
(ii) C₂₋₅ alkenyl, and
   C₂₋₅ alkenyl substituted by substituent(s) independently selected from the group consisting of:
      carbocyclic aryl, and
      carbocyclic aryl substituted by substituent(s) independently selected from the group consisting of:
         nitro,
         halogen,
         C₁₋₅ alkyl,
         C₁₋₅ alkyl substituted by halogen,
         C₁₋₅ alkoxy, and
         C₁₋₅ alkoxy substituted by halogen,
(iii) C₃₋₆ cycloalkyl, and
   C₃₋₆ cycloalkyl substituted by substituent(s) independently selected from the group consisting of:
      C₁₋₅ alkyl,
      C₁₋₅ alkyl substituted by carbocyclic aryl,
      carbocyclic arylcarbonylamino, and
      carbocyclic aryl,
(iv) carbocyclyl, and
   carbocyclyl substituted by nitro,
(v) carbocyclic aryl, and
   carbocyclic aryl substituted by substituent(s) independently selected from the group consisting of:
      halogen,
      cyano,
      nitro,
      C₁₋₉ alkyl, and
      C₁₋₉ alkyl substituted by substituent(s) independently selected from the group consisting of:
         halogen,
         oxo,
         mono-carbocyclic arylaminocarbonyl,
         di-carbocyclic arylaminocarbonyl,
         mono-carbocyclic arylaminocarbonyl substituted by C₁₋₅ alkoxy,
         di-carbocyclic arylaminocarbonyl substituted by C₁₋₅ alkoxy,
         carbocyclic aryloxy,
         carbocyclic aryl, and
         carbocyclic aryl substituted by substituent(s) independently selected from the group consisting of:
            halogen,
            C₁₋₅ alkyl, and
            C₁₋₅ alkyl substituted by halogen,
         heterocyclyl, and
         heterocyclyl substituted by C₁₋₅ alkyl,
      C₂₋₅ alkenyl,
      C₁₋₇ alkoxy,
      C₁₋₇ alkoxy substituted by halogen,
      C₁₋₇ alkoxy substituted by carbocyclic aryl,
      C₃₋₆ cycloalkoxy,
      carbocyclic aryloxy, and
      carbocyclic aryloxy substituted by substituent(s) independently selected from the group consisting of:
         halogen,
         nitro, and
         C₁₋₅ alkoxy
      heterocyclyloxy, and
      heterocyclyloxy substituted by substituent(s) independently selected from the group consisting of:
         halogen,
         C₁₋₅ alkyl, and
         C₁₋₅ alkyl substituted by halogen,
      C₁₋₅ alkoxycarbonyl,
      mono-C₁₋₅ alkylaminocarbonyl,
      di-C₁₋₅ alkylaminocarbonyl,
      mono-C₁₋₅ alkylaminocarbonyl substituted by carbocyclic aryl,
      di-C₁₋₅ alkylaminocarbonyl substituted by carbocyclic aryl,
      mono-carbocyclic arylaminocarbonyl,
      di-carbocyclic arylaminocarbonyl,
      mono-carbocyclic arylaminocarbonyl substituted by C₁₋₅ alkyl,
      di-carbocyclic arylaminocarbonyl substituted by C₁₋₅ alkyl,
      mono-C₁₋₅ alkylamino,
      di-C₁₋₅ alkylamino,
      C₁₋₅ alkylthio,
      C₁₋₅ alkylthio substituted by halogen,
      C₁₋₅ alkylsulfonyl,
      carbocyclic aryl, and
      carbocyclic aryl substituted by substituent(s) independently selected from the group consisting of:
         C₁₋₇ alkyl, and
         C₁₋₇ alkyl substituted by halogen,
(vi) heterocyclyl, and
   heterocyclyl substituted by substituent(s) independently selected from the group consisting of:
      halogen,
      C₁₋₅ alkyl, and
      C₁₋₅ alkyl substituted by substituent(s) independently selected from the group consisting of:
         halogen,
         oxo,
         carbocyclic aryl,
         carbocyclic aryl substituted by halogen,
         heterocyclyl, and
         heterocyclyl substituted by substituent(s) independently selected from the group consisting of:
            halogen,
            C₁₋₅ alkyl, and
            C₁₋₅ alkyl substituted by halogen,
      C₁₋₅ alkoxy,
      C₁₋₅ alkylthio,
      carbocyclic arylthio,
      C₁₋₅ alkylsulfonyl,
      carbocyclic arylsulfonyl,
      carbocyclic arylsulfonyl substituted by halogen,
      carbocyclic arylsulfonyl substituted by C₁₋₅ alkyl,
      C₁₋₅ alkoxycarbonyl,
      carbocyclic aryl, and
      carbocyclic aryl substituted by substituent(s) independently selected from the group consisting of:
         halogen,
         nitro, and
         C₁₋₅ alkyl,
      heterocyclyl, and
      heterocyclyl substituted by substituent(s) independently selected from the group consisting of:
         halogen,
         C₁₋₅ alkyl, and
         C₁₋₅ alkyl substituted by halogen;
   wherein carbocyclic aryl is phenyl, naphthyl, or anthranyl;
   carbocyclyl is 1-oxo-indanyl, 9H-fluorenyl, 9-oxo-fluorenyl, anthraquinonyl, C-fluoren-9-ylidene, indanyl, or menthyl;
   heterocyclyl is 1,2,3,4-tetrahydro-isoquinolyl, 1,2,3-thiadiazolyl, 1,2,3-triazolyl, 1,3-dioxo-isoindolyl, 1H-indolyl, 1H-pyrrolyl, 2,3-dihydro-1-oxo-isoindolyl, 2,3-dihydro-benzo[1,4]dioxinyl, 2H-benzopyranyl, 2-oxo-benzopyranyl, 2-oxo-pyrrolidinyl, 4-oxo-benzopyranyl, 9H-xanthenyl, benzo[1,3]dioxolyl, benzo[2,1,3]oxadiazolyl, benzo[1,2,5]oxadiazolyl, benzo[b]thienyl, furyl, isoxazolyl, morpholinyl, oxazolyl, pyrazolyl, pyridyl, pyrimidyl, pyrrolidyl, quinolyl, quinoxalyl, thiazolyl, thienyl, imidazolyl, or piperazyl;
   halogen is fluoro, chloro, bromo, or iodo;

or a pharmaceutically acceptable salt, hydrate, or solvate thereof.

In some embodiments, compounds of the present invention are of Formula (I) wherein R₂ is halogen, C₁₋₅ alkyl, C₁₋₅ alkoxy, —N(R₂ₐ)(R_{2b}), or heterocyclyl; wherein R₂ₐ and R_{2b} are each independently hydrogen, C₁₋₅ alkyl, C₁₋₅ alkyl substituted by hydroxy, C₁₋₅ alkyl substituted by carbocyclic aryl, C₁₋₅ alkyl substituted by heterocyclyl, C₃₋₆ cycloalkyl, or carbocyclic aryl; L is selected from the group consisting of Formulae (IIIa) and (IVa); wherein R₃ and R₄ are each independently hydrogen or C₁₋₅ alkyl; and A and B are each independently a single bond, —CH₂—, or —(CH₂)₂—; Z₁ is hydrogen, halogen, C₁₋₅ alkyl, C₁₋₅ alkyl substituted by halogen, C₁₋₅ alkoxy, or C₁₋₅ alkylthio; Z₂ is hydrogen, halogen, or C₁₋₅ alkyl; or R₂ and Z₂ are bonded to each other to form a ring and —R₂-Z₂- is —NR₆—CH═CH—; wherein R₆ is hydrogen or C₁₋₅ alkyl; and Y represents:
(i) —C(O)NR₅—, —C(S)NR₅—, —C(O)O—, —S(O)₂—, —C(O)—, or —(CH₂)ₘ— when L is selected from the group consisting of Formula (IIIa); or
(ii) —C(O)NR₅— or —C(O)O— when L is selected from the group consisting of Formula (IVa);
   wherein R₅ is hydrogen or C₁₋₅ alkyl; and m is 0, 1, or 2;

or a pharmaceutically acceptable salt, hydrate, or solvate thereof.

In some embodiments, compounds of the present invention are of Formula (I) wherein R₁ is selected from the group consisting of:
(i) C₁₋₅ alkyl substituted by substituent(s) independently selected from the group consisting of:
   hydroxy,
   carbocyclic aryl,
   carbocyclic aryl substituted by halogen, and
   C₁₋₅ alkylthio,
(ii) C₃₋₆ cycloalkyl, and
(iii) carbocyclic aryl, and
   carbocyclic aryl substituted by substituent(s) independently selected from the group consisting of:
      halogen,
      nitro,
      cyano,
      C₁₋₅ alkyl,
      C₁₋₅ alkyl substituted by halogen,
      C₁₋₅ alkoxy,
      C₁₋₅ alkoxy substituted by halogen,
      C₁₋₅ alkoxy substituted by carbocyclic aryl,
      carbocyclic aryloxy, and
      carbocyclic aryloxy substituted by C₁₋₅ alkoxy,
(iv) heterocyclyl, and
   heterocyclyl substituted by substituent(s) independently selected from the group consisting of:
      halogen,
      C₁₋₅ alkyl,
      carbocyclic aryl, and
      carbocyclic aryl substituted by halogen;
R₂ is —N(R₂ₐ)(R_{2b}) or heterocyclyl; wherein R₂ₐ and R_{2b} are each independently hydrogen or C₁₋₅ alkyl;
Z₁ is hydrogen, C₁₋₅ alkyl, or C₁₋₅ alkylthio; Z₂ is hydrogen or C₁₋₅ alkyl; or R₂ and Z₂ are bonded to each other to form a ring and —R₂-Z₂- is —NR₆—CH═CH—; wherein R₆ is hydrogen or C₁₋₅ alkyl;
L is Formula (IIIa) or (IVa), wherein R₃ and R₄ are hydrogen, A is a single bond and B is a single bond or —CH₂—;
   and
Y represents:
(i) —C(O)NH—, —C(S)NH, —C(O)—, or —CH₂— when L is selected from the group consisting of Formula (IIIa); or
(ii) —C(O)NH— when L is selected from the group consisting of Formula (IVa);
wherein carbocyclic aryl is phenyl or naphthyl;
heterocyclyl is furyl, 1H-indolyl, morpholinyl, oxazolyl, piperidyl, pyridyl, pyrrolidyl, or 9H-xanthenyl;
halogen is fluoro, chloro, or bromo;
or a pharmaceutically acceptable salt, hydrate, or solvate thereof.

In some embodiments, compounds of the present invention are of Formula (I) wherein R₁ is selected from the group consisting of:
(i) carbocyclic aryl, and
   carbocyclic aryl substituted by substituent(s) independently selected from the group consisting of:
      halogen,
      C₁₋₅ alkyl,
      C₁₋₅ alkyl substituted by halogen,
      C₁₋₅ alkoxy, and
      C₁₋₅ alkoxy substituted by halogen,
(ii) heterocyclyl, and
   heterocyclyl substituted by halogen;
      and
Z₁ is hydrogen, C₁₋₅ alkyl, or C₁₋₅ alkylthio; Z₂ is hydrogen or C₁₋₅ alkyl;
wherein carbocyclic aryl is phenyl;
heterocyclyl is furyl, pyridyl, or pyrrolidyl;
halogen is fluoro, chloro, or bromo;
or a pharmaceutically acceptable salt, hydrate, or solvate thereof.

In some embodiments, compounds of the present invention are of Formula (I) wherein the compound is selected from the group consisting of:
N-(cis-4-{[6-(dimethylamino)pyrimidin-4-yl]amino}cyclohexyl)-3,4-difluorobenzamide;
N-(cis-4-{[6-(dimethylamino)-2-methylpyrimidin-4-yl]amino}cyclohexyl)-4-fluorobenzamide;
4-chloro-N-(cis-4-{[6-(dimethylamino)-2-methylpyrimidin-4-yl]amino}cyclohexyl)-3-fluorobenzamide;
N-(cis-4-{[6-(dimethylamino)-2-methylpyrimidin-4-yl]amino}cyclohexyl)-3,5-difluorobenzamide;
3-chloro-N-(cis-4-{[6-(dimethylamino)-2-methylpyrimidin-4-yl]amino}cyclohexyl)-4-(trifluoromethoxy)benzamide;
3-chloro-4-fluoro-N-(cis-4-{[2-methyl-6-(methylamino)pyrimidin-4-yl]amino}cyclohexyl)benzamide;
N-(cis-4-{[6-(dimethylamino)-2-methylpyrimidin-4-yl]amino}cyclohexyl)-3-fluorobenzamide;
4-chloro-N-(cis-4-{[6-(dimethylamino)-2-methylpyrimidin-4-yl]amino}cyclohexyl)benzamide;
N-(cis-4-{[6-(dimethylamino)-2-methylpyrimidin-4-yl]amino}cyclohexyl)-3-fluoro-5-(trifluoromethyl)benzamide;
N-(cis-4-{[6-(dimethylamino)-2-methylpyrimidin-4-yl]amino}cyclohexyl)-3,5-bis(trifluoromethyl)benzamide;
3-chloro-4-fluoro-N-{cis-4-[(2-methyl-6-piperidin-1-ylpyrimidin-4-yl)amino]cyclohexyl}benzamide;
3-chloro-4-fluoro-N-{cis-4-[(2-methyl-6-morpholin-4-ylpyrimidin-4-yl)amino]cyclohexyl}benzamide;
3-chloro-4-fluoro-N-{cis-4-[(7-methyl-7H-pyrrolo[2,3-d]pyrimidin-4-yl)amino]cyclohexyl}benzamide;
3,4,5-trifluoro-N-{cis-4-[(7-methyl-7H-pyrrolo[2,3-d]pyrimidin-4-yl)amino]cyclohexyl}benzamide;
3,4,5-trifluoro-N-(cis-4-{[2-methyl-6-(methylamino)pyrimidin-4-yl]amino}cyclohexyl)benzamide;
cis-N-(3,4-difluorophenyl)-4-{[6-(dimethylamino)-2-methylpyrimidin-4-yl]amino}cyclohexanecarboxamide;
1-(4-chlorophenyl)-N-(cis-4-{[6-(dimethylamino)-2-methylpyrimidin-4-yl]amino}cyclohexyl)cyclopentanecarboxamide;
3-(2-chloro-6-fluorophenyl)-N-(cis-4-{[6-(dimethylamino)-2-methylpyrimidin-4-yl]amino}cyclohexyl)-5-methylisoxazole-4-carboxamide;
N-(cis-4-{[6-(dimethylamino)-2-methylpyrimidin-4-yl]amino}cyclohexyl)-2-(4-methoxyphenoxy)-5-nitrobenzamide;
N-(cis-4-{[6-(dimethylamino)-2-methylpyrimidin-4-yl]amino}cyclohexyl)-5-iodo-2-furamide;
N-(cis-4-{[6-(dimethylamino)-2-methylpyrimidin-4-yl]amino}cyclohexyl)-2-(ethylthio)-2,2-diphenylacetamide;
N-(cis-4-{[6-(dimethylamino)-2-methylpyrimidin-4-yl]amino}cyclohexyl)-9H-xanthene-9-carboxamide;
N-(cis-4-{[6-(dimethylamino)-2-methylpyrimidin-4-yl]amino}cyclohexyl)-N′-[1-(1-naphthyl)ethyl]urea;
N-(cis-4-{[6-(dimethylamino)-2-methylpyrimidin-4-yl]amino}cyclohexyl)-N′-(3,4,5-trimethoxyphenyl)urea;
N-(5-chloro-2,4-dimethoxyphenyl)-M-(cis-4-{[6-(dimethylamino)-2-methylpyrimidin-4-yl]amino}cyclohexyl)urea;
N-(cis-4-{[6-(dimethylamino)-2-methylpyrimidin-4-yl]amino}cyclohexyl)-N′-(2,4,6-tribromophenyl)urea;
N-(cis-4-{[6-(dimethylamino)-2-methylpyrimidin-4-yl]amino}cyclohexyl)-N′-mesitylthiourea;
N-(2,6-diethylphenyl)-N′-(cis-4-{[6-(dimethylamino)-2-methylpyrimidin-4-yl]amino}cyclohexyl)thiourea;
N-(2,4-dichloro-6-methylphenyl)-N′-(cis-4-{[6-(dimethylamino)-2-methylpyrimidin-4-yl]amino}cyclohexyl)thiourea;
N-(5-chloro-2,4-dimethoxyphenyl)-N′-(cis-4-{[6-(dimethylamino)-2-methylpyrimidin-4-yl]amino}cyclohexyl)thiourea;
N-[4-bromo-2-(trifluoromethyl)phenyl]-N′-(cis-4-{[6-dimethylamino)-2-methylpyrimidin-4-yl]amino}cyclohexyl)thiourea;
N-(cis-4-{[6-(dimethylamino)-2-methylpyrimidin-4-yl]amino}cyclohexyl)-3-nitrobenzamide;
N-[cis-4-(6-dimethylamino-2-methyl-pyrimidin-4-ylamino)-cyclohexyl]-3,4-diethoxy-benzamide;
N-[cis-4-(6-dimethylamino-2-methyl-pyrimidin-4-ylamino)-cyclohexyl]-3-ethoxy-benzamide;
N-[cis-4-(6-dimethylamino-2-methyl-pyrimidin-4-ylamino)-cyclohexyl]-3,5-diethoxy-benzamide;
N-[cis-4-(6-dimethylamino-2-methyl-pyrimidin-4-ylamino)-cyclohexyl]-3-isopropoxy-benzamide;
3-bromo-N-[cis-4-(6-dimethylamino-2-methyl-pyrimidin-4-ylamino)-cyclohexyl]-4-fluoro-benzamide;
4-difluoromethoxy-N-[cis-4-(6-dimethylamino-2-methyl-pyrimidin-4-ylamino)-cyclohexyl]-benzamide;
4-chloro-N-[cis-4-(6-dimethylamino-2-methyl-pyrimidin-4-ylamino)-cyclohexyl]-3-methyl-benzamide;
3-difluoromethoxy-N-[cis-4-(6-dimethylamino-2-methyl-pyrimidin-4-ylamino)-cyclohexyl]-benzamide;
3-chloro-N-[cis-4-(6-dimethylamino-2-methyl-pyrimidin-4-ylamino)-cyclohexyl]-4-methyl-benzamide;
4-bromo-N-[cis-4-(6-dimethylamino-2-methyl-pyrimidin-4-ylamino)-cyclohexyl]-benzamide;
N-[cis-4-(6-dimethylamino-2-methyl-pyrimidin-4-ylamino)-cyclohexyl]-3,5-dimethoxy-benzamide;
4-cyano-N-[cis-4-(6-dimethylamino-2-methyl-pyrimidin-4-ylamino)-cyclohexyl]-benzamide;
N-[cis-4-(6-dimethylamino-2-methyl-pyrimidin-4-ylamino)-cyclohexyl]-4-methoxy-benzamide;
3-cyano-N-[cis-4-(6-dimethylamino-2-methyl-pyrimidin-4-ylamino)-cyclohexyl]-benzamide;
N-[cis-4-(6-dimethylamino-2-methyl-pyrimidin-4-ylamino)-cyclohexyl]-3-methoxy-benzamide;
N-[cis-4-(6-dimethylamino-2-methyl-pyrimidin-4-ylamino)-cyclohexyl]-4-fluoro-3-methyl-benzamide;
4-bromo-N-[cis-4-(6-dimethylamino-2-methyl-pyrimidin-4-ylamino)-cyclohexyl]-3-methyl-benzamide;
N-[cis-4-(6-dimethylamino-2-methyl-pyrimidin-4-ylamino)-cyclohexyl]-3-fluoro-4-methyl-benzamide;
N-[cis-4-(6-dimethylamino-2-methyl-pyrimidin-4-ylamino)-cyclohexyl]-3-ethyl-benzamide;
3-bromo-N-[cis-4-(6-dimethylamino-2-methyl-pyrimidin-4-ylamino)-cyclohexyl]-benzamide;
N-[cis-4-(6-dimethylamino-2-methyl-pyrimidin-4-ylamino)-cyclohexyl]-3-fluoro-4-trifluoromethyl-benzamide;
N-[cis-4-(6-dimethylamino-2-methyl-pyrimidin-4-ylamino)-cyclohexyl]-4-trifluoromethoxy-benzamide;
N-[cis-4-(6-dimethylamino-2-methyl-pyrimidin-4-ylamino)-cyclohexyl]-4-methyl-benzamide;
N-[cis-4-(6-dimethylamino-2-methyl-pyrimidin-4-ylamino)-cyclohexyl]-3-methyl-benzamide;
N-[cis-4-(6-dimethylamino-2-methyl-pyrimidin-4-ylamino)-cyclohexyl]-4-trifluoromethyl-benzamide;
2,2-difluoro-benzo[1,3]dioxole-5-carboxylic acid[cis-4-(6-dimethylamino-2-methyl-pyrimidin-4-ylamino)-cyclohexyl]-amide;
N-{cis-4-[(1H-indol-2-ylmethyl)-amino]-cyclohexyl}-2,N′,N′-trimethyl-pyrimidine-4,6-diamine;
2,N,N-trimethyl-N-[cis-4-(3-trifluoromethoxy-benzylamino)-cyclohexyl]-pyrimidine-4,6-diamine;
N-[cis-4-(3,4-difluoro-benzylamino)-cyclohexyl]-2,N′,N′-trimethyl-pyrimidine-4,6-diamine;
1-(3,4-dimethoxy-phenyl-3-[cis-4-(6-dimethylamino-2-methyl-pyrimidin-4-ylamino)-cyclohexyl]-urea;
1-[cis-4-(6-dimethylamino-2-methyl-pyrimidin-4-ylamino)-cyclohexyl]-3-(2-ethoxy-phenyl)-urea;
1-(4-benzyloxy-phenyl)-3-[cis-4-(6-dimethylamino-2-methyl-pyrimidin-4-ylamino)-cyclohexyl]-urea;
3,5-dibromo-N-[cis-4-(6-dimethylamino-2-methyl-pyrimidin-4-ylamino)-cyclohexyl]-benzamide;
3-bromo-4-chloro-N-[cis-4-(6-dimethylamino-2-methyl-pyrimidin-4-ylamino)-cyclohexyl]-benzamide;
4-chloro-N-[cis-4-(6-dimethylamino-2-methyl-pyrimidin-4-ylamino)-cyclohexyl]-3-trifluoromethyl-benzamide;
2-(3,5-bis-trifluoromethyl-phenyl)-N-[cis-4-(6-dimethylamino-2-methyl-pyrimidin-4-ylamino)-cyclohexyl]-2-hydroxy-acetamide;
N-[cis-4-(6-dimethylamino-2-methyl-pyrimidin-4-ylamino)-cyclohexyl methyl]-3-fluoro-4-trifluoromethyl-benzamide;
N-[cis-4-(6-dimethylamino-2-methyl-pyrimidin-4-ylamino)-cyclohexylmethyl]-3-trifluoromethoxy-benzamide;
N-[cis-4-(6-dimethylamino-2-methyl-pyrimidin-4-ylamino)-cyclohexylmethyl]-3-methoxy-benzamide;
4-chloro-N-[cis-4-(6-dimethylamino-2-methyl-pyrimidin-4-ylamino)-cyclohexylmethyl]-benzamide;
N-[cis-4-(6-dimethylamino-2-methyl-pyrimidin-4-ylamino)-cyclohexylmethyl]-3-trifluoromethyl-benzamide;
N-[cis-4-(6-dimethylamino-2-methyl-pyrimidin-4-ylamino)-cyclohexylmethyl]-4-trifluoromethyl-benzamide;
N-[cis-4-(6-dimethylamino-2-methyl-pyrimidin-4-ylamino)-cyclohexylmethyl]-3-methyl-benzamide;
N-[cis-4-(6-dimethylamino-2-methyl-pyrimidin-4-ylamino)-cyclohexylmethyl]-3,5-difluoro-benzamide;
N-[cis-4-(6-dimethylamino-2-methyl-pyrimidin-4-ylamino)-cyclohexylmethyl]-3-ethyl-benzamide;
2,2-difluoro-benzo[1,3]dioxole-5-carboxylic acid [cis-4-(6-dimethylamino-2-methyl-pyrimidin-4-ylamino)-cyclohexylmethyl]-amide;
N-[cis-4-(6-dimethylamino-2-methyl-pyrimidin-4-ylamino)-cyclohexylmethyl]-3-fluoro-4-methyl-benzamide;
N-[cis-4-(6-dimethylamino-2-methyl-pyrimidin-4-ylamino)-cyclohexylmethyl]-4-fluoro-benzamide;
3,4-dichloro-N-[cis-4-(6-dimethylamino-2-methyl-pyrimidin-4-ylamino)-cyclohexylmethyl]-benzamide;
4-bromo-N-[cis-4-(6-dimethylamino-2-methyl-pyrimidin-4-ylamino)-cyclohexylmethyl]-benzamide;
N-[cis-4-(6-dimethylamino-2-methyl-pyrimidin-4-ylamino)-cyclohexylmethyl]-3,4-difluoro-benzamide;
3,5-dichloro-N-[cis-4-(6-dimethylamino-2-methyl-pyrimidin-4-ylamino)-cyclohexylmethyl]-benzamide;
3-chloro-N-[cis-4-(6-dimethylamino-2-methyl-pyrimidin-4-ylamino)-cyclohexylmethyl]-4-fluoro-benzamide;
N-[cis-4-(6-dimethylamino-2-methyl-pyrimidin-4-ylamino)-cyclohexylmethyl]-4-fluoro-3-methyl-benzamide; and
3-chloro-N-[cis-4-(6-dimethylamino-2-methyl-pyrimidin-4-ylamino)-cyclohexylmethyl]-benzamide;

or a pharmaceutically acceptable salt, hydrate, or solvate thereof.

In some embodiments, compounds of the present invention are of Formula (I) wherein the compound is selected from the group consisting of:
N-(cis-4-{[6-(dimethylamino)-2-methylpyrimidin-4-yl]amino}cyclohexyl)-3,4-difluorobenzamide;
N-(cis-4-{[6-(dimethylamino)-2-ethylpyrimidin-4-yl]amino}cyclohexyl)-3,4-difluorobenzamide;
3-chloro-N-(cis-4-{[6-(dimethylamino)-2-methylpyrimidin-4-yl]amino}cyclohexyl)-4-fluorobenzamide;
3,4-dichloro-N-(cis-4-{[6-(dimethylamino)-2-methylpyrimidin-4-yl]amino}cyclohexyl)benzamide;
3-chloro-N-(cis-4-{[6-(dimethylamino)-2-methylpyrimidin-4-yl]amino}cyclohexyl)-5-fluorobenzamide;
N-(cis-4-{[6-(dimethylamino)-2-methylpyrimidin-4-yl]amino}cyclohexyl)-3,4,5-trifluorobenzamide;
5-bromo-N-(cis-{[6-(dimethylamino)-2-methylpyrimidin-4-yl]amino}cyclohexyl)nicotinamide;
N-(cis-4-{[6-(dimethylamino)-2-methylpyrimidin-4-yl]amino}cyclohexyl)-4-fluoro-3-(trifluoromethyl)benzamide;
N-(cis-4-{[6-(dimethylamino)-2-methylpyrimidin-4-yl]amino}cyclohexyl)-3-(trifluoromethyl)benzamide;
N-(cis-4-{[6-(dimethylamino)-2-methylpyrimidin-4-yl]amino}cyclohexyl)-3-(trifluoromethoxy)benzamide;
3,5-dichloro-N-(cis-4-{[6-(dimethylamino)-2-methylpyrimidin-4-yl]amino}cyclohexyl)benzamide;
3-chloro-N-(cis-4-{[6-(dimethylamino)-2-methylpyrimidin-4-yl]amino}cyclohexyl)benzamide;
3-chloro-4-fluoro-N-{cis-4-[(2-methyl-6-pyrrolidin-1-ylpyrimidin-4-yl)amino]cyclohexyl}benzamide;
N-(cis-4-{[6-(diethylamino)-2-ethylpyrimidin-4-yl]amino}cyclohexyl)-3,4,5-trifluorobenzamide;
cis-N-(3-chloro-4-fluorophenyl)-4-{[6-(dimethylamino)-2-methylpyrimidin-4-yl]amino}cyclohexanecarboxamide;
N-(cis-4-{[2-benzyl-6-(dimethylamino)pyrimidin-4-yl]amino}cyclohexyl)-3-chloro-4-fluorobenzamide;
cis-4-{[6-(dimethylamino)-2-methylpyrimidin-4-yl]amino}-N-(3,4,5-trifluorophenyl)cyclohexanecarboxamide;
N-(4-bromo-2,6-dimethylphenyl)-N′-(cis-4-{[6-(dimethylamino)-2-methylpyrimidin-4-yl]amino}cyclohexyl)urea;
N-(4-bromo-2,6-dimethylphenyl)-N′-(cis-4-{[6-(dimethylamino)-2-methylpyrimidin-4-yl]amino}cyclohexyl)thiourea;
N-(cis-4-{[6-(dimethylamino)-2-methylpyrimidin-4-yl]amino}cyclohexyl)-N′-(3,4,5-trimethoxyphenyl)thiourea;
N-(cis-4-{[6-(dimethylamino)-2-methylpyrimidin-4-yl]amino}cyclohexyl)-N′-(2,4,6-tribromophenyl)thiourea;
5-bromo-furan-2-carboxylic acid [cis-4-(6-dimethylamino-2-methyl-pyrimidin-4-ylamino)-cyclohexyl]-amide;
N-[cis-4-(3,5-dimethoxy-benzylamino)-cyclohexyl]-2,N′,N′-trimethyl-pyrimidine-4,6-diamine;
N-[cis-4-(3-bromo-benzylamino)-cyclohexyl]-2,N′,N′-trimethyl-pyrimidine-4,6-diamine;
1-[cis-4-(6-dimethylamino-2-methyl-pyrimidin-4-ylamino)-cyclohexyl]-3-(3-methoxy-phenyl)-urea;
1-(3,5-difluoro-phenyl)-3-[cis-4-(6-dimethylamino-2-methyl-pyrimidin-4-ylamino)-cyclohexyl]-urea;
N-[cis-4-(6-dimethylamino-2-methylsulfanyl-pyrimidin-4-ylamino)-cyclohexyl]-3,4-difluoro-benzamide;
N-[cis-4-(6-dimethylamino-5-methyl-pyrimidin-4-ylamino)-cyclohexyl]-3,4-difluoro-benzamide;
N-[cis-4-(6-dimethylamino-2-methyl-pyrimidin-4-ylamino)-cyclohexylmethyl]-3,5-bis-trifluoromethyl-benzamide; and
N-[cis-4-(6-dimethylamino-2-methyl-pyrimidin-4-ylamino)-cyclohexylmethyl]-4-trifluoromethoxy-benzamide;

or a pharmaceutically acceptable salt, hydrate, or solvate thereof.

In some embodiments, compounds of the present invention are of Formula (I) wherein R₁ represents:
(i) hydrogen, —CO₂^{t}Bu, or —CO₂Bn (Bn is a benzyl group) when L is selected from the group consisting of Formulae (III), (IIIa), and (IIIb); or
(ii) hydrogen, C₁₋₅ alkyl, substituted C₁₋₅ alkyl, Bn, or substituted Bn when L is selected from the group consisting of Formulae (IV), (IVa), and (IVb);

wherein R₃ and R₄ are each independently hydrogen or C₁₋₅ alkyl; and A and B are each independently a single bond, —CH₂—, or —(CH₂)₂—; R₂ is halogen, C₁₋₅ alkyl, C₁₋₅ alkoxy, —N(R₂ₐ)(R_{2b}), or heterocyclyl; wherein R₂ₐ and R_{2b} are each independently hydrogen, C₁₋₅ alkyl, C₁₋₅ alkyl substituted by hydroxy, C₁₋₅ alkyl substituted by carbocyclic aryl, C₁₋₅ alkyl substituted by heterocyclyl, C₃₋₆ cycloalkyl, or carbocyclic aryl; Z₁ is hydrogen, halogen, C₁₋₅ alkyl, C₁₋₅ alkyl substituted by halogen, C₁₋₅ alkoxy, or C₁₋₅ alkylthio; Z₂ is hydrogen, halogen, or C₁₋₅ alkyl; or R₂ and Z₂ are bonded to each other to form a ring and —R₂-Z₂- is —NR₆—CH═CH—; wherein R₆ is hydrogen or C₁₋₅ alkyl; and Y represents:
(i) a single bond when L is selected from the group consisting of Formulae (III), (IIIa), and (IIIb); or
(ii) —C(O)O— when L is selected from the group consisting of Formulae (IV), (IVa), and (IVb);

or a pharmaceutically acceptable salt, hydrate, or solvate thereof.

In some embodiments, compounds of the present invention are of Formula (I) wherein R₁ represents:
(i) hydrogen, —CO₂^{t}Bu, or —CO₂Bn (Bn is a benzyl group) when L is selected from the group consisting of Formula (IIIa); or
(ii) hydrogen, C₁₋₅ alkyl, substituted C₁₋₅ alkyl, Bn, or substituted Bn when L is selected from the group consisting of Formula (IVa);

wherein R₃ and R₄ are each hydrogen; and A and B are each independently a single bond or —CH₂—; R₂ is —N(R₂ₐ)(R_{2b}) or heterocyclyl; wherein R₂ₐ and R_{2b} are each independently hydrogen or C₁₋₅ alkyl; Z₁ is hydrogen, C₁₋₅ alkyl, or C₁₋₅ alkylthio; Z₂ is hydrogen or C₁₋₅ alkyl; or R₂ and Z₂ are bonded to each other to form a ring and —R₂-Z₂- is —NR₆—CH═CH—; wherein R₆ is hydrogen or C₁₋₅ alkyl; and Y represents:
(i) a single bond when L is selected from the group consisting of Formula (IIIa); or
(ii) —C(O)O— when L is selected from the group consisting of Formula (IVa);

heterocyclyl is furyl, 1H-indolyl, morpholinyl, oxazolyl, piperidyl, pyridyl, pyrrolidyl, or 9H-xanthenyl;

or a pharmaceutically acceptable salt, hydrate, or solvate thereof.

In some embodiments, compounds of the present invention are of Formula (I) wherein Q is Formula (IIb); R₂ is C₁₋₅ alkyl substituted by hydroxy, C₁₋₅ alkyl substituted by carbocyclic aryl, C₁₋₅ alkyl substituted by halogenated carbocyclic aryl, C₁₋₅ alkyl substituted by heterocyclyl, C₁₋₅ alkyl substituted by halogenated heterocyclyl, C₂₋₅ alkenyl, C₂₋₅ alkynyl, or —N(R₂ₐ)(R_{2b}); wherein R₂ₐ and R_{2b} are each independently hydrogen, C₁₋₅ alkyl, or C₁₋₅ alkyl substituted by substituent(s) independently selected from the group consisting of:
halogen,
hydroxy,
carboxy,
carbamoyl,
C₁₋₅ alkoxy,
amino,
C₃₋₆ cycloalkyl,
carbocyclic aryl,
carbocyclic aryl substituted by substituent(s) independently selected from the group consisting of:
   halogen,
   C₁₋₅ alkyl,
   C₁₋₅ alkoxy,
   C₁₋₅ alkyl substituted by halogen,
   C₁₋₅ alkoxy substituted by halogen, and
   SO₂NH₂,
heterocyclyl, and
heterocyclyl substituted by substituent(s) independently selected from the group consisting of:
   halogen,
   C₁₋₅ alkyl,
   C₁₋₅ alkoxy,
   C₁₋₅ alkyl substituted by halogen, and
   C₁₋₅ alkoxy substituted by halogen,
carbocyclic aryl, carbocyclic aryl substituted by substituent(s) independently selected from the group consisting of:
   halogen,
   C₁₋₅ alkyl,
   C₁₋₅ alkoxy,
   C₁₋₅ alkyl substituted by halogen, and
   C₁₋₅ alkoxy substituted by halogen,
heterocyclyl, or heterocyclyl substituted by substituent(s) independently selected from the group consisting of:
   halogen,
   C₁₋₅ alkyl,
   C₁₋₅ alkoxy,
   C₁₋₅ alkyl substituted by halogen, and
   C₁₋₅ alkoxy substituted by halogen;

or a pharmaceutically acceptable salt, hydrate, or solvate thereof.

In some embodiments, compounds of the present invention are of Formula (I) wherein R₁ is selected from the group consisting of:
(i) C₁₋₁₀ alkyl, and
   C₁₋₁₀ alkyl substituted by substituent(s) independently selected from the group consisting of:
      halogen,
      hydroxy,
      oxo,
      C₁₋₅ alkoxy,
      C₁₋₅ alkoxy substituted by carbocyclic aryl,
      C₁₋₅ alkylcarbonyloxy,
      C₁₋₅ alkoxycarbonyl,
      C₁₋₅ alkoxycarbonyl substituted by carbocyclic aryl,
      carbocyclic aryloxy, and
      carbocyclic aryloxy substituted by substituent(s) independently selected from the group consisting of:
         halogen,
         nitro,
         C₁₋₅ alkyl, and
         C₁₋₅ alkyl substituted by oxo,
      heterocyclyloxy,
      heterocyclyloxy substituted by C₁₋₅ alkyl,
      mono-carbocyclic arylamino,
      di-carbocyclic arylamino,
      carbocyclic arylsulfonylamino,
      carbocyclic arylsulfonylamino substituted by C₁₋₅ alkyl,
      C₁₋₅ alkylthio,
      C₁₋₅ alkylthio substituted by carbocyclic aryl,
      carbocyclic arylthio,
      carbocyclic arylthio substituted by halogen,
      carbocyclic arylthio substituted by C₁₋₅ alkyl,
      carbocyclic arylsulfonyl,
      carbocyclic arylsulfonyl substituted by halogen,
      heterocyclylthio,
      heterocyclylthio substituted by C₁₋₅ alkyl,
      C₃₋₆ cycloalkyl,
      C₃₋₆ cycloalkenyl,
      carbocyclyl,
      carbocyclyl substituted by C₁₋₅ alkoxy,
      carbocyclic aryl, and
      carbocyclic aryl substituted by substituent(s) independently selected from the group consisting of:
         halogen,
         nitro,
         C₁₋₅ alkyl, and
         C₁₋₅ alkyl substituted by substituent(s) independently selected from the group consisting of:
            halogen,
            carbocyclic aryl, and
            heterocyclyl,
         C₁₋₅ alkoxy,
         C₁₋₅ alkoxy substituted by halogen,
         C₁₋₅ alkoxy substituted by carbocyclic aryl, carbocyclic aryloxy,
         mono-carbocyclic arylaminocarbonyl, and
         mono-carbocyclic arylaminocarbonyl substituted by substituent(s) selected from the group consisting of:
            halogen,
            C₁₋₅ alkyl,
            C₁₋₅ alkoxy, and
            C₁₋₅ alkoxy substituted by halogen,
         di-carbocyclic arylaminocarbonyl, and
         di-carbocyclic arylaminocarbonyl substituted by substituent(s) selected from the group consisting of:
            halogen,
            C₁₋₅ alkyl,
            C₁₋₅ alkoxy, and
            C₁₋₅ alkoxy substituted by halogen,
         C₁₋₅ alkylthio,
         C₁₋₅ alkylthio substituted by halogen,
         C₁₋₅ alkylsulfonyl,
         carbocyclic aryl, and
         heterocyclyl,
      heterocyclyl, and
      heterocyclyl substituted by substituent(s) independently selected from the group consisting of:
         C₁₋₅ alkyl,
         C₁₋₅ alkoxy,
         C₁₋₅ alkoxy substituted by carbocyclic aryl,
         carbocyclic aryl, and
         carbocyclic aryl substituted by halogen,
(ii) C₂₋₅ alkenyl, and
   C₂₋₅ alkenyl substituted by substituent(s) independently selected from the group consisting of:
      carbocyclic aryl, and
      carbocyclic aryl substituted by substituent(s) independently selected from the group consisting of:
         nitro,
         halogen,
         C₁₋₅ alkyl,
         C₁₋₅ alkyl substituted by halogen,
         C₁₋₅ alkoxy, and
         C₁₋₅ alkoxy substituted by halogen,
(iii) C₃₋₆ cycloalkyl, and
   C₃₋₆ cycloalkyl substituted by substituent(s) independently selected from the group consisting of:
      C₁₋₅ alkyl,
      C₁₋₅ alkyl substituted by carbocyclic aryl,
      carbocyclic arylcarbonylamino, and
      carbocyclic aryl,
(iv) carbocyclyl, and
   carbocyclyl substituted by nitro,
(v) carbocyclic aryl, and
   carbocyclic aryl substituted by substituent(s) independently selected from the group consisting of:
      halogen,
      cyano,
      nitro,
      C₁₋₉ alkyl, and
      C₁₋₉ alkyl substituted by substituent(s) independently selected from the group consisting of:
         halogen,
         oxo,
         mono-carbocyclic arylaminocarbonyl,
         di-carbocyclic arylaminocarbonyl,
         mono-carbocyclic arylaminocarbonyl substituted by C₁₋₅ alkoxy,
         di-carbocyclic arylaminocarbonyl substituted by C₁₋₅ alkoxy,
         carbocyclic aryoxy,
         carbocyclic aryl, and
         carbocyclic aryl substituted by substituent(s) independently selected from the group consisting of:
            halogen,
            C₁₋₅ alkyl, and
            C₁₋₅ alkyl substituted by halogen,
         heterocyclyl, and
         heterocyclyl substituted by C₁₋₅ alkyl,
      C₂₋₅ alkenyl,
      C₁₋₇ alkoxy,
      C₁₋₇ alkoxy substituted by halogen,
      C₁₋₇ alkoxy substituted by carbocyclic aryl,
      C₃₋₆ cycloalkoxy,
      carbocyclic aryloxy, and
      carbocyclic aryloxy substituted by substituent(s) independently selected from the group consisting of:
         halogen,
         nitro, and
         C₁₋₅ alkoxy
      heterocyclyloxy, and
      heterocyclyloxy substituted by substituent(s) independently selected from the group consisting of:
         halogen,
         C₁₋₅ alkyl, and
         C₁₋₅ alkyl substituted by halogen,
      C₁₋₅ alkoxycarbonyl,
      mono-C₁₋₅ alkylaminocarbonyl,
      di-C₁₋₅ alkylaminocarbonyl,
      mono-C₁₋₅ alkylaminocarbonyl substituted by carbocyclic aryl,
      di-C₁₋₅ alkylaminocarbonyl substituted by carbocyclic aryl,
      mono-carbocyclic arylaminocarbonyl,
      di-carbocyclic arylaminocarbonyl,
      mono-carbocyclic arylaminocarbonyl substituted by C₁₋₅ alkyl,
      di-carbocyclic arylaminocarbonyl substituted by C₁₋₅ alkyl,
      mono-C₁₋₅ alkylamino,
      di-C₁₋₅ alkylamino,
      C₁₋₅ alkylthio,
      C₁₋₅ alkylthio substituted by halogen,
      C₁₋₅ alkylsulfonyl,
      carbocyclic aryl, and
      carbocyclic aryl substituted by substituent(s) independently selected from the group consisting of:
         C₁₋₇ alkyl, and
         C₁₋₇ alkyl substituted by halogen,
(vi) heterocyclyl, and
   heterocyclyl substituted by substituent(s) independently selected from the group consisting of:
      halogen,
      C₁₋₅ alkyl, and
      C₁₋₅ alkyl substituted by substituent(s) independently selected from the group consisting of:
         halogen,
         oxo,
         carbocyclic aryl,
         carbocyclic aryl substituted by halogen,
         heterocyclyl, and
         heterocyclyl substituted by substituent(s) independently selected from the group consisting of:
            halogen,
            C₁₋₅ alkyl, and
            C₁₋₅ alkyl substituted by halogen,
      C₁₋₅ alkoxy,
      C₁₋₅ alkylthio,
      carbocyclic arylthio,
      C₁₋₅ alkylsulfonyl,
      carbocyclic arylsulfonyl,
      carbocyclic arylsulfonyl substituted by halogen,
      carbocyclic arylsulfonyl substituted by C₁₋₅ alkyl,
      C₁₋₅ alkoxycarbonyl,
      carbocyclic aryl, and
      carbocyclic aryl substituted by substituent(s) independently selected from the group consisting of:
         halogen,
         nitro, and
         C₁₋₅ alkyl,
      heterocyclyl, and
      heterocyclyl substituted by substituent(s) independently selected from the group consisting of:
         halogen,
         C₁₋₅ alkyl, and
         C₁₋₅ alkyl substituted by halogen;
   wherein carbocyclic aryl is phenyl, naphthyl, or anthranyl;
   carbocyclyl is 1-oxo-indanyl, 9H-fluorenyl, 9-oxo-fluorenyl, anthraquinonyl, C-fluoren-9-ylidene, indanyl, or menthyl;
   heterocyclyl is 1,2,3,4-tetrahydro-isoquinolyl, 1,2,3-thiadiazolyl, 1,2,3-triazolyl, 1,3-dioxo-isoindolyl, 1H-indolyl, 1H-pyrrolyl, 2,3-dihydro-1-oxo-isoindolyl, 2,3-dihydro-benzo[1,4]dioxinyl, 2H-benzopyranyl, 2-oxo-benzopyranyl, 2-oxo-pyrrolidinyl, 4-oxo-benzopyranyl, 9H-xanthenyl, benzo[1,3]dioxolyl, benzo[2,1,3]oxadiazolyl, benzo[1,2,5]oxadiazolyl, benzo[b]thienyl, furyl, isoxazolyl, morpholinyl, oxazolyl, pyrazolyl, pyridyl, pyrimidyl, pyrrolidyl, quinolyl, quinoxalyl, thiazolyl, or thienyl;
      halogen is fluoro, chloro, bromo, or iodo;
   or a pharmaceutically acceptable salt, hydrate, or solvate thereof.

In some embodiments, compounds of the present invention are of Formula (I) wherein R₂ is C₁₋₅ alkyl substituted by carbocyclic aryl, C₁₋₅ alkyl substituted by halogenated carbocyclic aryl, C₁₋₅ alkyl substituted by heterocyclyl, C₁₋₅ alkyl substituted by halogenated heterocyclyl, carbocyclic aryl, carbocyclic aryl by halogen, heterocyclyl, heterocyclyl by halogen, or —N(R₂ₐ)(R_{2b}); wherein R₂ₐ and R_{2b} are each independently hydrogen, C₁₋₅ alkyl, C₁₋₅ alkyl substituted by hydroxy, or C₁₋₅ alkyl substituted by halogen; L is Formula (IIIa); wherein R₃ and R₄ are each independently hydrogen or C₁₋₅ alkyl; and A and B are each independently a single bond, —CH₂—, or —(CH₂)₂—; Z₃ and Z₄ are each independently hydrogen, halogen, C₁₋₅ alkyl, C₁₋₅ alkyl substituted by halogen, mono-C₁₋₅ alkyl amino, or di-C₁₋₅ alkyl amino; and Y is —C(O)—, —C(O)NR₅—, —C(S)NR₅—, or —(CH₂)ₘ—; wherein R₅ is hydrogen or C₁₋₅ alkyl; and m is 0, 1, or 2; Y is not —(CH₂)ₘ— provided that either R₂ₐ or R_{2b} is hydrogen;

or a pharmaceutically acceptable salt, hydrate, or solvate thereof.

In some embodiments, compounds of the present invention are of Formula (I) wherein R₁ is selected from the group consisting of:
(i) C₁₋₅ alkyl substituted by substituent(s) independently selected from the group consisting of:
   hydroxy,
   carbocyclic aryl,
   carbocyclic aryl substituted by halogen, and
   carbocyclic aryl substituted by halogenated C₁₋₅ alkyl,
(ii) carbocyclic aryl, and
   carbocyclic aryl substituted by substituent(s) independently selected from the group consisting of:
      halogen,
      cyano,
      C₁₋₅ alkyl,
      C₁₋₅ alkyl substituted by halogen,
      C₁₋₅ alkoxy, and
      C₁₋₅ alkoxy substituted by halogen,
(iii) heterocyclyl, and
   heterocyclyl substituted by halogen;
R₂ is C₁₋₅ alkyl substituted by carbocyclic aryl or —N(R₂ₐ)(R_{2b}); wherein R₂ₐ and R_{2b} are each independently hydrogen or C₁₋₅ alkyl;
L is Formula (IIIa); wherein R₃ and R₄ are each hydrogen; and A and B are each a single bond;
Z₃ and Z₄ are each independently hydrogen, C₁₋₅ alkyl, mono-C₁₋₅ alkyl amino, or di-C₁₋₅ alkyl amino;
   and
Y is —C(O)—;
wherein carbocyclic aryl is phenyl;
heterocyclyl is furyl or pyridyl;
halogen is fluoro, chloro, or bromo;
or a pharmaceutically acceptable salt, hydrate, or solvate thereof.

In some embodiments, compounds of the present invention are of Formula (I) wherein R₁ is selected from the group consisting of:
carbocyclic aryl, and
carbocyclic aryl substituted by substituent(s) independently selected from the group consisting of:
   halogen,
   cyano, and
   C₁₋₅ alkoxy;
Z₃ is hydrogen when Z₄ is C₁₋₅ alkyl; or Z₃ is C₁₋₅ alkyl, mono-C₁₋₅ alkyl amino, or di-C₁₋₅ alkyl amino when Z₄ is hydrogen;
or a pharmaceutically acceptable salt, hydrate, or solvate thereof.

In some embodiments, compounds of the present invention are of Formula (I) wherein the compound is selected from the group consisting of:
3-chloro-N-(cis-4-{[2-(dimethylamino)-6-methylpyrimidin-4-yl]amino}cyclohexyl)-4-fluorobenzamide;
N-(cis-4-{[2-(dimethylamino)-6-methylpyrimidin-4-yl]amino}cyclohexyl)-3,4-difluorobenzamide;
N-[cis-4-(2-dimethylamino-5-methyl-pyrimidin-4-ylamino)-cyclohexyl]-3-methoxy-benzamide;
N-[cis-4-(2-dimethylamino-5-methyl-pyrimidin-4-ylamino)-cyclohexyl]-3-trifluoromethyl-benzamide;
N-[cis-4-(2-dimethylamino-5-methyl-pyrimidin-4-ylamino)-cyclohexyl]-3,5-bis-trifluoromethyl-benzamide;
2,2-difluoro-benzo[1,3]dioxole-5-carboxylic acid [cis-4-(2-dimethylamino-5-methyl-pyrimidin-4-ylamino)-cyclohexyl]-amide;
4-cyano-N-[cis-4-(2-dimethylamino-5-methyl-pyrimidin-4-ylamino)-cyclohexyl]-benzamide;
4-chloro-N-[cis-4-(2-dimethylamino-5-methyl-pyrimidin-4-ylamino)-cyclohexyl]-benzamide;
N-[cis-4-(2-dimethylamino-5-methyl-pyrimidin-4-ylamino)-cyclohexyl]-3-ethyl-benzamide;
N-[cis-4-(2-dimethylamino-5-methyl-pyrimidin-4-ylamino)-cyclohexyl]-3,4-difluoro-benzamide;
5-bromo-N-[cis-4-(2-dimethylamino-5-methyl-pyrimidin-4-ylamino)-cyclohexyl]-nicotinamide;
5-bromo-furan-2-carboxylic acid [cis-4-(2-dimethylamino-5-methyl-pyrimidin-4-ylamino)-cyclohexyl]-amide;
3,5-dibromo-N-[cis-4-(2-dimethylamino-5-methyl-pyrimidin-4-ylamino)-cyclohexyl]-benzamide;
N-[cis-4-(2-dimethylamino-5-methyl-pyrimidin-4-ylamino)-cyclohexyl]-3-ethoxy-benzamide;
2-(3,5-bis-trifluoromethyl-phenyl)-N-[cis-4-(2-dimethylamino-5-methyl-pyrimidin-4-ylamino)-cyclohexyl]-2-hydroxy-acetamide;
2-(4-bromo-phenyl)-N-[cis-4-(2-dimethylamino-5-methyl-pyrimidin-4-ylamino)-cyclohexyl]-2-hydroxy-acetamide;
N-[cis-4-(2-dimethylamino-5-methyl-pyrimidin-4-ylamino)-cyclohexyl]-3,5-diethoxy-benzamide;
3-bromo-N-[cis-4-(2-dimethylamino-5-methyl-pyrimidin-4-ylamino)-cyclohexyl]-4-fluoro-benzamide;
N-[cis-4-(2-dimethylamino-6-methyl-pyrimidin-4-ylamino)-cyclohexyl]-3-ethoxy-benzamide;
N-[cis-4-(2-dimethylamino-6-methyl-pyrimidin-4-ylamino)-cyclohexyl]-3-trifluoromethyl-benzamide;
N-[cis-4-(2-dimethylamino-6-methyl-pyrimidin-4-ylamino)-cyclohexyl]-3,5-is-trifluoromethyl-benzamide;
2,2-difluoro-benzo[1,3]dioxole-5-carboxylic acid [cis-4-(2-dimethylamino-6-methyl-pyrimidin-4-ylamino)-cyclohexyl]-amide;
4-chloro-N-[cis-4-(2-dimethylamino-6-methyl-pyrimidin-4-ylamino)-cyclohexyl]-benzamide;
N-[cis-4-(2-dimethylamino-6-methyl-pyrimidin-4-ylamino)-cyclohexyl]-3-ethyl-benzamide;
N-[cis-4-(2-dimethylamino-6-methyl-pyrimidin-4-ylamino)-cyclohexyl]-4-methyl-benzamide;
5-bromo-N-[cis-4-(2-dimethylamino-6-methyl-pyrimidin-4-ylamino)-cyclohexyl]-nicotinamide;
5-bromo-furan-2-carboxylic acid [cis-4-(2-dimethylamino-6-methyl-pyrimidin-4-ylamino)-cyclohexyl]-amide;
3,5-dibromo-N-[cis-4-(2-dimethylamino-6-methyl-pyrimidin-4-ylamino)-cyclohexyl]-benzamide;
N-[cis-4-(2-dimethylamino-6-methyl-pyrimidin-4-ylamino)-cyclohexyl]-3-ethoxy-benzamide;
2-(3,5-bis-trifluoromethyl-phenyl)-N-[cis-4-(2-dimethylamino-6-methyl-pyrimidin-4-ylamino)-cyclohexyl]-2-hydroxy-acetamide;
2-(4-bromo-phenyl)-N-[cis-4-(2-dimethylamino-6-methyl-pyrimidin-4-ylamino)-cyclohexyl]-2-hydroxy-acetamide;
N-[cis-4-(2-dimethylamino-6-methyl-pyrimidin-4-ylamino)-cyclohexyl]-3,5-diethoxy-benzamide; and
3-bromo-N-[cis-4-(2-dimethylamino-6-methyl-pyrimidin-4-ylamino)-cyclohexyl]-4-fluoro-benzamide;

or a pharmaceutically acceptable salt, hydrate, or solvate thereof.

In some embodiments, compounds of the present invention are of Formula (I) wherein the compound is selected from the group consisting of:
3-chloro-N-(cis-4-{[2-(dimethylamino)pyrimidin-4-yl]amino}cyclohexyl)-4-fluorobenzamide;
N-(cis-4-{[2,6-bis(dimethylamino)pyrimidin-4-yl]amino}cyclohexyl)-3,4-difluorobenzamide;
N-(cis-4-{[2-benzyl-6-(dimethylamino)pyrimidin-4-yl]amino}cyclohexyl)-3-chloro-4-fluorobenzamide;
3,4-dichloro-N-[cis-4-(2-dimethylamino-6-methyl-pyrimidin-4-ylamino)-cyclohexyl]-benzamide;
4-cyano-N-[cis-4-(2-dimethylamino-6-methyl-pyrimidin-4-yl amino)-cyclohexyl]-benzamide;
N-[cis-4-(2-dimethylamino-6-methyl-pyrimidin-4-ylamino)-cyclohexyl]-3,4-diethoxy-benzamide;
3-chloro-N-[cis-4-(2-dimethylamino-6-methyl-pyrimidin-4-ylamino)-cyclohexyl]-5-fluoro-benzamide;
N-[cis-4-(2-dimethylamino-5-methyl-pyrimidin-4-ylamino)-cyclohexyl]-3,5-dimethoxy-benzamide;
3,4-dichloro-N-[cis-4-(2-dimethylamino-5-methyl-pyrimidin-4-ylamino)-cyclohexyl]-benzamide;
N-[cis-4-(2-dimethylamino-5-methyl-pyrimidin-4-ylamino)-cyclohexyl]-3,4-diethoxy-benzamide; and
3-chloro-N-[cis-4-(2-dimethylamino-5-methyl-pyrimidin-4-ylamino)-cyclohexyl]-5-fluoro-benzamide;

or a pharmaceutically acceptable salt, hydrate, or solvate thereof.

In some embodiments, compounds of the present invention are of Formula (I) wherein R₁ is selected from hydrogen, —CO₂^{t}Bu, or —CO₂Bn (Bn is a benzyl group); R₂ is C₁₋₅ alkyl substituted by carbocyclic aryl, C₁₋₅ alkyl substituted by halogenated carbocyclic aryl, C₁₋₅ alkyl substituted by heterocyclyl, C₁₋₅ alkyl substituted by halogenated heterocyclyl, carbocyclic aryl, carbocyclic aryl by halogen, heterocyclyl, heterocyclyl by halogen, or —N(R₂ₐ)(R_{2b}); wherein R₂ₐ and R_{2b} are each independently hydrogen, C₁₋₅ alkyl, C₁₋₅ alkyl substituted by hydroxy, or C₁₋₅ alkyl substituted by halogen; L is Formula (IIIa); wherein R₃ and R₄ are each independently hydrogen or C₁₋₅ alkyl; and A and B are each independently a single bond, —CH₂—, or —(CH₂)₂—; Z₃ and Z₄ are each independently hydrogen, halogen, C₁₋₅ alkyl, C₁₋₅ alkyl substituted by halogen, mono-C₁₋₅ alkyl amino, or di-C₁₋₅ alkyl amino; and Y is a single bond;

or a pharmaceutically acceptable salt, hydrate, or solvate thereof.

In some embodiments, compounds of the present invention are of Formula (I) wherein R₂ is C₁₋₅ alkyl substituted by carbocyclic aryl or —N(R₂ₐ)(R_{2b}); wherein R₂ₐ and R_{2b} are each independently hydrogen or C₁₋₅ alkyl; L is Formula (IIIa); wherein R₃ and R₄ are each hydrogen; and A and B are each a single bond; and Z₃ and Z₄ are each independently hydrogen, C₁₋₅ alkyl, mono-C₁₋₅ alkyl amino, or di-C₁₋₅ alkyl amino;

wherein carbocyclic aryl is phenyl;

heterocyclyl is furyl or pyridyl;

halogen is fluoro, chloro, or bromo;

or a pharmaceutically acceptable salt, hydrate, or solvate thereof.

One aspect of the present invention pertains to pharmaceutical compositions comprising a therapeutically effective amount of at least one compound, as described herein, in combination with a pharmaceutically acceptable carrier.

One aspect of the present invention pertains to methods for the prophylaxis or treatment of improving memory function, sleeping and arousal, anxiety, depression, mood disorders, seizure, obesity, diabetes, appetite and eating disorders, cardiovascular disease, hypertension, dyslipidemia, myocardial infarction, binge eating disorders including bulimia, anorexia, mental disorders including manic depression, schizophrenia, delirium, dementia, stress, cognitive disorders, attention deficit disorder, substance abuse disorders and dyskinesias including Parkinson's disease, epilepsy, and addiction comprising administering to an individual suffering from the condition a therapeutically effective amount of a compound, as described herein, or a pharmaceutical composition thereof.

One aspect of the present invention pertains to methods for the prophylaxis or treatment of an eating disorder, obesity or an obesity related disorder comprising administering to an individual suffering from the condition a therapeutically effective amount of a compound, as described herein, or a pharmaceutical composition thereof.

One aspect of the present invention pertains to methods for the prophylaxis or treatment of anxiety, depression, schizophrenia, addiction, or epilepsy comprising administering to an individual suffering from the condition a therapeutically effective amount of a compound, as described herein, or a pharmaceutical composition.

One aspect of the present invention pertains to compounds of the present invention, as described herein, or a pharmaceutical composition thereof, for use in a method of treatment of the human or animal body by therapy.

One aspect of the present invention pertains to compounds of the present invention, as described herein, or a pharmaceutical composition thereof, for use in a method of prophylaxis or treatment of an eating disorder, obesity or an obesity related disorder of the human or animal body by therapy.

One aspect of the present invention pertains to compounds of the present invention, as described herein, or a pharmaceutical composition thereof, for use in a method of prophylaxis or treatment of anxiety, depression, schizophrenia, addiction, or epilepsy of the human or animal body by therapy.

One aspect of the present invention pertains to compounds of the present invention, as described herein, for the manufacture of a medicament for use in the prophylaxis or treatment of an eating disorder, obesity or obesity related disorders.

One aspect of the present invention pertains to compounds of the present invention, as described herein, for the manufacture of a medicament for use in the prophylaxis or treatment of anxiety, depression, schizophrenia, addiction, or epilepsy.

One aspect of the present invention pertains to methods of decreasing food intake of an individual comprising administering to the individual a therapeutically effective amount of a compound, as described herein, or a pharmaceutical composition thereof.

One aspect of the present invention pertains to methods of inducing satiety in an individual comprising administering to said individual a therapeutically effective amount of a compound, as described herein, or a pharmaceutical composition thereof.

One aspect of the present invention pertains to methods of controlling or reducing weight gain in an individual comprising administering to said individual a therapeutically effective amount of a compound, as described herein, or a pharmaceutical composition thereof.

One aspect of the present invention pertains to methods of modulating a MCH receptor in an individual comprising contacting the receptor with a compound, as described herein. In some embodiments, the compound is an antagonist. In some embodiments, the modulation of the MCH receptor is for the prophylaxis or treatment of an eating disorder, obesity or obesity related disorder. In some embodiments, the modulation of the MCH receptor reduces food intake of the individual. In some embodiments, the modulation of the MCH receptor induces satiety in the individual. In some embodiments, the modulation of the MCH receptor controls or reduces weight gain of the individual. In some embodiments, the modulation of the MCH receptor is for prophylaxis or treatment of anxiety, depression, schizophrenia, addiction, or epilepsy.

In some embodiments, the individual is a mammal.

In some embodiments, the mammal is a human.

In some embodiments, the human has a body mass index of about 18.5 to about 45. In some embodiments, the human has a body mass index of about 25 to about 45. In some embodiments, the human has a body mass index of about 30 to about 45. In some embodiments, the human has a body mass index of about 35 to about 45.

One aspect of the present invention pertains to methods of producing a pharmaceutical composition comprising admixing a compound, as described herein, and a pharmaceutically acceptable carrier.

One embodiment of the invention includes any compound of the invention which selectively binds an MCH receptor, such selective binding is preferably demonstrated by a Ki for one or more other GPCR(s), preferably NPY, being at least 10-fold greater than the Ki for any particular MCH receptor, preferable MCHR1.

As used herein, the term “alkyl” is intended to denote hydrocarbon compounds including straight chain and branched chain, including for example but not limited to methyl, ethyl, n-propyl, isopropyl, n-butyl, sec-butyl, tert-butyl, n-pentyl, isopentyl, tert-pentyl, n-hexyl, and the like.

The term “alkoxy” is intended to denote substituents of the formula

—O-alkyl.

At various places in the present specification substituents of compounds of the invention are disclosed in groups. It is specifically intended that the invention include each and every individual subcombination of the members of such groups.

G-protein coupled receptors (GPCRs) represent a major class of cell surface receptors with which many neurotransmitters interact to mediate their effects. GPCRs are predicted to have seven membrane-spanning domains and are coupled to their effectors via G-proteins linking receptor activation with intracellular biochemical sequelae such as stimulation of adenylyl cyclase. Melanin Concentrating Hormone (MCH), a cyclic peptide, has been identified as the endogenous ligand of the orphan G-protein coupled receptor SLC-1. See, for example, Shimomura et al., Biochem. Biophys. Res. Commun. 261, 622-26 (1999). Studies have indicated that MCH acts as a neurotransmitter/modulator/regulator to alter a number of behavioral responses.

Mammalian MCH (19 amino acids) is highly conserved between rat, mouse, and human, exhibiting 100% amino acid identity, but its physiological roles are less clear. MCH has been reported to participate in a variety of processes including feeding, water balance, energy metabolism, general arousal/attention state, memory and cognitive functions, and psychiatric disorders. For reviews, see 1. Baker, Int. Rev. Cytol. 126:1-47 (1991); 2. Baker, TEM 5:120-126 (1994); 3. Nahon, Critical Rev. in Neurobiol 221:221-262, (1994); 4. Knigge et al., Peptides 18 (7):1095-1097, (1996). The role of MCH in feeding or body weight regulation is supported by Qu et al., Nature 380:243-247, (1996), demonstrating that MCH is over expressed in the hypothalamus of ob/ob mice compared with ob/+mice, and that fasting further increased MCH mRNA in both obese and normal mice during fasting. MCH also stimulated feeding in normal rats when injected into the lateral ventricles as reported by Rossi et al., Endocrinology 138:351-355, (1997). MCH also has been reported to functionally antagonize the behavioral effects of α-MSH; see: Miller et al., Peptides 14:1-10, (1993); Gonzalez et al, Peptides 17:171-177, (1996); and Sanchez et al., Peptides 18:3933-396, (1997). In addition, stress has been shown to increase POMC mRNA levels while decreasing the MCH precursor preproMCH (ppMCH) mRNA levels; Presse et al., Endocrinology 131:1241-1250, (1992). Thus MCH can serve as an integrative neuropeptide involved in the reaction to stress, as well as in the regulation of feeding and sexual activity; Baker, Int. Rev. Cytol. 126:1-47, (1991); Knigge et al., Peptides 17:1063-1073, (1996).

The localization and biological activities of MCH peptide suggest that the modulation of MCH receptor activity can be useful in a number of therapeutic applications. MCH is expressed in the lateral hypothalamus, a brain area implicated in the regulation of thirst and hunger: Grillon et al., Neuropeptides 31:131-136, (1997); recently orexins A and B, which are potent orexigenic agents, have been shown to have very similar localization to MCH in the lateral hypothalamus; Sakurai et al., Cell 92:573-585 (1998). MCH mRNA levels in this brain region are increased in rats after 24 hours of food-deprivation; Herve and Fellmann, Neuropeptides 31:237-242 (1997); after insulin injection, a significant increase in the abundance and staining intensity of MCH immunoreactive perikarya and fibres was observed concurrent with a significant increase in the level of MCH mRNA; Bahjaoui-Bouhaddi et al., Neuropeptides 24:251-258, (1994). Consistent with the ability of MCH to stimulate feeding in rats; Rossi et al., Endocrinology 138:351-355, (1997); is the observation that MCH mRNA levels are upregulated in the hypothalami of obese ob/ob mice; Qu et al., Nature 380:243-247, (1996); and decreased in the hypothalami of rats treated with leptin, whose food intake and body weight gains are also decreased; Sahu, Endocrinology 139:795-798, (1998). MCH appears to act as a functional antagonist of the melanocortin system in its effects on food intake and on hormone secretion within the HPA (hypothalamopituitary/adrenal axis); Ludwig et al., Am. J. Physiol. Endocrinol. Metab. 274:E627-E633, (1998). Together these data suggest a role for endogenous MCH in the regulation of energy balance and response to stress, and provide a rationale for the development of specific compounds acting at MCH receptors for use in the treatment of obesity and stress-related disorders.

Accordingly, a MCH receptor antagonist is desirable for the prophylaxis or treatment of obesity or obesity related disorders. An obesity related disorder is a disorder that has been directly or indirectly associated to obesity, such as, type II diabetes, syndrome X, impaired glucose tolerance, dyslipidaemia, hypertension, coronary heart disease and other cardiovascular disorders including atherosclerosis, insulin resistance associated with obesity and psoriasis, for treating diabetic complications and other diseases such as polycystic ovarian syndrome (PCOS), certain renal diseases including diabetic nephropathy, glomerulonephritis, glomerular sclerosis, nephrotic syndrome, hypertensive nephrosclerosis, end-stage renal diseases and microalbuminuria as well as certain eating disorders.

In species studied to date, a major portion of the neurons of the MCH cell group occupies a rather constant location in those areas of the lateral hypothalamus and subthalamus where they lie and may be a part of some of the so-called “extrapyramidal” motor circuits. These involve substantial striato- and pallidofugal pathways involving the thalamus and cerebral cortex, hypothalamic areas, and reciprocal connections to subthalamic nucleus, substantia nigra, and mid-brain centers; Bittencourt et al., J. Comp. Neurol. 319:218-245, (1992). In their location, the MCH cell group may offer a bridge or mechanism for expressing hypothalamic visceral activity with appropriate and coordinated motor activity. Clinically it can be of some value to consider the involvement of this MCH system in movement disorders, such as Parkinson's disease and Huntingdon's Chorea in which extrapyramidal circuits are known to be involved.

Human genetic linkage studies have located authentic hMCH loci on chromosome 12 (12q23-24) and the variant hMCH loci on chromosome 5 (5q12-13) (Pedeutour et al., 1994). Locus 12q23-24 coincides with a locus to which autosomal dominant cerebellar ataxia type II (SCA2) has been mapped; Auburger et al., Cytogenet. Cell. Genet. 61:252-256, (1992); Twells et al., Cytogenet. Cell. Genet. 61:262-265, (1992). This disease comprises neurodegenerative disorders, including an olivopontocerebellar atrophy. Furthermore, the gene for Darier's disease, has been mapped to locus 12q23-24; Craddock et al., Hum. Mol. Genet. 2:1941-1943, (1993). Dariers' disease is characterized by abnormalities I keratinocyte adhesion and mental illnesses in some families. In view of the functional and neuroanatomical patterns of the MCH neural system in the rat and human brains, the MCH gene can represent a good candidate for SCA2 or Darier's disease. Interestingly, diseases with high social impact have been mapped to this locus. Indeed, the gene responsible for chronic or acute forms of spinal muscular atrophies has been assigned to chromosome 5q12-13 using genetic linkage analysis; Melki et al., Nature (London) 344:767-768, (1990); Westbrook et al., Cytogenet. Cell. Genet. 61:225-231, (1992). Furthermore, independent lines of evidence support the assignment of a major schizophrenia locus to chromosome 5q11.2-13.3; Sherrington et al., Nature (London) 336:164-167, (1988); Bassett et al., Lancet 1:799-801, (1988); Gilliam et al., Genomics 5:940-944, (1989). The above studies suggest that MCH can play a role in neurodegenerative diseases and disorders of emotion.

Additional therapeutic applications for MCH-related compounds are suggested by the observed effects of MCH in other biological systems. For example, MCH can regulate reproductive functions in male and female rats. MCH transcripts and MCH peptide were found within germ cells in testes of adult rats, suggesting that MCH can participate in stem cell renewal and/or differentiation of early spermatocytes; Hervieu et al., Biology of Reduction 54:1161-1172, (1996). MCH injected directly into the medial preoptic area (MPOA) or ventromedial nucleus (VMN) stimulated sexual activity in female rats; Gonzalez et al., Peptides 17:171-177, (1996). In ovariectomized rats primed with estradiol, MCH stimulated luteinizing hormone (LH) release while anti-MCH antiserum inhibited LH release; Gonzalez et al., Neuroendocrinology 66:254-262, (1997). The zona incerta, which contains a large population of MCH cell bodies, has previously been identified as a regulatory site for the pre-ovulatory LH surge; MacKenzie et al., Neuroendocrinology 39:289-295, (1984). MCH has been reported to influence release of pituitary hormones including ACTH and oxytocin. MCH analogues can also be useful in treating epilepsy. In the PTZ seizure model, injection of MCH prior to seizure induction prevented seizure activity in both rats and guinea pigs, suggesting that MCH-containing neurons can participate in the neural circuitry underlying PTZ-induced seizure; Knigge and Wagner, Peptides 18:1095-1097, (1997). MCH has also been observed to affect behavioral correlates of cognitive functions. MCH treatment hastened extinction of the passive avoidance response in rats; McBride et al., Peptides 15:757-759, (1994); raising the possibility that MCH receptor antagonists can be beneficial for memory storage and/or retention. A possible role for MCH in the modulation or perception of pain is supported by the dense innervation of the periaqueductal grey (PAG) by MCH-positive fibers. Finally, MCH can participate in the regulation of fluid intake. ICV infusion of MCH in conscious sheep produced diuretic, natriuretic, and kaliuretic changes in response to increased plasma volume; Parkes, J. Neuroendocrinol. 8:57-63, (1996). Together with anatomical data reporting the presence of MCH in fluid regulatory areas of the brain, the results indicate that MCH can be an important peptide involved in the central control of fluid homeostasis in mammals.

In a recent citation MCHR1 antagonists surprisingly demonstrated their use as an anti-depressants, and/or anti-anxiety agents. MCHR1 antagonists have been reported to show antidepressant and anxiolytic activities in rodent models, such as, social interaction, forced swimming test and ultrasonic vocalization. Therefore, MCHR1 antagonists could be useful to independently treat subjects with depression and/or anxiety. Also, MCHR1 antagonists could be useful to treat subjects that suffer from depression and/or anxiety and obesity.

This invention provides a method of treating an abnormality in a subject wherein the abnormality is alleviated by decreasing the activity of a mammalian MCH1 receptor which comprises administering to the subject an amount of a compound which is a mammalian MCH1 receptor antagonist effective to treat the abnormality. In separate embodiments, the abnormality is a regulation of a steroid or pituitary hormone disorder, an epinephrine release disorder, an anxiety disorder, genta gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neuroendocrine disorder, a cognitive disorder, a memory disorder, a sensory modulation and transmission disorder, a motor coordination disorder, a sensory integration disorder, a motor integration disorder, a dopaminergic function disorder, a sensory transmission disorder, an olfaction disorder, a sympathetic innervation disorder, an affective disorder, a stress-related disorder, a fluid-balance disorder, a seizure disorder, pain, psychotic behavior, morphine tolerance, opiate addiction or migraine.

Compositions of the invention can conveniently be administered in unit dosage form and can be prepared by any of the methods well known in the pharmaceutical art, for example, as described in Remington's Pharmaceutical Sciences (Mack Pub. Co., Easton, Pa., 1980).

The compounds of the invention can be employed as the sole active agent in a pharmaceutical or can be used in combination with other active ingredients which could facilitate the therapeutic effect of the compound.

Compounds of the present invention or a solvate or physiologically functional derivative thereof can be used as active ingredients in pharmaceutical compositions, specifically as a MCH receptor antagonists. By the term “active ingredient” is defined in the context of a “pharmaceutical composition” and shall mean a component of a pharmaceutical composition that provides the primary pharmaceutical benefit, as opposed to an “inactive ingredient” which would generally be recognized as providing no pharmaceutical benefit. The term “pharmaceutical composition” shall mean a composition comprising at one active ingredient and at least one ingredient that is not an active ingredient (for example and not limitation, a filler, dye, or a mechanism for slow release), whereby the composition is amenable to use for a specified, efficacious outcome in a mammal (for example, and not limitation, a human).

Pharmaceutical compositions, including, but not limited to, pharmaceutical compositions, comprising at least one compound of the present invention and/or an acceptable salt or solvate thereof (e.g., a pharmaceutically acceptable salt or solvate) as an active ingredient combined with at least one carrier or excipient (e.g., pharmaceutical carrier or excipient) can be used in the treatment of clinical conditions for which a MCH receptor antagonist is indicated. At least one compound of the present invention can be combined with the carrier in either solid or liquid form in a unit dose formulation. The pharmaceutical carrier must be compatible with the other ingredients in the composition and must be tolerated by the individual recipient. Other physiologically active ingredients can be incorporated into the pharmaceutical composition of the invention if desired, and if such ingredients are compatible with the other ingredients in the composition. Formulations can be prepared by any suitable method, typically by uniformly mixing the active compound(s) with liquids or finely divided solid carriers, or both, in the required proportions, and then, if necessary, forming the resulting mixture into a desired shape.

Conventional excipients, such as binding agents, fillers, acceptable wetting agents, tabletting lubricants, and disintegrants can be used in tablets and capsules for oral administration. Liquid preparations for oral administration can be in the form of solutions, emulsions, aqueous or oily suspensions, and syrups. Alternatively, the oral preparations can be in the form of dry powder that can be reconstituted with water or another suitable liquid vehicle before use. Additional additives such as suspending or emulsifying agents, non-aqueous vehicles (including edible oils), preservatives, and flavorings and colorants can be added to the liquid preparations. Parenteral dosage forms can be prepared by dissolving the compound of the invention in a suitable liquid vehicle and filter sterilizing the solution before filling and sealing an appropriate vial or ampoule. These are just a few examples of the many appropriate methods well known in the art for preparing dosage forms.

It is noted that when the MCH receptor antagonists are utilized as active ingredients in a pharmaceutical composition, these are not intended for use only in humans, but in other non-human mammals as well. Indeed, recent advances in the area of animal health-care mandate that consideration be given for the use of MCH receptor antagonists for the treatment of obesity in domestic animals (e.g., cats and dogs), and MCH receptor antagonists in other domestic animals where no disease or disorder is evident (e.g., food-oriented animals such as cows, chickens, fish, etc.). Those of ordinary skill in the art are readily credited with understanding the utility of such compounds in such settings.

Pharmaceutically acceptable salts of the compounds of the invention can be prepared by reacting the free acid or base forms of these compounds with the appropriate base or acid in water, in an organic solvent, or in a mixture of the two; generally, nonaqueous media like ether, ethyl acetate, ethanol, isopropanol, dioxane, or acetonitrile are preferred. For instance, when the compound (I) possesses an acidic functional group, it can form an inorganic salt such as an alkali metal salt (e.g., sodium salt, potassium salt, etc.), an alkaline earth metal salt (e.g. calcium salt, magnesium salt, barium salt, etc.), and an ammonium salt. When the compound (I) possesses a basic functional group, it can form an inorganic salt (e.g., hydrochloride, sulfate, phosphate, hydrobromate, etc.) or an organic salt (e.g., acetate, maleate, fumarate, succinate, methanesulfonate, p-toluenesulfonate, citrate, tartrate, etc.).

When a compound of the invention contains optical isomers, stereoisomers, regio isomers, rotational isomers, a single substance and a mixture of them are included as a compound of the invention. For example, when a chemical formula is represented as showing no stereochemical designation(s), such as Formula (III), then all possible stereoisomer, optical isomers and mixtures thereof are considered within the scope of that formula. Accordingly, Formula (IIIa), specifically designates the cis relationship between the two amino groups on the cyclohexyl ring and therefore this formula is also fully embraced by Formula (III).

### Preparation of Compound of Formula (I)—General Synthetic Methods

The novel substituted pyrimidines of the present invention can be readily prepared according to a variety of synthetic manipulations, all of which would be familiar to one skilled in the art. Preferred methods for the preparation of compounds of the present invention include, but are not limited to, those described in Scheme 1-8.

The pyrimidine (C) can be prepared as shown in Scheme 1. 4,6-Dihydroxypyrimidine (A), which is commercially available or is condensed from malonic acid derivatives and amidine derivatives, wherein Z₁ and Z₂ are as defined above, is converted to 4,6-dihalo-pyrimidine (B) by a halogenating agent with or without a base (wherein X is halogen such as chloro, bromo, or iodo). The halogenating agent includes phosphorous oxychloride (POCl₃), phosphorous oxybromide (POBr₃), or phosphorus pentachloride (PCl₅). The base includes a tertiary amine (preferably N,N-diisopropylethylamine, etc.) or an aromatic amine (preferably N,N-dimethylaniline, etc.). Reaction temperature ranges from about 100° C. to 200° C., preferably about 140° C. to 180° C. The introduction of R₂ substituent to 4,6-dihalo-pyrimidine (B) gives the pyrimidine (C). Also the pyrimidine (C) can be prepared from commercially available 2,4,6-trihalo-pyrimidine (D), wherein Z₂ is as defined above and X is halogen such as chloro, bromo, or iodo, following the introduction of R₂ substituent and Z₁ substituent.

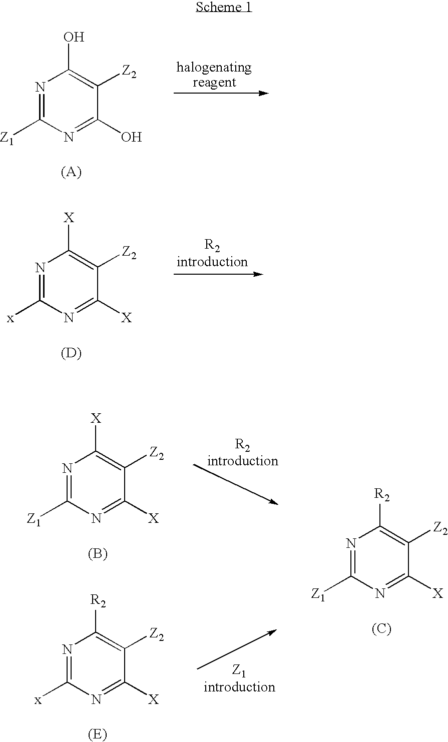

The common intermediate (H) of the novel substituted pyrimidines can be prepared as shown in Scheme 2. The pyrimidine (C) is substituted by the mono-protected diamine (F), wherein R₃, R₄, A, and B are as defined above and P is a protective group, with or without a base in an inert solvent to provide the coupling adduct (G). The base includes an alkali metal carbonate (preferably sodium carbonate or potassium carbonate, etc.), an alkali metal hydroxide (preferably sodium hydroxide, etc.), or a tertiary amine (preferably N,N-diisopropylethylamine, triethylamine, or N-methylmorpholine, etc.). The inert solvent includes lower alkyl alcohol solvents (preferably methanol, ethanol, 2-propanol, or butanol, etc.) or amide solvents (preferably N,N-dimethylformamide or 1-methyl-pyrrolidin-2-one, etc.). Reaction temperature ranges from about 50° C. to 200° C., preferably about 80° C. to 150° C. Also this reaction can be carried out under microwave conditions.

Representative protecting groups suitable for a wide variety of synthetic transformations are disclosed in Greene and Wuts, Protective Groups in Organic Synthesis, second edition, John Wiley & Sons, New York, 1991, the disclosure of which is incorporated herein by reference in its entirety. The deprotection of the protective group leads to the common intermediate (H) of the novel substituted pyrimidines.

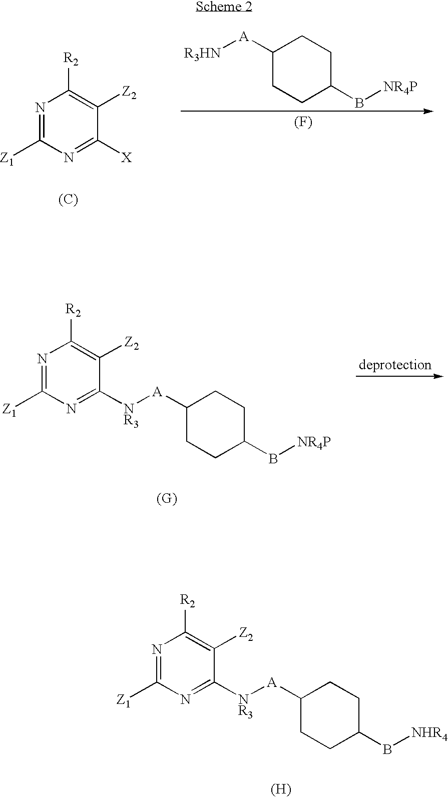

The conversion of the common intermediate (H) to the novel substituted pyrimidines (I), (J), and (V)-(X) of the present invention is outlined in Scheme 3.

The amine (H) is reacted with a carboxylic acid (R₁CO₂H) and a dehydrating condensing agent in an inert solvent with or without a base to provide the novel amide (I) of the present invention. The dehydrating condensing agent includes dicyclohexylcarbodiimide (DCC), 1-ethyl-3-(3-dimethylaminopropyl)carbodiimide hydrochloride (EDC.HCl), bromo-tris-pyrrolidino-phosnium hexafluorophosphate (PyBroP), O-(7-azabenzotriazol-1-yl)-1,1,3,3-tetramethyluronium hexafluorophosphate (HATU), or 1-cyclohexyl-3-methylpolystyrene-carbodiimide. The base includes a tertiary amine (preferably N,N-diisopropylethylamine or triethylamine, etc.). The inert solvent includes lower halocarbon solvents (preferably dichloromethane, dichloroethane, or chloroform, etc.), ethereal solvents (preferably tetrahydrofuran or dioxane), nitrile solvents (preferably acetonitrile, etc.), or amide solvents (preferably N,N-dimethylformamide, etc.). In case of need, 1-hydroxybenzotriazole (HOBT), HOBT-6-carboxaamidomethyl polystyrene, or 1-hydroxy-7-azabenzotriazole (HOAT) can be used as a reactant agent. Reaction temperature ranges from about −20° C. to 50° C., preferably about 0° C. to 40° C.

Alternatively, the novel amide (I) of the present invention can be obtained by amidation reaction using an acid chloride (R₁COCl) and a base in an inert solvent. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine, imidazole, poly-(4-vinylpyridine), etc.). The inert solvent includes lower halocarbon solvents (preferably dichloromethane, dichloroethane, or chloroform, etc.), ethereal solvents (preferably tetrahydrofuran or dioxane), amide solvents (preferably N,N-dimethylformamide, etc.), or aromatic solvents (preferably toluene or pyridine, etc.). Reaction temperature ranges from about −20° C. to 50° C., preferably about 0° C. to 40° C.

The novel amide (I) of the present invention is reacted with a reducing agent in an inert solvent to provide the novel amine (J) of the present invention. The reducing agent includes alkali metal aluminum hydrides (preferably lithium aluminum hydride), alkali metal borohydrides (preferably lithium borohydride), alkali metal trialkoxyaluminum hydrides (preferably lithium tri-tert-butoxyaluminum hydride), dialkylaluminum hydrides (preferably di-isobutylaluminum hydride), borane, dialkylboranes (preferably di-isoamyl borane), alkali metal trialkylboron hydrides (preferably lithium triethylboron hydride). The inert solvent includes ethereal solvents (preferably tetrahydrofuran or dioxane) or aromatic solvents (preferably toluene, etc.). Reaction temperature ranges from about −78° C. to 200° C., preferably about 50° C. to 120° C.

Alternatively, the novel amine (J) of the present invention can be obtained by reductive amination reaction using aldehyde (R₁CHO) and a reducing agent in an inert solvent with or without an acid. The reducing agent includes sodium triacetoxyborohydride, sodium cyanoborohydride, sodium borohydride, or boran-pyridine complex, preferably sodium triacetoxyborohydride or sodium cyanoborohydride. The inert solvent includes lower alkyl alcohol solvents (preferably methanol or ethanol, etc.), lower halocarbon solvents (preferably dichloromethane, dichloroethane, or chloroform, etc.), ethereal solvents (preferably tetrahydrofuran or dioxane), or aromatic solvents (preferably toluene, etc.). The acid includes an inorganic acid (preferably hydrochloric acid or sulfuric acid) or an organic acid (preferably acetic acid). Reaction temperature ranges from about −20° C. to 120° C., preferably about 0° C. to 100° C. Also this reaction can be carried out under microwave conditions.

The amine (I) is reacted with a sulfonyl halide (R₁SO₂X), wherein X is halogen such as chloro, bromo, or iodo, and a base in an inert solvent to provide the novel sulfonamide (V) of the present invention. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine or imidazole, etc.). The inert solvent includes lower halocarbon solvents (preferably dichloromethane, dichloroethane, or chloroform, etc.), ethereal solvents (preferably tetrahydrofuran or dioxane), alcohol solvents (preferably 2-propanol, etc.), or aromatic solvents (preferably toluene or pyridine, etc.). Reaction temperature ranges from about −20° C. to 50° C., preferably about 0° C. to 40° C.

The novel urea (W) or thiourea (W) of the present invention can be obtained by urea reaction or thiourea reaction using an isocyanate (R₁NCO) or isothiocyanate (R₁NCS) in an inert solvent with or without a base. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine or imidazole, etc.). The inert solvent includes lower halocarbon solvents (preferably dichloromethane, dichloroethane, or chloroform, etc.), ethereal solvents (preferably tetrahydrofuran or dioxane), aromatic solvents (preferably benzene or toluene, etc.), or polar solvents (preferably N,N-dimethylformamide or dimethyl sulfoxide, etc.). Reaction temperature ranges from about −20° C. to 120° C., preferably about 0° C. to 100° C.

The novel urethane (X) of the present invention can be obtained by urethane reaction using R₁OCOX, wherein X is halogen such as chloro, bromo, or iodo, in an inert solvent with or without a base. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine, imidazole, or poly-(4-vinylpyridine), etc.). The inert solvent includes lower halocarbon solvents (preferably dichloromethane, dichloroethane, or chloroform, etc.), ethereal solvents (preferably tetrahydrofuran or dioxane), aromatic solvents (preferably benzene or toluene, etc.), or polar solvents (preferably N,N-dimethylformamide or dimethyl sulfoxide, etc.). Reaction temperature ranges from about −20° C. to 120° C., preferably about 0° C. to 100° C.

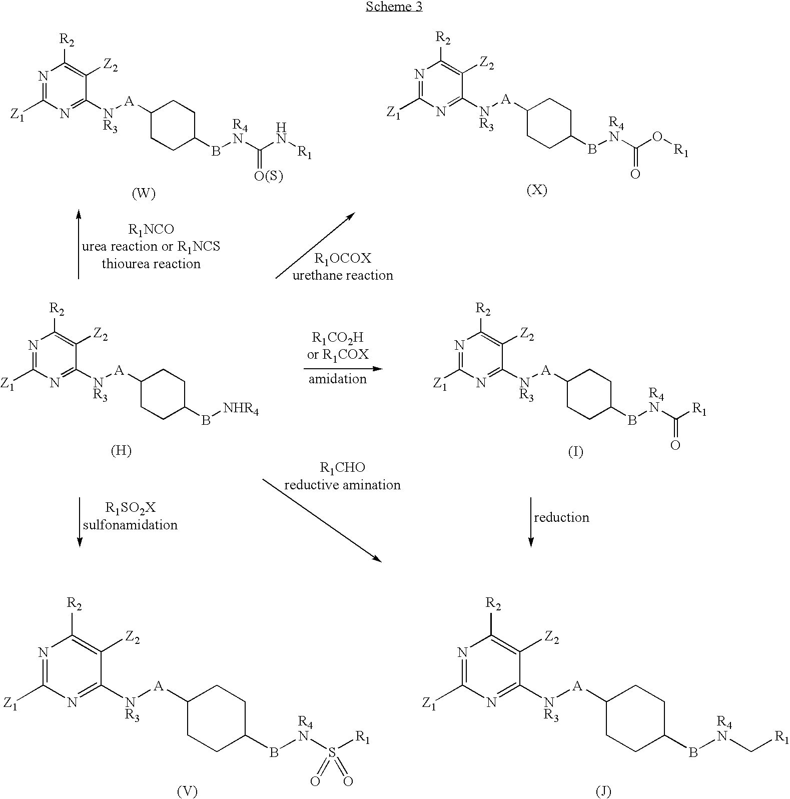

Also the novel substituted pyrimidine (M) of the present invention can be prepared as shown in Scheme 4.

First 4,6-dihalo-pyrimidine (B) is substituted by the amine (K) which has been already installed by the desired R₁ substituent, wherein R₃, R₄, A, B, Y, and R₁ are as defined above, with or without a base in an inert solvent to provide the coupling adduct (L). The base includes an alkali metal carbonate (preferably sodium carbonate or potassium carbonate, etc.), an alkali metal hydroxide (preferably sodium hydroxide, etc.), or a tertiary amine (preferably N,N-diisopropylethylamine, triethylamine, or N-methylmorpholine, etc.). The inert solvent includes lower alkyl alcohol solvents (preferably methanol, ethanol, 2-propanol, or butanol, etc.) or amide solvents (preferably N,N-dimethylformamide or 1-methyl-pyrrolidin-2-one, etc.). Reaction temperature ranges from about 50° C. to 200° C., preferably about 80° C. to 150° C. Also this reaction can be carried out under microwave conditions. The introduction of R₂ substituent leads to the novel substituted pyrimidine (M) of the present invention.

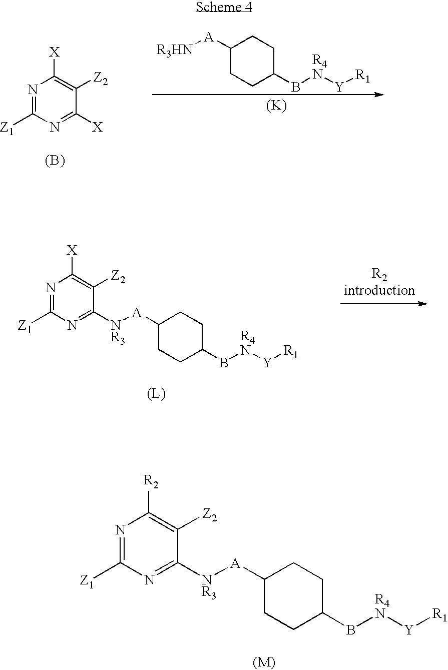

The common intermediate (R) of the novel substituted pyrimidines can be prepared as shown in Scheme 5.

Commercially available 2,4-dihydroxypyrimidine (N), wherein Z₃ and Z₄ are as defined above, is converted to 2,4-dihalo-pyrimidine (O) by a halogenating agent with or without a base (wherein X is halogen such as chloro, bromo, or iodo). The halogenating agent includes phosphorous oxychloride (POCl₃), phosphorous oxybromide (POBr₃), or phosphorus pentachloride (PCl₅). The base includes a tertiary amine (preferably N,N-diisopropylethylamine, etc.) or an aromatic amine (preferably N,N-dimethylaniline, etc.). Reaction temperature ranges from about 100° C. to 200° C., preferably about 140° C. to 180° C. The introduction of R₂ substituent to 2,4-dihalo-pyrimidine (O) gives the pyrimidine (P). The pyrimidine (P) is substituted by the mono-protected diamine (F), wherein R₃, R₄, A, and B are as defined above and P is a protective group, with or without a base in an inert solvent to provide the coupling adduct (Q). The base includes an alkali metal carbonate (preferably sodium carbonate or potassium carbonate, etc.), an alkali metal hydroxide (preferably sodium hydroxide, etc.), or a tertiary amine (preferably N,N-diisopropylethylamine, triethylamine, or N-methylmorpholine, etc.). The inert solvent includes lower alkyl alcohol solvents (preferably methanol, ethanol, 2-propanol, or butanol, etc.) or amide solvents (preferably N,N-dimethylformamide or 1-methyl-pyrrolidin-2-one, etc.). Reaction temperature ranges from about 50° C. to 200° C., preferably about 80° C. to 150° C. Also this reaction can be carried out under microwave conditions.

It is understood that regioisomers can be formed using certain methods described herein, such as Scheme 5, and that these regioisomers could be separated by using methods known to one skilled in the art.

Representative protecting groups suitable for a wide variety of synthetic transformations are disclosed in Greene and Wuts, Protective Groups in Organic Synthesis, second edition, John Wiley & Sons, New York, 1991, the disclosure of which is incorporated herein by reference in its entirety. The deprotection of the protective group leads to the common intermediate (R) of the novel substituted pyrimidines.

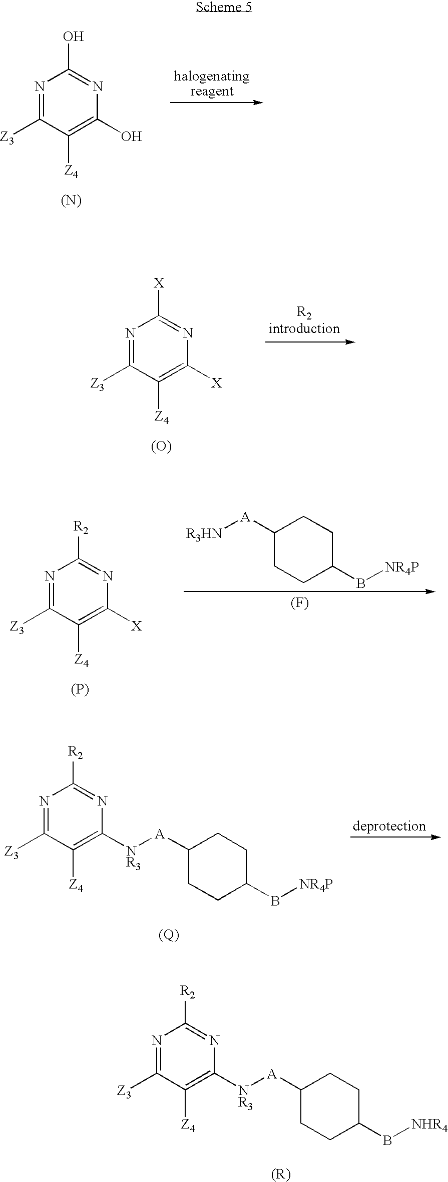

The conversion of the common intermediate (R) to the novel substituted pyrimidines (S), (T), and (V)-(A′) of the present invention is outlined in Scheme 6.

The amine (R) is reacted with a carboxylic acid (R₁CO₂H) and a dehydrating condensing agent in an inert solvent with or without a base to provide the novel amide (S) of the present invention. The dehydrating condensing agent includes dicyclohexylcarbodiimide (DCC), 1-ethyl-3-(3-dimethylaminopropyl)carbodiimide hydrochloride (EDC.HCl), bromo-tris-pyrrolidino-phosnium hexafluorophosphate (PyBroP), O-(7-azabenzotriazol-1-yl)-1,1,3,3-tetramethyluronium hexafluorophosphate (HATU), or 1-cyclohexyl-3-methylpolystyrene-carbodiimide. The base includes a tertiary amine (preferably N,N-diisopropylethylamine or triethylamine, etc.). The inert solvent includes lower halocarbon solvents (preferably dichloromethane, dichloroethane, or chloroform, etc.), ethereal solvents (preferably tetrahydrofuran or dioxane), nitrile solvents (preferably acetonitrile, etc.), or amide solvents (preferably N,N-dimethylformamide, etc.). In case of need, 1-hydroxybenzotriazole (HOBT), HOBT-6-carboxaamidomethyl polystyrene, or 1-hydroxy-7-azabenzotriazole (HOAT) can be used as a reactant agent. Reaction temperature ranges from about −20° C. to 50° C., preferably about 0° C. to 40° C.

Alternatively, the novel amide (S) of the present invention can be obtained by amidation reaction using an acid chloride (R₁COCl) and a base in an inert solvent. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine, imidazole, poly-(4-vinylpyridine), etc.). The inert solvent includes lower halocarbon solvents (preferably dichloromethane, dichloroethane, or chloroform, etc.), ethereal solvents (preferably tetrahydrofuran or dioxane), amide solvents (preferably N,N-dimethylformamide, etc.), or aromatic solvents (preferably toluene or pyridine, etc.). Reaction temperature ranges from about −20° C. to 50° C., preferably about 0° C. to 40° C.

The novel amide (S) of the present invention is reacted with a reducing agent in an inert solvent to provide the novel amine (T) of the present invention. The reducing agent includes alkali metal aluminum hydrides (preferably lithium aluminum hydride), alkali metal borohydrides (preferably lithium borohydride), alkali metal trialkoxyaluminum hydrides (preferably lithium tri-tert-butoxyaluminum hydride), dialkylaluminum hydrides (preferably di-isobutylaluminum hydride), borane, dialkylboranes (preferably di-isoamyl borane), alkali metal trialkylboron hydrides (preferably lithium triethylboron hydride). The inert solvent includes ethereal solvents (preferably tetrahydrofuran or dioxane) or aromatic solvents (preferably toluene, etc.). Reaction temperature ranges from about −78° C. to 200° C., preferably about 50° C. to 120° C.

Alternatively, the novel amine (T) of the present invention can be obtained by reductive amination reaction using aldehyde (R₁CHO) and a reducing agent in an inert solvent with or without an acid. The reducing agent includes sodium triacetoxyborohydride, sodium cyanoborohydride, sodium borohydride, or boran-pyridine complex, preferably sodium triacetoxyborohydride or sodium cyanoborohydride. The inert solvent includes lower alkyl alcohol solvents (preferably methanol or ethanol, etc.), lower halocarbon solvents (preferably dichloromethane, dichloroethane, or chloroform, etc.), ethereal solvents (preferably tetrahydrofuran or dioxane), or aromatic solvents (preferably toluene, etc.). The acid includes an inorganic acid (preferably hydrochloric acid or sulfuric acid) or an organic acid (preferably acetic acid). Reaction temperature ranges from about −20° C. to 120° C., preferably about 0° C. to 100° C. Also this reaction can be carried out under microwave conditions.

The amine (R) is reacted with a sulfonyl halide (R₁SO₂X), wherein X is halogen such as chloro, bromo, or iodo, and a base in an inert solvent to provide the novel sulfonamide (Y) of the present invention. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine or imidazole, etc.). The inert solvent includes lower halocarbon solvents (preferably dichloromethane, dichloroethane, or chloroform, etc.), ethereal solvents (preferably tetrahydrofuran or dioxane), alcohol solvents (preferably 2-propanol, etc.), or aromatic solvents (preferably toluene or pyridine, etc.). Reaction temperature ranges from about −20° C. to 50° C., preferably about 0° C. to 40° C.

The novel urea (Z) or thiourea (Z) of the present invention can be obtained by urea reaction or thiourea reaction using an isocyanate (R₁NCO) or isothiocyanate (R₁NCS) in an inert solvent with or without a base. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine or imidazole, etc.). The inert solvent includes lower halocarbon solvents (preferably dichloromethane, dichloroethane, or chloroform, etc.), ethereal solvents (preferably tetrahydrofuran or dioxane), aromatic solvents (preferably benzene or toluene, etc.), or polar solvents (preferably N,N-dimethylformamide or dimethyl sulfoxide, etc.). Reaction temperature ranges from about −20° C. to 120° C., preferably about 0° C. to 100° C.

The novel urethane (A′) of the present invention can be obtained by urethane reaction using R₁OCOX, wherein X is halogen such as chloro, bromo, or iodo, in an inert solvent with or without a base. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine, imidazole, or poly-(4-vinylpyridine), etc.). The inert solvent includes lower halocarbon solvents (preferably dichloromethane, dichloroethane, or chloroform, etc.), ethereal solvents (preferably tetrahydrofuran or dioxane), aromatic solvents (preferably benzene or toluene, etc.), or polar solvents (preferably N,N-dimethylformamide or dimethyl sulfoxide, etc.). Reaction temperature ranges from about −20° C. to 120° C., preferably about 0° C. to 100° C.

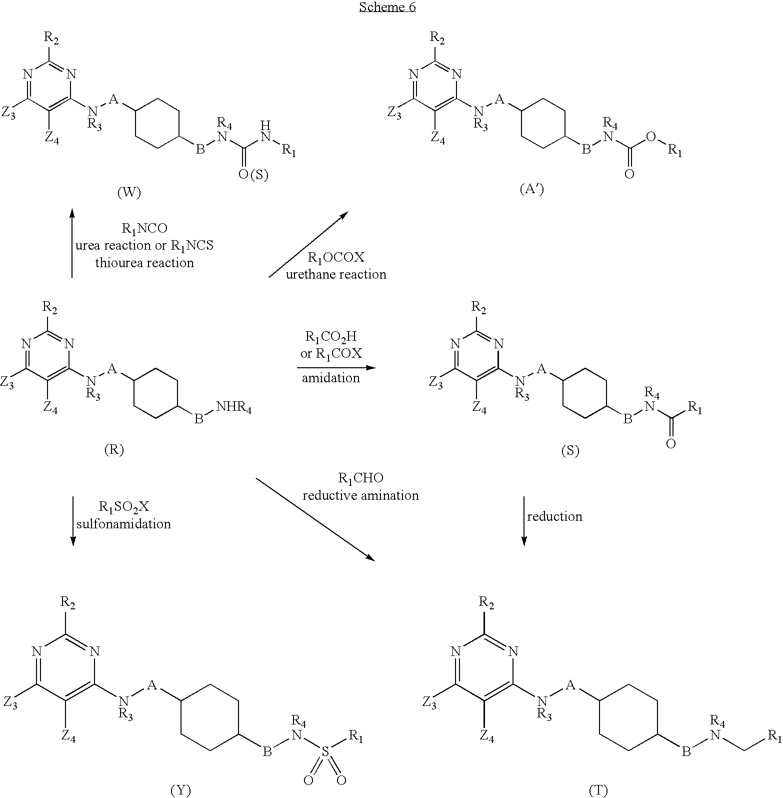

Alternatively, the novel pyrimidines (M) and (U) of the present invention are directly synthesized from the pyrimidine core (C), which is synthesized in Scheme 1 and the pyrimidine core (P), which is synthesized in Scheme 5, as shown in Scheme 7. This coupling is performed with or without a base in an inert solvent. The base includes an alkali metal carbonate (preferably sodium carbonate or potassium carbonate, etc.), an alkali metal hydroxide (preferably sodium hydroxide, etc.), or a tertiary amine (preferably N,N-diisopropylethylamine, triethylamine, or N-methylmorpholine, etc.). The inert solvent includes lower alkyl alcohol solvents (preferably methanol, ethanol, 2-propanol, or butanol, etc.) or amide solvents (preferably N,N-dimethylformamide or 1-methyl-pyrrolidin-2-one, etc.). Reaction temperature ranges from about 50° C. to 200° C., preferably about 80° C. to 180° C. Also this reaction can be carried out under microwave conditions.

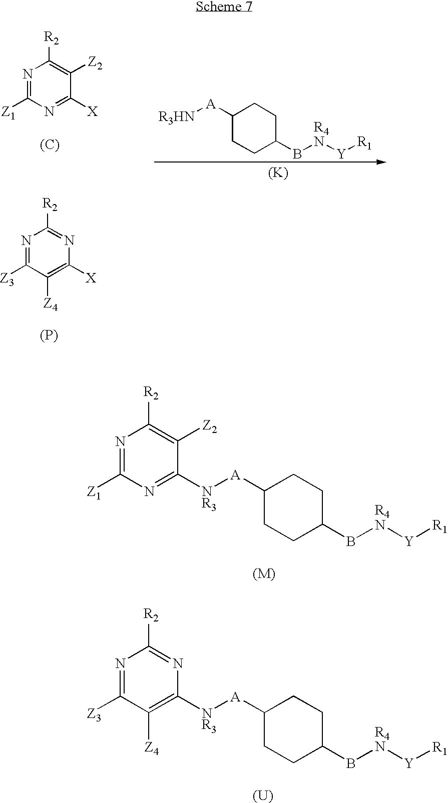

The common intermediate (C′) of the novel amide (D′) and the novel ester (E′) in the present invention is prepared from condensation between the pyrimidine core (C) which is synthesized in Scheme 1 and the carboxylic acid (B′), wherein R₃, A, and B are as defined above, as shown in Scheme 8.

The carboxylic acid (C′) is reacted with an amine (R₁NH₄) and a dehydrating condensing agent in an inert solvent with or without a base to provide the novel amide (D′) of the present invention. The dehydrating condensing agent includes dicyclohexylcarbodiimide (DCC), 1-ethyl-3-(3-dimethylaminopropyl)carbodiimide hydrochloride (EDC.HCl), bromo-tris-pyrrolidino-phosnium hexafluorophosphate (PyBroP), O-(7-azabenzotriazol-1-yl)-1,1,3,3-tetramethyluronium hexafluorophosphate (HATU), or 1-cyclohexyl-3-methylpolystyrene-carbodiimide. The base includes a tertiary amine (preferably N,N-diisopropylethylamine or triethylamine, etc.). The inert solvent includes lower halocarbon solvents (preferably dichloromethane, dichloroethane, or chloroform, etc.), ethereal solvents (preferably tetrahydrofuran or dioxane), nitrile solvents (preferably acetonitrile, etc.), or amide solvents (preferably N,N-dimethylformamide, etc.). In case of need, 1-hydroxybenzotriazole (HOBT), HOBT-6-carboxaamidomethyl polystyrene, or 1-hydroxy-7-azabenzotriazole (HOAT) can be used as a reactant agent. Reaction temperature ranges from about −20° C. to 50° C., preferably about 0° C. to 40° C.

Alternatively, the novel amide (D′) of the present invention can be obtained by amidation reaction via an acid chloride prepared from the carboxylic acid (C′) and a base in an inert solvent. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine, imidazole, poly-(4-vinylpyridine), etc.). The inert solvent includes lower halocarbon solvents (preferably dichloromethane, dichloroethane, or chloroform, etc.), ethereal solvents (preferably tetrahydrofuran or dioxane), amide solvents (preferably N,N-dimethylformamide, etc.), or aromatic solvents (preferably toluene or pyridine, etc.). Reaction temperature ranges from about −20° C. to 50° C., preferably about 0° C. to 40° C.

The carboxylic acid (C′) is reacted with an alcohol (R₁OH) and a dehydrating condensing agent in an inert solvent with or without a base to provide the novel ester (E′) of the present invention. The dehydrating condensing agent includes dicyclohexylcarbodiimide (DCC), 1-ethyl-3-(3-dimethylaminopropyl)carbodiimide hydrochloride (EDC.HCl), bromo-tris-pyrrolidino-phosnium hexafluorophosphate (PyBroP), O-(7-azabenzotriazol-1-yl)-1,1,3,3-tetramethyluronium hexafluorophosphate (HATU), or 1-cyclohexyl-3-methylpolystyrene-carbodiimide. The base includes a tertiary amine (preferably N,N-diisopropylethylamine or triethylamine, etc.). The inert solvent includes lower halocarbon solvents (preferably dichloromethane, dichloroethane, or chloroform, etc.), ethereal solvents (preferably tetrahydrofuran or dioxane), nitrile solvents (preferably acetonitrile, etc.), or amide solvents (preferably N,N-dimethylformamide, etc.). In case of need, 1-hydroxybenzotriazole (HOBT), HOBT-6-carboxaamidomethyl polystyrene, or 1-hydroxy-7-azabenzotriazole (HOAT) can be used as a reactant agent. Reaction temperature ranges from about −20° C. to 50° C., preferably about 0° C. to 40° C.

Alternatively, the novel ester (E′) of the present invention can be obtained by esterification via an acid chloride prepared from the carboxylic acid (C′) and a base in an inert solvent. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine, imidazole, poly-(4-vinylpyridine), etc.). The inert solvent includes lower halocarbon solvents (preferably dichloromethane, dichloroethane, or chloroform, etc.), ethereal solvents (preferably tetrahydrofuran or dioxane), amide solvents (preferably N,N-dimethylformamide, etc.), or aromatic solvents (preferably toluene or pyridine, etc.). Reaction temperature ranges from about −20° C. to 50° C., preferably about 0° C. to 40° C.

Alternatively, the novel pyrimidines (D′) and (E′) of the present invention are directly synthesized from the pyrimidine core (C), which is synthesized in Scheme 1. This coupling is performed with or without a base in an inert solvent. The base includes an alkali metal carbonate (preferably sodium carbonate or potassium carbonate, etc.), an alkali metal hydroxide (preferably sodium hydroxide, etc.), or a tertiary amine (preferably N,N-diisopropylethylamine, triethylamine, or N-methylmorpholine, etc.). The inert solvent includes lower alkyl alcohol solvents (preferably methanol, ethanol, 2-propanol, or butanol, etc.) or amide solvents (preferably N,N-dimethylformamide or 1-methyl-pyrrolidin-2-one, etc.). Reaction temperature ranges from about 50° C. to 200° C., preferably about 80° C. to 180° C. Also this reaction can be carried out under microwave conditions.

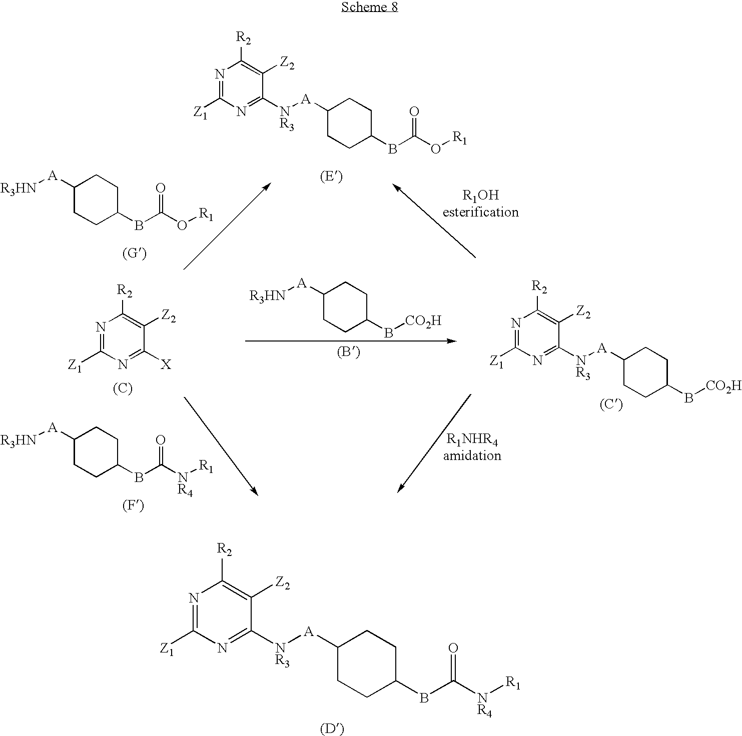

### EXAMPLES

The compounds of the invention and their synthesis are further illustrated by the following examples. The following examples are provided to further define the invention without, however, limiting the invention to the particulars of these examples. “Ambient temperature” as referred to in the following example is meant to indicate a temperature falling between 0° C. and 40° C. The following compounds are named by Beilstein Auto Nom Version 4.0, CS Chem Draw Ultra Version 7.0.1, CS Chem Draw Ultra Version 6.0.2, CS Chem Draw Ultra Version 6.0, or ACD Name Version 7.0.

Abbreviations used in the instant specification, particularly the Schemes and Examples, are as follows:

¹H NMR: proton nuclear magnetic resonance spectrum

AcOH: acetic acid

APCI: atmospheric pressure chemical ionization

(Boc)₂O: di-tertiary-butyl dicarbonate

BuLi: butyl lithium

BuOH: butanol

Cbz: carbobenzoxy

CDCl₃: deuterated chloroform

CH₂Cl₂: dichloromethane

CHCl₃: chloroform

CI: chemical ionization

DCM: dichloromethane

DIEA: diisopropylethylamine

DMSO: dimethyl sulfoxide

EDC-HCl: 1-(3-dimethylaminopropyl)-3-ethylcarbodiimide hydrochloride

EI: electron ionization

ESI: electrospray ionization

Et₃N: triethylamine

Et₂O: diethyl ether

EtOAc: acetic acid ethyl ester

EtOH: ethanol

FAB: fast atom bombardment

HOBt-H₂O: 1-hydroxybenzotriazole hydrate

H₂SO₄: sulfuric acid

HCl: hydrogen chloride

IPA: isopropanol

iPr₂NEt: diisopropylethylamine

K₂CO₃: potassium carbonate

Me₂NH: dimethylamine

MeNH₂: methylamine

MeOH: methanol

MgSO₄: magnesium sulfate

NaBH(OAc)₃: sodium triacetoxyborohydride

NaBH₃CN: sodium cyanoborohydride

NaBH₄: sodium borohydride

NaH: sodium hydride

NaHCO₃: sodium hydrogencarbonate

NH₃: ammonia

NH₄Cl: ammonium chloride

Pd/C: palladium carbon

POCl₃: phosphoryl chloride

SOCl₂: thionyl chloride

TFA: trifluoroacetic acid

THF: tetrahydrofuran

ZCl: benzyloxycarbonyl chloride

ZnBr₂: zinc bromide

s: singlet

d: doublet

t: triplet

q: qualtet

dd: doublet doublet

dt: doublet triplet

ddd: doublet doublet doublet

brs: broad singlet

m: multiplet

J: coupling constant

Hz: Hertz

### Example 1

### N′-(cis-4-{[4-Bromo-2-(trifluoromethoxy)benzyl]amino}cyclohexyl)-N,N-dimethylpyrimidine-4,6-diamine dihydrochloride

### Step A: Synthesis of (6-chloro-pyrimidin-4-yl)-dimethyl-amine

To a solution of 4,6-dichloro-pyrimidine (10.0 g) in THF (10 mL) were added iPr₂NEt (10.4 g) and 50% aqueous Me₂NH (6.05 g). The mixture was stirred at ambient temperature for 28 hr and poured into saturated aqueous NaHCO₃. The aqueous layer was extracted with CHCl₃ (three times). The combined organic layer was dried over MgSO₄, filtered, and concentrated under reduced pressure. The residue was suspended in Et₂O. The precipitate was collected by filtration, washed with Et₂O and dried under reduced pressure to give (6-chloro-pyrimidin-4-yl)-dimethyl-amine (6.37 g).

ESI MS m/e 157, M⁺; ¹H NMR (300 MHz, CDCl₃) δ 3.12 (s, 6H), 6.41 (s, 1H), 8.37 (s, 1H).

### Step B: Synthesis of N-(cis-4-bromo-2-trifluoromethoxy-benzyl)-cyclohexane-1,4-diamine

To a solution of (4-amino-cyclohexyl)-carbamic acid tert-butyl ester (6.72 g) in CHCl₃ (67 mL) were added 4-bromo-2-trifluoromethoxy-benzaldehyde (8.44 g), acetic acid (1.88 g), and NaBH(OAc)₃ (9.97 g). The mixture was stirred at ambient temperature for 4 hr and poured into saturated aqueous NaHCO₃. The aqueous layer was extracted with CHCl₃ (three times). The combined organic layer was dried over MgSO₄, filtrated, concentrated under reduced pressure, and purified by flash chromatography (silica gel, 33% EtOAc in hexane) to give [cis-4-(4-bromo-2-trifluoromethoxy-benzylamino)-cyclohexyl]-carbamic acid tert-butyl ester. To a solution of the above material (3.00 g) in EtOAc (30 mL) was added 4 M hydrogen chloride in EtOAc (60 mL). The mixture was stirred at ambient temperature for 1 hr and concentrated under reduced pressure. The residue was alkalized with saturated aqueous NaHCO₃ and the aqueous layer was extracted with CHCl₃ (seven times). The combined organic layer was dried over MgSO₄, filtered, and concentrated under reduced pressure to give N-(cis-4-bromo-2-trifluoromethoxy-benzyl)-cyclohexane-1,4-diamine (2.39 g).

ESI MS m/e 367, M⁺; ¹H NMR (300 MHz, CDCl₃) δ 1.22-1.96 (m, 8H), 2.51-2.71 (m, 1H), 2.87-3.13 (m, 1H), 3.74 (brs, 2H), 7.28-7.50 (m, 3H).

### Step C: Synthesis of N′(cis-4-{[4-bromo-2-(trifluoromethoxy)benzyl]amino}cyclohexyl)-N,N-dimethylpyrimidine-4,6-diamine dihydrochloride

A mixture of N-(cis-4-bromo-2-trifluoromethoxy-benzyl)-cyclohexane-1,4-diamine (466 mg), (6-chloro-pyrimidin-4-yl)-dimethyl-amine (200 mg), and ethylene glycol (0.5 mL) was stirred at reflux for 4 hr in a sealed tube. The mixture was poured into saturated aqueous NaHCO₃ and the aqueous layer was extracted with CHCl₃ (three times). The combined organic layer was dried over MgSO₄, filtered, concentrated under reduced pressure, and purified by flash chromatography (NH-silica gel, 50% EtOAc in hexane and silica gel, 5% MeOH in CHCl₃) to give N′-(cis-4-{[4-bromo-2-(trifluoromethoxy)benzyl]amino}cyclohexyl)-N,N-dimethylpyrimidine-4,6-diamine. To a solution of the above material in EtOAc (2 mL) was added 4 M hydrogen chloride in EtOAc (10 mL). The mixture was stirred at ambient temperature for 1 hr and concentrated under reduced pressure. The residue was suspended in Et₂O (20 mL) and the suspension was stirred at ambient temperature for 4 hr. The precipitate was collected by filtration, washed with Et₂O, and dried under reduced pressure to give N′-(cis-4-{[4-bromo-2-(trifluoromethoxy)benzyl]amino}cyclohexyl)-N,N-dimethylpyrimidine-4,6-diamine dihydrochloride (67 mg).

ESI MS m/e 488, M (free)+H⁺; ¹H NMR (300 MHz, CDCl₃) δ 1.64-1.86 (m, 2H), 1.96-2.34 (m, 8H), 2.98-3.44 (m, 8H), 4.27 (s, 2H), 7.40-7.59 (m, 3H), 8.06-8.24 (m, 2H).

### Example 2

### N-(cis-4-{[6-(1-Dimethylamino)pyrimidin-4-yl]amino}cyclohexyl)-3,4-difluorobenzamide hydrochloride

### Step A: Synthesis of (cis-4-{[1-(3,4-difluoro-phenyl)-methanoyl]-amino}-cyclohexyl)-carbamic acid tert-butyl ester

To a solution of 3,4-difluoro-benzoic acid (4.10 g) and (cis-4-amino-cyclohexyl)-carbamic acid tert-butyl ester (5.05 g) in DMF (50 mL) were added Et₃N (90 mL), HOBt-H₂O (5.41 g), and EDC-HCl (4.97 g). The mixture was stirred at ambient temperature for 17 hr. To the reaction mixture was added water (200 mL) and the suspension was stirred at ambient temperature for 10 min. The precipitate was collected by filtration, washed with H₂O and EtOH, and dried at 80° C. under reduced pressure to give (cis-4-{[1-(3,4-difluoro-phenyl)-methanoyl]-amino}-cyclohexyl)-carbamic acid tert-butyl ester (5.20 g).

ESI MS m/e 377, M+Na⁺; ¹H NMR (300 MHz, CDCl₃) δ 1.45 (s, 9H), 1.53-1.95 (m, 8 H), 3.60-3.74 (m, 1H), 4.00-4.16 (m, 1H), 4.50-4.68 (m, 1H), 5.95-6.09 (m, 1H), 7.15-7.28 (m, 1H), 7.43-7.68 (m, 2H).

### Step B: Synthesis of N-(cis-4-amino-cyclohexyl)-3,4-difluoro-benzamide

A solution of (cis-4-{[1-(3,4-difluoro-phenyl)-methanoyl]-amino}-cyclohexyl)-carbamic acid tert-butyl ester (5.20 g) in EtOAc (52 mL) was cooled on an ice-bath and 4 M hydrogen chloride in EtOAc (104 mL) was added. The mixture was stirred at ambient temperature for 1 hr and concentrated under reduced pressure. The residue was dissolved in 1 M aqueous NaOH and the aqueous layer was extracted with CHCl₃ (three times). The combined organic layer was dried over MgSO₄, filtered, concentrated under reduced pressure, and dried at 60° C. under reduced pressure to give N-(cis-4-amino-cyclohexyl)-3,4-difluoro-benzamide (3.00 g).

ESI MS m/e 255, M+H⁺; ¹H NMR (300 MHz, CDCl₃) δ 1.15-1.52 (m, 3H), 1.59-1.89 (m, 5H), 2.94-3.06 (m, 1H), 4.06-4.20 (m, 1H), 6.01-6.18 (m, 1H), 7.13-7.26 (m, 1H), 7.43-7.50 (m, 1H), 7.57-7.67 (m, 1H).

### Step C: Synthesis of N-(cis-4-{[6-(dimethylamino)pyrimidin-4-yl]amino}cyclohexyl)-3,4-difluorobenzamide hydrochloride

To a solution of N-(cis-4-amino-cyclohexyl)-3,4-difluoro-benzamide (442 mg) was added (6-chloro-pyrimidin-4-yl)-dimethyl-amine obtained in step A of example 1 (250 mg). The mixture was stirred at 180° C. for 8 hr in a sealed tube. To the mixture was added saturated aqueous NaHCO₃ and the aqueous layer was extracted with CHCl₃ (three times). The combined organic layer was dried over MgSO₄, filtered, concentrated under reduced pressure, and purified by flash chromatography (NH-silica gel, 33% to 50% EtOAc in hexane and silica gel, 3% MeOH in CHCl₃) to give N-(cis-4-{[6-(dimethylamino)pyrimidin-4-yl]amino}cyclohexyl)-3,4-difluorobenzamide. To a solution of the above material in EtOAc (10 mL) was added 4 M hydrogen chloride in EtOAc (0.2 mL). The mixture was stirred at ambient temperature for 1 hr and concentrated under reduced pressure. The residue was suspended in Et₂O (20 mL) and the suspension was stirred at ambient temperature for 4 hr. The precipitate was collected by filtration, washed with Et₂O, and dried at 70° C. under reduced pressure to give N-(cis-4-{[6-(dimethylamino)pyrimidin-4-yl]amino}cyclohexyl)-3,4-difluorobenzamide hydrochloride (99 mg).

ESI MS m/e 398, M (free)+Na⁺; ¹H NMR (300 MHz, CDCl₃) δ 1.69-2.15 (m, 8H), 3.00-3.42 (m, 6H), 3.69-3.81 (m, 1H), 4.03-4.21 (m, 1H), 5.26 (s, 1H), 6.66-6.80 (m, 1H), 7.13-7.26 (m, 1H), 7.51-7.62 (m, 1H), 7.68-7.80 (m, 1H), 8.01 (s, 1H), 8.68-8.91 (m, 1H), 13.84-14.09 (m, 1H).

### Example 3

### N-[cis-4-({[6-(Dimethylamino)pyrimidin-4-yl]amino}methyl)cyclohexyl]-3,4-difluorobenzamide hydrochloride

### Step A: Synthesis of (cis-4-hydroxymethyl-cyclohexyl)-carbamic acid tert-butyl ester

A suspension of cis-4-amino-cyclohexanecarboxylic acid (244 g) in MeOH (2.45 L) was cooled to −8° C. Thionyl chloride (45.0 mL) was added dropwise. The mixture was stirred at ambient temperature for 4.5 hr and concentrated under reduced pressure to give a white solid. To a suspension of the above solid in CHCl₃ (3.00 L) were added triethylamine (261 mL) and (Boc)₂O (409 g) successively. The mixture was stirred at ambient temperature for 5 hr and poured into water. The aqueous layer was extracted with CHCl₃ (three times). The combined organic layer was dried over MgSO₄, filtrated, concentrated under reduced pressure, and purified by flash chromatography (silica gel, CHCl₃ to 10% MeOH in CHCl₃) to give a colorless oil (531 g). To a suspension cooled at −4° C. of lithium aluminum hydride (78.3 g) in Et₂O (7.9 L) was added a solution of the above oil (530.9 g) in Et₂O (5.3 L) below 0° C. The resulting suspension was stirred at ambient temperature for 2 hr. The mixture was cooled on an ice-bath, quenched with cold water, and filtrated through a pad of celite. The filtrate was dried over MgSO₄, filtrated, and concentrated under reduced pressure. The precipitate was suspended in hexane (300 mL), filtrated, washed with hexane, and dried under reduced pressure to give (cis-4-hydroxymethyl-cyclohexyl)-carbamic acid tert-butyl ester (301 g).

ESI MS m/e 252, M+Na⁺; ¹H NMR (300 MHz, CDCl₃) δ 1.16-1.36 (m, 2H), 1.45 (s, 9H), 1.52-1.77 (m, 7H), 3.51 (d, J=6.2 Hz, 2H), 3.75 (brs, 1H), 4.30-4.82 (m, 1H).

### Step B: Synthesis of [cis-4-(benzyloxycarbonylamino-methyl)-cyclohexyl]-carbamic acid tert-butyl ester

To a solution of (cis-4-hydroxymethyl-cyclohexyl)-carbamic acid tert-butyl ester (17.7 g) in THF (245 mL) were added triphenylphosphine (20.2 g) and phthalimide (11.4 g) successively. The resulting suspension was cooled on an ice-bath and 40% diethyl azodicarboxylate in toluene (33.6 mL) was added over 1 hr. The mixture was stirred at ambient temperature for 2.5 days, concentrated under reduced pressure, and purified by flash chromatography (silica gel, 33% EtOAc in hexane) to give a white solid. To a suspension of the above solid (27.5 g) in EtOH (275 mL) was added hydrazine hydrate (5.76 g). The mixture was stirred at reflux for 2.25 hr, cooled to ambient temperature, and concentrated under reduced pressure. The precipitate was dissolved in 10% aqueous sodium hydroxide (350 mL). The aqueous layer was extracted with CHCl₃ (three times). The combined organic layer was dried over MgSO₄, filtrated, and concentrated under reduced pressure. To a solution of the above residue in CHCl₃ (275 mL) was added triethylamine (8.54 g). The resulting solution was cooled to 0° C. and ZCl (14.4 g) was added below 5° C. The mixture was stirred at ambient temperature for 16 hr and poured into saturated aqueous NaHCO₃. The aqueous layer was extracted with CHCl₃ (three times). The combined organic layer was dried over MgSO₄, filtrated, concentrated under reduced pressure, and purified by flash chromatography (silica gel, 2% MeOH in CHCl₃) to give [cis-4-(benzyloxycarbonylamino-methyl)-cyclohexyl]-carbamic acid tert-butyl ester (25.3 g).

ESI MS m/e 385, M+Na⁺; ¹H NMR (300 MHz, CDCl₃) δ 1.13-1.31 (m, 2H), 1.44 (s, 9H), 1.48-1.75 (m, 7H), 3.10 (t, J=6.4 Hz, 2H), 3.72 (brs, 1H), 4.42-4.76 (m, 1H), 4.76-4.92 (m, 1H), 5.09 (s, 2H), 7.27-7.38 (m, 5H).

### Step C: Synthesis of (cis-4-amino-cyclohexylmethyl)-carbamic acid benzyl ester

To a solution of [cis-4-(benzyloxycarbonylamino-methyl)-cyclohexyl]-carbamic acid tert-butyl ester (12.9 g) in EtOAc (129 mL) was added 4 M hydrogen chloride in EtOAc (129 mL). The mixture was stirred at ambient temperature for 3 hr, filtrated, washed with EtOAc, and dried under reduced pressure. To the residue was added saturated aqueous NaHCO₃. The aqueous layer was extracted with CHCl₃ (five times). The combined organic layer was dried over MgSO₄, filtrated, concentrated under reduced pressure, and dried under reduced pressure to give (cis-4-amino-cyclohexylmethyl)-carbamic acid benzyl ester (8.88 g).

ESI MS m/e 263, M+H⁺; ¹H NMR (300 MHz, CDCl₃) δ 1.36-1.98 (m, 9H), 2.96-3.32 (m, 3H), 5.12 (brs, 3H), 7.36 (s, 5H).

### Step D: Synthesis of [cis-4-(3,4-difluoro-benzoylamino)-cyclohexylmethyl]-carbamic acid benzyl ester

To a solution of (cis-4-amino-cyclohexylmethyl)-carbamic acid benzyl ester (2.00 g) in CHCl₃ (16 mL) were added Et₃N (2.23 mL) and 3,4-difluoro-benzoyl chloride (1.48 g) in CHCl₃ (4 mL). The mixture was stirred at ambient temperature for 12 hr and poured into saturated aqueous NaHCO₃. The aqueous layer was extracted with CHCl₃ (three times). The combined organic layer was dried over MgSO₄, filtered, concentrated under reduced pressure, and purified by medium-pressure liquid chromatography (NH-silica gel, 20% to 50% EtOAc in hexane) to give [cis-4-(3,4-difluoro-benzoylamino)-cyclohexylmethyl]-carbamic acid benzyl ester (2.66 g).

ESI MS m/e 425, M⁺; ¹H NMR (300 MHz, CDCl₃) δ 1.22-1.44 (m, 2H), 1.57-1.88 (m, 6H), 3.07-3.25 (m, 2H), 4.08-4.28 (m, 1H), 4.78-4.93 (m, 1H), 5.10 (s, 2H), 6.02-6.24 (m, 1H), 7.13-7.39 (m, 6H), 7.43-7.52 (m, 1H), 7.58-7.68 (m, 1H).

### Step E: Synthesis of N-(cis-4-aminomethyl-cyclohexyl)-3,4-difluoro-benzamide

To a solution of [cis-4-(3,4-difluoro-benzoylamino)-cyclohexylmethyl]-carbamic acid benzyl ester (2.60 g) in MeOH (26 mL) was added 10% Pd/C (260 mg). The mixture was stirred at ambient temperature under hydrogen atmosphere for 84 hr. The mixture was filtrated through a pad of celite, concentrated under reduced pressure, and purified by medium-pressure liquid chromatography (NH-silica gel, 9% to 17% EtOAc in hexane and silica gel, 1% MeOH in CHCl₃) to give N-(cis-4-aminomethyl-cyclohexyl)-3,4-difluoro-benzamide (1.43 g).

ESI MS m/e 269, M+H⁺; ¹H NMR (300 MHz, CDCl₃) δ 1.13-1.86 (m, 9H), 2.64 (d, J=6.5 Hz, 2H), 4.16-4.28 (m, 1H), 6.09-6.30 (m, 1H), 7.15-7.27 (m, 1H), 7.46-7.53 (m, 1H), 7.58-7.67 (m, 1H).

### Step F: Synthesis of N-[cis-4-({[6-(dimethylamino)pyrimidin-4-yl]amino}methyl)cyclohexyl]-3,4-difluorobenzamide hydrochloride

To a solution of N-(cis-4-aminomethyl-cyclohexyl)-3,4-difluoro-benzamide (373 mg) in BuOH (1 mL) was added (6-chloro-pyrimidin-4-yl)-dimethyl-amine obtained in step A of example 1 (200 mg). The mixture was heated in a microwave synthesizer at 220° C. for 20 min. To the mixture was added saturated aqueous NaHCO₃ and the aqueous layer was extracted with CHCl₃ (three times). The combined organic layer was dried over MgSO₄, filtrated, concentrated under reduced pressure, and purified by medium-pressure liquid chromatography (NH-silica gel, 20% to 50% EtOAc in hexane) to give N-[cis-4-({[6-(dimethylamino)pyrimidin-4-yl]amino}methyl)cyclohexyl]-3,4-difluorobenzamide. To a solution of the above material in EtOAc (10 mL) was added 4 M hydrogen chloride in EtOAc (0.5 mL). The mixture was stirred at ambient temperature for 30 min and concentrated under reduced pressure. A suspension of the above material in Et₂O (12 mL) was stirred at ambient temperature for 2 hr. The precipitate was collected by filtration, washed with Et₂O, and dried at 70° C. under reduced pressure to give N-[cis-4-({[6-(dimethylamino) pyrimidin-4-yl]amino}-methyl)cyclohexyl]-3,4-difluorobenzamide hydrochloride (106 mg).

ESI MS m/e 390, M (free)+H⁺; ¹H NMR (300 MHz, CDCl₃) δ 1.31-2.14 (m, 8H), 2.96-3.46 (m, 8H), 4.40-4.61 (m, 1H), 5.18 (s, 1H), 7.14-7.35 (m, 2H), 7.83-8.09 (m, 3H), 8.79-9.14 (m, 1H).

### Example 4

### N-[(cis-4-{[6-(Dimethylamino)pyrimidin-4-yl]amino}cyclohexyl)methyl]-3,4-difluorobenzamide hydrochloride

### Step A: Synthesis of {cis-4-[(3,4-difluoro-benzoylamino)-methyl]-cyclohexyl}-carbamic acid tert-butyl ester

To a solution of [cis-4-(benzyloxycarbonylamino-methyl)-cyclohexyl]-carbamic acid tert-butyl ester obtained in step B of example 3 (5.00 g) in MeOH (50 mL) was added 10% Pd/C (500 mg). The mixture was stirred at ambient temperature under hydrogen atmosphere for 84 hr, filtrated through a pad of celite, and concentrated under reduced pressure to give a pale brown oil. To a solution of the above oil in CHCl₃ (40 mL) were added Et₃N (4.03 mL) and 3,4-difluoro-benzoyl chloride (2.68 g) in CHCl₃ (10 mL). The mixture was stirred at ambient temperature for 12 hr. To the mixture was added saturated aqueous NaHCO₃ and the aqueous layer was extracted with CHCl₃ (three times). The combined organic layer was dried over MgSO₄, filtered, concentrated under reduced pressure, and purified by medium-pressure liquid chromatography (NH-silica gel, 50% EtOAc in hexane) to give {cis-4-[(3,4-difluoro-benzoylamino)-methyl]-cyclohexyl}-carbamic acid tert-butyl ester (3.48 g).

ESI MS m/e 391, M+Na⁺; ¹H NMR (300 MHz, CDCl₃) δ 1.19-1.81 (m, 16H), 3.33-3.43 (m, 2H), 3.68-3.79 (m, 1H), 4.54-4.73 (m, 1H), 6.10-6.21 (m, 1H), 7.17-7.27 (m, 1H), 7.46-7.54 (m, 1H), 7.59-7.68 (m, 1H).

### Step B: Synthesis of N-(cis-4-amino-cyclohexylmethyl)-3,4-difluoro-benzamide

To a solution of {cis-4-[(3,4-difluoro-benzoylamino)-methyl]-cyclohexyl}-carbamic acid tert-butyl ester (3.48 g) in EtOAc (35 mL) was added 4 M hydrogen chloride in EtOAc (35 mL). The mixture was stirred at ambient temperature for 12 hr and concentrated under reduced pressure. The residue was dissolved in 1 M aqueous NaOH and the aqueous layer was extracted with CHCl₃ (three times). The combined organic layer was dried over MgSO₄, filtrated, concentrated under reduced pressure to give N-(cis-4-amino-cyclohexylmethyl)-3,4-difluoro-benzamide (2.50 g).

ESI MS m/e 269, M+H⁺; ¹H NMR (300 MHz, CDCl₃) δ 1.16-1.81 (m, 9H), 2.93-3.08 (m, 1H), 3.32-3.42 (m, 2H), 6.41-6.57 (m, 1H), 7.14-7.27 (m, 1H), 7.48-7.57 (m, 1H), 7.60-7.71 (m, 1H).

### Step C: Synthesis of N-[(cis-4-{[6-(dimethylamino)pyrimidin-4-yl]amino}cyclohexyl)methyl]-3,4-difluorobenzamide hydrochloride

To a solution of N-(cis-4-amino-cyclohexylmethyl)-3,4-difluoro-benzamide (469 mg) in BuOH (1 mL) was added (6-chloro-pyrimidin-4-yl)-dimethyl-amine obtained in step A of example 1 (250 mg). The mixture was heated in a microwave synthesizer at 220° C. for 20 min. To the mixture was added saturated aqueous NaHCO₃ and the aqueous layer was extracted with CHCl₃ (three times). The combined organic layer was dried over MgSO₄, filtrated, concentrated under reduced pressure, and purified by medium-pressure liquid chromatography (NH-silica gel, 20% to 50% EtOAc in hexane) to give N-[(cis-4-{[6-(dimethylamino)pyrimidin-4-yl]amino}cyclohexyl)methyl]-3,4-difluorobenzamide. To a solution of the above material in EtOAc (10 mL) was added 4 M hydrogen chloride in EtOAc (0.5 mL). The mixture was stirred at ambient temperature for 30 min and concentrated under reduced pressure. A suspension of the residue in Et₂O (12 mL) was stirred at ambient temperature for 2 hr. The precipitate was collected by filtration, washed with Et₂O, and dried at 70° C. under reduced pressure to give N-[(cis-4-{[6-(dimethylamino)pyrimidin-4-yl]amino}-cyclohexyl)methyl]-3,4-difluorobenzamide hydrochloride (82 mg).

ESI MS m/e 390, M (free)+H⁺; ¹H NMR (300 MHz, CDCl₃) δ 1.50-2.04 (m, 9H), 2.93-3.57 (m, 8H), 3.67-3.85 (m, 1H), 5.23 (s, 1H), 6.85-7.35 (m, 2H), 7.73-8.05 (m, 3H), 8.75-9.01 (m, 1H), 13.64-13.95 (m, 1H).

### Example 5

### N-(cis-4-{[6-(Dimethylamino)-2-methylpyrimidin-4-yl]amino}cyclohexyl)-3,4-difluorobenzamide hydrochloride

### Step A: Synthesis of 4,6-dichloro-2-methyl-pyrimidine

A suspension of 2-methyl-pyrimidine-4,6-diol (20.0 g) in POCl₃ (162 mL) was stirred at reflux for 4 hr and cooled to ambient temperature. The mixture was poured into ice water (3 L). The aqueous layer was extracted with CHCl₃ (three times). The combined organic layer was dried over MgSO₄, filtrated, and concentrated under reduced pressure to give 4,6-dichloro-2-methyl-pyrimidine (22.37 g).

CI MS m/e 163, M⁺; ¹H NMR (300 MHz, CDCl₃) δ 2.71 (s, 3H), 7.25 (s, 1H).

### Step B: Synthesis of (6-chloro-2-methyl-pyrimidin-4-yl)-dimethyl-amine

To a solution of 4,6-dichloro-2-methyl-pyrimidine (11.1 g) in THF (110 mL) were added iPr₂NEt (14.2 mL) and 50% aqueous Me₂NH (8.5 mL). The mixture was stirred at ambient temperature for 2 hr. To the mixture was added 50% aqueous Me₂NH (3.5 mL) and stirred at ambient temperature for 7 hr and concentrated under reduced pressure. To the residue was added saturated aqueous NaHCO₃ and the aqueous layer was extracted with CHCl₃ (three times). The combined organic layer was dried over MgSO₄, filtered, concentrated under reduced pressure, and dried under reduced pressure to give (6-chloro-2-methyl-pyrimidin-4-yl)-dimethyl-amine (11.6 g).

ESI MS m/e 172, M+H⁺; ¹H NMR (300 MHz, CDCl₃) δ 2.49 (s, 3H), 3.10 (s, 6H), 6.24 (s, 1H).

### Step C: Synthesis of N-(cis-4-{[6-(dimethylamino)-2-methylpyrimidin-4-yl]amino}cyclohexyl)-3,4-difluorobenzamide hydrochloride

To a solution of N-(cis-4-amino-cyclohexyl)-3,4-difluoro-benzamide obtained in step B of example 2 (407 mg) in BuOH (1 mL) was added (6-chloro-2-methyl-pyrimidin-4-yl)-dimethyl-amine (250 mg). The mixture was heated in a microwave synthesizer at 200° C. for 20 min and 230° C. for 20 min. To the mixture was added saturated aqueous NaHCO₃ and the aqueous layer was extracted with CHCl₃ (three times). The combined organic layer was dried over MgSO₄, filtrated, concentrated under reduced pressure, and purified by medium-pressure liquid chromatography (NH-silica gel, 20% to 50% EtOAc in hexane) to give N-(cis-4-{[6-(dimethylamino)-2-methylpyrimidin-4-yl]amino}-cyclohexyl)-3,4-difluorobenzamide. To a solution of the above material in EtOAc (10 mL) was added 4 M hydrogen chloride in EtOAc (0.2 mL). The mixture was stirred at ambient temperature for 1 hr and concentrated under reduced pressure. A suspension of the residue in Et₂O (12 mL) was stirred at ambient temperature for 2 hr. The precipitate was collected by filtration, washed with Et₂O, and dried at 70° C. under reduced pressure to give N-(cis-4-{[6-(dimethylamino-2-methylpyrimidin-4-yl]amino}cyclohexyl)-3,4-difluorobenzamide hydrochloride (325 mg).

ESI MS m/e 412, M (free)+Na⁺; ¹H NMR (300 MHz, CDCl₃) δ 1.63-2.03 (m, 8H), 2.49 (s, 3H), 2.91-3.43 (m, 6H), 3.67-3.79 (m, 1H), 4.03-4.22 (m, 1H), 5.15 (s, 1H), 6.89-7.02 (m, 1H), 7.14-7.27 (m, 1H), 7.56-7.64 (m, 1H), 7.69-7.81 (m, 1H), 8.40-8.55 (m, 1H).

### Example 6

### 3-Chloro-N-(cis-4-{[6-(dimethylamino)-2-methylpyrimidin-4-yl]amino}cyclohexyl)-4-fluorobenzamide hydrochloride

### Step A: Synthesis of cis-N-benzyl-cyclohexane-1,4-diamine

To a solution of (cis-4-amino-cyclohexyl)-carbamic acid tert-butyl ester (5.00 g) in CHCl₃ (100 mL) were added benzaldehyde (2.48 g) and acetic acid (1.40 g). The mixture was stirred at ambient temperature for 1 hr. To the mixture was added NaBH(OAc)₃ (7.42 g) and the mixture was stirred at ambient temperature for 15 hr. The reaction was quenched with saturated aqueous NaHCO₃ and the aqueous layer was extracted with CHCl₃ (three times). The combined organic layer was dried over MgSO₄, filtrated, concentrated under reduced pressure, and purified by medium-pressure liquid chromatography (silica gel, 2% to 9% MeOH in CHCl₃) to give (cis-4-benzylamino-cyclohexyl)-carbamic acid tert-butyl ester (76.9 g). To a solution of the above material (76.9 g) in EtOAc (77 mL) was added 4 M hydrogen chloride in EtOAc (38.5 mL). The mixture was stirred at ambient temperature for 10 hr and concentrated under reduced pressure. The residue was dissolved in 2M aqueous NaOH (150 mL) and the aqueous layer was extracted with CHCl₃ (three times). The combined organic layer was dried over MgSO₄, filtered, concentrated under reduced pressure, and dried under reduced pressure to give cis-N-benzyl-cyclohexane-1,4-diamine (4.12 g).

ESI MS m/e 205, M+H⁺; ¹H NMR (300 MHz, CDCl₃) δ 1.42-1.72 (m, 8H), 2.63-2.74 (m, 1H), 2.80-2.91 (m, 1H), 3.77 (s, 2H), 7.20-7.39 (m, 5H).

### Step B: Synthesis of N-(cis-4-benzylamino-cyclohexyl)-2,N′,N′-trimethyl-pyrimidine-4,6-diamine

To a solution of (6-chloro-2-methyl-pyrimidin-4-yl)-dimethyl-amine obtained in step B of example 5 (763 mg) in BuOH (0.8 mL) was added cis-N-benzyl-cyclohexane-1,4-diamine (1.00 g). The mixture was heated in a microwave synthesizer at 220° C. for 25 min. To the mixture was added saturated aqueous NaHCO₃ and the aqueous layer was extracted with CHCl₃ (three times). The combined organic layer was dried over MgSO₄, filtrated, concentrated under reduced pressure, and purified by medium-pressure liquid chromatography (NH-silica gel, 9% to 60% EtOAc in hexane) to give N-(cis-4-benzylamino-cyclohexyl)-2,N′,N′-trimethyl-pyrimidine-4,6-diamine (952 mg).

ESI MS m/e 340, M+H⁺; ¹H NMR (300 MHz, CDCl₃) δ 1.47-1.92 (m, 8H), 2.35 (s, 3H), 2.63-2.74 (m, 1H), 3.04 (s, 6H), 3.56-3.69 (m, 1H), 3.79 (s, 2H), 4.67-4.80 (m, 1H), 5.14 (s, 1H), 7.20-7.36 (m, 5H).

### Step C: Synthesis of N-(cis-4-amino-cyclohexyl)-2,N′,N′-trimethyl-pyrimidine-4,6-diamine

To a solution of N-(cis-4-benzylamino-cyclohexyl)-2,N,N′-trimethyl-pyrimidine-4,6-diamine (940 mg) in MeOH (9.4 mL) was added 20% Pd(OH)₂ (188 mg). The mixture was stirred at 50° C. under hydrogen atmosphere for 10 hr. The mixture was filtrated through a pad of celite, concentrated under reduced pressure, and purified by medium-pressure liquid chromatography (NH-silica gel, 2% to 5% MeOH in CHCl₃) to give N-(cis-4-amino-cyclohexyl)-2,N′,N′-trimethyl-pyrimidine-4,6-diamine (566 mg).

ESI MS m/e 250, M+H⁺; ¹H NMR (300 MHz, CDCl₃) δ 1.05-1.89 (m, 10H), 2.35 (s, 3H), 2.75-2.90 (m, 1H), 3.05 (s, 6H), 3.54-3.70 (m, 1H), 4.68-4.82 (m, 1H), 5.14 (s, 1H).

### Step D: Synthesis of 3-chloro-N-(cis-4-{[6-(dimethylamino)-2-methylpyrimidin-4-yl]amino}-cyclohexyl)-4-fluorobenzamide hydrochloride

To a solution of 3-chloro-4-fluoro-benzoic acid (192 mg) and N-(cis-4-amino-cyclohexyl)-2,N′,N′-trimethyl-pyrimidine-4,6-diamine (250 mg) in DMF (4 mL) were added Et₃N (0.34 mL), HOBt-H₂O (230 mg), and EDC-HCl (211 mg). The mixture was stirred at ambient temperature for 12 hr. To the mixture was added water (20 mL) and the aqueous layer was extracted with CHCl₃ (three times). The combined organic layer was dried over MgSO₄, filtrated, concentrated under reduced pressure, and purified by medium-pressure liquid chromatography (NH-silica gel, 25% to 50% EtOAc in hexane) to give 3-chloro-N-(cis-4-{[6-(dimethylamino)-2-methylpyrimidin-4-yl]amino}-cyclohexyl)-4-fluorobenzamide. To a solution of the above material in EtOAc (10 mL) was added 4 M hydrogen chloride in EtOAc (0.2 mL). The mixture was stirred at ambient temperature for 1 hr and concentrated. The residue was suspended in Et₂O (20 mL) and the suspension was stirred at ambient temperature for 2 hr. The precipitate was collected by filtration, washed with Et₂O, and dried at 70° C. under reduced pressure to give 3-chloro-N-(cis-4-{[6-(dimethylamino)-2-methylpyrimidin-4-yl]amino}-cyclohexyl)-4-fluorobenzamide hydrochloride (196 mg).

ESI MS m/e 406, M (free)+H⁺; ¹H NMR (300 MHz, CDCl₃) δ 1.62-2.00 (m, 8H), 2.49 (s, 3H), 2.99-3.40 (m, 6H), 3.67-3.79 (m, 1H), 4.02-4.20 (m, 1H), 5.15 (s, 1H), 6.59-6.70 (m, 1H), 7.11-7.26 (m, 1H), 7.67-7.79 (m, 1H), 7.89-8.02 (m, 1H), 8.48-8.61 (m, 1H).

### Example 7

### N-(cis-4-{[6-(Dimethylamino)-2-methylpyrimidin-4-yl]amino}cyclohexyl)-4-fluorobenzamide hydrochloride

To a solution of N-(cis-4-amino-cyclohexyl)-2,N′,N′-trimethyl-pyrimidine-4,6-diamine obtained in step C of example 6 (250 mg) in CHCl₃ (3 mL) were added Et₃N (0.29 mL) and 4-fluoro-benzoyl chloride (174 mg). The mixture was stirred at ambient temperature for 12 hr. The reaction was quenched with saturated aqueous NaHCO₃ and the aqueous layer was extracted with CHCl₃ (three times). The combined organic layer was dried over MgSO₄, filtered, concentrated under reduced pressure, and purified by medium-pressure liquid chromatography (NH-silica gel, 25% to 50% EtOAc in hexane) to give N-(cis-4-{[6-(dimethylamino)-2-methylpyrimidin-4-yl]amino}-cyclohexyl)-4-fluorobenzamide. To a solution of the above material in EtOAc (10 mL) was added 4 M hydrogen chloride in EtOAc (0.2 mL). The mixture was stirred at ambient temperature for 1 hr and concentrated under reduced pressure. The residue was suspended in Et₂O (20 mL) and the suspension was stirred at ambient temperature for 2 hr. The precipitate was collected by filtration, washed with Et₂O, and dried at 70° C. under reduced pressure to give N-(cis-4-{[6-(dimethylamino)-2-methylpyrimidin-4-yl]amino}cyclohexyl)-4-fluorobenzamide hydrochloride (255 mg).

ESI MS m/e 372, M (free)+H⁺; ¹H NMR (300 MHz, CDCl₃) δ 1.66-2.03 (m, 8H), 2.49 (s, 3H), 2.93-3.43 (m, 6H), 3.64-3.78 (m, 1H), 4.04-4.20 (m, 1H), 5.14 (s, 1H), 6.43-6.56 (m, 1H), 7.05-7.15 (m, 2H), 7.75-7.91 (m, 2H), 8.47-8.63 (m, 1H).

### Example 8

### 3,4-Dichloro-N-(cis-4-{[6-(dimethylamino)-2-methylpyrimidin-4-yl]amino}cyclohexyl)-benzamide hydrochloride

Using the procedure for the step A of example 7, the title compound was obtained.

ESI MS m/e 422, M (free)⁺; ¹H NMR (300 MHz, CDCl₃) δ 1.63-2.02 (m, 8H), 2.49 (s, 3H), 2.96-3.38 (m, 6H), 3.67-3.80 (m, 1H), 4.02-4.21 (m, 1H), 5.14 (s, 1H), 6.69-6.80 (m, 1H), 7.47-7.53 (m, 1H), 7.62-7.70 (m, 1H), 7.93-8.00 (m, 1H), 8.48-8.59 (m, 1H), 13.70-13.90 (m, 1H).

### Example 9

### 4-Chloro-N-(cis-4-{[6-(dimethylamino)-2-methylpyrimidin-4-yl]amino}cyclohexyl)-3-fluorobenzamide hydrochloride

Using the procedure for the step D of example 6, the title compound was obtained.

ESI MS m/e 406, M (free)+H⁺; ¹H NMR (300 MHz, CDCl₃) δ 1.66-2.07 (m, 8H), 2.48 (s, 3H), 2.94-3.40 (m, 6H), 3.66-3.79 (m, 1H), 4.00-4.21 (m, 1H), 5.14 (s, 1H), 6.88-7.00 (m, 1H), 7.40-7.48 (m, 1H), 7.52-7.60 (m, 1H), 7.65-7.73 (m, 1H), 8.45-8.54 (m, 1H), 13.66-13.86 (m, 1H).

### Example 10

### 3-Chloro-N-(cis-4-{[6-(dimethylamino)-2-methylpyrimidin-4-yl]amino}cyclohexyl)-5-fluorobenzamide hydrochloride

Using the procedure for the step D of example 6, the title compound was obtained.

ESI MS m/e 406, M (free)+H⁺; ¹H NMR (300 MHz, CDCl₃) δ 1.61-2.07 (m, 8H), 2.49 (s, 3H), 2.96-3.41 (m, 6H), 3.65-3.79 (m, 1H), 4.00-4.22 (m, 1H), 5.14 (s, 1H), 6.78-6.88 (m, 1H), 7.16-7.23 (m, 1H), 7.42-7.50 (m, 1H), 7.60-7.64 (m, 1H), 8.36-8.62 (m, 1H), 13.75-13.95 (m, 1H).

### Example 11

### N-(cis-4-{[6-(Dimethylamino)-2-methylpyrimidin-4-yl]amino}cyclohexyl)-3,4,5-trifluorobenzamide hydrochloride

Using the procedure for the step D of example 6, the title compound was obtained.

ESI MS m/e 408, M (free)+H⁺; ¹H NMR (300 MHz, CDCl₃) δ 1.64-2.04 (m, 8H), 2.48 (s, 3H), 2.92-3.42 (m, 6H), 3.65-3.79 (m, 1H), 4.00-4.20 (m, 1H), 5.15 (s, 1H), 6.73-6.84 (m, 1H), 7.48-7.58 (m, 2H), 8.47-8.60 (m, 1H), 13.70-13.86 (m, 1H).

### Example 12

### 5-Bromo-N-(cis-4-{[6-(dimethylamino)-2-methylpyrimidin-4-yl]amino}cyclohexyl)-nicotinamide dihydrochloride

Using the procedure for the step D of example 6, the title compound was obtained.

ESI MS m/e 433, M (free)⁺; ¹H NMR (300 MHz, CDCl₃) δ 1.67-2.18 (m, 8H), 2.49 (s, 3H), 2.91-3.45 (m, 6H), 3.60-3.80 (m, 1H), 4.10-4.28 (m, 1H), 5.11-5.20 (m, 1H), 7.70-7.87 (m, 1H), 8.33-8.49 (m, 1H), 8.60-8.67 (m, 1H), 8.90-9.02 (m, 1H), 9.17-9.30 (m, 1H).

### Example 13

### N-(cis-4-{[6-(Dimethylamino)-2-methylpyrimidin-4-yl]amino}cyclohexyl)-3,5-difluorobenzamide hydrochloride

Using the procedure for the step A of example 7, the title compound was obtained.

ESI MS m/e 390, M (free)+H⁺; ¹H NMR (300 MHz, CDCl₃) δ 1.63-2.03 (m, 8H), 2.48 (s, 3H), 2.99-3.45 (m, 6H), 3.69-3.79 (m, 1H), 4.03-4.19 (m, 1H), 5.14 (s, 1H), 6.58-6.71 (m, 1H), 6.86-6.98 (m, 1H), 7.28-7.44 (m, 2H), 8.50-8.64 (m, 1H), 13.75-13.93 (m, 1H).

### Example 14

### N-(cis-4-{[6-(Dimethylamino)-2-methylpyrimidin-4-yl]amino}cyclohexyl)-4-fluoro-3-(trifluoromethyl)benzamide hydrochloride

Using the procedure for the step A of example 7, the title compound was obtained.

ESI MS m/e 440, M (free)+H⁺; ¹H NMR (300 MHz, CDCl₃) δ 1.65-2.03 (m, 8H), 2.49 (s, 3H), 2.97-3.40 (m, 6H), 3.67-3.81 (m, 1H), 4.02-4.23 (m, 1H), 5.15 (s, 1H), 6.63-6.79 (m, 1H), 7.19-7.31 (m, 1H), 7.97-8.08 (m, 1H), 8.13-8.20 (m, 1H), 8.50-8.60 (m, 1H), 13.74-13.88 (m, 1H).

### Example 15

### N-(cis-4-{[6-(Dimethylamino)-2-methylpyrimidin-4-yl]amino}cyclohexyl)-3-fluoro-4-(trifluoromethyl)benzamide hydrochloride

Using the procedure for the step A of example 7, the title compound was obtained.

ESI MS m/e 462, M (free)+Na⁺; ¹H NMR (300 MHz, CDCl₃) δ 1.64-2.06 (m, 8H), 2.49 (s, 3H), 2.97-3.39 (m, 6H), 3.67-3.81 (m, 1H), 4.02-4.23 (m, 1H), 5.15 (s, 1H), 6.76-6.95 (m, 1H), 7.52-7.81 (m, 2H), 8.47-8.62 (m, 1H), 13.71-13.85 (m, 1H).

### Example 16

### 3-Chloro-N-(cis-4-{[6-(dimethylamino)-2-methylpyrimidin-4-yl]amino}cyclohexyl)-4-(trifluoromethoxy)benzamide hydrochloride

Using the procedure for the step A of example 7, the title compound was obtained.

ESI MS m/e 494, M (free)+Na⁺; ¹H NMR (300 MHz, CDCl₃) δ 1.60-2.06 (m, 8H), 2.49 (s, 3H) 2.95-3.40 (m, 6H), 3.70-3.78 (m, 1H), 4.02-4.24 (m, 1H), 5.15 (s, 1H), 6.59-6.72 (m, 1H), 7.34-7.41 (m, 1H), 7.71-7.80 (m, 1H), 7.96-8.04 (m, 1H), 8.48-8.62 (m, 1H), 13.75-13.90 (m, 1H).

### Example 17

### N-(cis-4-{[6-(Dimethylamino)-2-methylpyrimidin-4-yl]amino}cyclohexyl)-3-(trifluoromethyl)-benzamide hydrochloride

Using the procedure for the step A of example 7, the title compound was obtained.

ESI MS m/e 444, M (free)+Na⁺; ¹H NMR (300 MHz, CDCl₃) δ 1.66-2.17 (m, 8H), 2.49 (s, 3H), 2.97-3.38 (m, 6H), 3.65-3.80 (m, 1H), 4.06-4.23 (m, 1H), 5.15 (s, 1H), 6.59-6.71 (m, 1H), 7.52-7.62 (m, 1H), 7.69-7.80 (m, 1H), 7.93-8.02 (m, 1H), 8.13 (s, 1H), 8.51-8.68 (m, 1H), 13.81-13.96 (m, 1H).

### Example 18

### N-(cis-4-{[6-(Dimethylamino)-2-methylpyrimidin-4-yl]amino}cyclohexyl)-3-(trifluoromethoxy)benzamide hydrochloride

Using the procedure for the step A of example 7, the title compound was obtained.

ESI MS m/e 438, M (free)+Na⁺; ¹H NMR (300 MHz, CDCl₃) δ 1.68-2.06 (m, 8H), 2.49 (s, 3H), 2.94-3.44 (m, 6H), 3.67-3.81 (m, 1H), 4.03-4.23 (m, 1H), 5.14 (s, 1H), 6.51-6.66 (m, 1H), 7.29-7.37 (m, 1H), 7.42-7.53 (m, 1H), 7.65-7.74 (m, 2H), 8.46-8.69 (m, 1H), 13.79-13.95 (m, 1H).

### Example 19

### N-(cis-4-{[6-(Dimethylamino)-2-methylpyrimidin-4-yl]amino}cyclohexyl)-4-(trifluoromethyl)benzamide hydrochloride

Using the procedure for the step A of example 7, the title compound was obtained.

ESI MS m/e 422, M (free)+H⁺; ¹H NMR (300 MHz, CDCl₃) δ 1.64-2.06 (m, 8H), 2.49 (s, 3H), 2.97-3.39 (m, 6H), 3.65-3.81 (m, 1H), 4.05-4.23 (m, 1H), 5.15 (s, 1H), 6.71-6.84 (m, 1H), 7.69 (d, J=8.2 Hz, 2H), 7.95 (d, J=8.2 Hz, 2H), 8.48-8.62 (m, 1H).

### Example 20

### N-(cis-4-{[6-(Dimethylamino)-2-methylpyrimidin-4-yl]amino}cyclohexyl)-4-(trifluoromethoxy)benzamide hydrochloride

Using the procedure for the step A of example 7, the title compound was obtained.

ESI MS m/e 460, M (free)+Na⁺; ¹H NMR (300 MHz, CDCl₃) δ 1.63-2.02, (m, 8H), 2.48 (s, 3H), 2.89-3.42 (m, 6H), 3.66-3.78 (m, 1H), 4.03-4.25 (m, 1H), 5.14 (s, 1H), 6.72-6.86 (m, 1H), 7.26 (d, J=7.6 Hz, 2H), 7.89 (d, J=8.9 Hz, 2H), 8.45-8.59 (m, 1H).

### Example 21

### 3,5-Dichloro-N-(cis-4-{[6-(dimethylamino)-2-methylpyrimidin-4-yl]amino}cyclohexyl)-benzamide hydrochloride

Using the procedure for the step A of example 7, the title compound was obtained.

ESI MS m/e 444, M (free)+Na⁺; ¹H NMR (300 MHz, CDCl₃) δ 1.65-2.02 (m, 8H), 2.49 (s, 3H), 2.93-3.42 (m, 6H), 3.68-3.79 (m, 1H), 4.02-4.19 (m, 1H), 5.14 (s, 1H), 6.47-6.57 (m, 1H), 7.45-7.48 (m, 1H), 7.68 (d, J=1.8 Hz, 2H), 8.52-8.65 (m, 1H).

### Example 22

### N-(cis-4-{[6-(Dimethylamino)-2-methylpyrimidin-4-yl]amino}cyclohexyl)-2-fluorobenzamide hydrochloride

Using the procedure for the step A of example 7, the title compound was obtained.

ESI MS m/e 394, M (free)+Na⁺; ¹H NMR (300 MHz, CDCl₃) δ 1.65-2.06 (m, 8H), 2.48 (s, 3H), 2.93-3.40 (m, 6H), 3.63-3.71 (m, 1H), 4.08-4.24 (m, 1H), 5.12 (s, 1H), 6.69-6.85 (m, 1H), 7.06-7.30 (m, 2H), 7.39-7.53 (m, 1H), 7.95-8.05 (m, 1H), 8.51-8.61 (m, 1H).

### Example 23

### N-(cis-4-{[6-(Dimethylamino)-2-methylpyrimidin-4-yl]amino}cyclohexyl)-3-fluorobenzamide hydrochloride

Using the procedure for the step A of example 7, the title compound was obtained.

ESI MS m/e 394, M (free)+Na⁺; ¹H NMR (300 MHz, CDCl₃) δ 1.64-2.05 (m, 8H), 2.49 (s, 3H), 2.99-3.45 (m, 6H), 3.66-3.77 (m, 1H), 4.04-4.23 (m, 1H), 5.14 (s, 1H), 6.40-6.53 (m, 1H), 7.13-7.22 (m, 1H), 7.34-7.45 (m, 1H), 7.52-7.58 (m, 2H), 8.52-8.62 (m, 1H).

### Example 24

### 3-Chloro-N-(cis-4-{[6-(dimethylamino)-2-methylpyrimidin-4-yl]amino}cyclohexyl)-benzamide hydrochloride

Using the procedure for the step A of example 7, the title compound was obtained.

ESI MS m/e 388, M (free)+H⁺; ¹H NMR (300 MHz, CDCl₃) δ 1.68-2.03 (m, 8H), 2.49 (s, 3H), 2.97-3.37 (m, 6H), 3.66-3.77 (m, 1H), 4.02-4.21 (m, 1H), 5.14 (s, 1H), 6.48-6.57 (m, 1H), 7.32-7.49 (m, 2H), 7.63-7.69 (m, 1H), 7.81-7.85 (m, 1H), 8.53-8.62 (m, 1H), 13.86-13.97 (m, 1H).

### Example 25

### 4-Chloro-N-(cis-4-{[6-(dimethylamino)-2-methylpyrimidin-4-yl]amino}cyclohexyl)-benzamide hydrochloride

Using the procedure for the step A of example 7, the title compound was obtained.

ESI MS m/e 388, M (free)+H⁺; ¹H NMR (300 MHz, CDCl₃) δ 1.67-2.07 (m, 8H), 2.49 (s, 3H), 2.98-3.38 (m, 6H), 3.67-3.79 (m, 1H), 4.01-4.21 (m, 1H), 5.14 (s, 1H), 6.42-6.55 (m, 1H), 7.37-7.43 (m, 2H), 7.73-7.80 (m, 2H), 8.52-8.63 (m, 1H), 13.82-13.98 (m, 1H).

### Example 26

### N-(cis-4-{[6-(Dimethylamino)-2-methylpyrimidin-4-yl]amino}cyclohexyl)-3-fluoro-5-(trifluoromethyl)benzamide hydrochloride

Using the procedure for the step A of example 7, the title compound was obtained.

ESI MS m/e 462, M (free)+Na⁺; ¹H NMR (300 MHz, CDCl₃) δ 1.70-2.05 (m, 8H), 2.48 (s, 3H), 2.93-3.45 (m, 6H), 3.67-3.79 (m, 1H), 4.04-4.23 (m, 1H), 5.15 (s, 1H), 6.71-6.84 (m, 1H), 7.40-7.47 (m, 1H), 7.72-7.79 (m, 1H), 7.90 (s, 1H), 8.49-8.63 (m, 1H).

### Example 27

### N-(cis-4-{([6-(Dimethylamino)-2-methylpyrimidin-4-yl]amino}cyclohexyl)-3,5-bis-(trifluoromethyl)benzamide hydrochloride

Using the procedure for the step A of example 7, the title compound was obtained.

ESI MS m/e 512, M (free)+Na⁺; ¹H NMR (300 MHz, CDCl₃) δ 1.66-2.09 (m, 8H), 2.48 (s, 3H), 2.91-3.44 (m, 6H), 3.67-3.83 (m, 1H), 4.04-4.27 (m, 1H), 5.15 (s, 1H), 6.92-7.05 (m, 1H), 7.98 (s, 1H), 8.32 (s, 2H), 8.50-8.64 (m, 1H).

### Example 28

### N-[cis-4-({[6-(Dimethylamino)-2-methylpyrimidin-4-yl]amino}methyl)cyclohexyl]-3,4-difluorobenzamide hydrochloride

Using the procedure for the step F of example 3, the title compound was obtained.

ESI MS m/e 404, M (free)+H⁺; ¹H NMR (300 MHz, CDCl₃) δ 1.50-2.08 (m, 9H), 2.46 (s, 3H), 2.88 (s, 8H), 4.43-4.58 (m, 1H), 5.06 (s, 1H), 7.10-7.35 (m, 2H), 7.88-8.08 (m, 2H), 8.58-8.78 (m, 1H), 13.44-13.62 (m, 1H).

### Example 29

### N-[(cis-4-{[6-(Dimethylamino)-2-methylpyrimidin-4-yl]amino}cyclohexyl)methyl]-3,4-difluorobenzamide hydrochloride

Using the procedure for the step C of example 4, the title compound was obtained.

ESI MS m/e 404, M (free)+H⁺; ¹H NMR (300 MHz, CDCl₃) δ 1.50-2.01 (m, 9H), 2.47 (s, 3H), 2.89-3.56 (m, 8H), 3.66-3.86 (m, 1H), 5.12 (s, 1H), 6.82-6.98 (m, 1H), 7.11-7.32 (m, 1H), 7.72-7.97 (m, 2H), 8.61-8.75 (m, 1H), 13.61-13.89 (m, 1H).

### Example 30

### 3,4-Difluoro-N-(cis-4-{[2-methyl-6-(methylamino)pyrimidin-4-yl]amino}cyclohexyl)-benzamide hydrochloride

### Step A: Synthesis of (6-chloro-2-methyl-pyrimidin-4-yl)-methyl-amine

To a solution of 4,6-dichloro-2-methyl-pyrimidine obtained in step A of example 5 (11.1 g) in THF (110 mL) were added iPr₂NEt (14.2 mL) and 40% aqueous MeNH₂ (10.1 mL). The mixture was stirred at ambient temperature for 7 hr and concentrated under reduced pressure. To the residue was added saturated aqueous NaHCO₃ and the aqueous layer was extracted with CHCl₃ (three times). The combined organic layer was dried over MgSO₄, filtered, concentrated under reduced pressure, and dried under reduced pressure to give (6-chloro-2-methyl-pyrimidin-4-yl)-methyl-amine (10.7 g).

ESI MS m/e 157, M⁺; ¹H NMR (200 MHz, CDCl₃) δ 2.48 (s, 3H), 2.93 (d, J=5.2 Hz, 3 H), 5.20-5.70 (m, 1H), 6.18 (s, 1H).

### Step B: Synthesis of 3,4-difluoro-N-(cis-4-{[2-methyl-6-(methylamino)pyrimidin-4-yl]amino}-cyclohexyl)-benzamide hydrochloride

Using the procedure for the step C of example 5, the title compound was obtained.

ESI MS m/e 376, M (free)+H⁺; ¹H NMR (300 MHz, CDCl₃) δ 1.58-2.13 (m, 8H), 2.37 (s, 3H), 2.82-3.19 (m, 3H), 3.56-3.86 (m, 1H), 3.98-4.27 (m, 1H), 5.03-5.30 (m, 1H), 6.07-6.52 (m, 1H), 6.71-6.96 (m, 1H), 7.11-7.33 (m, 1H), 7.49-7.82 (m, 2H), 8.34-8.60 (m, 1H).

### Example 31

### 3-Chloro-4-fluoro-N-(cis-4-{[2-methyl-6-(methylamino)pyrimidin-4-yl]amino}cyclohexyl)-benzamide hydrochloride

### Step A: Synthesis of N-(cis-4-amino-cyclohexyl)-3-chloro-4-fluoro-benzamide

To a solution of 3-chloro-4-fluoro-benzoic acid (26.9 g) and cis-(4-amino-cyclohexyl)-carbamic acid tert-butyl ester (30.0 g) in DMF (300 mL) were added Et₃N (46.8 mL), HOBt-H₂O (32.2 g), and EDC-HCl (29.5 g). The mixture was stirred at ambient temperature for 20 hr. To the mixture was added water (1.20 L) and the aqueous layer was extracted with CHCl₃ (three times). The combined organic layer was dried over MgSO₄, filtered, and concentrated under reduced pressure. A solution of the residue in EtOAc (650 mL) was cooled on an ice-bath and 4 M hydrogen chloride in EtOAc (325 mL) was added. The mixture was stirred at ambient temperature for 16 hr and concentrated under reduced pressure. The residue was dissolved in 1 M aqueous NaOH (300 mL) and the aqueous layer was extracted with CHCl₃ (three times). The combined organic layer was dried over MgSO₄, filtered, concentrated under reduced pressure, and dried under reduced pressure to give N-(cis-4-amino-cyclohexyl)-3-chloro-4-fluoro-benzamide (44.4 g).

ESI MS m/e 271, M (free)+H⁺; ¹H NMR (300 MHz, CDCl₃) δ 1.37-1.92 (m, 8H), 2.94-3.08 (m, 1H), 4.06-4.22 (m, 1H), 6.13-6.31 (m, 1H), 7.19 (t, J=8.5 Hz, 1H), 7.61-7.70 (m, 1H), 7.79-7.87 (m, 1H).

### Step B: Synthesis of 3-chloro-4-fluoro-N-(cis-4-{[2-methyl-6-(methylamino)pyrimidin-4-yl]-amino}cyclohexyl)-benzamide hydrochloride

To a solution of N-(cis-4-amino-cyclohexyl)-3-chloro-4-fluoro-benzamide (472 mg) in BuOH (1 mL) was added (6-chloro-2-methyl-pyrimidin-4-yl)-methyl-amine obtained in step A of example 30 (250 mg). The mixture was heated in a microwave synthesizer at 220° C. for 20 min. To the mixture was added saturated aqueous NaHCO₃ and the aqueous layer was extracted with CHCl₃ (three times). The combined organic layer was dried over MgSO₄, filtrated, concentrated under reduced pressure, and purified by medium-pressure liquid chromatography (NH-silica gel, 50% EtOAc in hexane) to give 3-chloro-4-fluoro-N-(cis-4-{[2-methyl-6-(methylamino)pyrimidin-4-yl]-amino}cyclohexyl)-benzamide. To a solution of the above material in EtOAc (10 mL) was added 4 M hydrogen chloride in EtOAc (0.2 mL). The mixture was stirred at ambient temperature for 1 hr and concentrated under reduced pressure. A suspension of the residue in Et₂O (12 mL) was stirred at ambient temperature for 2 hr. The precipitate was collected by filtration, washed with Et₂O, and dried at 70° C. under reduced pressure to give 3-chloro-4-fluoro-N-(cis-4-{[2-methyl-6-(methylamino)pyrimidin-4-yl]-amino}cyclohexyl)-benzamide hydrochloride (64 mg).

ESI MS m/e 392, M (free)+H⁺; ¹H NMR (300 MHz, DMSO-d₆) δ 1.54-1.90 (m, 8H), 2.29-2.43 (m, 3H), 2.74-2.94 (m, 3H), 3.80-3.96 (m, 2H), 5.44-5.64 (m, 1H), 7.53 (t, J=8.9 Hz, 1H), 7.86-7.94 (m, 2H), 8.07-8.13 (m, 2H), 8.31-8.47 (m, 1H).

### Example 32

### N-(cis-4-{[6-(Dimethylamino)-2-ethylpyrimidin-4-yl]amino}cyclohexyl)-3,4-difluorobenzamide hydrochloride

### Step A: Synthesis of (2,6-dichloro-pyrimidin-4-yl)-dimethyl-amine

To a solution of 2,4,6-trichloro-pyrimidine (10.0 g) in THF (50 mL) were added 50% aqueous Me₂NH (4.92 g) and iPr₂NEt (8.46 g). The mixture was stirred at ambient temperature for 1.5 hr and concentrated under reduced pressure. The residue was poured into saturated aqueous NaHCO₃ and the aqueous layer was extracted with CHCl₃ (three times). The combined organic layer was dried over MgSO₄, filtered, concentrated under reduced pressure, and purified flash chromatography (NH-silica gel, 3% EtOAc in hexane) to give (2,6-dichloro-pyrimidin-4-yl)-dimethyl-amine (6.03 g).

ESI MS m/e 192, M+H⁺; ¹H NMR (300 MHz, CDCl₃) δ 2.77-3.46 (m, 6H), 6.34 (s, 1H).

### Step B: Synthesis of (6-chloro-2-ethyl-pyrimidin-4-yl)-dimethyl-amine

A solution of ZnBr₂ (3.87 g) in THF (60 mL) was cooled to 60° C. and 1 M EtMgBr in THF (17.2 mL) was added. The mixture was stirred at −6° C. for 1 hr and warmed to ambient temperature. To the mixture were added tetrakis-(triphenylphosphine)-palladium (903 mg) and (2,6-dichloro-pyrimidin-4-yl)-dimethyl-amine in THF (60 mL) and the mixture was stirred at reflux for 5 days. To the mixture was added saturated aqueous NH₄Cl and the aqueous layer was extracted with CHCl₃ (three times). The combined organic layer was dried over MgSO₄, filtered, concentrated under reduced pressure, and purified by medium-pressure liquid chromatography (silica gel, 17% to 33% EtOAc in hexane) to give (2-chloro-6-ethyl-pyrimidin-4-yl)-dimethyl-amine (352 mg) and (6-chloro-2-ethyl-pyrimidin-4-yl)-dimethyl-amine (622 mg).

### (2-chloro-6-ethyl-pyrimidin-4-yl)-dimethyl-amine

ESI MS m/e 208, M (free)+Na⁺; ¹H NMR (300 MHz, CDCl₃) δ 1.25 (t, J=7.6 Hz, 3H), 2.54-2.66 (m, 2H), 3.11 (s, 6H), 6.15 (s, 1H).

### (6-chloro-2-ethyl-pyrimidin-4-yl)-dimethyl-amine

ESI MS m/e 186, M+H⁺; ¹H NMR (300 MHz, CDCl₃) δ 1.29 (t, J=7.6 Hz, 3H), 2.74 (q, J=7.7 Hz, 2H), 3.10 (s, 6H), 6.24 (s, 1H).

### Step C: Synthesis of N-(cis-4-{[6-(dimethylamino)-2-ethylpyrimidin-4-yl]amino}cyclohexyl)-3,4-difluorobenzamide hydrochloride

Using the procedure for the step C of example 5, the title compound was obtained.

ESI MS m/e 404, M (free)+H⁺; ¹H NMR (300 MHz, CDCl₃) δ 1.37 (t, J=7.5 Hz, 3H), 1.64-2.03 (m, 8H), 2.76 (q, J=7.5 Hz, 2H), 2.97-3.42 (m, 6H), 3.65-3.80 (m, 1H), 4.02-4.21 (m, 1H), 5.14 (s, 1H), 6.42-6.66 (m, 1H), 7.12-7.27 (m, 1H), 7.45-7.60 (m, 1H), 7.65-7.81 (m, 1H), 8.60-8.73 (m, 1H), 13.61-13.77 (m, 1H).

### Example 33

### N-(cis-4-{[2,6-bis(Dimethylamino)pyrimidin-4-yl]amino}cyclohexyl)-3,4-difluorobenzamide hydrochloride

### Step A: Synthesis of 6-chloro-N,N,N′,N′-tetramethyl-pyrimidine-2,4-diamine

To a suspension of (2,6-dichloro-pyrimidin-4-yl)-dimethyl-amine obtained in step A of example 32 (1.60 g) in IPA (2 mL) was added 50% aqueous Me₂NH (789 mg). The mixture was stirred at reflux for 3.5 hr in a sealed tube. The mixture was poured into saturated aqueous NaHCO₃ and the aqueous layer was extracted with CHCl₃ (three times). The combined organic layer was dried over MgSO₄, filtered, concentrated under reduced pressure, and purified by medium-pressure liquid chromatography (silica gel, 20% EtOAc in hexane) to give 2-chloro-N,N,N′,N′-tetramethyl-pyrimidine-4,6-diamine (203 mg) and 6-chloro-N,N,N′,N′-tetramethyl-pyrimidine-2,4-diamine (1.43 g).

### 2-chloro-N,N′,N′-tetramethyl-pyrimidine-4,6-diamine

ESI MS m/e 201, M (free)+H⁺; ¹H NMR (300 MHz, CDCl₃) δ 3.05 (s, 12H), 5.15 (s, 1H).

### 6-chloro-N,N,N′,N′-tetramethyl-pyrimidine-2,4-diamine

ESI MS m/e 201, M+H⁺; ¹H NMR (300 MHz, CDCl₃) δ 3.04 (s, 6H), 3.13 (s, 6H), 5.76 (s, 1H).

### Step B: Synthesis of N-(cis-4-{[2,6-bis(dimethylamino)pyrimidin-4-yl]amino}cyclohexyl)-3,4-difluorobenzamide hydrochloride

Using the procedure for the step C of example 5, the title compound was obtained.

ESI MS m/e 419, M (free)+H⁺; ¹H NMR (300 MHz, CDCl₃) δ 1.58-2.16 (m, 8H), 2.97-3.45 (m, 12H), 3.62-3.74 (m, 1H), 4.03-4.21 (m, 1H), 4.81 (s, 1H), 6.76-6.90 (m, 1H), 7.13-7.26 (m, 1H), 7.55-7.64 (m, 1H), 7.70-7.79 (m, 1H), 8.57-8.70 (m, 1H), 11.86-11.94 (m, 1H).

### Example 34

### N-(cis-4-{[2-(Ethylamino)pyrimidin-4-yl]amino}cyclohexyl)-3,4-difluorobenzamide hydrochloride

### Step A: Synthesis of (4-chloro-pyrimidin-2-yl)-ethyl-amine

To a solution of 2,4-dichloro-pyrimidine (5.00 g) in THF (50 mL) was added 70% aqueous EtNH₂ (5.40 g). The mixture was stirred at ambient temperature for 1 hr and concentrated under reduced pressure. The residue was dissolved in CHCl₃ and the solution was poured into saturated aqueous NaHCO₃. The two layers were separated and the aqueous layer was extracted with CHCl₃ (twice). The combined organic layer was dried over MgSO₄, filtered, concentrated under reduced pressure, and purified by flash chromatography (silica gel, 17% to 50% EtOAc in hexane) to give (2-chloro-pyrimidin-4-yl)-ethyl-amine (3.69 g) and (4-chloro-pyrimidin-2-yl)-ethyl-amine (1.28 g).

### (2-chloro-pyrimidin-4-yl)-ethyl-amine

ESI MS m/e 157, M⁺; ¹H NMR (500 MHz, CDCl₃) δ 1.26 (t, J=7.3 Hz, 3H), 3.16-3.62 (m, 2H), 4.80-5.95 (m, 1H), 6.23 (d, J=5.8 Hz, 1H), 8.02-8.22 (m, 1H).

### (4-chloro-pyrimidin-2-yl)-ethyl-amine

CI MS m/e 158, M+H⁺; ¹H NMR (500 MHz, CDCl₃) δ 1.23 (t, J=7.5 Hz, 3H), 3.42-3.49 (m, 2H), 5.30-5.62 (m, 1H), 6.54 (d, J=5.2 Hz, 1H), 8.02-8.22 (m, 1H).

### Step B: Synthesis of N-(cis-4-{[2-(ethylamino)pyrimidin-4-yl]amino}cyclohexyl)-3,4-difluorobenzamide hydrochloride

Using the procedure for the step C of example 5, the title compound was obtained.

ESI MS m/e 376, M (free)+H⁺; ¹H NMR (300 MHz, CDCl₃) δ 1.22 (t, J=7.1 Hz, 3H), 1.61 (s, 8H), 3.31-3.56 (m, 2H), 4.05-4.47 (m, 2H), 6.31-6.56 (m, 1H), 6.75-6.95 (m, 1H), 7.07-7.34 (m, 2H), 7.48-7.87 (m, 3H), 8.01-8.24 (m, 1H), 12.39-12.52 (m, 1H).

### Example 35

### N-[cis-4-({2-[Ethyl(methyl)amino]pyrimidine-4-yl}amino)cyclohexyl]-3,4-difluorobenzamide hydrochloride

### Step A: Synthesis of (4-chloro-pyrimidin-2-yl)-ethyl-methyl-amine

To a solution of 2,4-dichloro-pyrimidine (5.00 g) in THF (50 mL) was added ethyl-methyl-amine (2.08 g). The mixture was stirred at ambient temperature for 1 hr and concentrated under reduced pressure. The residue was dissolved in CHCl₃ and the solution was poured into saturated aqueous NaHCO₃. The two layers were separated and the aqueous layer was extracted with CHCl₃ (twice). The combined organic layer was dried over MgSO₄, filtered, concentrated under reduced pressure, and purified by flash chromatography (silica gel, 17% to 50% EtOAc in hexane) to give (2-chloro-pyrimidin-4-yl)-ethyl-methyl-amine (4.49 g) as (4-chloro-pyrimidin-2-yl)-ethyl-methyl-amine (0.91 g).

### (2-chloro-pyrimidin-4-yl)-ethyl-methyl-amine

CI MS m/e 172, M (free)+H⁺; ¹H NMR (500 MHz, CDCl₃) δ 1.18 (t, J=3.0 Hz, 3H), 3.06 (brs, 3H), 3.35-3.70 (m, 2H), 6.29 (d, J=4.8 Hz, 1H), 7.99 (d, J=6.1 Hz, 1H).

### (4-chloro-pyrimidin-2-yl)-ethyl-methyl-amine

CI MS m/e 172, M+H⁺; ¹H NMR (500 MHz, CDCl₃) δ 1.17 (t, J=3.0 Hz, 3H), 3.10 (s, 3H), 3.66 (q, J=7.0 Hz, 2H), 6.45 (d, J=5.0 Hz, 1H), 8.14 (d, J=5.0 Hz, 1H).

### Step B: Synthesis of N-[cis-4-({2-[ethyl(methyl)amino]pyrimidin-4-yl}amino)cyclohexyl]-3,4-difluorobenzamide hydrochloride

Using the procedure for the step C of example 5, the title compound was obtained.

ESI MS m/e 390, M (free)+H⁺; ¹H NMR (300 MHz, CDCl₃) δ 1.11-1.29 (m, 3H), 1.63-2.20 (m, 8H), 3.23 (brs, 3H), 3.61-3.76 (m, 2H), 4.06-4.42 (m, 2H), 6.53-6.68 (m, 1H), 6.88-7.24 (m, 2H), 7.39-7.52 (m, 1H), 7.59-7.86 (m, 2H), 8.39-8.54 (m, 1H), 12.26-12.44 (m, 1H).

### Example 36

### 3,4-Difluoro-N-[cis-4-({2-[(2-hydroxyethyl)(methyl)amino]pyrimidin-4-yl}amino)-cyclohexyl]benzamide hydrochloride

### Step A: Synthesis of 2-[(4-chloro-pyrimidin-2-yl)-methyl-amino]-ethanol

To a solution of 2,4-dichloro-pyrimidine (5.00 g) in THF (50 mL) was added 2-methylamino-ethanol (2.65 g). The mixture was stirred at ambient temperature for 1 hr and concentrated under reduced pressure. The residue was dissolved in CHCl₃ and the solution was poured into saturated aqueous NaHCO₃. The two layers were separated and the aqueous layer was extracted with CHCl₃ (twice). The combined organic layer was dried over MgSO₄, filtered, concentrated under reduced pressure, and purified by flash chromatography (silica gel, 17% to 50% EtOAc in hexane) to give 2-[(2-chloro-pyrimidin-4-yl)-methyl-amino]-ethanol (3.50 g) and 2-[(4-chloro-pyrimidin-2-yl)-methyl-amino]-ethanol (827 mg).

### 2-[(2-chloro-pyrimidin-4-yl)-methyl-amino]-ethanol

ESI MS m/e 188, M (free)+H⁺; ¹H NMR (500 MHz, CDCl₃) δ 2.91 (brs, 3H), 3.13 (s, 3H), 3.64-3.92 (m, 4H), 6.46-6.49 (m, 1H), 7.99 (d, J=6.1 Hz, 1H).

### 2-[(4-chloro-pyrimidin-2-yl)-methyl-amino]-ethanol

ESI MS m/e 210, M+Na⁺; ¹H NMR (500 MHz, CDCl₃) δ 3.23 (s, 3H), 3.76-3.92 (m, 4 H), 6.52 (d, J=5.2 Hz, 1H), 8.12 (d, J=4.6 Hz, 1H).

### Step B: Synthesis of 3,4-difluoro-N-[cis-4-({2-[(2-hydroxyethyl)(methyl)amino]pyrimidin-4-yl}amino)-cyclohexyl]benzamide hydrochloride

Using the procedure for the step C of example 5, the title compound was obtained.

ESI MS m/e 406, M (free)+H⁺; ¹H NMR (300 MHz, DMSO-d₆) δ 1.59-1.96 (m, 8H), 3.16 (s, 3H) 3.57-3.71 (m, 2H), 3.80-4.07 (m, 3H), 4.20-4.30 (m, 1H), 6.20-6.34 (m, 1H), 7.49-7.80 (m, 3H), 7.88-7.99 (m, 1H), 8.31-8.40 (m, 1H), 8.64-8.79 (m, 1H).

### Example 37

### 3-Chloro-4-fluoro-N-{cis-4-[(2-methyl-6-piperidin-1-ylpyrimidin-4-yl)amino]cyclohexyl}-benzamide hydrochloride

To a solution of 4,6-dichloro-2-methyl-pyrimidine obtained in step A of example 5 (3.00 g) in THF (30 mL) were added N-(cis-4-amino-cyclohexyl)-3-chloro-4-fluoro-benzamide obtained in step A of example 31 (5.98 g) and iPrNEt₂ (3.85 mL). The mixture was stirred at reflux for 60 hr and poured into saturated aqueous NaHCO₃ The aqueous layer was extracted with CHCl₃ (three times). The combined organic layer was dried over MgSO₄, filtrated, concentrated under reduced pressure, and purified by medium-pressure liquid chromatography (NH-silica gel, 20% EtOAc in hexane) to give 3-chloro-N-[cis-4-(6-chloro-2-methyl-pyrimidin-4-ylamino)-cyclohexyl]-4-fluoro-benzamide (6.34 g). To a solution of above solid (250 mg) in BuOH (1 mL) were added piperidine (80 mg) and iPrNEt₂ (121 mg). The mixture was heated in a microwave synthesizer at 220° C. for 10 min and 230° C. for 20 min and poured into saturated aqueous NaHCO₃. The aqueous layer was extracted with CHCl₃ (three times). The combined organic layer was dried over MgSO₄, filtrated, concentrated under reduced pressure, and purified by medium-pressure liquid chromatography (NH-silica gel, 20% EtOAc in hexane) to give 3-chloro-4-fluoro-N-{cis-4-[(2-methyl-6-piperidin-1-ylpyrimidin-4-yl)amino]cyclohexyl}-benzamide. To a solution of the above material in EtOAc (10 mL) was added 4 M hydrogen chloride in EtOAc (0.2 mL). The mixture was stirred at ambient temperature for 1 hr and concentrated under reduced pressure. A suspension of the residue in Et₂O (12 mL) was stirred at ambient temperature for 2 hr. The precipitate was collected by filtration, washed with Et₂O, and dried at 70° C. under reduced pressure to give 3-chloro-4-fluoro-N-{cis-4-[(2-methyl-6-piperidin-1-ylpyrimidin-4-yl)amino]cyclohexyl}-benzamide hydrochloride (6 mg).

ESI MS m/e 446, M (free)+H⁺; ¹H NMR (300 MHz, CDCl₃) δ 1.28-2.10 (m, 14H), 2.46 (s, 3H), 2.92-3.11 (m, 1H), 3.27-3.89 (m, 4H), 4.00-4.21 (m, 1H), 5.16-5.31 (m, 1H), 6.69-6.88 (m, 1H), 7.13-7.27 (m, 1H), 7.60-8.03 (m, 2H), 8.40-8.55 (m, 1H).

### Example 38

### 3-Chloro-4-fluoro-N-(cis-4-{[6-(1H-imidazol-1-yl)-2-methylpyrimidin-4-yl]amino}cyclohexyl)-benzamide dihydrochloride

Using the procedure for the step A of example 37, the title compound was obtained.

ESI MS m/e 451, M (free)+Na⁺⁶; ¹H NMR (300 MHz, CDCl₃) δ 1.69-2.21 (m, 8H), 2.56-2.87 (m, 3H), 4.04-4.58 (m, 2H), 6.41-6.70 (m, 1H), 7.10-7.25 (m, 1H), 7.42-7.51 (m, 1H), 7.58-7.80 (m, 1H), 7.84-8.22 (m, 3H).

### Example 39

### 3-Chloro-4-fluoro-N-{cis-4-[(2-methyl-6-morpholin-4-ylpyrimidin-4-yl)amino]cyclohexyl}-benzamide hydrochloride

Using the procedure for the step A of example 37, the title compound was obtained.

ESI MS m/e 470, M (free)+Na⁺; ¹H NMR (300 MHz, CDCl₃) δ 1.65-2.02 (m, 8H), 2.49 (s, 3H), 3.58-3.92 (m, 9H), 4.03-4.22 (m, 1H), 5.25 (s, 1H), 6.51-6.62 (m, 1H), 7.18 (t, J=8.5 Hz, 1H), 7.67-7.74 (m, 1H), 7.91-7.96 (m, 1H), 8.63-8.75 (m, 1H).

### Example 40

### 3-Chloro-4-fluoro-N-{cis-4-[(2-methyl-6-pyrrolidin-1-ylpyrimidin-4-yl)amino]cyclohexyl}-benzamide hydrochloride

Using the procedure for the step A of example 37, the title compound was obtained.

ESI MS m/e 432, M (free)+H⁺; ¹H NMR (300 MHz, CDCl₃) δ 1.41-2.24 (m, 12H), 2.48 (s, 3H), 3.09-3.56 (m, 3H), 3.60-3.78 (m, 2H), 3.99-4.18 (m, 1H), 5.02 (s, 1H), 6.52-6.66 (m, 1H), 7.18 (t, J=8.6 Hz, 1H), 7.63-7.77 (m, 1H), 7.88-7.99 (m, 1H), 8.40-8.55 (m, 1H).

### Example 41

### 3-Chloro-4-fluoro-N-(cis-4-{[2-methyl-6-(4-methylpiperazin-1-yl)pyrimidin-4-yl]amino}-cyclohexyl)benzamide dihydrochloride

Using the procedure for the step A of example 37, the title compound was obtained.

ESI MS m/e 461, M (free)+H⁺; ¹H NMR (300 MHz, DMSO-d₆) δ 1.63-1.88 (m, 8H), 2.37-2.46 (m, 3H), 2.73-2.83 (m, 3H), 2.97-3.15 (m, 2H), 3.24-3.62 (m, 6H), 3.78-4.01 (m, 2H), 5.99 (s, 1H), 7.52 (t, J=8.9 Hz, 1H), 7.81-7.97 (m, 1H), 8.04-8.16 (m, 2H), 8.40-8.54 (m, 1H).

### Example 42

### N⁴-(cis-4-{[Bromo-2-(trifluoromethoxy)benzyl]amino}cyclohexyl)-N²,N²-dimethylpyrimidine-2,4-diamine dihydrochloride

### Step A: Synthesis of (4-chloro-pyrimidin-2-yl)-dimethyl-amine

To a solution of 2,4-dichloro-pyrimidine (15.0 g) in THF (150 mL) was added 50% aqueous Me₂NH (22.7 g). The mixture was stirred at ambient temperature for 2 hr and poured into saturated aqueous NaHCO₃. The aqueous layer was extracted with CHCl₃ (three times). The combined organic layer was dried over MgSO₄, filtrated, concentrated under reduced pressure, and purified by flash chromatography (NH-silica, 20% EtOAc in hexane) to give (2-chloro-pyrimidin-4-yl)-dimethyl-amine (8.66 g) and (4-chloro-pyrimidin-2-yl)-dimethyl-amine (0.87 g).

### (2-chloro-pyrimidin-4-yl)-dimethyl-amine

CI MS m/e 158, M+H⁺; ¹H NMR (300 MHz, CDCl₃) δ 3.12 (s, 6H), 6.32 (d, J=6.1 Hz, 1H), 8.00 (d, J=6.1 Hz, 1H).

### (4-chloro-pyrimidin-2-yl)-dimethyl-amine

ESI MS m/e 157, M⁺; ¹H NMR (300 MHz, CDCl₃) δ3.21 (s, 6H), 6.50 (d, J=5.1 Hz, 1H), 8.18 (d, J=5.1 Hz, 1H).

### Step B: Synthesis of N⁴-(cis-4-{[4-bromo-2-(trifluoromethoxy)benzyl]amino}cyclohexyl)-N²,N²-dimethylpyrimidine-2,4-diamine dihydrochloride

A mixture of N-(cis-4-bromo-2-trifluoromethoxy-benzyl)-cyclohexane-1,4-diamine obtained in step B of example 1 (466 mg), (4-chloro-pyrimidin-2-yl)-dimethyl-amine (200 mg), and BuOH (11 mL) was stirred at reflux for 13 hr. The mixture was poured into saturated aqueous NaHCO₃ and the aqueous layer was extracted with CHCl₃ (three times). The combined organic layer was dried over MgSO₄, filtered, concentrated under reduced pressure, and purified by flash chromatography (NH-silica gel, 20% EtOAc in) to give N⁴-(cis-4-{[4-bromo-2-(trifluoromethoxy)benzyl]amino}-cyclohexyl)-N²,N²-dimethylpyrimidine-2,4-diamine. To a solution of the above material in EtOAc (2 mL) was added 4 M hydrogen chloride in EtOAc (10 mL). The mixture was stirred at ambient temperature for 1 hr and concentrated under reduced pressure. The residue was suspended in Et₂O (20 mL) and the suspension was stirred at ambient temperature for 4 hr. The precipitate was collected by filtration, washed with Et₂O, and dried under reduced pressure to give N⁴-(cis-4-{[4-bromo-2-(trifluoromethoxy)benzyl]-amino}cyclohexyl)-N²,N²-dimethylpyrimidine-2,4-diamine dihydrochloride (294 mg).

ESI MS m/e 488, M (free)+H⁺; ¹H NMR (300 MHz, CDCl₃) δ 1.42-1.67 (m, 2H), 2.03-2.39 (m, 6H), 2.79-3.38 (m, 7H), 4.13-4.36 (m, 3H), 6.89-7.00 (m, 1H), 7.42-7.46 (m, 1H), 7.50-7.57 (m, 1H), 7.90-8.01 (m, 1H), 8.12 (d, J=8.4 Hz, 1H), 8.90-9.00 (m, 1H), 9.98-10.18 (m, 2H), 12.21-12.37 (m, 1H).

### Example 43

### N-(cis-4-{[2-(Dimethylamino)-6-methylpyrimidin-4-yl]amino}cyclohexyl)-3,4-difluorobenzamide hydrochloride

### Step A: Synthesis of (4-chloro-6-methyl-pyrimidin-2-yl)-dimethyl-amine

To a solution of 2,4-dichloro-6-methylpyrimidine (20.0 g) in THF (200 mL) was added 50% aqueous Me₂NH (13.3 g) and the mixture was stirred at ambient temperature for 24 hr. To the mixture was added saturated aqueous NaHCO₃ and the aqueous layer was extracted with CHCl₃ (three times). The combined organic layer was dried over MgSO₄, filtered, concentrated under reduced pressure, and purified flash chromatography (NH-silica gel, 5% to 16% EtOAc in hexane) to give (2-chloro-6-methyl-pyrimidin-4-yl)-dimethyl-amine (14.4 g) and (4-chloro-6-methyl-pyrimidin-2-yl)-dimethyl-amine (6.57 g).

### (2-chloro-6-methyl-pyrimidin-4-yl)-dimethyl-amine

ESI MS m/e 194, M⁺+Na⁺; ¹H NMR (300 MHz, CDCl₃) δ2.34 (s, 3H), 3.10 (s, 6H), 6.16 (s, 1H).

### (4-chloro-6-methyl-pyrimidin-2-yl)-dimethyl-amine

CI MS m/e 172, M+H⁺; ¹H NMR (300 MHz, CDCl₃) δ 2.29 (s, 3H), 3.16 (s, 6H), 6.34 (s, 1H).

### Step B: Synthesis of N-(cis-4-{[2-(dimethylamino)-6-methylpyrimidin-4-yl]amino}cyclohexyl)-3,4-difluorobenzamide hydrochloride

To a solution of N-(cis-4-amino-cyclohexylmethyl)-3,4-difluoro-benzamide (652 mg) in BuOH (1 mL) was added (4-chloro-6-methyl-pyrimidin-2-yl)-dimethyl-amine (400 mg). The mixture was stirred at reflux for 8 days. To the mixture was added saturated aqueous NaHCO₃ and the aqueous layer was extracted with CHCl₃ (three times). The combined organic layer was dried over MgSO₄, filtrated, concentrated under reduced pressure, and purified by medium-pressure liquid chromatography (NH-silica gel, 10% to 20% EtOAc in hexane) to give N-(cis-4-{[2-(dimethylamino)-6-methylpyrimidin-4-yl]amino}cyclohexyl)-3,4-difluorobenzamide. To a solution of the above material in EtOAc (5 mL) was added 4 M hydrogen chloride in EtOAc (10 mL). The mixture was stirred at ambient temperature for 1 hr and concentrated under reduced pressure. A suspension of the residue in Et₂O (20 mL) was stirred at ambient temperature for 4 hr. The precipitate was collected by filtration, washed with Et₂O, and dried at 80° C. under reduced pressure to give N-(cis-4-{[2-(dimethylamino)-6-methylpyrimidin-4-yl]amino}cyclohexyl)-3,4-difluorobenzamide hydrochloride (507 mg).

¹H NMR (300 MHz, CDCl₃) δ 1.62-2.21 (m, 8H), 2.39 (s, 3H), 3.15-3.45 (m, 6H), 4.09-4.43 (m, 2H), 6.28-6.37 (m, 1H), 7.06-7.24 (m, 1H), 7.61-7.87 (m, 2H), 8.24-8.37 (m, 1H), 11.55-11.67 (m, 1H).

### Example 44

### 3-Chloro-N-(cis-4-{[2-(dimethylamino)pyrimidin-4-yl]amino}cyclohexyl)-4-fluorobenzamide hydrochloride

Using the procedure for the step B of example 31, the title compound was obtained.

ESI MS m/e 392, M (free)+H⁺; ¹H NMR (300 MHz, CDCl₃) δ 1.58-2.20 (m, 8H), 3.07 (s, 6H), 4.03-4.48 (m, 2H), 6.52-6.73 (m, 1H), 6.95-6.95 (m, 2H), 7.36-7.51 (m, 1H), 7.72-7.85 (m, 1H), 7.94-8.05 (m, 1H), 8.50-8.69 (m, 1H), 12.20-12.41 (m, 1H).

### Example 45

### 3-Chloro-N-(cis-4-{([2-(dimethylamino)-6-methylpyrimidin-4-yl]amino}cyclohexyl)-4-fluorobenzamide hydrochloride

Using the procedure for the step B of example 31, the title compound was obtained.

ESI MS m/e 406, M (free)+H⁺; ¹H NMR (300 MHz, CDCl₃) δ 1.56-2.22 (m, 11H), 3.05-3.45 (m, 6H), 4.07-4.42 (m, 2H), 6.25-6.40 (m, 1H), 7.03-7.26 (m, 2H), 7.73-8.07 (m, 2 H), 8.30-8.44 (m, 1H), 11.51-11.64 (m, 1H).

### Example 46

### 3-Chloro-N-(cis-4-{[2-(dimethylamino)-5-methylpyrimidin-4-yl]amino}cyclohexyl)-4-fluorobenzamide hydrochloride

### Step A: Synthesis of 4-chloro-2-dimethylamino-5-methylpyrimidine

To a solution of 2,4-dichloro-5-methylpyrimidine (20.0 g) in THF (200 mL) was added 50% aqueous Me₂NH (13.3 g). The mixture was stirred at ambient temperature for 5 days and concentrated under reduced pressure. The residue was poured into saturated aqueous NaHCO₃. The aqueous layer was extracted with CHCl₃ (three times). The combined organic layer was dried over MgSO₄, filtered, concentrated under reduced pressure, and purified by flash chromatography (NH-silica gel, 2% EtOAc in hexane) to give 2-chloro-4-dimethylamino-5-methylpyrimidine (19.9 g) and 4-chloro-2-dimethylamino-5-methylpyrimidine (1.53 g).

### 2-chloro-4-dimethylamino-5-methylpyrimidine

ESI MS m/e 172, M+H⁺; ¹H NMR (300 MHz, CDCl₃) δ 2.27 (s, 3H), 3.15 (s, 6H), 7.82 (s, 1H).

### 4-chloro-2-dimethylamino-5-methylpyrimidine

ESI MS m/e 194, M+Na⁺; ¹H NMR (300 MHz, CDCl₃) δ 2.14 (s, 3H), 3.15 (s, 6H), 8.06 (s, 1H).

### Step B: Synthesis of 3-chloro-N-(cis-4-{[2-(dimethylamino)-5-methylpyrimidin-4-yl]amino}-cyclohexyl)-4-fluorobenzamide hydrochloride

Using the procedure for the step B of example 31, the title compound was obtained.

ESI MS m/e 406, M (free)+H⁺; ¹H NMR (300 MHz, DMSO-d₆) δ 1.56-2.02 (m, 8H), 2.04 (s, 3H), 3.16 (s, 6H), 3.90-4.18 (m, 2H), 7.47-7.66 (m, 3H), 7.91-8.00 (m, 1H), 8.13-8.21 (m, 1H), 8.28-8.36 (m, 1H), 12.39-12.48 (m, 1H).

### Example 47

### 3-Chloro-N-(cis-4-{[6-(dimethylamino)-2-(trifluoromethyl)pyrimidin-4-yl]amino}cyclohexyl)-4-fluorobenzamide hydrochloride

### Step A: Synthesis of 2-trifluoromethyl-pyrimidine-4,6-diol

To a suspension of 60% NaH in oil (11.7 g) in toluene (98 mL) was added BuOH (21.8 g). The mixture was stirred at ambient temperature for 16 hr. To the mixture were added malonamide (10.0 g) and trifluoro-acetic acid ethyl ester (13.9 g). The mixture was stirred at 100° C. for 3.5 hr and ambient temperature for 16 hr. The organic layer was extracted with water (two times) and the aqueous layer was filtrated through activated carbon. To the aqueous layer was added conc. HCl (pH 1) and the suspension was stirred at 4° C. for 2 hr. The precipitate was collected by filtration and dried at 80° C. under reduced pressure to give 2-trifluoromethyl-pyrimidine-4,6-diol (3.25 g).

ESI MS m/e 178, M−H⁺; ¹H NMR (300 MHz, CDCl₃) δ 6.00 (s, 1H), 12.48 (brs, 2H).

### Step B: Synthesis of (6-chloro-2-trifluoromethyl-pyrimidin-4-yl)-dimethyl-amine

To a suspension of 2-trifluoromethyl-pyrimidine-4,6-diol (3.25 g) in POCl₃ (7.89 mL) was added Et₃N (5.00 mL). The mixture was stirred at 120° C. for 3 hr, cooled to ambient temperature, and poured into ice water. The aqueous layer was extracted with CHCl₃ (three times). The combined organic layer was dried over MgSO₄, filtrated, and concentrated under reduced pressure to give 4,6-dichloro-2-trifluoromethyl-pyrimidine. To the solution of the above material (1.00 g) in THF (10 mL) were added iPr₂NEt (0.98 mL) and 50% aqueous Me₂NH (0.48 mL). The mixture was stirred at ambient temperature for 60 hr. To the solution was added saturated aqueous NaHCO₃ and the aqueous layer was extracted with CHCl₃ (three times). The combined organic layer was dried over MgSO₄, filtered, concentrated under reduced pressure, and purified by medium-pressure liquid chromatography (silica gel, 5% to 25% EtOAc in hexane) to give (6-chloro-2-trifluoromethyl-pyrimidin-4-yl)-dimethyl-amine (728 mg).

ESI MS m/e 225 M⁺; ¹H NMR (300 MHz, CDCl₃) δ 2.77-3.61 (m, 6H), 6.50 (s, 1H).

### Step C: Synthesis of 3-chloro-N-(cis-4-{[6-(dimethylamino)-2-(trifluoromethyl)pyrimidin-4-yl]amino}cyclohexyl)-4-fluorobenzamide hydrochloride

Using the procedure for the step B of example 31, the title compound was obtained.

ESI MS m/e 482, M (free)+H⁺; ¹H NMR (300 MHz, CDCl₃) δ 1.66-2.08 (m, 8H), 3.20 (s, 6H), 3.68-3.83 (m, 1H), 4.04-4.21 (m, 1H), 5.30 (s, 1H), 6.34-6.46 (m, 1H), 7.18 (t, J=8.5 Hz, 1H), 7.63-7.73 (m, 2H), 7.87-7.93 (m, 1H).

### Example 48

### 5-Bromo-furan-2-carboxylic acid [cis-4-(6-dimethylamino-2-methyl-pyrimidin-4-ylamino)-cyclohexyl]-amide trifluoroacetate

### Step A: Synthesis of [cis-4-(6-chloro-2-methyl-pyrimidin-4-ylamino)-cyclohexyl]-carbamic acid tert-butyl ester

To a solution of 4,6-dichloro-2-methyl-pyrimidine (4.87 g, 0.030 mol) in 50 mL MeOH were added DIEA (10.4 mL, 0.059 mol) and cis-(4-amino-cyclohexyl)-carbamic acid tert-butyl ester (6.4 g, 0.030 mol). The mixture was stirred at reflux overnight and the solvent concentrated. The resulting oil was subjected to chromatography (0-70% ethyl acetate in hexanes) to yield [cis-4-(6-chloro-2-methyl-pyrimidin-4-ylamino)-cyclohexyl]-carbamic acid tert-butyl ester (9.7 g, 0.028 mol, 95%) as a white solid.

ESI MS (M+H)⁺; ¹H NMR (400 MHz, CD₃OD) δ 6.38 (s, 1H), 4.14 (m, 1H), 3.56 (m, 1H), 2.40 (s, 3H), 1.78-1.63 (m, 8H), 1.47 (s, 9H).

### Step B: Synthesis of [cis-4-(6-dimethylamino-2-methyl-pyrimidin-4-ylamino)-cyclohexyl]-carbamic acid tert-butyl ester

To a solution [cis-4-(6-chloro-2-methyl-pyrimidin-4-ylamino)-cyclohexyl]-carbamic acid tert-butyl ester (0.5 g, 0.0015 mol) in 2 mL 2-propanol were added dimethylamine (2.20 mL, 0.0044 mol) and DIEA (511 uL, 0.0029 mol). The mixture was heated in a microwave synthesizer at 160° C. for 2 hours. The reaction was repeated 17 more times (9 g total material) and the reaction mixtures were pooled. The solvent was evaporated and the material subjected to chromatography (2-4% 2M NH₃ in MeOH/CH₂Cl₂) to yield [cis-4-(6-dimethylamino-2-methyl-pyrimidin-4-ylamino)-cyclohexyl]-carbamic acid tert-butyl ester (7.5 g, 0.021 mol, 81%) as a white solid.

ESI MS 350.4 (M+H)⁺; ¹H NMR (400 MHz, CD₃OD) δ 5.35 (s, 1H), 3.72 (m, 1H), 3.54 (m, 1H), 3.05 (s, 6H), 2.30 (s, 3H), 1.75-1.61 (m, 8H), 1.47 (s, 9H).

### Step C: Synthesis of N-(cis-4-amino-cyclohexyl)-2,N′,N′-trimethyl-pyrimidine-4,6-diamine

To a solution of [cis-4-(6-dimethylamino-2-methyl-pyrimidin-4-ylamino)-cyclohexyl]-carbamic acid tert-butyl ester (7.5 g, 0.021 mol) in 50 mL CH₂Cl₂ was added TFA (3.3 mL, 0.043 mol). The solution was stirred at room temperature for 4 hours (or until the reaction was completed as judged by TLC). The excess solvent was evaporated off and the resulting oil was dissolved in 30 mL CH₂Cl₂. The organic layer was extracted with 30 mL of a dilute NaOH (aq)/NaHCO₃ (aq) solution (the aqueous layer was confirmed to remain basic during the extraction using pH paper indicator). The aqueous layer was back extracted twice with CH₂Cl₂ and the organic layers combined, dried over MgSO₄, and concentrated to yield N-(cis-4-amino-cyclohexyl)-2,N′,N′-trimethyl-pyrimidine-4,6-diamine (5.3 g, 0.021 mol, 99%) as a white solid.

ESI MS 250.2 (M+H)⁺; ¹H NMR (400 MHz, CD₃OD) δ 5.37 (s, 1H), 3.78 (m, 1H), 3.06 (s, 6H), 2.84 (m, 1H), 2.30 (s, 3H), 1.82-1.69 (m, 6H), 1.55-1.50 (m, 2H).

### Step D: Synthesis of 5-bromo-furan-2-carboxylic acid [cis-4-(6-dimethylamino-2-methyl-pyrimidin-4-ylamino)-cyclohexyl]-amide trifluoroacetate

To a solution of N-(cis-4-amino-cyclohexyl)-2,N′,N′-trimethyl-pyrimidine-4,6-diamine (30 mg, 0.12 mmol) in 0.5 mL DMF were added 5-bromo-2-furoic acid (23 mg, 0.12 mmol), pyridine (14.6 uL, 0.18 mmol), and HATU (54.9 mg, 0.14 mmol). The reaction mixture was stirred overnight and then 0.5 mL DMSO was added to the mixture. The compound was then subject to purification by prep LCMS to yield 5-bromo-furan-2-carboxylic acid [cis-4-(6-dimethylamino-2-methyl-pyrimidin-4-ylamino)-cyclohexyl]-amide trifluoroacetate (25 mg, 0.047 mmol, 39%) as a white solid TFA salt.

ESI MS 422.2 (M+H)⁺; ¹H NMR (400 MHz, CD₃OD) δ 7.15 (d, 1H, J=3.6 Hz), 6.64 (d, 1H, J=3.6 Hz), 5.60 (s, 1H), 4.01 (m, 1H), 3.87 (m, 1H), 3.16 (s, 6H), 2.49 (s, 3H), 1.89-1.80 (m, 8H).

### Example 49

### 5-Bromo-N-[cis-4-(6-dimethylamino-2-methyl-pyrimidin-4-ylamino)-cyclohexyl]-nicotinamide trifluoroacetate

Using the procedure of Step D of Example 48, the title compound was obtained (35 mg, 53%) as a white solid.

ESI MS 433.0 (M+H)⁺; ¹H NMR (400 MHz, CD₃OD) δ 8.95 (d, 1H, J=1.6 Hz), 8.84 (d, 1H, J=2.0 Hz), 8.58 (m, 1H), 8.43 (t, 1H, J=2.0 Hz), 5.60 (s, 1H), 4.05 (m, 1H), 3.88 (m, 1H), 3.22 (s, 6H), 2.49 (s, 3H), 1.93-1.84 (m, 8H).

### Example 50

### N-[cis-4-(6-Dimethylamino-2-methyl-pyrimidin-4-ylamino)-cyclohexyl]-3,5-bis-trifluoromethyl-benzamide trifluoroacetate

To a solution of N-(cis-4-amino-cyclohexyl)-2,N′,N′-trimethyl-pyrimidine-4,6-diamine (30 mg, 0.12 mmol) in 0.5 mL DMF were added pyridine (14.6 uL, 0.18 mmol) and 3,5-bis(trifluoromethyl)benzoyl chloride (21.8 uL, 0.12 mmol). The reaction mixture was stirred overnight and then 0.5 mL of DMSO was added to the mixture. The compound was then subject to purification by prep LCMS to yield N-[cis-4-(6-dimethylamino-2-methyl-pyrimidin-4-ylamino)-cyclohexyl]-3,5-bis-trifluoromethyl-benzamide trifluoroacetate (12 mg, 0.020 mmol, 17%) as a white solid TFA salt.

ESI MS 490.4 (M+H)⁺; ¹H NMR (400 MHz, CD₃OD) δ 8.46 (s, 2H), 8.19 (s, 1H), 5.42 (s, 1H), 4.06 (m, 1H), 3.86 (m, 1H), 3.09 (s, 6H), 2.34 (s, 3H), 1.93-1.79 (m, 9H).

### Example 51

### N-[cis-4-(6-Dimethylamino-2-methyl-pyrimidin-4-ylamino)-cyclohexyl]-3,5-difluoro-benzamide trifluoroacetate

Using the procedure of Step A of Example 50, the title compound was obtained (22 mg, 0044 mmol, 36%) as a white solid.

ESI MS 390.2 (M+H)⁺; ¹H NMR (400 MHz, CD₃OD) δ 7.50-7.46 (m, 2H), 7.22-7.16 (m, 1H), 5.60 (s, 1H), 4.02 (m, 1H), 3.87 (m, 1H), 3.22 (s, 6H), 2.49 (s, 3H), 1.90-1.81 (m, 8H).

### Example 52

### N-[cis-4-(3,5-Dimethoxy-benzylamino)-cyclohexyl]-2,N′,N′-trimethyl-pyrimidine-4,6-diamine bis-trifluoroacetate

To a solution of N-(cis-4-amino-cyclohexyl)-2,N′,N′-trimethyl-pyrimidine-4,6-diamine (24.9 mg, 0.1 mmol) in 0.5 mL MeOH was added 3,5-dimethoxybenzaldehyde (16.6 mg, 0.1 mmol). The mixture was stirred at room temperature for a half an hour and then sodium triacetoxyborohydride (84.8 mg, 0.4 mmol) was added. The mixture was stirred at room temperature overnight and then 0.5 mL of DMSO was added to the mixture. The compound was then subject to purification by prep LCMS to yield N-[cis-4-(3,5-dimethoxy-benzylamino)-cyclohexyl]-2,N′,N′-trimethyl-pyrimidine-4,6-diamine bis-trifluoroacetate (27 mg, 0.043 mmol, 43%) as a white solid TFA salt.

ESI MS 400.5 (M+H)⁺; ¹H NMR (400 MHz, CD₃OD) δ 6.72 (d, 2H, J=2.0 Hz), 6.59 (t, 1H, J=2.0 Hz), 5.59 (s, 1H), 4.22 (s, 2H), 3.97 (m, 1H), 3.84 (m, 1H), 3.79 (s, 6H), 3.22 (s, 6H), 2.48 (s, 3H), 2.11-2.02 (m, 4H), 1.95-1.81 (m, 4H).

### Example 53

### N-[cis-4-(3-Bromo-benzylamino)-cyclohexyl]-2,N′,N′-trimethyl-pyrimidine-4,6-diamine bis-trifluoroacetate

Using the procedure of Step A of Example 52, the title compound was obtained (35 mg, 0054 mmol, 54%) as a white solid.

ESI MS 418.0 (M+H)⁺; ¹H NMR (400 MHz, CD₃OD) δ 7.78 (s, 1H), 7.68 (d, 1H, J=8.0 Hz), 7.55 (d, 1H, 7.6 Hz), 7.43 (t, 1H, J=8.0 Hz), 5.60 (s, 1H), 4.29 (s, 2H), 3.21 (s, 6H), 2.48 (s, 3H), 2.12-2.03 (m, 4H), 1.95-1.85 (m, 4H).

### Example 54

### 1-[cis-4-(6-Dimethylamino-2-methyl-pyrimidin-4-ylamino)-cyclohexyl]-3-(3-methoxy-phenyl)-urea trifluoroacetate

To a solution of N-(cis-4-amino-cyclohexyl)-2,N′,N′-trimethyl-pyrimidine-4,6-diamine (24.9 mg, 0.1 mmol) in 0.5 mL DMSO was added 3-methoxyphenyl isocyanate (11.8 uL, 0.09 mmol). The mixture was stirred at room temperature overnight and then 0.5 mL of DMSO was added to the mixture. The compound was then subject to purification by prep LCMS to yield 1-[cis-4-(6-dimethylamino-2-methyl-pyrimidin-4-ylamino)-cyclohexyl]-3-(3-methoxy-phenyl)-urea trifluoroacetate (19 mg, 0.037 mmol, 41%) as a white solid TFA salt.

ESI MS 399.2 (M+H)⁺; ¹H NMR (400 MHz, CD₃OD) δ 7.15 (s, 1H), 7.14 (t, 1H, J=2.4 Hz), 6.86 (dd, 1H, J₁=8.0 Hz, J₂=2.0 Hz), 6.57 (dd, 1H, J₁=8.0 Hz, J₂=2.4 Hz), 5.57 (s, 1H), 3.84 (m, 1H), 3.79 (s, 3H), 3.78 (m, 1H), 3.21 (s, 6H), 2.47 (s, 3H), 1.90-1.75 (m, 8H).

### Example 55

### 1-(3,5-Difluoro-phenyl)-3-[cis-4-(6-dimethylamino-2-methyl-pyrimidin-4-ylamino)-cyclohexyl]-urea trifluoroacetate

Using the procedure of Step A of Example 54, the title compound was obtained (22 mg, 0.043 mmol, 47%) as a white solid.

ESI MS 405.4 (M+H)⁺; ¹H NMR (400 MHz, CD₃OD) δ 7.07-7.04 (m, 2H), 6.54-6.50 (m, 1H), 5.60 (s, 1H), 3.83 (m, 1H), 3.82 (m, 1H), 3.18 (s, 6H), 2.48 (s, 3H), 1.90-1.83 (m, 4H), 1.79-1.75 (m, 4H).

### Example 56

### N-[cis-4-(6-Dimethylamino-2-methylsulfanyl-pyrimidin-4-ylamino)-cyclohexyl]-3,4-difluoro-benzamide trifluoroacetate

### Step A: Synthesis of cis-[4-(3,4-difluoro-benzoylamino)-cyclohexyl]-carbamic acid tert-butyl ester

To a solution of cis-(4-amino-cyclohexyl)-carbamic acid tert-butyl ester (3 g, 0.014 mol) in CH₂Cl₂ (50 mL) was added DIEA (3.6 mL, 0.021 mol). The mixture was cooled on an ice bath and 3,4-difluorobenzoyl chloride (1.9 mL, 0.015 mol) was slowly added. The mixture was brought to room temperature and stirred for 1 hour. The solvent was then concentrated and the resulting oil subjected to chromatography (0-70% ethyl acetate in hexanes). Upon evaporation of solvents, a precipitate crashed out which was filtered and washed with 70% cold ether in hexanes to yield cis-[4-(3,4-difluoro-benzoylamino)-cyclohexyl]-carbamic acid tert-butyl ester (4.4 g, 0.012 mol, 89%) as a white solid.

ESI 355.4 M+H⁺; ¹H NMR (400 MHz, CD₃OD) δ 7.78-7.72 (m, 1H), 7.68-7.64 (m, 1H), 7.39-7.33 (m, 1H), 3.93 (m, 1H), 3.61 (m, 1H), 1.78-1.68 (m, 8H), 1.45 (s, 9H).

### Step B: Synthesis of cis-N-(4-amino-cyclohexyl)-3,4-difluoro-benzamide

To a solution of cis-[4-(3,4-difluoro-benzoylamino)-cyclohexyl]-carbamic acid tert-butyl ester (4.4 g, 0.012 mol) in CH₂Cl₂ (50 mL) was added TFA (1.9 mL, 0.025 mol). The solution was stirred at room temperature for 4 hours (or until the reaction was complete as judged by TLC). The excess solvent was evaporated off and the resulting oil was dissolved in 30 mL CH₂Cl₂. The organic layer was extracted with 30 mL of a dilute NaOH (aq)/NaHCO₃ (aq) solution (the aqueous layer was confirmed to remain basic during the extraction using pH paper indicator). The aqueous layer was back extracted twice with CH₂Cl₂ and the organic layers combined, dried over MgSO₄, and concentrated to yield cis-N-(4-amino-cyclohexyl)-3,4-difluoro-benzamide (2.9 g, 0.011 mol, 90%) as a white solid.

ESI 255.4 M+H⁺; ¹H NMR (400 MHz, CD₃OD) δ 8.17 (d, 1H, J=4.8 Hz), 7.93-7.88 (m, 1H), 7.80-7.70 (m, 4H), 7.58-7.51 (m, 1H), 3.86 (m, 1H), 3.12 (m, 1H), 1.91-1.87 (m, 2H), 1.73-1.60 (m, 6H).

### Step C: Synthesis of cis-N-[4-(6-chloro-2-methylsulfanyl-pyrimidin-4-ylamino)-cyclohexyl]-3,4-difluoro-benzamide

To a solution of 4,6-dichloro-2-(methylthio)-pyrimidine (19.5 mg, 0.1 mmol) in IPA (0.6 mL) were added DIEA (35 uL, 0.2 mmol) and cis-N-(4-amino-cyclohexyl)-3,4-difluoro-benzamide (25.4 mg, 0.1 mmol). The mixture was then heated in a microwave at 170° C. for 30 minutes. The reaction mixture was cooled and concentrated and the resulting oil was purified by column (0-100% ethyl acetate in hexanes) to yield cis-N-[4-(6-chloro-2-methylsulfanyl-pyrimidin-4-ylamino)-cyclohexyl]-3,4-difluoro-benzamide (37 mg, 0.090 mmol, 90%) as a colorless oil.

ESI MS 413.2 (M+H)⁺; ¹H NMR (400 MHz, CD₃OD) δ 8.23 (m, 1H), 7.81-7.76 (m, 1H), 7.72-7.68 (m, 1H), 7.43-7.36 (m, 1H), 6.27 (s, 1H), 4.17 (m, 1H), 4.00 (m, 1H), 2.51 (s, 3H), 1.94-1.79 (m, 8H).

### Step D: Synthesis of N-[cis-4-(6-dimethylamino-2-methylsulfanyl-pyrimidin-4-ylamino)-cyclohexyl]-3,4-difluoro-benzamide trifluoroacetate

To a solution of cis-N-[4-(6-chloro-2-methylsulfanyl-pyrimidin-4-ylamino)-cyclohexyl]-3,4-difluoro-benzamide (73 mg, 0.18 mmol) in IPA (0.8 mL) were added DIEA (62 uL, 0.35 mmol) and dimethylamine (265 uL, 0.53 mmol). The mixture was then heated in a microwave at 170° C. for 1 hour. The reaction mixture was cooled and concentrated and the resulting oil was re-dissolved into 1 mL DMSO and purified by prep LCMS to yield N-[cis-4-(6-dimethylamino-2-methylsulfanyl-pyrimidin-4-ylamino)-cyclohexyl]-3,4-difluoro-benzamide trifluoroacetate (18.4 mg, 0.034 mmol, 19%) as a TFA salt.

ESI MS 422.2 (M+H)⁺; ¹H NMR (400 MHz, CD₃OD) δ 8.28 (m, 1H), 7.82-7.76 (m, 1H), 7.73-7.69 (m, 1H), 7.43-7.36 (m, 1H), 4.88 (s, 1H), 4.02 (m, 1H), 3.89 (m, 1H), 3.11 (s, 6H), 2.66 (s, 3H), 1.92-1.79 (m, 8H).

### Example 57

### N-[cis-4-(6-Dimethylamino-pyrimidin-4-ylamino)-cyclohexyl]-3,4-difluoro-benzamide trifluoroacetate

To a solution of 4,6-dichloropyrimidine (149 mg, 0.1 mmol) in IPA (1 mL) were added DIEA (35 uL, 0.2 mmol) and cis-N-(4-amino-cyclohexyl)-3,4-difluoro-benzamide from Step B Example 56 (25.4 mg, 0.1 mmol). The mixture was then heated in a microwave at 170° C. for 15 minutes. The reaction mixture was cooled and then DIEA (35 uL, 0.2 mmol) and dimethylamine (150 uL, 0.3 mmol) were added. The mixture was then heated in a microwave at 170° C. for 1 hour. The reaction mixture was cooled and concentrated and the resulting oil was re-dissolved into 1 mL DMSO and purified by prep LCMS to yield N-[cis-4-(6-dimethylamino-pyrimidin-4-ylamino)-cyclohexyl]-3,4-difluoro-benzamide trifluoroacetate (11.7 mg, 0.024 mmol, 24%) as a TFA salt.

ESI MS 376.3 (M+H)⁺; ¹H NMR (400 MHz, CD₃OD) δ 8.27 (m, 1H), 8.18 (s, 1H), 7.82-7.76 (m, 1H), 7.73-7.69 (m, 1H), 7.43-7.36 (m, 1H), 5.71 (s, 1H), 4.02 (m, 1M), 3.88 (m, 1H), 3.23 (s, 6H), 1.90-1.84 (m, 8H).

### Example 58

### N-[cis-4-(6-Dimethylamino-5-methyl-pyrimidin-4-ylamino)-cyclohexyl]-3,4-difluoro-benzamide trifluoroacetate

To a solution of 2-methyl-4,6-dichloropyrimidine (326 mg, 0.2 mmol) in IPA (1 mL) were added DIEA (70 uL, 0.4 mmol) and cis-N-(4-amino-cyclohexyl)-3,4-difluoro-benzamide from Step B Example 56 (50.8 mg, 0.2 mmol). The mixture was then heated in a microwave at 170° C. for 15 minutes. The reaction mixture was cooled and then DIEA (70 uL, 0.4 mmol) and dimethylamine (300 uL, 0.3 mmol) were added. The mixture was then heated in a microwave at 170° C. for 1 hour. The reaction mixture was cooled and concentrated and the resulting oil was re-dissolved into 1 mL DMSO and purified by prep LCMS to yield N-[cis-4-(6-dimethylamino-5-methyl-pyrimidin-4-ylamino)-cyclohexyl]-3,4-difluoro-benzamide trifluoroacetate (32.2 mg, 0.064 mmol, 64%) as a TFA salt.

ESI MS 390.2 (M+H)⁺; ¹H NMR (400 MHz, CD₃OD) δ 8.20 (s, 1H), 8.17 (m, 1H), 7.81-7.78 (m, 1H), 7.72-7.71 (m, 1H), 7.42-7.40 (m, 1H), 4.10 (m, 1H), 4.09 (m, 1H), 3.16 (s, 6H), 2.16 (s, 3H), 2.02-1.82 (m, 8H).

### Example 59

### 3,4-Dichloro-N-[cis-4-(2-dimethylamino-6-methyl-pyrimidin-4-ylamino)-cyclohexyl]-benzamide trifluoroacetate

### Step A: Synthesis of cis-[4-(2-chloro-6-methyl-pyrimidin-4-ylamino)-cyclohexyl]-carbamic acid tert-butyl ester

To a solution of 2,4-dichloro-6-methylpyrimidine (3.7 g, 0.023 mol) in 30 mL methanol were added DIEA (5.89 mL, 0.034 mmol) and cis-(4-amino-cyclohexyl)-carbamic acid tert-butyl ester (5.3 g, 0.025 mol). The mixture was refluxed overnight, cooled, and concentrated. The resulting oil was subjected to chromatography (0-100% ethyl acetate in hexanes) to yield cis-[4-(2-chloro-6-methyl-pyrimidin-4-ylamino)-cyclohexyl]-carbamic acid tert-butyl ester (5.1 g, 0.015 mol, 66%) as a white solid.

ESI MS 341.4 (M+H)⁺; ¹H NMR (400 MHz, CD₃OD) δ 6.31 (s, 1H), 4.12 (m, 1H), 3.56 (m, 1H), 2.26 (s, 3H), 1.78-1.67 (m, 8H), 1.48 (s, 9H).

### Step B: Synthesis of cis-[4-(2-dimethylamino-6-methyl-pyrimidin-4-ylamino)-cyclohexyl]-carbamic acid tert-butyl ester

To a solution cis-[4-(2-chloro-6-methyl-pyrimidin-4-ylamino)-cyclohexyl]-carbamic acid tert-butyl ester (0.5 g, 0.0015 mol) in 2 mL 2-propanol were added dimethylamine (1.47 mL, 0.0029 mol) and DIEA (511 uL, 0.0029 mol). The mixture was heated in a microwave synthesizer at 170° C. for 1 hour. The reaction was repeated 9 more times (5 g total material) and the reaction mixtures were pooled. The solvent was evaporated and the material subjected to chromatography (2-4% 2M NH₃ in MeOH/CH₂Cl₂) to yield cis-[4-(2-dimethylamino-6-methyl-pyrimidin-4-ylamino)-cyclohexyl]-carbamic acid tert-butyl ester (2.2 g, 0.0063 mol, 43%) as a white solid.

ESI MS 350.2 (M+H)⁺; ¹H NMR (400 MHz, CD₃OD) δ 5.68 (s, 1H), 3.95 (m, 1H), 3.54 (m, 1H), 3.11 (s, 6H), 2.16 (s, 3H), 1.77-1.64 (m, 8H), 1.47 (s, 9H).

### Step C: Synthesis of cis-4-(2-dimethylamino-6-methyl-pyrimidin-4-ylamino)-1-amino-cyclohexane

To a solution of cis-[4-(2-dimethylamino-6-methyl-pyrimidin-4-ylamino)-cyclohexyl]-carbamic acid tert-butyl ester (2.2 g, 0.0063 mol) in 15 mL CH₂Cl₂ was added TFA (0.97 mL, 0.013 mol). The solution was stirred at room temperature for 4 hours (or until the reaction was complete as judged by TLC). The excess solvent was evaporated off and the resulting oil was dissolved in 30 mL CH₂Cl₂. The organic layer was extracted with 30 mL of a dilute NaOH (aq)/NaHCO₃ (aq) solution (the aqueous layer was confirmed to remain basic during the extraction using pH paper indicator). The aqueous layer was back extracted twice with CH₂Cl₂ and the organic layers combined, dried over MgSO₄, and concentrated to yield cis-4-(2-dimethylamino-6-methyl-pyrimidin-4-ylamino)-1-amino-cyclohexane (1.3 g, 0.0052 mol, 83%) as a white solid.

ESI MS 250.2 (M+H)⁺, ¹H NMR (400 MHz, CD₃OD) δ 5.70 (s, 1H), 4.00 (m, 1H), 3.11 (s, 6H), 2.84 (m, 1H), 2.16 (s, 3H), 1.86-1.80 (m, 2H), 1.76-1.66 (m, 4H), 1.57-1.49 (m, 2H).

### Step D: Synthesis of 3,4-dichloro-N-[cis-4-(2-dimethylamino-6-methyl-pyrimidin-4-ylamino)-cyclohexyl]-benzamide trifluoroacetate

To a solution of cis-4-(2-dimethylamino-6-methyl-pyrimidin-4-ylamino)-1-amino-cyclohexane (20 mg, 0.080 mmol) in 0.5 mL DMF was added pyridine (9.7 uL, 0.12 mmol) and 3,4-dichlorobenzoyl chloride (11.1 uL, 0.076 mmol). The reaction mixture was stirred overnight and then 0.5 mL of DMSO was added to the mixture. The compound was then subject to purification by prep LCMS to yield 3,4-dichloro-N-[cis-4-(2-dimethylamino-6-methyl-pyrimidin-4-ylamino)-cyclohexyl]-benzamide trifluoroacetate (10 mg, 0.019 mmol, 24%) as a TFA salt.

ESI MS 422.2 (M+H)⁺; ¹H NMR (400 MHz, CD₃OD) δ 8.00 (d, 1H, J=2.0 Hz), 7.76 (dd, J₁=8.4 Hz, J₂=2.0 Hz), 7.65 (d, 1H, J=8.4 Hz), 6.01 (s, 1H), 4.23 (m, 1H), 4.00 (m, 1H), 3.26 (s, 6H), 2.34 (s, 3H), 1.98-1.81 (m, 8H).

### Example 60

### 4-Cyano-N-[cis-4-(2-dimethylamino-6-methyl-pyrimidin-4-ylamino)-cyclohexyl]-benzamide trifluoroacetate

Using the procedure of Step D of Example 59, the title compound was obtained (11 mg, 0022 mmol, 29%).

ESI MS 379.2 (M+H)⁺; ¹H NMR (400 MHz, CD₃OD) δ 7.97 (d, 2H, J=8.0 Hz), 7.86 (d, 2H, J=8.4 Hz), 6.01 (s, 1H), 4.23 (m, 1H), 4.03 (m, 1H), 3.26 (s, 6H), 2.34 (s, 3H), 1.99-1.82 (m, 8H).

### Example 61

### N-[cis-4-(2-Dimethylamino-6-methyl-pyrimidin-4-ylamino)-cyclohexyl]-3,4-diethoxy-benzamide trifluoroacetate

To a solution of cis-4-(2-dimethylamino-6-methyl-pyrimidin-4-ylamino)-1-amino-cyclohexane (20 mg, 0.080 mmol) in 0.5 mL DMF were added 3,4-diethoxy-benzoic acid (16.0 mg, 0.076 mmol), pyridine (9.7 uL, 0.12 mmol), and HATU (36.6 mg, 0.096 mmol). The reaction mixture was stirred overnight and then 0.5 mL DMSO was added to the mixture. The compound was then subject to purification by prep LCMS to yield N-[cis-4-(2-dimethylamino-6-methyl-pyrimidin-4-ylamino)-cyclohexyl]-3,4-diethoxy-benzamide trifluoroacetate (11 mg, 0.020 mmol, 26%) as a TFA salt.

ESI MS 442.4 (M+H)⁺; ¹H NMR (400 MHz, CD₃OD) δ 7.47-7.44 (m, 2H), 7.02-7.00 (m, 1H), 6.01 (s, 1H), 4.23 (m, 1H), 4.15 (q, 4H, J=7.0 Hz), 4.00 (m, 1H), 3.26 (s, 3H), 2.34 (s, 3H), 1.99-1.81 (m, 8H), 1.45 (t, 6H, J=7.2 Hz).

### Example 62

### 3-Chloro-N-[cis-4-(2-dimethylamino-6-methyl-pyrimidin-4-ylamino)-cyclohexyl]-5-fluoro-benzamide trifluoroacetate

Using the procedure of Step A of Example 61, the title compound was obtained (12 mg, 0023 mmol, 30%).

ESI MS 406.4 (M+H)⁺; ¹H NMR (400 MHz, CD₃OD) δ 7.71 (s, 1H), 7.57-7.53 (m, 1H), 7.45-7.42 (m, 1H), 6.00 (s, 1H), 4.23 (m, 1H), 4.00 (m, 1H), 3.26 (s, 6H), 2.34 (s, 3H), 1.99-1.82 (m, 8H).

### Example 63

### N-[cis-4-(2-Dimethylamino-5-methyl-pyrimidin-4-ylamino)-cyclohexyl]-3,5-dimethoxy-benzamide trifluoroacetate

### Step A: Synthesis of cis-[4-(2-chloro-5-methyl-pyrimidin-4-ylamino)-cyclohexyl]-carbamic acid tert-butyl ester

To a solution of 2,4-dichloro-5-methylpyrimidine (1.0 g, 6.13 mmol) in 2 mL 2-propanol were added DIEA (1.6 mL, 9.20 mmol) and cis-(4-amino-cyclohexyl)-carbamic acid tert-butyl ester (1.45 g, 6.75 mmol). The mixture was heated in a microwave synthesizer at 150° C. for 15 minutes. The solvent was evaporated and the material subjected to chromatography (0-70% ethyl acetate in hexanes) to yield cis-[4-(2-chloro-5-methyl-pyrimidin-4-ylamino)-cyclohexyl]-carbamic acid tert-butyl ester (1.7 g, 4.86 mmol, 79%) as a white solid.

ESI MS 341.2 (M+H)⁺, ¹H NMR (400 MHz, CD₃OD) δ 7.76 (s, 1H), 4.12 (m, 1H), 3.67 (m, 1H), 2.05 (s, 3H), 1.82-1.70 (m, 8H), 1.48 (s, 9H).

### Step B: Synthesis of cis-[4-(2-dimethylamino-5-methyl-pyrimidin-4-ylamino)-cyclohexyl]-carbamic acid tert-butyl ester

To a solution cis-[4-(2-chloro-5-methyl-pyrimidin-4-ylamino)-cyclohexyl]-carbamic acid tert-butyl ester (0.5 g, 0.0015 mol) in 2 mL 2-propanol were added dimethylamine (1.47 mL, 0.0029 mol) and DIEA (511 uL, 0.0029 mol). The mixture was heated in a microwave synthesizer at 170° C. for 1 hour. The reaction was repeated 2 more times (1.5 g total material) and the reaction mixtures were pooled. The solvent was evaporated and the material subjected to chromatography (2-4% 2M NH₃ in MeOH/CH₂Cl₂) to yield cis-[4-(2-dimethylamino-5-methyl-pyrimidin-4-ylamino)-cyclohexyl]-carbamic acid tert-butyl ester (1.3 g, 0.0037 mol, 85%) as a white solid.

ESI MS 350.2 (M+H)⁺; ¹H NMR (400 MHz, CD₃OD) δ 7.53 (s, 1H), 4.13 (m, 1H), 3.63 (m, 1H), 3.09 (s, 6H), 1.94 (s, 3H), 1.83-1.70 (m, 8H), 1.48 (s, 9H).

### Step C: Synthesis of cis-4-(2-dimethylamino-5-methyl-pyrimidin-4-ylamino)-1-amino-cyclohexane

To a solution of cis-[4-(2-dimethylamino-5-methyl-pyrimidin-4-ylamino)-cyclohexyl]-carbamic acid tert-butyl ester (1.3 g, 0.0037 mol) in 10 mL CH₂Cl₂ was added TFA (0.57 mL, 0.0074 mol). The solution was stirred at room temperature for 4 hours (or until the reaction was complete as judged by TLC). The excess solvent was evaporated off and the resulting oil was dissolved in 30 mL CH₂Cl₂. The organic layer was extracted with 30 mL of a dilute NaOH (aq)/NaHCO₃ (aq) solution (the aqueous layer was confirmed to remain basic during the extraction using pH paper indicator). The aqueous layer was back extracted twice with CH₂Cl₂ and the organic layers combined, dried over MgSO₄, and concentrated to yield cis-4-(2-dimethylamino-5-methyl-pyrimidin-4-ylamino)-1-amino-cyclohexane (0.88 g, 0.0035 mol, 95%) as a white solid.

ESI MS 250.2 (M+H)⁺; ¹H NMR (400 MHz, CD₃OD) δ 7.53 (s, 1H), 4.17 (m, 1H), 3.09 (s, 6H), 2.94 (m, 1H), 1.96 (s, 3H), 1.86-1.71 (m, 6H), 1.62-1.59 (m, 2H).

### Step D: Synthesis of N-[cis-4-(2-dimethylamino-5-methyl-pyrimidin-4-ylamino)-cyclohexyl]-3,5-dimethoxy-benzamide trifluoroacetate

To a solution of cis-4-(2-dimethylamino-5-methyl-pyrimidin-4-ylamino)-1-amino-cyclohexane (20 mg, 0.080 mmol) in 0.5 mL DMF were added pyridine (9.7 uL, 0.12 mmol) and 3,5-dimethoxybenzoyl chloride (15.3 mg, 0.076 mmol). The reaction mixture was stirred overnight and then 0.5 mL of DMSO was added to the mixture. The compound was then subject to purification by prep LCMS to yield N-[cis-4-(2-dimethylamino-5-methyl-pyrimidin-4-ylamino)-cyclohexyl]-3,5-dimethoxy-benzamide trifluoroacetate (14 mg, 0.027 mmol, 35%) as a TFA salt.

ESI MS 414.4 (M+H)⁺; ¹H NMR (400 MHz, CD₃OD) δ 8.00 (s, 1H), 7.48 (s, 1H), 7.19 (d, 1H, J=2.4 Hz), 6.69 (t, 1H, J=2.4 Hz), 4.31 (m, 1H), 4.10 (m, 1H), 3.85 (s, 6H), 3.23 (s, 6H), 2.32 (s, 3H), 2.10-1.82 (m, 8H).

### Example 64

### 3,4-Dichloro-N-[cis-4-(2-dimethylamino-5-methyl-pyrimidin-4-ylamino)-cyclohexyl]-benzamide trifluoroacetate

Using the procedure of Step D of Example 63, the title compound was obtained (15 mg, 0028 mmol, 37%).

ESI MS 422.2 (M+H)⁺; ¹H NMR (400 MHz, CD₃OD) δ 8.24 (m, 1H), 8.02 (d, 1H, J=2.0 Hz), 7.78 (dd, 1H, J₁=8.4 Hz, J₂=2.0 Hz), 7.67 (d, 1H, J=8.4 Hz), 7.48 (s, 1H), 4.31 (m, 1H), 4.10 (m, 1H), 3.23 (s, 6H), 2.10 (s, 3H), 2.00-1.82 (m, 8H).

### Example 65

### N-[cis-4-(2-Dimethylamino-5-methyl-pyrimidin-4-ylamino)-cyclohexyl]-3,4-diethoxy-benzamide trifluoroacetate

To a solution of cis-4-(2-dimethylamino-5-methyl-pyrimidin-4-ylamino)-1-amino-cyclohexane (20 mg, 0.080 mmol) in 0.5 mL DMF were added 3,4-diethoxy-benzoic acid (16.0 mg, 0.076 mmol), pyridine (9.7 uL, 0.12 mmol), and HATU (36.6 mg, 0.096 mmol). The reaction mixture was stirred overnight and then 0.5 mL DMSO was added to the mixture. The compound was then subject to purification by prep LCMS to yield N-[cis-4-(2-dimethylamino-5-methyl-pyrimidin-4-ylamino)-cyclohexyl]-3,4-diethoxy-benzamide trifluoroacetate (12 mg, 0.022 mmol, 28%) as a TFA salt.

ESI MS 442.4 (M+H)⁺; ¹H NMR (400 MHz, CD₃OD) δ 7.49-7.46 (m, 3H), 7.02 (d, 1H, J=8.0 Hz), 4.31 (m, 1H), 4.16 (q, 4H, J=7.0 Hz), 4.10 (m, 1H), 3.23 (s, 6H), 2.10 (s, 3H), 2.01-1.81 (m, 8H), 1.46 (t, 6H, J=7.0 Hz).

### Example 66

### 3-Chloro-N-[cis-4-(2-dimethylamino-5-methyl-pyrimidin-4-ylamino)-cyclohexyl]-5-fluoro-benzamide trifluoroacetate

Using the procedure of Step A of Example 65, the title compound was obtained (12 mg, 0023 mmol, 30%).

ESI MS 406.2 (M+H)⁺; ¹H NMR (400 MHz, CD₃OD) δ 7.73 (s, 1H), 7.59-7.56 (m, 1H), 7.48 (s, 1H), 7.46-7.43 (m, 1H), 4.31 (m, 1H), 4.10 (m, 1H), 3.23 (s, 6H), 2.10 (s, 3H), 2.03-1.81 (m, 8H).

### Example 67

### N-[cis-4-(6-Dimethylamino-2-methyl-pyrimidin-4-ylamino)-cyclohexylmethyl]-3,5-bis-trifluoromethyl-benzamide trifluoroacetate

### Step A: Synthesis of cis-(4-amino-cyclohexylmethyl)-carbamic acid benzyl ester

To a solution of cis-(4-aminomethyl-cyclohexyl)-carbamic acid tert-butyl ester (25 g, 0.11 mol) in CH₂Cl₂ (300 mL) was added DIEA (22.9 mL, 0.13 mol). The mixture was cooled on an ice bath and benzyl chloroformate (17.3 mL, 0.12 mol) was slowly added. The mixture was removed from the ice bath and stirred overnight. The solvent was removed in vacuo and the resulting oil dissolved in MeOH (250 mL). Concentrated HCl (75 mL) was slowly added to the mixture with stirring. The reaction was allowed to stir for 4 more hours and then the solvent was removed in vacuo resulting in a precipitate. A copious amount of water (2 L) was added to dissolve the resulting HCl salt precipitate, which was then made basic with slow addition of a concentrated NaOH solution. The aqueous layer was extracted 3 times with ethyl acetate (1 L). The organic layers were combined, dried over MgSO₄, and concentrated to yield cis-(4-amino-cyclohexylmethyl)-carbamic acid benzyl ester (24.5 g, 0.093 mol, 85%) as an oil.

ESI MS m/e 263.2 (M+H)⁺; ¹H NMR (400 MHz, DMSO-d₆) δ 7.36-7.25 (m, 5H), 4.99 (s, 2H), 2.90 (t, J=6.4 Hz, 2H), 2.81 (m, 1H), 143-1.34 (m, 8H).

### Step B: Synthesis of cis-[4-(6-chloro-2-methyl-pyrimidin-4-ylamino)-cyclohexylmethyl]-carbamic acid benzyl ester

To a solution of 4,6-dichloro-2-methyl-pyrimidine (1.0 g, 6.1 mmol) in 2 mL 2-propanol were added DIEA (1.6 mL, 9.2 mmol) and cis-(4-amino-cyclohexylmethyl)-carbamic acid benzyl ester (1.8 g, 6.7 mmol). The mixture was heated in a microwave synthesizer at 160° C. for 20 minutes. The reaction was repeated 2 more times (3 g total material) and the reaction mixtures were pooled. The solvent was evaporated and the material subjected to chromatography (0-100% ethyl acetate in hexanes) to yield cis-[4-(6-chloro-2-methyl-pyrimidin-4-ylamino)-cyclohexylmethyl]-carbamic acid benzyl ester (6.5 g, 0.017 mol, 91%) as a white solid.

ESI MS m/e 389.2 (M+H)⁺; ¹H NMR (400 MHz, CDCl₃) δ 7.35-7.26 (m, 5H), 6.17 (s, 1H), 5.09 (s, 2H), 4.89 (m, 1H), 3.10 (t, J=6.0 Hz, 2H), 2.46 (s, 3H), 1.80-1.67 (m, 2H), 1.66-1.60 (m, 4H), 1.30-1.22 (m, 2H).

### Step C: Synthesis of cis-[4-(6-dimethylamino-2-methyl-pyrimidin-4-ylamino)-cyclohexylmethyl]-carbamic acid benzyl ester

To a solution of cis-[4-(6-chloro-2-methyl-pyrimidin-4-ylamino)-cyclohexylmethyl]-carbamic acid benzyl ester (0.5 g, 1.3 mmol) in 2 mL 2-propanol were added DIEA (224 uL, 1.3 mmol) and dimethylamine (1.3 mL, 2.6 mmol). The mixture was heated in a microwave synthesizer at 170° C. for 30 minutes. The reaction was repeated 7 more times (8 g total material) and the reaction mixtures pooled. The solvent was evaporated and the material subjected to chromatography (0-100% ethyl acetate in hexanes to remove starting material, followed by <5% MeOH in CH₂Cl₂) to yield cis-[4-(6-dimethylamino-2-methyl-pyrimidin-4-ylamino)-cyclohexylmethyl]-carbamic acid benzyl ester (3.8 g, 9.6 mmol, 94%) as a white solid.

ESI MS m/e 398.2 (M+H)⁺; ¹H NMR (400 MHz, CDCl₃) δ7.6-7.26 (m, 5H), 5.10 (s, 1H), 5.09 (s, 2H), 5.06 (m, 1H), 3.69 (m, 1H), 3.09 (m, 8H), 2.40 (s, 3H), 1.87-1.83 (m, 2H), 1.65-1.56 (m, 4H), 1.42-1.36 (m, 2H).

### Step D: Synthesis of cis-N-(4-aminomethyl-cyclohexyl)-2,N′,N′-trimethyl-pyrimidine-4,6-diamine

To a solution of cis-[4-(6-dimethylamino-2-methyl-pyrimidin-4-ylamino)-cyclohexylmethyl]-carbamic acid benzyl ester (3.8 g, 9.6 mmol) in EtOH (100 mL) was added 10% Pd/C (380 mg). The reaction mixture was stirred at room temperature under an H₂(g) atmosphere for 15 hours. The H₂(g) atmosphere was removed and the mixture washed through a plug of celite with ethyl acetate. The solvent was concentrated and the material was subjected to chromatography (2-4% 2M NH₃ in MeOH/C₁H₂Cl₂) to yield cis-N-(4-aminomethyl-cyclohexyl)-2,N′,N′-trimethyl-pyrimidine-4,6-diamine (1.7 g, 6.5 mmol, 64%) as a white solid.

ESI MS m/e 264.2 (M+H)⁺; ¹H NMR (400 MHz, DMSO) δ 6.29 (m, 1H), 5.33 (s, 1H), 3.87 (m, 1H), 2.91 (s, 6H), 2.42 (s, 2H), 2.15 (s, 3H), 1.55-1.29 (m, 8H).

### Step E: Synthesis of N-[cis-4-(6-dimethylamino-2-methyl-pyrimidin-4-ylamino)-cyclohexylmethyl]-3,5-bis-trifluoromethyl-benzamide trifluoroacetate

To a solution of cis-N-(4-aminomethyl-cyclohexyl)-2,N′,N′-trimethyl-pyrimidine-4,6-diamine (26 mg, 0.10 mmol) in 0.5 mL DMF were added pyridine (12.1 uL, 0.15 mmol) and 3,5-bis(trifluoromethyl)benzoyl chloride (18.1 uL, 0.10 mmol). The reaction mixture was stirred overnight and then 0.5 mL of DMSO was added to the mixture. The compound was then subject to purification by prep LCMS to yield N-[cis-4-(6-dimethylamino-2-methyl-pyrimidin-4-ylamino)-cyclohexylmethyl]-3,5-bis-trifluoromethyl-benzamide trifluoroacetate (11.9 mg, 0.019 mmol, 19%) as a white solid TFA salt.

ESI MS m/e 504.2 (M+H)⁺; ¹H NMR (400 MHz, CD₃OD) δ 9.03 (m, 1H), 8.47 (s, 2H), 8.20 (s, 1H), 5.58 (s, 1H), 3.88 (s, 1H), 3.43 (t, J=6.4 Hz, 2H), 3.20 (s, 6H), 2.48 (s, 3H), 1.90-1.75 (m, 6H), 1.54-1.46 (m, 2H).

### Example 68

### N-[cis-4-(6-Dimethylamino-2-methyl-pyrimidin-4-ylamino)-cyclohexylmethyl]-4-trifluoromethoxy-benzamide trifluoroacetate

Using the procedure of Step E of Example 67, the title compound was obtained (187 mg, 0.033 mmol, 33%) as a white solid.

ESI MS m/e 452.2 (M+H)⁺; ¹H NMR (400 MHz, CD₃OD) δ 8.65 (m, 1H), 7.96 (d, J=9.4 Hz, 2H), 7.40 (d, J=8.4 Hz), 5.58 (s, 1H), 3.87 (s, 1H), 3.39 (t, J=6.4 Hz), 3.19 (s, 6H), 2.48 (s, 3H), 1.88-1.75 (m, 6H), 1.53-1.44 (m, 2H).

### Examples 69-72

Compounds 69 to 72 were prepared in a similar manner as described in Example 48 using the appropriate carboxylic acid and amine intermediate of Step D.

### Examples 73-107

Compounds 73 to 107 were prepared in a similar manner as described in Example 50 using the appropriate acid chloride and amine intermediate of Step A.

### Examples 108-110

Compounds 108 to 110 were prepared in a similar manner as described in Example 52 using the appropriate aldehyde and amine intermediate of Step A.

### Examples 111-113

Compounds 111 to 113 were prepared in a similar manner as described in Example 54 using the appropriate isocyanate and amine intermediate of Step A.

### Examples 114-117

Compounds 114 to 117 were prepared in a similar manner as described in Example 48 using the appropriate carboxylic acid and amine intermediate of Step D.

### Examples 118-125

Compounds 118 to 125 were prepared in a similar manner as described in Example 63 using the appropriate acid chloride and amine intermediate of Step D.

### Examples 126-133

Compounds 126 to 133 were prepared in a similar manner as described in Example 65 using the appropriate carboxylic acid and amine intermediate of Step A.

### Examples 134-140

Compounds 134 to 140 were prepared in a similar manner as described in Example 59 using the appropriate acid chloride and amine intermediate of Step D.

### Examples 141-148

Compounds 141 to 148 were prepared in a similar manner as described in Example 61 using the appropriate carboxylic acid and amine intermediate of Step A.

### Examples 149-167

Compounds 149 to 167 were prepared in a similar manner as described in Example 67 using the appropriate acid chloride and amine intermediate of Step E.

| Ex. No. | compound name | MS |
|---|---|---|
| 69 | N-[cis-4-(6-Dimethylamino-2-methyl-pyrimidin-4-ylamino)-cyclohexyl]- | 442.4 (M + H) |
| | 3,4-diethoxy-benzamide | |
| 70 | N-[cis-4-(6-Dimethylamino-2-methyl-pyrimidin-4-ylamino)-cyclohexyl]- | 398.2 (M + H) |
| | 3-ethoxy-benzamide | |
| 71 | N-[cis-4-(6-Dimethylamino-2-methyl-pyrimidin-4-ylamino)-cyclohexyl]- | 442.2 (M + H) |
| | 3,5-diethoxy-benzamide | |
| 72 | N-[cis-4-(6-Dimethylamino-2-methyl-pyrimidin-4-ylamino)-cyclohexyl]- | 412.4 (M + H) |
| | 3-isopropoxy-benzamide | |
| 73 | 3-Bromo-N-[cis-4-(6-dimethylamino-2-methyl-pyrimidin-4-ylamino)- | 450.2 (M + H) |
| | cyclohexyl]-4-fluoro-benzamide | |
| 74 | 4-Difluoromethoxy-N-[cis-4-(6-dimethylamino-2-methyl-pyrimidin-4- | 420.2 (M + H) |
| | ylamino)-cyclohexyl]-benzamide | |
| 75 | 4-Chloro-N-[cis-4-(6-dimethylamino-2-methyl-pyrimidin-4-ylamino)- | 402 (M + H) |
| | cyclohexyl]-3-methyl-benzamide | |
| 76 | 3-Chloro-N-[cis-4-(6-dimethylamino-2-methyl-pyrimidin-4-ylamino)- | 406.2 (M + H) |
| | cyclohexyl]-5-fluoro-benzamide | |
| 77 | 3-Difluoromethoxy-N-[cis-4-(6-dimethylamino-2-methyl-pyrimidin-4- | 420.2 (M + H) |
| | ylamino)-cyclohexyl]-benzamide | |
| 78 | 3-Chloro-N-[cis-4-(6-dimethylamino-2-methyl-pyrimidin-4-ylamino)- | 402.2 (M + H) |
| | cyclohexyl]-4-methyl-benzamide | |
| 79 | 4-Bromo-N-[cis-4-(6-dimethylamino-2-methyl-pyrimidin-4-ylamino)- | 432.2 (M + H) |
| | cyclohexyl]-benzamide | |
| 80 | N-[cis-4-(6-Dimethylamino-2-methyl-pyrimidin-4-ylamino)-cyclohexyl]- | 414.6 (M + H) |
| | 3,5-dimethoxy-benzamide | |
| 81 | 3,4-Dichloro-N-[cis-4-(6-dimethylamino-2-methyl-pyrimidin-4-ylamino)- | 422.2 (M + H) |
| | cyclohexyl]-benzamide | |
| 82 | 4-Cyano-N-[cis-4-(6-dimethylamino-2-methyl-pyrimidin-4-ylamino)- | 379.2 (M + H) |
| | cyclohexyl]-benzamide | |
| 83 | N-[cis-4-(6-Dimethylamino-2-methyl-pyrimidin-4-ylamino)-cyclohexyl]- | 384.2 (M + H) |
| | 4-methoxy-benzamide | |
| 84 | 3-Cyano-N-[cis-4-(6-dimethylamino-2-methyl-pyrimidin-4-ylamino)- | 379.2 (M + H) |
| | cyclohexyl]-benzamide | |
| 85 | 3,5-Dichloro-N-[cis-4-(6-dimethylamino-2-methyl-pyrimidin-4-ylamino)- | 422.2 (M + H) |
| | cyclohexyl]-benzamide | |
| 86 | N-[cis-4-(6-Dimethylamino-2-methyl-pyrimidin-4-ylamino)-cyclohexyl]- | 384.2 (M + H) |
| | 3-methoxy-benzamide | |
| 87 | N-[cis-4-(6-Dimethylamino-2-methyl-pyrimidin-4-ylamino)-cyclohexyl]- | 386.2 (M + H) |
| | 4-fluoro-3-methyl-benzamide | |
| 88 | N-[cis-4-(6-Dimethylamino-2-methyl-pyrimidin-4-ylamino)-cyclohexyl]- | 440.4 (M + H) |
| | 3-fluoro-5-trifluoromethyl-benzamide | |
| 89 | N-[cis-4-(6-Dimethylamino-2-methyl-pyrimidin-4-ylamino)-cyclohexyl]- | 372.2 (M + H) |
| | 4-fluoro-benzamide | |
| 90 | 4-Bromo-N-[cis-4-(6-dimethylamino-2-methyl-pyrimidin-4-ylamino)- | 446.2 (M + H) |
| | cyclohexyl]-3-methyl-benzamide | |
| 91 | N-[cis-4-(6-Dimethylamino-2-methyl-pyrimidin-4-ylamino)-cyclohexyl]- | 386.2 (M + H) |
| | 3-fluoro-4-methyl-benzamide | |
| 92 | 4-Chloro-N-[cis-4-(6-dimethylamino-2-methyl-pyrimidin-4-ylamino)- | 388.4 (M + H) |
| | cyclohexyl]-benzamide | |
| 93 | N-[cis-4-(6-Dimethylamino-2-methyl-pyrimidin-4-ylamino)-cyclohexyl]- | 372.2 (M + H) |
| | 3-fluoro-benzamide | |
| 94 | N-[cis-4-(6-Dimethylamino-2-methyl-pyrimidin-4-ylamino)-cyclohexyl]- | 438.4 (M + H) |
| | 3-trifluoromethoxy-benzamide | |
| 95 | N-[cis-4-(6-Dimethylamino-2-methyl-pyrimidin-4-ylamino)-cyclohexyl]- | 382.4 (M + H) |
| | 3-ethyl-benzamide | |
| 96 | 3-Bromo-N-[cis-4-(6-dimethylamino-2-methyl-pyrimidin-4-ylamino)- | 432.3 (M + H) |
| | cyclohexyl]-benzamide | |
| 97 | N-[cis-4-(6-Dimethylamino-2-methyl-pyrimidin-4-ylamino)-cyclohexyl]- | 422.1 (M + H) |
| | 3-trifluoromethyl-benzamide | |
| 98 | N-[cis-4-(6-Dimethylamino-2-methyl-pyrimidin-4-ylamino)-cyclohexyl]- | 440.6 (M + H) |
| | 3-fluoro-4-trifluoromethyl-benzamide | |
| 99 | 3-Chloro-N-[cis-4-(6-dimethylamino-2-methyl-pyrimidin-4-ylamino)- | 388.5 (M + H) |
| | cyclohexyl]-benzamide | |
| 100 | N-[cis-4-(6-Dimethylamino-2-methyl-pyrimidin-4-ylamino)-cyclohexyl]- | 440.6 (M + H) |
| | 4-fluoro-3-trifluoromethyl-benzamide | |
| 101 | N-[cis-4-(6-Dimethylamino-2-methyl-pyrimidin-4-ylamino)-cyclohexyl]- | 390.2 (M + H) |
| | 3,4-difluoro-benzamide | |
| 102 | 3-Chloro-N-[cis-4-(6-dimethylamino-2-methyl-pyrimidin-4-ylamino)- | 406.3 (M + H) |
| | cyclohexyl]-4-fluoro-benzamide | |
| 103 | N-[cis-4-(6-Dimethylamino-2-methyl-pyrimidin-4-ylamino)-cyclohexyl]- | 438.1 (M + H) |
| | 4-trifluoromethoxy-benzamide | |
| 104 | N-[cis-4-(6-Dimethylamino-2-methyl-pyrimidin-4-ylamino)-cyclohexyl]- | 368.3 (M + H) |
| | 4-methyl-benzamide | |
| 105 | N-[cis-4-(6-Dimethylamino-2-methyl-pyrimidin-4-ylamino)-cyclohexyl]- | 368.2 (M + H) |
| | 3-methyl-benzamide | |
| 106 | N-[cis-4-(6-Dimethylamino-2-methyl-pyrimidin-4-ylamino)-cyclohexyl]- | 422.3 (M + H) |
| | 4-trifluoromethyl-benzamide | |
| 107 | 2,2-Difluoro-benzo[1,3]dioxole-5-carboxylic acid [cis-4-(6- | 434.1 (M + H) |
| | dimethylamino-2-methyl-pyrimidin-4-ylamino)-cyclohexyl]-amide | |
| 108 | N-{cis-4-[(1H-Indol-2-ylmethyl)-amino]-cyclohexyl}-2,N′,N′-trimethyl- | 379.4 (M + H) |
| | pyrimidine-4,6-diamine | |
| 109 | 2,N,N-Trimethyl-N′-[cis-4-(3-trifluoromethoxy-benzylamino)- | 424.2 (M + H) |
| | cyclohexyl]-pyrimidine-4,6-diamine | |
| 110 | N-[cis-4-(3,4-Difluoro-benzylamino)-cyclohexyl]-2,N′,N′-trimethyl- | 376.6 (M + H) |
| | pyrimidine-4,6-diamine | |
| 111 | 1-(3,4-Dimethoxy-phenyl)-3-[cis-4-(6-dimethylamino-2-methyl- | 429.4 (M + H) |
| | pyrimidin-4-ylamino)-cyclohexyl]-urea | |
| 112 | 1-[cis-4-(6-Dimethylamino-2-methyl-pyrimidin-4-ylamino)-cyclohexyl]- | 413.5 (M + H) |
| | 3-(2-ethoxy-phenyl)-urea | |
| 113 | 1-(4-Benzyloxy-phenyl)-3-[cis-4-(6-dimethylamino-2-methyl-primidin- | 475.5 (M + H) |
| | 4-ylamino)-cyclohexyl]-urea | |
| 114 | 3,5-Dibromo-N-[cis-4-(6-dimethylamino-2-methyl-pyrimidin-4-ylamino)- | 510.2 (M + H) |
| | cyclohexyl]-benzamide | |
| 115 | 3-Bromo-4-chloro-N-[cis-4-(6-dimethylamino-2-methyl-pyrimidin-4- | 466.2 (M + H) |
| | ylamino)-cyclohexyl]-benzamide | |
| 116 | 4-Chloro-N-[cis-4-(6-dimethylamino-2-methyl-pyrimidin-4-ylamino)- | 456.2 (M + H) |
| | cyclohexyl]-3-trifluoromethyl-benzamide | |
| 117 | 2-(3,5-Bis-trifluoromethyl-phenyl)-N-[cis-4-(6-dimethylamino-2-methyl- | 520.2 (M + H) |
| | pyrimidin-4-ylamino)-cyclohexyl]-2-hydroxy-acetamide | |
| 118 | N-[cis-4-(2-Dimethylamino-5-methyl-pyrimidin-4-ylamino)-cyclohexyl]- | 384.2 (M + H) |
| | 3-methoxy-benzamide | |
| 119 | N-[cis-4-(2-Dimethylamino-5-methyl-pyrimidin-4-ylamino)-cyclohexyl]- | 422.2 (M + H) |
| | 3-trifluoromethyl-benzamide | |
| 120 | N-[cis-4-(2-Dimethylamino-5-methyl-pyrimidin-4-ylamino)-cyclohexyl]- | 490.4 (M + H) |
| | 3,5-bis-trifluoromethyl-benzamide | |
| 121 | 2,2-Difluoro-benzo[1,3]dioxole-5-carboxylic acid [cis-4-(2- | 434.2 (M + H) |
| | dimethylamino-5-methyl-pyrimidin-4-ylamino)-cyclohexyl]-amide | |
| 122 | 4-Cyano-N-[cis-4-(2-dimethylamino-5-methyl-pyrimidin-4-ylamino)- | 379.4 (M + H) |
| | cyclohexyl]-benzamide | |
| 123 | 4-Chloro-N-[cis-4-(2-dimethylamino-5-methyl-pyrimidin-4-ylamino)- | 388.2 (M + H) |
| | cyclohexyl]-benzamide | |
| 124 | N-[cis-4-(2-Dimethylamino-5-methyl-pyrimidin-4-ylamino)-cyclohexyl]- | 382.4 (M + H) |
| | 3-ethyl-benzamide | |
| 125 | N-[cis-4-(2-Dimethylamino-5-methyl-pyrimidin-4-ylamino)-cyclohexyl]- | 390.4 (M + H) |
| | 3,4-difluoro-benzamide | |
| 126 | 5-Bromo-N-[cis-4-(2-dimethylamino-5-methyl-pyrimidin-4-ylamino)- | 433.2 (M + H) |
| | cyclohexyl]-nicotinamide | |
| 127 | 5-Bromo-furan-2-carboxylic acid [cis-4-(2-dimethylamino-5-methyl- | 422.2 (M + H) |
| | pyrimidin-4-ylamino)-cyclohexyl]-amide | |
| 128 | 3,5-Dibromo-N-[cis-4-(2-dimethylamino-5-methyl-pyrimidin-4-ylamino)- | 510.2 (M + H) |
| | cyclohexyl]-benzamide | |
| 129 | N-[cis-4-(2-Dimethylamino-5-methyl-pyrimidin-4-ylamino)-cyclohexyl]- | 398.2 (M + H) |
| | 3-ethoxy-benzamide | |
| 130 | 2-(3,5-Bis-trifluoromethyl-phenyl)-N-[cis-4-(2-dimethylamino-5-methyl- | 520.4 (M + H) |
| | pyrimidin-4-ylamino)-cyclohexyl]-2-hydroxy-acetamide | |
| 131 | 2-(4-Bromo-phenyl)-N-[cis-4-(2-dimethylamino-5-methyl-pyrimidin-4- | 462.2 (M + H) |
| | ylamino)-cyclohexyl]-2-hydroxy-acetamide | |
| 132 | N-[cis-4-(2-Dimethylamino-5-methyl-pyrimidin-4-ylamino)-cyclohexyl]- | 442.6 (M + H) |
| | 3,5-diethoxy-benzamide | |
| 133 | 3-Bromo-N-[cis-4-(2-dimethylamino-5-methyl-pyrimidin-4-ylamino)- | 450 (M + H) |
| | cyclohexyl]-4-fluoro-benzamide | |
| 134 | N-[cis-4-(2-Dimethylamino-6-methyl-pyrimidin-4-ylamino)-cyclohexyl]- | 384.2 (M + H) |
| | 3-ethoxy-benzamide | |
| 135 | N-[cis-4-(2-Dimethylamino-6-methyl-pyrimidin-4-ylamino)-cyclohexyl]- | 422.2 (M + H) |
| | 3-trifluoromethyl-benzamide | |
| 136 | N-[cis-4-(2-Dimethylamino-6-methyl-pyrimidin-4-ylamino)-cyclohexyl]- | 490.4 (M + H) |
| | 3,5-bis-trifluoromethyl-benzamide | |
| 137 | 2,2-Difluoro-benzo[1,3]dioxole-5-carboxylic acid [cis-4-(2- | 434.4 (M + H) |
| | dimethylamino-6-methyl-pyrimidin-4-ylamino)-cyclohexyl]-amide | |
| 138 | 4-Chloro-N-[cis-4-(2-dimethylamino-6-methyl-pyrimidin-4-ylamino)- | 388.2 (M + H) |
| | cyclohexyl]-benzamide | |
| 139 | N-[cis-4-(2-Dimethylamino-6-methyl-pyrimidin-4-ylamino)-cyclohexyl]- | 382.4 (M + H) |
| | 3-ethyl-benzamide | |
| 140 | N-[cis-4-(2-Dimethylamino-6-methyl-pyrimidin-4-ylamino)-cyclohexyl]- | 368.2 (M + H) |
| | 4-methyl-benzamide | |
| 141 | 5-Bromo-N-[cis-4-(2-dimethylamino-6-methyl-pyrimidin-4-ylamino)- | 433.2 (M + H) |
| | cyclohexyl]-nicotinamide | |
| 142 | 5-Bromo-furan-2-carboxylic acid [cis-4-(2-dimethylamino-6-methyl- | 422 (M + H) |
| | pyrimidin-4-ylamino)-cyclohexyl]-amide | |
| 143 | 3,5-Dibromo-N-[cis-4-(2-dimethylamino-6-methyl-pyrimidin-4-ylamino)- | 510 (M + H) |
| | cyclohexyl]-benzamide | |
| 144 | N-[cis-4-(2-Dimethylamino-6-methyl-pyrimidin-4-ylamino)-cyclohexyl]- | 398.2 (M + H) |
| | 3-ethoxy-benzamide | |
| 145 | 2-(3,5-Bis-trifluoromethyl-phenyl)-N-[cis-4-(2-dimethylamino-6-methyl- | 520.4 (M + H) |
| | pyrimidin-4-ylamino)-cyclohexyl]-2-hydroxy-acetamide | |
| 146 | 2-(4-Bromo-phenyl)-N-[cis-4-(2-dimethylamino-6-methyl-pyrimidin-4- | 462.2 (M + H) |
| | ylamino)-cyclohexyl]-2-hydroxy-acetamide | |
| 147 | N-[cis-4-(2-Dimethylamino-6-methyl-pyrimidin-4-ylamino)-cyclohexyl]- | 442.4 (M + H) |
| | 3,5-diethoxy-benzamide | |
| 148 | 3-Bromo-N-[cis-4-(2-dimethylamino-6-methyl-pyrimidin-4-ylamino)- | 450 (M + H) |
| | cyclohexyl]-4-fluoro-benzamide | |
| 149 | N-[cis-4-(6-Dimethylamino-2-methyl-pyrimidin-4-ylamino)- | 454.2 (M + H) |
| | cyclohexylmethyl]-3-fluoro-4-trifluoromethyl-benzamide | |
| 150 | N-[cis-4-(6-Dimethylamino-2-methyl-pyrimidin-4-ylamino)- | 452.2 (M + H) |
| | cyclohexylmethyl]-3-trifluoromethoxy-benzamide | |
| 151 | N-[cis-4-(6-Dimethylamino-2-methyl-pyrimidin-4-ylamino)- | 398.2 (M + H) |
| | cyclohexylmethyl]-3-methoxy-benzamide | |
| 152 | 4-Chloro-N-[cis-4-(6-dimethylamino-2-methyl-pyrimidin-4-ylamino)- | 402.2 (M + H) |
| | cyclohexylmethyl]-benzamide | |
| 153 | N-[cis-4-(6-Dimethylamino-2-methyl-pyrimidin-4-ylamino)- | 436.2 (M + H) |
| | cyclohexylmethyl]-3-trifluoromethyl-benzamide | |
| 154 | N-[cis-4-(6-Dimethylamino-2-methyl-pyrimidin-4-ylamino)- | 436.2 (M + H) |
| | cyclohexylmethyl]-4-trifluoromethyl-benzamide | |
| 155 | N-[cis-4-(6-Dimethylamino-2-methyl-pyrimidin-4-ylamino)- | 382.4 (M + H) |
| | cyclohexylmethyl]-3-methyl-benzamide | |
| 156 | N-[cis-4-(6-Dimethylamino-2-methyl-pyrimidin-4-ylamino)- | 404 (M + H) |
| | cyclohexylmethyl]-3,5-difluoro-benzamide | |
| 157 | N-[cis-4-(6-Dimethylamino-2-methyl-pyrimidin-4-ylamino)- | 396.2 (M + H) |
| | cyclohexylmethyl]-3-ethyl-benzamide | |
| 158 | 2,2-Difluoro-benzo[1,3]dioxole-5-carboxylic acid [cis-4-(6- | 448.2 (M + H) |
| | dimethylamino-2-methyl-pyrimidin-4-ylamino)-cyclohexylmethyl]- | |
| | amide | |
| 159 | N-[cis-4-(6-Dimethylamino-2-methyl-pyrimidin-4-ylamino)- | 400.2 (M + H) |
| | cyclohexylmethyl]-3-fluoro-4-methyl-benzamide | |
| 160 | N-[cis-4-(6-Dimethylamino-2-methyl-pyrimidin-4-ylamino)- | 386.2 (M + H) |
| | cyclohexylmethyl]-4-fluoro-benzamide | |
| 161 | 3,4-Dichloro-N-[cis-4-(6-dimethylamino-2-methyl-pyrimidin-4-ylamino)- | 436.2 (M + H) |
| | cyclohexylmethyl]-benzamide | |
| 162 | 4-Bromo-N-[cis-4-(6-dimethylamino-2-methyl-pyrimidin-4-ylamino)- | 446.2 (M + H) |
| | cyclohexylmethyl]-benzamide | |
| 163 | N-[cis-4-(6-Dimethylamino-2-methyl-pyrimidin-4-ylamino)- | 404.2 (M + H) |
| | cyclohexylmethyl]-3,4-difluoro-benzamide | |
| 164 | 3,5-Dichloro-N-[cis-4-(6-dimethylamino-2-methyl-pyrimidin-4-ylamino)- | 436.2 (M + H) |
| | cyclohexylmethyl]-benzamide | |
| 165 | 3-Chloro-N-[cis-4-(6-dimethylamino-2-methyl-pyrimidin-4-ylamino)- | 420.2 (M + H) |
| | cyclohexylmethyl]-4-fluoro-benzamide | |
| 166 | N-[cis-4-(6-Dimethylamino-2-methyl-pyrimidin-4-ylamino)- | 400.2 (M + H) |
| | cyclohexylmethyl]-4-fluoro-3-methyl-benzamide | |
| 167 | 3-Chloro-N-[cis-4-(6-dimethylamino-2-methyl-pyrimidin-4-ylamino)- | 402 (M + H) |
| | cyclohexylmethyl]-benzamide | |

### Example 168

### N-{cis-4-[(6-Amino-2-methylpyrimidin-4-yl)amino]cyclohexyl}-3,4,5-trifluorobenzamide hydrochloride

### Step A: Synthesis of N-(cis-4-aminocyclohexyl)-3,4,5-trifluorobenzamide

To a solution of tert-butyl (cis-4-aminocyclohexyl)carbamate (44.3 g) in DMF (450 mL) were added 3,4,5-trifluorobenzoic acid (40.1 g), Et₃N (69.2 mL), HOBt-H₂O (47.5 g), and EDC-HCl (43.6 g). The mixture was stirred at ambient temperature for 12 h. To the mixture was added water (1 L) and the suspension was stirred at ambient temperature for 2 h. The precipitate was collected by filtration, washed with water and hexane, and dried at 80° C. under reduced pressure to give a pale brown solid (82.7 g). To a suspension of the above solid in EtOAc (800 mL) was added 4 M hydrogen chloride in EtOAc (600 mL) under 10° C. The mixture was stirred at ambient temperature for 6 h and concentrated under reduced pressure. The residue was dissolved in CHCl₃ (300 mL) and poured into 1 M aqueous NaOH (500 mL). The aqueous layer was extracted with CHCl₃ three times. The combined organic layer was dried over MgSO₄, filtered, and concentrated under reduced pressure to give the title compound (65.3 g).

¹H NMR (300 MHz, CDCl₃, δ): 1.38-1.91 (m, 8H), 2.97-3.09 (m, 1H), 4.04-4.20 (m, 1H), 6.15-6.27 (m, 1H), 7.35-7.50 (m, 2H); ESI MS m/z 273 (M⁺+1, 100%).

### Step B: Synthesis of 6-chloro-2-methylpyrimidin-4-amine

To a solution of 4,6-dichloro-2-methyl-pyrimidine obtained in step A of example 5 (15.0 g) in 2-propanol (30 mL) was added 28% aqueous NH₃ (30 mL). The mixture was stirred at reflux for 6 hr in a sealed tube and cooled to ambient temperature. The precipitate was collected by filtration, washed with 2-propanol, and dried at 80° C. under reduced pressure to give the title compound (7.58 g).

¹H NMR (300 MHz, DMSO-d₆, δ): 2.29 (s, 3H), 6.27 (s, 1H), 7.12 (brs, 2H); ESI MS m/z 144 (M⁺+1, 100%).

### Step C: Synthesis of {cis-4-[(6-amino-2-methylpyrimidin-4-yl)amino]cyclohexyl}-3,4,5-trifluorobenzamide hydrochloride

To a suspension of N-(cis-4-aminocyclohexyl)-3,4,5-trifluorobenzamide (1.20 g) in BuOH (2 mL) was added 6-chloro-2-methylpyrimidin-4-amine (534 mg). The mixture was heated in a microwave synthesizer at 220° C. for 30 nm. The mixture was diluted with CHCl₃ and added to saturated aqueous NaHCO₃. The aqueous layer was extracted with CHCl₃ (three times). The combined organic layer was dried over MgSO₄, filtrated, concentrated under reduced pressure, and purified by medium-pressure liquid chromatography (NH-silica gel, 20% to 80% EtOAc in hexane) to give a oil. To a solution of the above oil in EtOAc (10 mL) was added 4 M hydrogen chloride in EtOAc (0.5 mL). The mixture was stirred at ambient temperature for 30 min and concentrated under reduced pressure. A suspension of the residue in Et₂O (10 mL) was stirred at ambient temperature for 2 h. The precipitate was collected by filtration, washed with Et₂O, and dried at 80° C. under reduced pressure to give the title compound (627 mg).

¹H NMR (300 MHz, DMSO-d₆, δ): 1.60-1.75 (m, 8H), 2.36 (s, 3H), 3.80-4.13 (m, 2H), 5.43-5.78 (m, 1H), 7.16-7.70 (m, 1H), 7.74-7.95 (m, 2H), 8.37-8.48 (m, 1H), 13.29-13.55 (m, 1H); ESI MS m/z 380 [M (free)⁺+1, 100%].

### Example 169

### 3,4,5-Trifluoro-N-(cis-4-{[2-methyl-6-(methylamino)pyrimidin-4-yl]amino}cyclohexyl)-benzamide hydrochloride

### Step A: Synthesis of 6-chloro-N,2-dimethylpyrimidin-4-amine

To a solution of 4,6-dichloro-2-methyl-pyrimidine obtained in step A of example 5 (15.0 g) in THF (150 mL) was added 40% aqueous MeNH₂ (17.9 g) and the mixture was stirred at ambient temperature for 3 h. The mixture was diluted with CHCl₃ and added to saturated aqueous NaHCO₃. The aqueous layer was extracted with CHCl₃ (three times). The combined organic layer was dried over MgSO₄, filtrated, concentrated under reduced pressure, and dried under reduced pressure to give the title compound (13.6 g).

¹H NMR (300 MHz, CDCl₃, δ): 2.48 (s, 3H), 2.93 (d, J=5.1 Hz, 3H), 5.02-5.29 (m, 1H), 6.18 (s, 1H); ESI MS m/z 158 (M⁺+1, 100%).

### Step B: Synthesis of 3,4,5-trifluoro-N-(cis-4-{[2-methyl-6-(methylamino)pyrimidin-4-yl]amino}cyclohexyl)benzamide hydrochloride

The title compound (312 mg) was prepared from N-(cis-4-aminocyclohexyl)-3,4,5-trifluorobenzamide obtained in step A of example 168 (952 mg) and 6-chloro-N,2-dimethylpyrimidin-4-amine (500 mg) using the procedure for the step C of example 168.

¹H NMR (300 MHz, CDCl₃, δ): 1.55-1.91 (m, 8H), 2.22-2.46 (m, 3H), 2.71-2.94 (m, 3H), 3.73-4.11 (m, 2H), 5.36-5.67 (m, 2H), 7.74-7.90 (m, 2H), 8.09-8.52 (m, 2H); ESI MS m/z 394 [M (free)⁺+1, 100%].

### Example 170

### N-(cis-4-{[6-(Dimethylamino)-2-methylpyrimidin-4-yl]amino}cyclohexyl)-3,4,5-trifluorobenzamide methanesulfonate

To a solution of N-(cis-4-{[6-(dimethylamino)-2-methylpyrimidin-4-yl]amino}cyclohexyl)-3,4,5-trifluorobenzamide (3.00 g) obtained in example 11 in EtOH (21 mL) was added MsOH (743 mg). The mixture was stirred at ambient temperature for 1 h and 4° C. for 4 h. The precipitate was collected by filtration, washed with cold EtOH, and dried at 80° C. under reduced pressure to give the title compound (3.16 g).

¹H NMR (300 MHz, CDCl₃, δ): 1.60-2.08 (m, 8H), 2.48 (s, 3H), 2.92 (s, 3H), 3.07 (brs, 3H), 3.30 (brs, 3H), 3.71-3.80 (m, 1H), 4.07-4.24 (m, 1H), 5.18 (s, 1H), 7.65-7.83 (m, 4H), 12.63 (brs, 1H); ESI MS m/z 408 [M (free)⁺+1, 100%].

### Example 171

### 3-Chloro-N-{cis-4-[(2,6-dimethylpyrimidin-4-yl)amino]cyclohexyl}-4-fluorobenzamide hydrochloride

### Step A: Synthesis of 4-chloro-2,6-dimethylpyrimidine

A solution of ZnBr₂ (4.14 g) in THF (15 mL) was cooled to −60° C. and 3 M methylmagnesiumbromide in Et₂O (6.13 mL) was added. The mixture was stirred at −60° C. for 1 hr and warmed to ambient temperature. To the mixture were added tetrakis-(triphenylphosphine)-palladium (1.06 g) and 4,6-dichloro-2-methyl-pyrimidine obtained in step A of example 5 (3.0 g) in THF (15 mL). The mixture was stirred at 60° C. for 8 h. To the mixture was added saturated aqueous NH₄Cl and the aqueous layer was extracted with CHCl₃ (three times). The combined organic layer was dried over MgSO₄, filtered, concentrated under reduced pressure, and purified by medium-pressure liquid chromatography (silica gel, 5% to 16% EtOAc in hexane) to give the title compound (940 mg).

¹H NMR (300 MHz, CDCl₃, δ): 2.49 (s, 3H), 2.68 (s, 3H), 7.05 (s, 1H); CI MS m/z 143 (M⁺+1, 100%).

### Step B: Synthesis of 3-chloro-N-{cis-4-[(2,6-dimethylpyrimidin-4-yl)amino]cyclohexyl}-4-fluorobenzamide hydrochloride

The title compound (454 mg) was prepared from N-(cis-4-amino-cyclohexyl)-3-chloro-4-fluoro-benzamide obtained in step A of example 31 (520 mg) and 4-chloro-2,6-dimethylpyrimidine (250 mg) using the procedure for the step C of example 168.

¹H NMR (600 MHz, CDCl₃, δ): 1.68-2.16 (m, 8H), 2.38 (brs, 3H), 2.62 (s, 3H), 4.10-4.22 (m, 1H), 4.43-4.53 (m, 1H), 6.80-6.91 (m, 1H), 7.08-7.18 (m, 2H), 7.75-7.86 (m, 1H), 7.92-8.12 (m, 1H), 8.90-9.06 (m, 1H); ESI MS m/z 377 [M (free)⁺+1, 100%].

### Example 172

### N-{cis-4-[(6-Chloro-2-methylpyrimidin-4-yl)amino]cyclohexyl}-3,4,5-trifluorobenzamide

To a suspension of N-(cis-4-aminocyclohexyl)-3,4,5-trifluorobenzamide obtained in step A of example 168 (16.7 g) in BuOH (9.1 mL) were added 4,6-dichloro-2-methyl-pyrimidine obtained in step A of example 5 (9.10 g) and iPrNEt₂ (10.7 mL). The mixture was stirred at reflux for 1.5 h. The mixture was diluted with CHCl₃ and added to saturated aqueous NaHCO₃. The aqueous layer was extracted with CHCl₃ (three times). The combined organic layer was dried over MgSO₄, filtrated, concentrated under reduced pressure, and purified by medium-pressure liquid chromatography (NH-silica gel, 33% to 66% EtOAc in hexane) to give the title compound (21.0 g).

¹H NMR (300 MHz, CDCl₃, δ): 1.56-2.03 (m, 8H), 2.47 (s, 3H), 3.74-3.92 (m, 1H), 4.03-4.18 (m, 1H), 5.08-5.24 (m, 1H), 6.08 (d, J=7.3 Hz, 1H), 6.18 (s, 1H), 7.33-7.50 (m, 2H);

ESI MS m/z 399 (M⁺+1, 100%).

### Example 173

### N-(cis-4-{[6-(Cyclopropylamino)-2-methylpyrimidin-4-yl]amino}cyclohexyl)-3,4,5-trifluorobenzamide hydrochloride

To a suspension of N-{cis-4-[(6-chloro-2-methylpyrimidin-4-yl)amino]cyclohexyl}-3,4,5-trifluorobenzamide obtained in example 172 (250 mg) in 3-methyl-butan-1-ol (0.5 mL) was added cyclopropylamine (43 mg). The mixture was stirred at 190° C. for 1.5 h in a sealed tube. The mixture was diluted with CHCl₃ and added to saturated aqueous NaHCO₃. The aqueous layer was extracted with CHCl₃ (three times). The combined organic layer was dried over MgSO₄, filtrated, concentrated under reduced pressure, and purified by medium-pressure liquid chromatography (NH-silica gel, 20% to 50% EtOAc in hexane and silica gel, 2% to 9% MeOH in CHCl₃) to give a colorless oil. To a solution of the above oil in EtOAc (10 mL) was added 4 M hydrogen chloride in EtOAc (0.5 mL). The mixture was stirred at ambient temperature for 30 min and concentrated under reduced pressure. A suspension of the residue in Et₂O (10 mL) was stirred at ambient temperature for 2 hr. The precipitate was collected by filtration, washed with Et₂O, and dried at 80° C. under reduced pressure to give the title compound (90 mg).

¹H NMR (300 MHz, CDCl₃, δ): 0.62-0.74 (m, 2H), 0.88-1.00 (m, 2H), 1.72-2.02 (m, 8H), 2.45 (s, 3H), 2.50-2.64 (m, 1H), 3.71-3.87 (m, 1H), 4.03-4.19 (m, 1H), 5.52 (s, 1H), 6.80-6.96 (m, 1H), 7.48-7.62 (m, 2H); ESI MS m/z 420 [M (free)⁺+1, 100%].

### Example 174

### 3,4,5-Trifluoro-N-[cis-4-({2-methyl-6-[methyl(phenyl)amino]pyrimidin-4-yl}amino)-cyclohexyl]benzamide hydrochloride

The title compound (210 mg) was prepared from N-{cis-4-[(6-chloro-2-methylpyrimidin-4-yl)amino]cyclohexyl}-3,4,5-trifluorobenzamide obtained in example 172 (250 mg) and N-methylaniline (81 mg) using the procedure for the example 173.

¹H NMR (300 MHz, CDCl₃, δ): 1.50-1.91 (m, 8H), 2.55 (s, 3H), 3.31-3.40 (m, 1H), 3.54 (s, 3H), 3.95-4.09 (m, 1H), 4.96 (s, 1H), 6.81 (d, J=8.4 Hz, 1H), 7.21-7.27 (m, 2H), 7.40-7.58 (m, 4H), 8.43 (d, J=8.4 Hz, 1H); ESI MS m/z 470 [M (free)⁺+1, 100%].

### Example 175

### N-[cis-4-({6-[Benzyl(methyl)amino]-2-methylpyrimidin-4-yl}amino)cyclohexyl]-3,4,5-trifluorobenzamide hydrochloride

The title compound (121 mg) was prepared from N-{cis-4-[(6-chloro-2-methylpyrimidin-4-yl)amino]cyclohexyl}-3,4,5-trifluorobenzamide obtained in example 172 (250 mg) and N-methylbenzylamine (91 mg) using the procedure for the example 173.

¹H NMR (300 MHz, CDCl₃, δ): 1.57-2.07 (m, 8H), 2.51 (s, 3H), 2.98 (s, 3H), 3.28-3.45 (m, 1H), 3.68-3.81 (m, 1H), 3.98-4.20 (m, 1H), 4.94-5.23 (m, 2H), 6.93-7.04 (m, 1H), 7.12-7.24 (m, 2H), 7.30-7.42 (m, 3H), 7.48-7.61 (m, 2H), 8.54-8.67 (m, 1H), 13.78-13.89 (m, 1H); ESI MS m/z 484 [M (free)⁺+1, 100%].

### Example 176

### N-[cis-4-({6-[Ethyl(methyl)amino]-2-methylpyrimidin-4-yl}amino)cyclohexyl]-3,4,5-trifluorobenzamide hydrochloride

The title compound (71 mg) was prepared from N-{cis-4-[(6-chloro-2-methylpyrimidin-4-yl)amino]cyclohexyl}-3,4,5-trifluorobenzamide obtained in example 172 (250 mg) and N-ethylmethylamine (44 mg) using the procedure for the example 173.

¹H NMR (300 MHz, CDCl₃, δ): 1.06-1.35 (m, 3H), 1.62-2.11 (m, 8H), 2.48 (s, 3H), 2.96-3.49 (m, 4H), 3.67-3.85 (m, 2H), 4.01-4.20 (m, 1H), 5.04-5.20 (m, 1H), 6.98 (d, J=8.5 Hz, 1H), 7.47-7.63 (m, 2H), 8.36-8.55 (m, 1H), 13.57-13.77 (m, 1H); ESI MS m/z 422 [M (free)⁺+1, 100%].

### Example 177

### N-(cis-4-{[6-(Dimethylamino)-2-ethylpyrimidin-4-yl]amino}cyclohexyl)-3,4,5-trifluorobenzamide hydrochloride

The title compound (126 mg) was prepared from N-{cis-4-[(6-chloro-2-methylpyrimidin-4-yl)amino]cyclohexyl}-3,4,5-trifluorobenzamide obtained in step A of example 168 (403 mg) and (6-chloro-2-ethyl-pyrimidin-4-yl)-dimethyl-amine in step B of example 32 (250 mg) using the procedure for the step C of example 168.

¹H NMR (300 MHz, CDCl₃, δ): 1.36 (t, J=7.5 Hz, 3H), 1.65-2.02 (m, 8H), 2.75 (q, J=7.5 Hz, 2H), 2.97-3.41 (m, 6H), 3.68-3.77 (m, 1H), 4.02-4.17 (m, 1H), 5.15 (s, 1H), 6.89 (d, J=8.7 Hz, 1H), 7.48-7.60 (m, 2H), 8.58 (d, J=8.5 Hz, 1H), 13.48-13.72 (m, 1H); ESI MS m/z 422 [M (free)⁺+1, 100%].

### Example 178

### 3-Chloro-N-(cis-4-{[6-(dimethylamino)-2-phenylpyrimidin-4-yl]amino}cyclohexyl)-4-fluorobenzamide hydrochloride

### Step A: Synthesis of 6-chloro-N,N-dimethyl-2-phenylpyrimidin-4-amine

To a solution of 4,6-dichloro-2-phenylpyrimidine (2.00 g) in THF (10 mL) was added 50% aqueous Me₂NH (2.30 mL) and the mixture was stirred at ambient temperature for 3 h. The mixture was diluted with CHCl₃ and added to saturated aqueous NaHCO₃. The aqueous layer was extracted with CHCl₃ (three times). The combined organic layer was dried over MgSO₄, filtrated, concentrated under reduced pressure, and dried under reduced pressure to give the title compound (2.05 g).

¹H NMR (300 MHz, CDCl₃, δ): 3.19 (brs, 6H), 6.34 (s, 1H), 7.39-7.49 (m, 3H), 8.35-8.45 (m, 2H); ESI MS m/z 234 (M⁺+1, 100%).

### Step B: Synthesis of 3-chloro-N-(cis-4-{[6-(dimethylamino)-2-phenylpyrimidin-4-yl]amino}cyclohexyl)-4-fluorobenzamide hydrochloride

The title compound (85 mg) was prepared from 6-chloro-N,N-dimethyl-2-phenylpyrimidin-4-amine (250 mg) and N-(cis-4-amino-cyclohexyl)-3-chloro-4-fluoro-benzamide obtained in step A of example 31 (319 mg) using the procedure for the step C of example 168.

¹H NMR (300 MHz, CDCl₃, δ): 1.69-2.13 (m, 8H), 3.05-3.53 (m, 6H), 3.75-3.84 (m, 1H), 4.07-4.23 (m, 1H), 5.26 (s, 1H), 6.56-6.67 (m, 1H), 7.18 (t, J=8.6 Hz, 1H), 7.51-7.75 (m, 4H), 7.95 (d, J=8.5 Hz, 1H), 8.48 (d, J=6.5 Hz, 2H), 9.25-9.37 (m, 1H), 13.71-13.88 (m, 1H); ESI MS m/z 468 [M (free)⁺+1, 100%].

### Example 179

### N-(cis-4-{[2-Benzyl-6-(dimethylamino)pyrimidin-4-yl]amino}cyclohexyl)-3-chloro-4-fluorobenzamide hydrochloride

### Step A: Synthesis of 2-benzyl-6-chloro-N,N-dimethylpyrimidin-4-amine

The title compound (2.02 g) was prepared from 2-benzyl-4,6-dichloropyrimidine (2.00 g) and 50% aqueous Me₂NH (2.20 mL) using the procedure for the step A of example 178.

¹H NMR (300 MHz, CDCl₃, δ): 3.06 (s, 6H), 4.02 (s, 2H), 6.23 (s, 1H), 7.16-7.43 (m, 5H); ESI MS m/z 248 (M⁺+1, 100%).

### Step B: Synthesis of N-(cis-4-{[2-benzyl-6-(dimethylamino)pyrimidin-4-yl]amino}cyclohexyl)-3-chloro-4-fluorobenzamide hydrochloride

The title compound (132 mg) was prepared from 2-benzyl-6-chloro-N,N-dimethylpyrimidin-4-amine (250 mg) and N-(cis-4-amino-cyclohexyl)-3-chloro-4-fluoro-benzamide obtained in step A of example 31 (301 mg) using the procedure for the step C of example 168.

¹H NMR (300 MHz, CDCl₃, δ): 1.65-2.04 (m, 8H), 2.94-3.38 (m, 6H), 3.63-3.75 (m, 1H), 3.98 (s, 2H), 4.02-4.21 (m, 1H), 5.11 (s, 1H), 6.63 (d, J=8.1 Hz, 1H), 7.14-7.38 (m, 4H), 7.46-7.54 (m, 2H), 7.67-7.75 (m, 1H), 7.91-7.97 (m, 1H), 8.57 (d, J=7.9 Hz, 1H); ESI MS m/z 482 [M (free)⁺+1, 100%].

### Example 180

### 3-Chloro-N-(cis-4-{[6-(dimethylamino)-2,5-dimethylpyrimidin-4-yl]amino}cyclohexyl)-4-fluorobenzamide hydrochloride

### Step A: Synthesis of 2,5-dimethylpyrimidine-4,6-diol

To a solution of Na (1.39 g) in EtOH (42 mL) were added diethyl methylmalonate (5.00 g) and acetamidine hydrochloride (2.71 g). The mixture was stirred at reflux for 2.5 h and cooled to ambient temperature. The precipitate was collected by filtration, washed with EtOH, and dried at 80° C. under reduced pressure to give a white solid. To a solution of the above solid in H₂O (30 mL) was added conc. HCl (2.5 mL) and the mixture was stirred at 4° C. for 1 h. The precipitate was collected by filtration, washed with H₂O (twice), EtOH (twice), and Et₂O (twice), and dried at 80° C. under reduced pressure to give the title compound (3.02 g).

¹H NMR (300 MHz, DMSO-d₆, δ): 1.69 (s, 3H), 2.19 (s, 3H), 11.42-11.66 (m, 2H); ESI MS m/z 139 (M⁻−1, 100%).

### Step B: Synthesis of 4,6-dichloro-2,5-dimethylpyrimidine

A mixture of 2,5-dimethylpyrimidine-4,6-diol (3.02 g), POCl₃ (4.2 mL), and N,N-dimethylaniline (3.0 mL) was stirred at reflux for 1.5 hr and cooled to ambient temperature. The mixture was poured into ice water (20 mL) and stirred for 2 h. The precipitate was collected by filtration, washed with H₂O and hexane, and dried at 60° C. to give the title compound (1.66 g).

¹H NMR (300 MHz, CDCl₃, δ): 2.45 (s, 3H), 2.66 (s, 3H); CI MS m/z 177 (M⁺, 100%).

### Step C: Synthesis of 6-chloro-N,N,2,5-tetramethylpyrimidin-4-amine

The title compound (1.65 g) was prepared from 4,6-dichloro-2,5-dimethylpyrimidine (1.66 g) and 50% aqueous Me₂NH (2.40 mL) using the procedure for the step A of example 178.

¹H NMR (300 MHz, CDCl₃, δ): 2.25 (s, 3H), 2.48 (s, 3H), 3.02 (s, 6H); ESI MS m/z 186 (M⁺+1, 100%).

### Step D: Synthesis of 3-chloro-N-(cis-4-{[6-(dimethylamino)-2,5-dimethylpyrimidin-4-yl]amino}cyclohexyl)-4-fluorobenzamide hydrochloride

The title compound (231 mg) was prepared from 6-chloro-N,N,2,5-tetramethylpyrimidin-4-amine (300 mg) and N-(cis-4-amino-cyclohexyl)-3-chloro-4-fluoro-benzamide obtained in step A of example 31 (481 mg) using the procedure for the step C of example 168.

¹H NMR (300 MHz, CDCl₃, δ): 1.63-2.19 (m, 11H) 2.56 (brs, 3H), 3.18 (s, 6H), 3.92-4.27 (m, 2H), 6.82-6.94 (m, 1H), 7.10-7.25 (m, 2H), 7.80-7.88 (m, 1H), 8.03 (d, J=6.2 Hz, 1H); ESI MS m/z 420 [M (free)⁺+1, 100%].

### Example 181

### 3-Chloro-N-(cis-4-{[6-(dimethylamino)-5-fluoro-2-methylpyrimidin-4-yl]amino}cyclohexyl)-4-fluorobenzamide hydrochloride

### Step A: Synthesis of 5-fluoro-2-methylpyrimidine-4,6-diol

The title compound (3.21 g) was prepared from diethyl fluoromalonate (5.27 g) and acetamidine hydrochloride (2.80 g) using the procedure for the step A of example 180.

¹H NMR (300 MHz, DMSO-d₆, δ): 2.22 (d, J=0.9 Hz, 3H); ESI MS m/z 143 (M⁻−1, 100%).

### Step B: Synthesis of 4,6-dichloro-5-fluoro-2-methylpyrimidine

The title compound (3.13 g) was prepared from 5-fluoro-2-methylpyrimidine-4,6-diol (3.20 g) using the procedure for the step B of example 180.

¹H NMR (200 MHz, CDCl₃, δ): 2.69 (d, J=1.3 Hz, 3H); CI MS m/z 181 (M⁺+1, 100%).

### Step C: Synthesis of 6-chloro-5-fluoro-N,N,2-trimethylpyrimidin-4-amine

The title compound (2.02 g) was prepared from 4,6-dichloro-5-fluoro-2-methylpyrimidine (3.10 g) using the procedure for the step C of example 180.

¹H NMR (300 MHz, CDCl₃, δ): 2.44 (d, J=0.9 Hz, 3H), 3.22 (d, J=2.5 Hz, 6H); ESI MS m/z 190 (M⁺+1, 100%).

### Step D: Synthesis of 3-chloro-N-(cis-4-{[6-(dimethylamino)-5-fluoro-2-methylpyrimidin-4-yl]amino}cyclohexyl)-4-fluorobenzamide hydrochloride

The title compound (135 mg) was prepared from 6-chloro-5-fluoro-N,N,2-trimethylpyrimidin-4-amine (300 mg) and N-(cis-4-amino-cyclohexyl)-3-chloro-4-fluoro-benzamide obtained in step A of example 31 (471 mg) using the procedure for the step C of example 168.

¹H NMR (300 MHz, CDCl₃, δ): 1.70-2.13 (m, 8H), 2.48 (s, 3H), 3.29 (d, J=3.1 Hz, 6H), 4.06-4.21 (m, 2H), 6.52-6.70 (m, 1H), 7.12-7.25 (m, 1H), 7.66-8.02 (m, 3H); ESI MS m/z 424 [M (free)⁺+1, 100%].

### Example 182

### 3-Chloro-N-(cis-4-{[6-(dimethylamino)-2-methylpyrimidin-4-yl]amino}cyclohexyl)-4-fluorobenzenesulfonamide hydrochloride

The title compound (271 mg) was prepared from N-(cis-4-amino-cyclohexyl)-2,N′,N′-trimethyl-pyrimidine-4,6-diamine obtained in step C of example 6 (250 mg) and 3-chloro-4-fluorobenzenesulfonyl chloride (275 mg) using the procedure for the example 7.

¹H NMR (300 MHz, CDCl₃, δ): 1.57-1.96 (m, 8H), 2.47 (s, 3H), 2.94-3.39 (m, 7H), 3.50-3.61 (m, 1H), 5.08 (s, 1H), 5.83 (d, J=6.7 Hz, 1H), 7.21-7.31 (m, 1H), 7.85-7.93 (m, 1H), 8.00-8.06 (m, 1H), 8.38 (d, J=−8.2 Hz, 1H); ESI MS m/z 442 [M (free)⁺+1, 100%].

### Example 183

### N-(3-Chloro-4-fluorophenyl)-N′-(cis-4-{[6-(dimethylamino)-2-methylpyrimidin-4-yl]amino}cyclohexyl)thiourea hydrochloride

To a solution of N-(cis-4-amino-cyclohexyl)-2,N′,N′-trimethyl-pyrimidine-4,6-diamine obtained in step C of example 6 (250 mg) in DMSO (2 mL) was added 3-chloro-4-fluorophenyl isothiocyanate (206 mg) in DMSO (1 mL). The mixture was stirred at ambient temperature for 14 h and poured into water. The precipitate was collected by filtration, washed with water, and purified by medium-pressure liquid chromatography (NH-silica gel, 20% to 50% EtOAc in hexane). To a solution of the above material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 3 h. The precipitate was collected by filtration, washed with Et₂O, and dried at 80° C. under reduced pressure to give the title compound (186 mg).

¹H NMR (300 MHz, CDCl₃, δ): 1.70-2.12 (m, 8H), 2.40 (s, 3H), 2.95-3.40 (m, 6H), 3.46-3.61 (m, 1H), 4.38-4.54 (m, 1H), 5.09 (brs, 1H), 6.99-7.13 (m, 1H), 7.37-7.57 (m, 2H), 7.65-7.77 (m, 1H), 7.88-8.01 (m, 1H), 9.16-9.29 (m, 1H), 13.26-13.42 (m, 1H); ESI MS m/z 437 [M (free)⁺+1, 100%].

### Example 184

### 4-Bromophenyl (cis-4-{[6-(dimethylamino)-2-methylpyrimidin-4-yl]amino}cyclohexyl)carbamate

To a solution of N-(cis-4-amino-cyclohexyl)-2,N′,N′-trimethyl-pyrimidine-4,6-diamine obtained in step C of example 6 (250 mg) in CHCl₃ (3 mL) were added Et₃N (0.21 mL) and 4-bromophenyl chloroformate (283 mg). The mixture was stirred at ambient temperature for 14 hr. The reaction was quenched with saturated aqueous NaHCO₃ and the aqueous layer was extracted with CHCl₃ (three times). The combined organic layer was dried over MgSO₄, filtered, concentrated under reduced pressure, and purified by medium-pressure liquid chromatography (silica gel, 2% to 9% MeOH in CHCl₃) to give the title compound (100 mg).

¹H NMR (300 MHz, CDCl₃, δ): 1.54-1.95 (m, 8H), 2.36 (s, 3H), 3.06 (s, 6H), 3.58-3.81 (m, 2H), 4.66-4.77 (m, 1H), 4.96-5.04 (m, 1H), 5.15 (s, 1H), 7.03 (d, J=9.0 Hz, 2H), 7.46 (d, J=8.9 Hz, 2H); ESI MS m/z 448 (M⁺+1, 100%).

### Example 185

### 3-Chloro-N-{cis-4-[(2,6-dimethoxypyrimidin-4-yl)amino]cyclohexyl}-4-fluorobenzamide hydrochloride

The title compound (16 mg) was prepared from 6-chloro-2,4-dimethoxypyrimidine (250 mg) and N-(cis-4-amino-cyclohexyl)-3-chloro-4-fluoro-benzamide obtained in step A of example 31 (426 mg) using the procedure for the step C of example 168.

¹H NMR (300 MHz, CDCl₃, δ): 1.66-2.04 (m, 8H), 3.64-3.78 (m, 1H), 4.03 (s, 3H), 4.06-4.22 (m, 4H), 5.52 (s, 1H), 6.71-6.86 (m, 1H), 7.12-7.24 (m, 1H), 7.68-7.79 (m, 1H), 7.95 (d, J=8.2 Hz, 1H), 9.14-9.28 (m, 1H); ESI MS m/z 409 [M (free)⁺+1, 40%], 423 [M (free)⁺+15, 100%].

### Example 186

### 3-Chloro-4-fluoro-N-[cis-4-(7H-pyrrolo[2,3-d]pyrimidin-4-ylamino)cyclohexyl]benzamide hydrochloride

The title compound (113 mg) was prepared from 4-chloro-7H-pyrrolo[2,3-d]pyrimidine (300 mg) and N-(cis-4-amino-cyclohexyl)-3-chloro-4-fluoro-benzamide obtained in step A of example 31 (582 mg) using the procedure for the step C of example 168.

¹H NMR (300 MHz, DMSO-d₆, δ): 1.61-2.09 (m, 8H), 3.91-4.17 (m, 2H), 7.01-7.12 (m, 1H), 7.35-7.47 (m, 1H), 7.49-7.59 (m, 1H), 7.88-7.98 (m, 1H), 8.11-8.18 (m, 1H), 8.25-8.41 (m, 2H), 9.10-9.33 (m, 1H), 12.58-12.78 (m, 1H); ESI MS m/z 388 [M (free)⁺+1, 100%].

### Example 187

### 3-Chloro-4-fluoro-N-{cis-4-[(7-methyl-7H-pyrrolo[2,3-d]pyrimidin-4-yl)amino]cyclohexyl}-benzamide hydrochloride

### Step A: Synthesis of 4-chloro-7-methyl-7H-pyrrolo[2,3-d]pyrimidine

To a solution of 4-chloro-7H-pyrrolo[2,3-d]pyrimidine (1.00 g) in DMF (10 mL) under N₂ was added 60% NaH in oil (287 mg) and the mixture was stirred at ambient temperature for 10 min. Iodomethane (0.45 mL) was added to the mixture and the mixture was stirred at ambient temperature for 3 h. The reaction was quenched with saturated aqueous NH₄Cl and the aqueous layer was extracted with EtOAc (three times). The combined organic layer was dried over MgSO₄, filtered, concentrated under reduced pressure, and purified by medium-pressure liquid chromatography (silica gel, 50% EtOAc in hexane) to give the title compound (999 mg).

¹H NMR (300 MHz, CDCl₃, δ): 3.90 (s, 3H), 6.61 (d, J=3.6 Hz, 1H), 7.22 (d, J=3.6 Hz, 1H), 8.65 (s, 1H); ESI MS m/z 168 [M (free)⁺+1, 100%].

### Step B: Synthesis of 3-chloro-4-fluoro-N-{cis-4-[(7-methyl-7H-pyrrolo[2,3-d]pyrimidin-4-yl)amino]cyclohexyl}benzamide hydrochloride

The title compound (765 mg) was prepared from 4-chloro-7-methyl-7H-pyrrolo[2,3-d]pyrimidine (400 mg) and N-(cis-4-amino-cyclohexyl)-3-chloro-4-fluoro-benzamide obtained in step A of example 31 (711 mg) using the procedure for the step C of example 168.

¹H NMR (300 MHz, DMSO-d₆, δ): 1.64-2.11 (m, 8H), 3.81 (s, 3H), 3.91-4.23 (m, 2H), 7.00-7.17 (m, 1H), 7.40-7.59 (m, 2H), 7.87-7.98 (m, 1H), 8.14 (dd, J=7.1, 2.2 Hz, 1H), 8.29-8.41 (m, 2H), 9.17-9.37 (m, 1H); ESI MS m/z 402 [M (free)⁺+1, 100%].

### Example 188

### 3,4,5-Trifluoro-N-{cis-4-[(7-methyl-7H-pyrrolo[2,3-d]pyrimidin-4-yl)amino]cyclohexyl}benzamide hydrochloride

The title compound (168 mg) was prepared from N-(cis-4-aminocyclohexyl)-3,4,5-trifluorobenzamide obtained in step A of example 168 (487 mg) and 4-chloro-7-methyl-7H-pyrrolo[2,3-d]pyrimidine (250 mg) using the procedure for the step C of example 168.

¹H NMR (300 MHz, DMSO-d₆, δ): 1.60-2.15 (m, 8H), 3.81 (s, 3H), 3.90-4.26 (m, 2H), 6.94-7.17 (m, 1H), 7.35-7.53 (m, 1H), 7.73-7.98 (m, 2H), 8.22-8.47 (m, 2H), 9.14-9.42 (m, 1H); ESI MS m/z 404 [M (free)⁺+1, 100%].

### Example 189

### 3-Chloro-N-{cis-4-[(7-ethyl-7H-pyrrolo[2,3-d]pyrimidin-4-yl)amino]cyclohexyl}-4-fluorobenzamide hydrochloride

### Step A: Synthesis of 4-chloro-7-ethyl-7H-pyrrolo[2,3-d]pyrimidine

The title compound (577 mg) was prepared from 4-chloro-7H-pyrrolo[2,3-d]pyrimidine (500 mg) and iodoethane (0.31 mL) using the procedure for the step A of example 187.

¹H NMR (300 MHz, CDCl₃, δ): 1.50 (t, J=7.3 Hz, 3H), 4.34 (q, J=7.3 Hz, 2H), 6.61 (d, J=3.6 Hz, 1H), 7.27 (d, J=3.6 Hz, 1H), 8.64 (s, 1H); ESI MS m/z 182 (M⁺+1, 100%).

### Step B: Synthesis of 3-chloro-N-{cis-4-[(7-ethyl-7H-pyrrolo[2,3-d]pyrimidin-4-yl)amino]cyclohexyl}-4-fluorobenzamide hydrochloride

The title compound (299 mg) was prepared from 4-chloro-7-ethyl-7H-pyrrolo[2,3-d]-pyrimidine (250 mg) and N-(cis-4-amino-cyclohexyl)-3-chloro-4-fluoro-benzamide obtained in step A of example 31 (410 mg) using the procedure for the step C of example 168.

¹H NMR (300 MHz, DMSO-d₆, δ): 1.37 (t, J=7.2 Hz, 3H), 1.63-2.08 (m, 8H), 3.92-4.20 (m, 2H), 4.26 (q, J=7.3 Hz, 2H), 7.03-7.13 (m, 1H), 7.47-7.59 (m, 2H), 7.88-7.97 (m, 1H), 8.14 (dd, J=7.2, 2.1 Hz, 1H), 8.27-8.39 (m, 2H), 9.18-9.35 (m, 1H); ESI MS m/z 416 [M (free)⁺+1, 100%].

### Example 190

### 3-Chloro-4-fluoro-N-{cis-4-[(9-methyl-9H-purin-6-yl)amino]cyclohexyl}benzamide hydrochloride

### Step A: Synthesis of 6-chloro-9-methyl-9H-purine

The title compound (1.08 g) was prepared from 6-chloro-9H-purine (2.00 g) and iodomethane (0.96 mL) using the procedure for the step A of example 187.

¹H NMR (300 MHz, CDCl₃, δ): 3.95 (s, 3H), 8.12 (s, 1H), 8.78 (s, 1H); ESI MS m/z 182 (M⁺+1, 100%).

### Step B: Synthesis of 3-chloro-4-fluoro-N-{cis-4-[(9-methyl-9H-purin-6-yl)amino]-cyclohexyl}benzamide hydrochloride

The title compound (170 mg) was prepared from 6-chloro-9-methyl-9H-purine (250 mg) and N-(cis-4-amino-cyclohexyl)-3-chloro-4-fluoro-benzamide obtained in step A of example 31 (410 mg) using the procedure for the step C of example 168.

¹H NMR (300 MHz, DMSO-d₆, δ): 1.61-2.06 (m, 8H), 3.83 (s, 3H), 3.86-4.31 (m, 2H), 4.72-4.98 (m, 1H), 7.48-7.59 (m, 1H), 7.86-7.95 (m, 1H), 8.11 (dd, J=7.3, 2.2 Hz, 1H), 8.20-8.61 (m, 3H); ESI MS m/z 403 [M (free)⁺+1, 90%], 425 [M (free)⁺+23, 100%].

### Example 191

### cis-N-(3-Chloro-4-fluorophenyl)-4-{[6-(dimethylamino)-2-methylpyrimidin-4-yl]amino}cyclohexanecarboxamide hydrochloride

### Step A: Synthesis of cis-4-{[6-(dimethylamino)-2-methylpyrimidin-4-yl]amino}-cyclohexanecarboxylic acid

To a suspension of (6-chloro-2-methyl-pyrimidin-4-yl)-dimethyl-amine obtained in step B of example 5 (20.0 g) in toluene (300 mL) under N₂ were added cis-4-amino-cyclohexanecarboxylic acid (16.7 g), biphenyl-2-yl(di-tert-butyl)phosphine (346 mg), palladium(II) acetate (260 mg), and sodium tert-butoxide (21.6 g). The mixture was stirred at reflux for 6 h and cooled to ambient temperature. To the mixture was added 1 M aqueous NaOH (300 mL) and the two layers were separated. The aqueous layer was washed with EtOAc. The aqueous layer was cooled on an ice-bath and c.HCl (15 mL) was added (pH=6). The precipitate was collected by filtration, washed with H₂O and EtOAc, and dried at 80° C. under reduced pressure to give the title compound (22.1 g).

¹H NMR (300 MHz, CDCl₃, δ): 1.64-2.16 (m, 8H), 2.35-2.48 (m, 4H), 3.10 (s, 6H), 3.46-3.59 (m, 1H), 5.11 (s, 1H), 8.74-8.84 (m, 1H); ESI MS m/z 279 (M⁺+1, 100%).

### Step B: Synthesis of cis-N-(3-chloro-4-fluorophenyl)-4-{[6-(dimethylamino)-2-methylpyrimidin-4-yl]amino}cyclohexanecarboxamide hydrochloride

To a suspension of cis-4-{[6-(dimethylamino)-2-methylpyrimidin-4-yl]amino}cyclohexanecarboxylic acid (180 mg) and 3-chloro-4-fluoroaniline (114 mg) in DMF (2 mL) were added Et₃N (0.22 mL), HOBt-H₂O (150 mg), and EDC-HCl (150 mg). The mixture was stirred at ambient temperature for 14 h. To the mixture was added water (20 mL) and the aqueous layer was extracted with CHCl₃ (three times). The combined organic layer was dried over MgSO₄, filtrated, concentrated under reduced pressure, and purified by medium-pressure liquid chromatography (NH-silica gel, 20% to 50% EtOAc in hexane) to give a colorless oil. To a solution of the above oil in EtOAc (10 mL) was added 4 M hydrogen chloride in EtOAc (0.5 mL). The mixture was stirred at ambient temperature for 1 h and concentrated. The residue was suspended in Et₂O (10 mL) and the suspension was stirred at ambient temperature for 4 h. The precipitate was collected by filtration, washed with Et₂O, and dried at 80° C. under reduced pressure to give the title compound (27 mg).

¹H NMR (300 MHz, CDCl₃, δ): 1.53-1.73 (m, 2H), 1.81-2.02 (m, 4H), 2.13-2.34 (m, 2H), 2.37-2.58 (m, 4H), 3.03-3.36 (m, 6H), 3.76-3.89 (m, 1H), 5.17 (s, 1H), 6.96-7.12 (m, 1H), 7.64-7.77 (m, 1H), 8.02-8.22 (m, 1H), 8.80-8.93 (m, 1H), 9.30-9.46 (m, 1H); ESI MS m/z 406 [M (free)⁺+1, 100%].

### Example 192

### cis-N-(3,4-Difluorophenyl)-4-{[6-(dimethylamino)-2-methylpyrimidin-4-yl]amino}cyclohexanecarboxamide hydrochloride

To a suspension of cis-4-{[6-(dimethylamino)-2-methylpyrimidin-4-yl]amino}-cyclohexanecarboxylic acid obtained in step A of example 191 (2.1 g) in CHCl₃ (21 mL) were added thionyl chloride (1.21 mL) and DMF (6 mg). The mixture was stirred at reflux for 1.5 h, concentrated under reduced pressure, and the residue was dissolved in CHCl₃ (4.9 mL). To a solution of 3,4-difluoroaniline (223 mg) in CHCl₃ (3 mL) were added Et₃N (0.42 mL) and above acid chloride in CHCl₃ (1 mL). The mixture was stirred at ambient temperature for 14 h and added to saturated aqueous NaHCO₃. The aqueous layer was extracted with CHCl₃ (three times). The combined organic layer was dried over MgSO₄, filtrated, concentrated under reduced pressure, and purified by medium-pressure liquid chromatography (NH-silica gel, 11% to 50% EtOAc in hexane) to give a colorless oil. To a solution of the above oil in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 hr. The precipitate was collected by filtration, washed with Et₂O, and dried at 80° C. under reduced pressure to give the title compound (102 mg).

¹H NMR (300 MHz, CDCl₃, δ): 1.51-2.37 (m, 8H), 2.40-2.55 (s, 4H), 3.07 (brs, 3H), 3.31 (brs, 3H), 3.77-3.91 (m, 1H), 5.18 (s, 1H), 6.98-7.12 (m, 1H), 7.56-7.66 (m, 1H), 7.96-8.07 (m, 1H), 8.82 (d, J=9.8 Hz, 1H), 9.21-9.28 (m, 1H), 13.10-13.26 (m, 1H); ESI MS m/z 390 [M (free)⁺+1, 100%].

### Example 193

### cis-4-{[6-(Dimethylamino)-2-methylpyrimidin-4-yl]amino}-N-(3,4,5-trifluorophenyl)-cyclohexanecarboxamide hydrochloride

The title compound (173 mg) was prepared from 3,4,5-trifluoroaniline (254 mg) using the procedure for the example 192.

¹H NMR (300 MHz, CDCl₃, δ): 1.54-1.72 (m, 2H), 1.81-2.01 (m, 4H), 2.15-2.36 (m, 2H), 2.40-2.55 (m, 4H), 3.07 (brs, 3H), 3.31 (brs, 3H), 3.80-3.90 (m, 1H), 5.18 (s, 1H), 7.69-7.81 (m, 2H), 8.79 (d, J=9.6 Hz, 1H), 9.37 (brs, 1H), 13.05 (brs, 1H); ESI MS m/z 408 [M (free)⁺+1, 100%].

### Example 194

### 3-Chloro-4-fluorophenyl cis-4-{[6-(dimethylamino)-2-methylpyrimidin-4-yl]amino}-cyclohexanecarboxylate hydrochloride

The title compound (4 mg) was prepared from 3-chloro-4-fluorophenol (254 mg) using the procedure for the example 192.

¹H NMR (300 MHz, CDCl₃, δ): 1.61-2.33 (m, 8H), 2.38-2.56 (m, 3H), 2.60-2.77 (m, 1H), 2.91-3.44 (m, 6H), 3.48-3.71 (m, 1H), 5.10 (s, 1H), 6.91-7.34 (m, 3H), 8.38-8.55 (m, 1H);

ESI MS m/z 407 [M (free)⁺+1, 100%].

### Example 195

### cis-N-(3,5-Dichlorophenyl)-4-{[6-(dimethylamino)-2-methylpyrimidin-4-yl]amino}-cyclohexanecarboxamide hydrochloride

The title compound (35 mg) was prepared from 3,5-dichlorophenol (282 mg) using the procedure for the example 192.

¹H NMR (300 MHz, CDCl₃, δ): 1.72-2.31 (m, 8H), 2.49 (s, 3H), 2.60-2.73 (m, 1H), 2.97-3.41 (m, 6H), 3.52-3.68 (m, 1H), 5.11 (s, 1H), 7.08 (d, J=1.9 Hz, 2H), 7.21-7.24 (m, 1H), 8.49 (d, J=7.1 Hz, 1H); ESI MS m/z 423 [M (free)⁺+1, 100%].

### Example 196

### 3,4-Difluorophenyl cis-4-{[6-(dimethylamino)-2-methylpyrimidin-4-yl]amino}-cyclohexanecarboxylate hydrochloride

The title compound (3 mg) was prepared from 3,4-difluorophenol (225 mg) using the procedure for the example 192.

¹H NMR (300 MHz, CDCl₃, δ): 1.69-2.32 (m, 8H), 2.49 (s, 3H), 2.58-2.77 (m, 1H), 2.93-3.41 (m, 6H), 3.51-3.67 (m, 1H), 5.11 (s, 1H), 6.82-7.24 (m, 3H), 8.32-8.58 (m, 1H); ESI MS m/z 391 [M (free)⁺+1, 100%].

### Example 197-274

To a suspension of poly(4-vinylpyridine) (150 μL) in CHCl₃ (200 μL) were added N-(cis-4-amino-cyclohexyl)-2,N′,N′-trimethyl-pyrimidine-4,6-diamine obtained in step C of example 6 (60 μmol) in CHCl₃ (200 μL) and acid chloride (120 μmol) in CHCl₃ (200 μL) at ambient temperature. After stirring at the same temperature for 14 h, the mixture was filtrated and concentrated under reduced pressure. To the residue were added CHCl₃ (685 μL) and PSA (300 μL). After the stirring at ambient temperature for 14 h, the mixture was purified by silica gel chromatography (NH-silica gel, 50% to 100% EtOAc in hexane and silica gel, CHCl₃ to 6% 2 M NH₃/MeOH in CHCl₃) to give the desired product. The product was determined by ESI-MS or APCI-MS.

### Example 275-352

To a suspension of 1-cyclohexyl-3-methylpolystyrene-carbodiimide (150 μL) in CHCl₃ (400 μL) were added N-(cis-4-amino-cyclohexyl)-2,N′,N′-trimethyl-pyrimidine-4,6-diamine obtained in step C of example 6 (30 μmol) in CHCl₃ (200 μL) and carboxylic acid (60 μmol) in CHCl₃ (200 μL) at ambient temperature. After stirring at the same temperature for 13 h, the mixture was filtrated through NH-silica gel. The filtrate was concentrated under reduced pressure, and the residue was purified by silica gel chromatography (silica gel, CHCl₃ to 6% 2 M NH₃/MeOH in CHCl₃) to give the desired product. The product was determined by ESI-MS or APCI-MS.

### Example 353-410

To a solution of half the weight of amide product obtained in example 197-274 in THF (200 μl) was added 1 M borane-THF complex in THF (300 μl). The mixture was stirred at 80° C. for 1 h, and concentrated under reduced pressure. To the residue were added 1 M aqueous HCl (300 μl) and THF (200 μl). The mixture was stirred at 80° C. for 1 h and concentrated under reduced pressure. To the residue was partitioned between CHCl₃ and 2 M aqueous sodium hydroxide. The aqueous layer was extracted with CHCl₃ (300 μL, twice) and EtOAc (300 μL). The combined organic layers were dried over MgSO₄, concentrated under reduced pressure, and purified by silica gel chromatography (silica gel, 33% EtOAc in hexane to 6% 2 M NH₃/MeOH in CHCl₃) to give the desired product. The product was determined by ESI-MS or APCI-MS.

### Example 411-451

To a solution of N-(cis-4-amino-cyclohexyl)-2,N′,N′-trimethyl-pyrimidine-4,6-diamine obtained in step C of example 6 (30 μmol) in DMSO (300 μL) was added isocyanate or isothiocyanate (60 μmol) in DMSO (200 μL) at ambient temperature. The mixture was stirred at the same temperature for 12 h and filtrated through a SCX. The filtrate was concentrated under reduced pressure, and the residue was purified by silica gel chromatography (silica gel, 50% EtOAc in hexane to 6% 2 M NH₃/MeOH in CHCl₃) to give the desired product. The product was determined by ESI-MS or APCI-MS.

### Example 452-522

To a suspension of poly(4-vinylpyridine) (75 μL) in CHCl₃ (200 μL) were added N-(cis-4-amino-cyclohexyl)-2,N′,N′-trimethyl-pyrimidine-4,6-diamine obtained in step C of example 6 (30 μmol) in CHCl₃ (200 μL) and chloroformate or sulfonylchloride (60 μmol) in CHCl₃ (200 μL) at ambient temperature. After stirring at the same temperature for 14 h, the mixture was filtrated and concentrated under reduced pressure. To the residue were added CHCl₃ (685 μL) and PSA (300 μL). After the stirring at ambient temperature for 14 h, the mixture was purified by silica gel chromatography (NH-silica gel, 50% to 100% EtOAc in hexane and silica gel, 33% EtOAc in hexane to 6% 2 M NH₃/MeOH in CHCl₃) to give the desired product. The product was determined by ESI-MS or APCI-MS.

| Ex. No. | compound name | MS | class |
|---|---|---|---|
| 197 | 2-[(cis-4-{[6-(dimethylamino)-2-methylpyrimidin-4- | 426 (M + H) | 3 |
| | yl]amino}cyclohexyl)amino]-2-oxo-1-phenylethyl acetate | | |
| 198 | N-(cis-4-{[6-(dimethylamino)-2-methylpyrimidin-4- | 484 (M + H) | 3 |
| | yl]amino}cyclohexyl)-9,10-dioxo-9,10-dihydroanthracene- | | |
| | 2-carboxamide | | |
| 199 | N-(cis-4-{[6-(dimethylamino)-2-methylpyrimidin-4- | 292 (M + H) | 3 |
| | yl]amino}cyclohexyl)acetamide | | |
| 200 | N-(cis-4-{[6-(dimethylamino)-2-methylpyrimidin-4- | 354 (M + H) | 2 |
| | yl]amino}cyclohexyl)benzamide | | |
| 201 | N-(cis-4-{[6-(dimethylamino)-2-methylpyrimidin-4- | 430 (M + H) | 3 |
| | yl]amino}cyclohexyl)biphenyl-4-carboxamide | | |
| 202 | 4-tert-butyl-N-(cis-4-{[6-(dimethylamino)-2-methylpyrimidin-4- | 410 (M + H) | 3 |
| | yl]amino}cyclohexyl)benzamide | | |
| 203 | N-(cis-4-{[6-(dimethylamino)-2-methylpyrimidin-4- | 409 (M) | 3 |
| | yl]amino}cyclohexyl)-1-benzothiophene-2-carboxamide | | |
| 204 | N-(cis-4-{[6-(dimethylamino)-2-methylpyrimidin-4- | 474 (M + H) | 3 |
| | yl]amino}cyclohexyl)-2-{4-[(phenylmethyl)oxy]phenyl}- | | |
| | acetamide | | |
| 205 | 4-bromo-N-(cis-4-{[6-(dimethylamino)-2-methylpyrimidin-4- | 432 (M + H) | 3 |
| | yl]amino}cyclohexyl)benzamide | | |
| 206 | N-(cis-4-{[6-(dimethylamino)-2-methylpyrimidin-4- | 398 (M + H) | 3 |
| | yl]amino}cyclohexyl)-2-[(phenylmethyl)oxy]acetamide | | |
| 207 | N-(cis-4-{[6-(dimethylamino)-2-methylpyrimidin-4- | 396 (M + H) | 3 |
| | y]amino}cyclohexyl)-2,1,3-benzoxadiazole-5-carboxamide | | |
| 208 | 4-chloro-N-(cis-4-{[6-(dimethylamino)-2-methylpyrimidin-4- | 388 (M + H) | 2 |
| | yl]amino}cyclohexyl)benzamide | | |
| 209 | 2-[(4-chlorophenyl)oxy]-N-(cis-4-{[6-(dimethylamino)-2- | 418 (M + H) | 3 |
| | methylpyrimidin-4-yl]amino}cyclohexyl)acetamide | | |
| 210 | (2E)-N-(cis-4-{[6-(dimethylamino)-2-methylpyrimidin-4- | 380 (M + H) | 3 |
| | yl]amino}cyclohexyl)-3-phenylprop-2-enamide | | |
| 211 | N-(cis-4-{[6-(dimethylamino)-2-methylpyrimidin-4- | 318 (M + H) | 3 |
| | yl]amino}cyclohexyl)cyclopropanecarboxamide | | |
| 212 | N-(cis-4-{[6-(dimethylamino)-2-methylpyrimidin-4- | 360 (M + H) | 3 |
| | yl]amino}cyclohexyl)cyclohexanecarboxamide | | |
| 213 | 2-(4-chlorophenyl)-N-(cis-4-{[6-(dimethylamino)-2- | 402 (M + H) | 3 |
| | methylpyrimidin-4-yl]amino}cyclohexyl)acetamide | | |
| 214 | 1-(4-chlorophenyl)-N-(cis-4-{[6-(dimethylamino)-2- | 456 (M + H) | 1 |
| | methylpyrimidin-4-yl]amino}cyclohexyl)- | | |
| | cyclopentanecarboxamide | | |
| 215 | 3-(2-chloro-6-fluorophenyl)-N-(cis-4-{[6-(dimethylamino)-2- | 487 (M + H) | 1 |
| | methylpyrimidin-4-yl]amino}cyclohexyl)-5- | | |
| | methylisoxazole-4-carboxamide | | |
| 216 | 4-[(4-chlorophenyl)sulfonyl]-N-(cis-4-{[6-(dimethylamino)-2- | 548 (M + H) | 3 |
| | methylpyrimidin-4-yl]amino}cyclohexyl)-3- | | |
| | methylthiophene-2-carboxamide | | |
| 217 | 4-(dimethylamino)-N-(cis-4-{[6-(dimethylamino)-2- | 397 (M + H) | 3 |
| | methylpyrimidin-4-yl]amino}cyclohexyl)benzamide | | |
| 218 | N-(cis-4-{[6-(dimethylamino)-2-methylpyrimidin-4- | 390 (M + H) | 1 |
| | yl]amino}cyclohexyl)-3,4-difluorobenzamide | | |
| 219 | N-(cis-4-{[6-(dimethylamino)-2-methylpyrimidin-4- | 414 (M + H) | 3 |
| | yl]amino}cyclohexyl)-3,4-bis(methyloxy)benzamide | | |
| 220 | N-(cis-4-{[6-(dimethylamino)-2-methylpyrimidin-4- | 398 (M + H) | 3 |
| | yl]amino}cyclohexyl)-4-(ethyloxy)benzamide | | |
| 221 | N-(cis-4-{[6-(dimethylamino)-2-methylpyrimidin-4- | 372 (M + H) | 3 |
| | yl]amino}cyclohexyl)-4-fluorobenzamide | | |
| 222 | N-(cis-4-{[6-(dimethylamino)-2-methylpyrimidin-4- | 344 (M + H) | 3 |
| | yl]amino}cyclohexyl)furan-2-carboxamide | | |
| 223 | N-(cis-4-{[6-(dimethylamino)-2-methylpyrimidin-4- | 345 (M + H) | 3 |
| | yl]amino}cyclohexyl)isoxazole-5-carboxamide | | |
| 224 | N-(cis-4-{[6-(dimethylamino)-2-methylpyrimidin-4- | 480 (M + H) | 3 |
| | yl]amino}cyclohexyl)-2-iodobenzamide | | |
| 225 | N-(cis-4-{[6-(dimethylamino)-2-methylpyrimidin-4- | 363 (M + H) | 3 |
| | yl]amino}cyclohexyl)morpholine-4-carboxamide | | |
| 226 | N-(cis-4-{[6-(dimethylamino)-2-methylpyrimidin-4- | 401 (M + H) | 3 |
| | yl]amino}cyclohexyl)-2-(methylthio)pyridine-3-carboxamide | | |
| 227 | methyl 4-{[(cis-4-{[6-(dimethylamino)-2-methylpyrimidin-4- | 412 (M + H) | 3 |
| | yl]amino}cyclohexyl)amino]carbonyl}benzoate | | |
| 228 | N-(cis-4-{[6-(dimethylamino)-2-methylpyrimidin-4- | 435 (M + H) | 3 |
| | yl]amino}cyclohexyl)-5-methyl-2-phenyl-2H-1,2,3-triazole-4- | | |
| | carboxamide | | |
| 229 | N-(cis-4-{[6-(dimethylamino)-2-methylpyrimidin-4- | 376 (M + H) | 3 |
| | yl]amino}cyclohexyl)-4-methyl-1,2,3-thiadiazole-5-carboxamide | | |
| 230 | N-(cis-4-{[6-(dimethylamino)-2-methylpyrimidin-4- | 521 (M + H) | 2 |
| | yl]amino}cyclohexyl)-2-(4-methoxyphenoxy)-5-nitrobenzamide | | |
| 231 | N-(cis-4-{[6-(dimethylamino)-2-methylpyrimidin-4- | 404 (M + H) | 3 |
| | yl]amino}cyclohexyl)naphthalene-2-carboxamide | | |
| 232 | N-(cis-4-{[6-(dimethylamino)-2-methylpyrimidin-4- | 399 (M + H) | 3 |
| | yl]amino}cyclohexyl)-3-nitrobenzamide | | |
| 233 | N-(cis-4-{[6-(dimethylamino)-2-methylpyrimidin-4- | 533 (M + H) | 3 |
| | yl]amino}cyclohexyl)-1-(4-nitrophenyl)-5-(trifluoromethyl)-1H- | | |
| | pyrazole-4-carboxamide | | |
| 234 | N-(cis-4-{[6-(dimethylamino)-2-methylpyrimidin-4- | 384 (M + H) | 3 |
| | yl]amino}cyclohexyl)-2-(phenyloxy)acetamide | | |
| 235 | N-(cis-4-{[6-(dimethylamino)-2-methylpyrimidin-4- | 368 (M + H) | 3 |
| | yl]amino}cyclohexyl)-2-phenylacetamide | | |
| 236 | (2R)—N-(cis-4-{[6-(dimethylamino)-2-methylpyrimidin-4- | 394 (M + H) | 3 |
| | yl]amino}cyclohexyl)-2-phenylcyclopropanecarboxamide | | |
| 237 | N-(cis-4-{[6-(dimethylamino)-2-methylpyrimidin-4- | 398 (M + H) | 3 |
| | yl]amino}cyclohexyl)-1,3-benzodioxole-5-carboxamide | | |
| 238 | N-(cis-4-{[6-(dimethylamino)-2-methylpyrimidin-4- | 488 (M + H) | 3 |
| | yl]amino}cyclohexyl)-1-phenyl-5-(trifluoromethyl)-1H-pyrazole-4- | | |
| | carboxamide | | |
| 239 | N-(cis-4-{[6-(dimethylamino)-2-methylpyrimidin-4- | 429 (M + H) | 3 |
| | yl]amino}cyclohexyl)-2-[(2-nitrophenyl)oxy]acetamide | | |
| 240 | N-(cis-4-{[6-(dimethylamino)-2-methylpyrimidin-4- | 406 (M + H) | 3 |
| | yl]amino}cyclohexyl)quinoxaline-2-carboxamide | | |
| 241 | N-(cis-4-{[6-(dimethylamino)-2-methylpyrimidin-4- | 422 (M + H) | 3 |
| | yl]amino}cyclohexyl)-3-(trifluoromethyl)benzamide | | |
| 242 | N-(cis-4-{[6-(dimethylamino)-2-methylpyrimidin-4- | 368 (M + H) | 3 |
| | yl]amino}cyclohexyl)-4-methylbenzamide | | |
| 243 | N-(cis-4-{[6-(dimethylamino)-2-methylpyrimidin-4- | 360 (M + H) | 3 |
| | yl]amino}cyclohexyl)thiophene-2-carboxamide | | |
| 244 | N-(cis-4-{[6-(dimethylamino)-2-methylpyrimidin-4- | 474 (M + H) | 3 |
| | yl]amino}cyclohexyl)-2-[(pentafluorophenyl)oxy]acetamide | | |
| 245 | 2-[3,4-bis(methyloxy)phenyl]-N-(cis-4-{[6-(dimethylamino)-2- | 428 (M + H) | 3 |
| | methylpyrimidin-4-yl]amino}cyclohexyl)acetamide | | |
| 246 | N-(cis-4-{[6-(dimethylamino)-2-methylpyrimidin-4- | 400 (M + H) | 3 |
| | yl]amino}cyclohexyl)-2-(phenylthio)acetamide | | |
| 247 | N-(cis-4-{[6-(dimethylamino)-2-methylpyrimidin-4- | 456 (M + H) | 3 |
| | yl]amino}cyclohexyl)-9-oxo-9H-fluorene-4-carboxamide | | |
| 248 | N-(cis-4-{[6-(dimethylamino)-2-methylpyrimidin-4- | 438 (M + H) | 3 |
| | yl]amino}cyclohexyl)-4-[(trifluoromethyl)oxy]benzamide | | |
| 249 | N-(cis-4-{[6-(dimethylamino)-2-methylpyrimidin-4- | 440 (M + H) | 3 |
| | yl]amino}cyclohexyl)-4-fluoro-2-(trifluoromethyl)benzamide | | |
| 250 | N-(cis-4-{[6-(dimethylamino)-2-methylpyrimidin-4- | 386 (M + H) | 3 |
| | yl]amino}cyclohexyl)-2-(4-fluorophenyl)acetamide | | |
| 251 | N-(cis-4-{[6-(dimethylamino)-2-methylpyrimidin-4- | 468 (M + H) | 3 |
| | yl]amino}cyclohexyl)-4-(heptyloxy)benzamide | | |
| 252 | N-(cis-4-{[6-(dimethylamino)-2-methylpyrimidin-4- | 424 (M + H) | 3 |
| | yl]amino}cyclohexyl)-4-pentylbenzamide | | |
| 253 | N-(cis-4-{[6-(dimethylamino)-2-methylpyrimidin-4- | 346 (M + H) | 3 |
| | yl]amino}cyclohexyl)cyclopentanecarboxamide | | |
| 254 | N-(cis-4-{[6-(dimethylamino)-2-methylpyrimidin-4- | 480 (M + H) | 3 |
| | yl]amino}cyclohexyl)-4-nonylbenzamide | | |
| 255 | N-(cis-4-{[6-(dimethylamino)-2-methylpyrimidin-4- | 440 (M + H) | 3 |
| | yl]amino}cyclohexyl)-2-{[4-(1,1-dimethylethyl)phenyl]- | | |
| | oxy}acetamide | | |
| 256 | 3-chloro-N-(cis-4-{[6-(dimethylamino)-2-methylpyrimidin-4- | 406 (M + H) | 1 |
| | yl]amino}cyclohexyl)-4-fluorobenzamide | | |
| 257 | 2-cyclopentyl-N-(cis-4-{[6-(dimethylamino)-2-methylpyrimidin-4- | 360 (M + H) | 3 |
| | yl]amino}cyclohexyl)acetamide | | |
| 258 | N-(cis-4-{[6-(dimethylamino)-2-methylpyrimidin-4- | 382 (M + H) | 3 |
| | yl]amino}cyclohexyl)-3-phenylpropanamide | | |
| 259 | 4-cyano-N-(cis-4-{[6-(dimethylamino)-2-methylpyrimidin-4- | 379 (M + H) | 3 |
| | yl]amino}cyclohexyl)benzamide | | |
| 260 | N-[4-(6-dimethylamino-2-methyl-pyrimidin-4-ylamino)- | 587 (M + H) | 3 |
| | cyclohexyl]-2-(naphthalene-1-sulfonylamino)-3- | | |
| | phenyl-propionamide | | |
| 261 | N-(cis-4-{[6-(dimethylamino)-2-methylpyrimidin-4- | 454 (M + H) | 3 |
| | yl]amino}cyclohexyl)-4-[(trifluoromethyl)thio]benzamide | | |
| 262 | (2E)—N-(cis-4-{[6-(dimethylamino)-2-methylpyrimidin-4- | 448 (M + H) | 3 |
| | yl]amino}cyclohexyl)-3-[3-(trifluoromethyl)phenyl]prop- | | |
| | 2-enamide | | |
| 263 | (2E)—N-(cis-4-{[6-(dimethylamino)-2-methylpyrimidin-4- | 425 (M + H) | 3 |
| | yl]amino}cyclohexyl)-3-(4-nitrophenyl)prop-2-enamide | | |
| 264 | 2-(2-bromophenyl)-N-(cis-4-{[6-(dimethylamino)-2- | 446 (M + H) | 3 |
| | methylpyrimidin-4-yl]amino}cyclohexyl)acetamide | | |
| 265 | (2E)-3-(2-chlorophenyl)-N-(cis-4-{[6-(dimethylamino)-2- | 414 (M + H) | 3 |
| | methylpyrimidin-4-yl]amino}cyclohexyl)prop-2-enamide | | |
| 266 | N-(cis-4-{[6-(dimethylamino)-2-methylpyrimidin-4- | 463 (M + H) | 3 |
| | yl]amino}cyclohexyl)-2-(phenylthio)pyridine-3-carboxamide | | |
| 267 | N-(cis-4-{[6-(dimethylamino)-2-methylpyrimidin-4- | 490 (M + H) | 3 |
| | yl]amino}cyclohexyl)-3-(1,1-dimethylethyl)-1-(phenylmethyl)-1H- | | |
| | pyrazole-5-carboxamide | | |
| 268 | 2-[(4-chlorophenyl)oxy]-N-(cis-4-{[6-(dimethylamino)-2- | 446 (M + H) | 3 |
| | methylpyrimidin-4-yl]amino}cyclohexyl)-2-methylpropanamide | | |
| 269 | (2E)—N-(cis-4-{[6-(dimethylamino)-2-methylpyrimidin-4- | 464 (M + H) | 3 |
| | yl]amino}cyclohexyl)-3-{4-[(trifluoromethyl)oxy]phenyl}prop- | | |
| | 2-enamide | | |
| 270 | 1-[(2,4-dichlorophenyl)methyl]-N-(cis-4-{[6-(dimethylamino)-2- | 558 (M + H) | 3 |
| | methylpyrimidin-4-yl]amino}cyclohexyl)-3-(1,1-dimethylethyl)- | | |
| | 1H-pyrazole-5-carboxamide | | |
| 271 | 6-chloro-N-(cis-4-{[6-(dimethylamino)-2-methylpyrimidin-4- | 442 (M + H) | 3 |
| | yl]amino}cyclohexyl)-2H-chromene-3-carboxamide | | |
| 272 | 5-chloro-N-(cis-4-{[6-(dimethylamino)-2-methylpyrimidin-4- | 392 (M + H) | 3 |
| | yl]amino}cyclohexyl)-1-methyl-1H-pyrazole-4-carboxamide | | |
| 273 | N-(cis-4-{[6-(dimethylamino)-2-methylpyrimidin-4- | 466 (M + H) | 3 |
| | yl]amino}cyclohexyl)-2-[(4-methyl-2-oxo-2H-chromen-8- | | |
| | yl)oxy]acetamide | | |
| 274 | N-(cis-4-{[6-(dimethylamino)-2-methylpyrimidin-4- | 437 (M + H) | 3 |
| | yl]amino}cyclohexyl)-2-(1,3-dioxo-1,3-dihydro-2H-isoindol-2- | | |
| | yl)acetamide | | |
| 275 | 2-[(4-acetylphenyl)oxy]-N-(cis-4-{[6-(dimethylamino)-2- | 426 (M + H) | 3 |
| | methylpyrimidin-4-yl]amino}cyclohexyl)acetamide | | |
| 276 | N-((1S)-2-{[(cis-4-{[6-(dimethylamino)-2-methylpyrimidin-4- | 479 (M + H) | 3 |
| | yl]amino}cyclohexyl)amino]carbonyl}cyclohexyl)benzamide | | |
| 277 | N-(cis-4-{[6-(dimethylamino)-2-methylpyrimidin-4- | 543 (M + H) | 3 |
| | yl]amino}cyclohexyl)-1-{[4-(1,1- | | |
| | dimethylethyl)phenyl]sulfonyl}prolinamide | | |
| 278 | 2-cyclohex-1-en-1-yl-N-(cis-4-{[6-(dimethylamino)-2- | 372 (M + H) | 3 |
| | methylpyrimidin-4-yl]amino}cyclohexyl)acetamide | | |
| 279 | 2-cyclohexyl-N-(cis-4-{[6-(dimethylamino)-2-methylpyrimidin-4- | 374 (M + H) | 3 |
| | yl]amino}cyclohexyl)acetamide | | |
| 280 | N-(cis-4-{[6-(dimethylamino)-2-methylpyrimidin-4- | 416 (M + H) | 3 |
| | yl]amino}cyclohexyl)-2-[(4-methylpyrimidin-2-yl)thio]acetamide | | |
| 281 | 3-[(4-chlorophenyl)sulfonyl]-N-(cis-4-{[6-(dimethylamino)-2- | 494 (M + H) | 3 |
| | methylpyrimidin-4-yl]amino}cyclohexyl)butanamide | | |
| 282 | N-(cis-4-{[6-(dimethylamino)-2-methylpyrimidin-4- | 457 (M + H) | 3 |
| | yl]amino}cyclohexyl)-5-oxo-1-(2-thienylmethyl)pyrrolidine-3- | | |
| | carboxamide | | |
| 283 | N-(cis-4-{[6-(dimethylamino)-2-methylpyrimidin-4- | 467 (M + H) | 3 |
| | yl]amino}cyclohexyl)-2,5-dimethyl-1-(2-thienylmethyl)-1H- | | |
| | pyrrole-3-carboxamide | | |
| 284 | N-(cis-4-{[6-(dimethylamino)-2-methylpyrimidin-4- | 476 (M + H) | 3 |
| | yl]amino}cyclohexyl)-2-(2-fluorobiphenyl-4-yl)propanamide | | |
| 285 | N-(cis-4-{[6-(dimethylamino)-2-methylpyrimidin-4- | 470 (M + H) | 3 |
| | yl]amino}cyclohexyl)-5-iodo-2-furamide | | |
| 286 | N-(cis-4-{[6-(dimethylamino)-2-methylpyrimidin-4- | 513 (M + H) | 3 |
| | yl]amino}cyclohexyl)-2-[4-(1-oxo-1,3-dihydro-2H-isoindol-2- | | |
| | yl)phenyl]propanamide | | |
| 287 | N-(cis-4-{[6-(dimethylamino)-2-methylpyrimidin-4- | 494 (M + H) | 3 |
| | yl]amino}cyclohexyl)-2-(2-iodophenyl)acetamide | | |
| 288 | N-(cis-4-{[6-(dimethylamino)-2-methylpyrimidin-4- | 450 (M + H) | 3 |
| | yl]amino}cyclohexyl)-5-(4-methylphenyl)thiophene- | | |
| | 3-carboxamide | | |
| 289 | N-(cis-4-{[6-(dimethylamino)-2-methylpyrimidin-4- | 465 (M + H) | 3 |
| | yl]amino}cyclohexyl)-2-(5-methyl-2-phenyl-1,3-thiazol-4- | | |
| | yl)acetamide | | |
| 290 | N-(cis-4-{[6-(dimethylamino)-2-methylpyrimidin-4- | 452 (M + H) | 3 |
| | yl]amino}cyclohexyl)-2-[6-(methyloxy)-3-oxo-2,3-dihydro-1H- | | |
| | inden-1-yl]acetamide | | |
| 291 | N-(cis-4-{[6-(dimethylamino)-2-methylpyrimidin-4- | 466 (M + H) | 3 |
| | yl]amino}cyclohexyl)-2-[7-(methyloxy)-2-oxo-2H-chromen-4- | | |
| | yl]acetamide | | |
| 292 | N-(cis-4-{[6-(dimethylamino)-2-methylpyrimidin-4- | 488 (M + H) | 3 |
| | yl]amino}cyclohexyl)-4-[4-(methylsulfonyl)phenyl]-4- | | |
| | oxobutanamide | | |
| 293 | N-(cis-4-{[6-(dimethylamino)-2-methylpyrimidin-4- | 423 (M + H) | 3 |
| | yl]amino}cyclohexyl)-5-(methyloxy)-1H-indole-2-carboxamide | | |
| 294 | N-(2,4-difluorophenyl)-2-{2-[(cis-4-{[6-(dimethylamino)-2- | 523 (M + H) | 3 |
| | methylpyrimidin-4-yl]amino}cyclohexyl)amino]-2- | | |
| | oxoethyl}benzamide | | |
| 295 | 2-(2-{[2,5-bis(methyloxy)phenyl]amino}-2-oxoethyl)-N-(cis-4-{[6 | 547 (M + H) | 3 |
| | (dimethylamino)-2-methylpyrimidin-4- | | |
| | yl]amino}cyclohexyl)benzamide | | |
| 296 | 2-{2-[(cis-4-{[6-(dimethylamino)-2-methylpyrimidin-4- | 529 (M + H) | 3 |
| | yl]amino}cyclohexyl)amino]-2-oxoethyl}-N-[4-(1- | | |
| | methylethyl)phenyl]benzamide | | |
| 297 | 2-{2-[(cis-4-{[6-(dimethylamino)-2-methylpyrimidin-4- | 571 (M + H) | 3 |
| | yl]amino}cyclohexyl)amino]-2-oxoethyl}-N-{4- | | |
| | [(trifluoromethyl)oxy]phenyl}benzamide | | |
| 298 | N-(cis-4-{[6-(dimethylamino)-2-methylpyrimidin-4- | 441 (M + H) | 3 |
| | yl]amino}cyclohexyl)-4-(4-nitrophenyl)butanamide | | |
| 299 | N-(cis-4-{[6-(dimethylamino)-2-methylpyrimidin-4- | 408 (M + H) | 3 |
| | yl]amino}cyclohexyl)-3-oxo-2,3-dihydro-1H-indene-1- | | |
| | carboxamide | | |
| 300 | N-(cis-4-{[6-(dimethylamino)-2-methylpyrimidin-4- | 460 (M + H) | 3 |
| | yl]amino}cyclohexyl)-2-[4-(phenyloxy)phenyl]acetamide | | |
| 301 | N-(cis-4-{[6-(dimethylamino)-2-methylpyrimidin-4- | 494 (M + H) | 3 |
| | yl]amino}cyclohexyl)-11-phenylundecanamide | | |
| 302 | N-(cis-4-([6-(dimethylamino)-2-methylpyrimidin-4- | 401 (M + H) | 3 |
| | yl]amino}cyclohexyl)-2-(pyridin-4-ylthio)acetamide | | |
| 303 | N-(cis-4-{[6-(dimethylamino)-2-methylpyrimidin-4- | 383 (M + H) | 3 |
| | yl]amino}cyclohexyl)-N²-phenylglycinamide | | |
| 304 | N-(cis-4-{[6-(dimethylamino)-2-methylpyrimidin-4- | 476 (M + H) | 3 |
| | yl]amino}cyclohexyl)-2-[(4-fluorophenyl)carbonyl]benzamide | | |
| 305 | N-(cis-4-{[6-(dimethylamino)-2-methylpyrimidin-4- | 458 (M + H) | 3 |
| | yl]amino}cyclohexyl)-2-(2-phenylethyl)benzamide | | |
| 306 | N-(cis-4-{[6-(dimethylamino)-2-methylpyrimidin-4- | 504 (M + H) | 1 |
| | yl]amino}cyclohexyl)-2-(ethylthio)-2,2-diphenylacetamide | | |
| 307 | N-(cis-4-{[6-(dimethylamino)-2-methylpyrimidin-4- | 498 (M + H) | 3 |
| | yl]amino}cyclohexyl)-4′-(trifluoromethyl)biphenyl-2- | | |
| | carboxamide | | |
| 308 | N-(cis-4-{[6-(dimethylamino)-2-methylpyrimidin-4- | 487 (M + H) | 3 |
| | yl]amino}cyclohexyl)-7-nitro-9H-fluorene-4-carboxamide | | |
| 309 | (2S)—N-(cis-4-{[6-(dimethylamino)-2-methylpyrimidin-4- | 486 (M + H) | 3 |
| | yl]amino}cyclohexyl)-2-[3-(phenylcarbonyl)phenyl]propanamide | | |
| 310 | 2-[(4-chlorophenyl)thio]-N-(cis-4-{[6-(dimethylamino)-2- | 566 (M + H) | 3 |
| | methylpyrimidin-4-yl]amino}cyclohexyl)-4-(4-methylphenyl)-4- | | |
| | oxobutanamide | | |
| 311 | N-(cis-4-{[6-(dimethylamino)-2-methylpyrimidin-4- | 550 (M + H) | 3 |
| | yl]amino}cyclohexyl)-4-(4-fluorophenyl)-2-[(4- | | |
| | methylphenyl)thio]-4-oxobutanamide | | |
| 312 | N-(cis-4-{[6-(dimethylamino)-2-methylpyrimidin-4- | 492 (M + H) | 3 |
| | yl]amino}cyclohexyl)-2-[4-(2-thienylcarbonyl)phenyl]- | | |
| | propanamide | | |
| 313 | N-(cis-4-{[6-(dimethylamino)-2-methylpyrimidin-4- | 452 (M + H) | 3 |
| | yl]amino}cyclohexyl)-2-{4-[(trifluoromethyl)oxy]phenyl}- | | |
| | acetamide | | |
| 314 | N-(cis-4-{[6-(dimethylamino)-2-methylpyrimidin-4- | 388 (M + H) | 3 |
| | yl]amino}cyclohexyl)-4,4,4-trifluoro-3-methylbutanamide | | |
| 315 | N-(cis-4-{[6-(dimethylamino)-2-methylpyrimidin-4- | 468 (M + H) | 3 |
| | yl]amino}cyclohexyl)-2-{4-[(trifluoromethyl)thio]phenyl}- | | |
| | acetamide | | |
| 316 | N-(cis-4-{[6-(dimethylamino)-2-methylpyrimidin-4- | 416 (M + H) | 3 |
| | yl]amino}cyclohexyl)-5-(2-thienyl)- | | |
| | pentanamide | | |
| 317 | N-(cis-4-{[6-(dimethylamino)-2-methylpyrimidin-4- | 461 (M + H) | 3 |
| | yl]amino}cyclohexyl)-N²-[(4-methylphenyl)sulfonyl]- | | |
| | glycinamide | | |
| 318 | N-(cis-4-{[6-(dimethylamino)-2-methylpyrimidin-4- | 513 (M + H) | 3 |
| | yl]amino}cyclohexyl)-2-{5-[phenylmethyl)oxy]-1H-indol-3- | | |
| | yl}acetamide | | |
| 319 | N-(cis-4-{[6-(dimethylamino)-2-methylpyrimidin-4- | 487 (M + H) | 3 |
| | yl]amino}cyclohexyl)-N′-(3-methylphenyl)benzene-1,2- | | |
| | dicarboxamide | | |
| 320 | N-(cis-4-{[6-(dimethylamino)-2-methylpyrimidin-4- | 512 (M + H) | 3 |
| | yl]amino}cyclohexyl)-3-methyl-4-oxo-2-phenyl-4H-chromene-8- | | |
| | carboxamide | | |
| 321 | phenylmethyl 3-[(cis-4-{[6-(dimethylamino)-2-methylpyrimidin-4- | 502 (M + H) | 3 |
| | yl]amino}cyclohexyl)amino]-3-oxo-2-phenylpropanoate | | |
| 322 | 2-{[3,5-bis(trifluoromethyl)phenyl]carbonyl}-N-(Cis-4-{[6- | 594 (M + H) | 3 |
| | (dimethylamino)-2-methylpyrimidin-4-yl]amino}cyclohexyl)- | | |
| | benzamide | | |
| 323 | N-(cis-4-{[6-(dimethylamino)-2-methylpyrimidin-4- | 528 (M + H) | 3 |
| | yl]amino}cyclohexyl)-2-[(3-methyl-1-benzothien-2- | | |
| | yl)carbonyl]benzamide | | |
| 324 | N-(cis-4-{[6-(dimethylamino)-2-methylpyrimidin-4- | 456 (M + H) | 3 |
| | yl]amino}cyclohexyl)-9-oxo-9H-fluorene-2-carboxamide | | |
| 325 | N-(cis-4-{[6-(dimethylamino)-2-methylpyrimidin-4- | 430 (M + H) | 3 |
| | yl]amino}cyclohexyl)biphenyl-2-carboxamide | | |
| 326 | N-(cis-4-{[6-(dimethylamino)-2-methylpyrimidin-4- | 446 (M + H) | 3 |
| | yl]amino}cyclohexyl)-4-(phenyloxy)benzamide | | |
| 327 | N-(cis-4-{[6-(dimethylamino)-2-methylpyrimidin-4- | 458 (M + H) | 3 |
| | yl]amino}cyclohexyl)-9H-xanthene-9-carboxamide | | |
| 328 | N-(cis-4-{[6-(dimethylamino)-2-methylpyrimidin-4- | 501 (M + H) | 3 |
| | yl]amino}cyclohexyl)-N′-[(1S)-1-phenylethyl]benzene-1,2- | | |
| | dicarboxamide | | |
| 329 | N-(cis-4-{[6-(dimethylamino)-2-methylpyrimidin-4- | 460 (M + H) | 3 |
| | yl]amino}cyclohexyl)-4-[(phenylmethyl)oxy]benzamide | | |
| 330 | N-(cis-4-{[6-(dimethylamino)-2-methylpyrimidin-4- | 472 (M + H) | 3 |
| | yl]amino}cyclohexyl)-2-[(4-methylphenyl)carbonyl]benzamide | | |
| 331 | N-(cis-4-{[6-(dimethylamino)-2-methylpyrimidin-4- | 460 (M + H) | 3 |
| | yl]amino}cyclohexyl)-[(2-phenyloxy)methyl]benzamide | | |
| 332 | N-(cis-4-{[6-(dimethylamino)-2-methylpyrimidin-4- | 523 (M + H) | 3 |
| | yl]amino}cyclohexyl)-N′-naphthalen-1-ylbenzene-1,2- | | |
| | dicarboxamide | | |
| 333 | N-(cis-4-{[6-(dimethylamino)-2-methylpyrimidin-4- | 454 (M + H) | 3 |
| | yl]amino}cyclohexyl)anthracene-2-carboxamide | | |
| 334 | N-(cis-4-{[6-(dimethylamino)-2-methylpyrimidin-4- | 528 (M + H) | 3 |
| | yl]amino}cyclohexyl)-4′-heptylbiphenyl-4-carboxamide | | |
| 335 | 2-[4-(4-chlorophenyl)-2-phenyl-1,3-thiazol-5-yl]-N-(cis-4-{[6- | 561 (M + H) | 3 |
| | (dimethylamino)-2-methylpyrimidin-4-yl]amino}- | | |
| | cyclohexyl)acetamide | | |
| 336 | N-(cis-4-{[6-(dimethylamino)-2-methylpyrimidin-4- | 414 (M + H) | 3 |
| | yl]amino}cyclohexyl)-2-[(phenylmethyl)thio]acetamide | | |
| 337 | N-(cis-4-{[6-(dimethylamino)-2-methylpyrimidin-4- | 396 (M + H) | 3 |
| | yl]amino}cyclohexyl)-4-phenylbutanamide | | |
| 338 | 2-(1-benzothien-3-yl)-N-(cis-4-{[6-(dimethylamino)-2- | 424 (M + H) | 3 |
| | methylpyrimidin-4-yl]amino}cyclohexyl)acetamide | | |
| 339 | 2-(2,3-dihydro-1H-inden-2-yl)-N-(cis-4-{[6-(dimethylamino)-2- | 408 (M + H) | 3 |
| | methylpyrimidin-4-yl]amino}cyclohexyl)acetamide | | |
| 340 | 4-[3,4-bis(methyloxy)phenyl]-N-(cis-4-{[6-(dimethylamino)-2- | 456 (M + H) | 3 |
| | methylpyrimidin-4-yl]amino}cyclohexyl)butanamide | | |
| 341 | 4-(2,3-dihydro-1,4-benzodioxin-6-yl)-N-(cis-4-{[6- | 454 (M + H) | 3 |
| | (dimethylamino)-2-methylpyrimidin-4-yl]amino}cyclohexyl)- | | |
| | butanamide | | |
| 342 | N-(cis-4-{[6-(dimethylamino)-2-methylpyrimidin-4- | 497 (M + H) | 3 |
| | yl]amino}cyclohexyl)-1-[(4-methylphenyl)sulfonyl]-1H-pyrrole-3- | | |
| | carboxamide | | |
| 343 | N-(cis-4-{[6-(dimethylamino)-2-methylpyrimidin-4- | 432 (M + H) | 3 |
| | yl]amino}cyclohexyl)-4-(methylsulfonyl)benzamide | | |
| 344 | 5-acetyl-N-(cis-4-{[6-(dimethylamino)-2-methylpyrimidin-4- | 402 (M + H) | 3 |
| | yl]amino}cyclohexyl)thiophene-2-carboxamide | | |
| 345 | 3-chloro-N-(cis-4-{[6-(dimethylamino)-2-methylpyrimidin-4- | 546 (M + H) | 3 |
| | yl]amino}cyclohexyl)-4-[(1-methylethyl)sulfonyl]-5- | | |
| | (methylthio)thiophene-2-carboxamide | | |
| 346 | N-(cis-4-{[6-(dimethylamino)-2-methylpyrimidin-4- | 438 (M + H) | 3 |
| | yl]amino}cyclohexyl)-5-(methylsulfonyl)thiophene-2- | | |
| | carboxamide | | |
| 347 | N-(cis-4-{[6-(dimethylamino)-2-methylpyrimidin-4- | 421 (M + H) | 3 |
| | yl]amino}cyclohexyl)-4-(1,3-oxazol-5-yl)benzamide | | |
| 348 | N-(cis-4-{[6-(dimethylamino)-2-methylpyrimidin-4- | 533 (M + H) | 3 |
| | yl]amino}cyclohexyl)-1-(phenylsulfonyl)-1H-indole- | | |
| | 3-carboxamide | | |
| 349 | N-(cis-4-{[6-(dimethylamino)-2-methylpyrimidin-4- | 382 (M + H) | 3 |
| | yl]amino}cyclohexyl)-2-oxo-2-phenylacetamide | | |
| 350 | N-(cis-4-{[6-(dimethylamino)-2-methylpyrimidin-4- | 424 (M + H) | 3 |
| | yl]amino}cyclohexyl)-2-oxo-2-(2,4,6-trimethylphenyl)acetamide | | |
| 351 | (2R,5S)-N-(cis-4-{[6-(dimethylamino)-2-methylpyrimidin-4- | 540 (M + H) | 3 |
| | yl]amino}cyclohexyl)-5-phenyl-2- | | |
| | (phenylcarbonyl)cyclohexanecarboxamide | | |
| 352 | N-(cis-4-{[6-(dimethylamino)-2-methylpyrimidin-4- | 454 (M + H) | 3 |
| | yl]amino}cyclohexyl)-2-(9H-fluoren-9-ylidene)acetamide | | |
| 353 | 2-{[(cis-4-{[6-(dimethylamino)-2-methylpyrimidin-4- | 470 (M + H) | 3 |
| | yl]amino}cyclohexyl)amino]methyl}anthracene-9,10-dione | | |
| 354 | N,N,2-trimethyl-N′-{cis-4- | 340 (M + H) | 3 |
| | [(phenylmethyl)amino]cyclohexyl}pyrimidine-4,6-diamine | | |
| 355 | N′-{cis-4-[(biphenyl-4-ylmethyl)amino]cyclohexyl}-N,N,2- | 416 (M + H) | 3 |
| | trimethylpyrimidine-4,6-diamine | | |
| 356 | N′-[cis-4-({[4-(1,1-dimethylethyl)phenyl]methyl}amino)- | 396 (M + H) | 3 |
| | cyclohexyl]-N,N,2-trimethylpyrimidine-4,6-diamine | | |
| 357 | N′-{cis-4-[(1-benzothien-2-ylmethyl)amino]cyclohexyl}-N,N,2- | 396 (M + H) | 3 |
| | trimethylpyrimidine-4,6-diamine | | |
| 358 | N′-(cis-4-{[(4-bromophenyl)methyl]amino}cyclohexyl)-N,N,2- | 418 (M + H) | 3 |
| | trimethylpyrimidine-4,6-diamine | | |
| 359 | N,N,2-trimethyl-N′-[cis-4-({2- | 384 (M + H) | 3 |
| | [(phenylmethyl)oxy]ethyl}amino)cyclohexyl]pyrimidine- | | |
| | 4,6-diamine | | |
| 360 | N′-(cis-4-{[(4-chlorophenyl)methyl]amino}cyclohexyl)-N,N,2- | 374 (M + H) | 3 |
| | trimethylpyrimidine-4,6-diamine | | |
| 361 | N′-[cis-4-({2-[(4-chlorophenyl)oxy]ethyl}amino)cyclohexyl]- | 404 (M + H) | 3 |
| | N,N,2-trimethylpyrimidine-4,6-diamine | | |
| 362 | N′-{cis-4-[(cyclopropylmethyl)amino]cyclohexyl}-N,N,2- | 304 (M + H) | 3 |
| | trimethylpyrimidine-4,6-diamine | | |
| 363 | N′-{cis-4-[(cyclohexylmethyl)amino]cyclohexyl}-N,N,2- | 346 (M + H) | 3 |
| | trimethylpyrimidine-4,6-diamine | | |
| 364 | N′-(cis-4-{[2-(4-chlorophenyl)ethyl]amino}cyclohexyl)-N,N,2- | 388 (M + H) | 3 |
| | trimethylpyrimidine-4,6-diamine | | |
| 365 | N′-[cis-4-({[1-(4-chlorophenyl)cyclopentyl]methyl}- | 442 (M + H) | 3 |
| | amino)cyclohexyl]-N,N,2-trimethylpyrimidine-4,6-diamine | | |
| 366 | N′-[cis-4-({[3-(2-chloro-6-fluorophenyl)-5-methylisoxazol-4- | 473 (M + H) | 3 |
| | yl]methyl}amino)cyclohexyl]-N,N,2-trimethylpyrimidine- | | |
| | 4,6-diamine | | |
| 367 | N′-{cis-4-[({4-[(4-chlorophenyl)sulfonyl]-3-methyl-2- | 534 (M + H) | 3 |
| | thienyl}methyl)amino]cyclohexyl}-N,N,2-trimethylpyrimidine-4,6- | | |
| | diamine | | |
| 368 | N′-[cis-4-({[4-(dimethylamino)phenyl]methyl}amino)- | 383 (M + H) | 3 |
| | cyclohexyl]-N,N,2-trimethylpyrimidine-4,6-diamine | | |
| 369 | N′-(cis-4-{[(3,4-difluorophenyl)methyl]amino}cyclohexyl)-N,N,2- | 376 (M + H) | 3 |
| | trimethylpyrimidine-4,6-diamine | | |
| 370 | N′-[cis-4-({[3,4-bis(methyloxy)phenyl]methyl}amino)- | 400 (M + H) | 3 |
| | cyclohexyl]-N,N,2-trimethylpyrimidine-4,6-diamine | | |
| 371 | N′-[cis-4-({[4-(ethyloxy)phenyl]methyl}amino)cyclohexyl]-N,N,2- | 384 (M + H) | 3 |
| | trimethylpyrimidine-4,6-diamine | | |
| 372 | N′-(cis-4-{[(4-fluorophenyl)methyl]amino}cyclohexyl)-N,N,2- | 358 (M + H) | 3 |
| | trimethylpyrimidine-4,6-diamine | | |
| 373 | N′-{cis-4-[(furan-2-ylmethyl)amino]cyclohexyl}-N,N,2- | 330 (M + H) | 3 |
| | trimethylpyrimidine-4,6-diamine | | |
| 374 | N′-{cis-4-[(isoxazol-5-ylmethyl)amino]cyclohexyl}-N,N,2- | 331 (M + H) | 3 |
| | trimethylpyrimidine-4,6-diamine | | |
| 375 | N′-(cis-4-{[(2-iodophenyl)methyl]amino}cyclohexyl)-N,N,2- | 466 (M + H) | 3 |
| | trimethylpyrimidine-4,6-diamine | | |
| 376 | N,N,2-trimethyl-N′-(cis-4-{[(5-methyl-2-phenyl-2H-1,2,3-triazol-4 | 421 (M + H) | 3 |
| | yl)methyl]amino}cyclohexyl)pyrimidine-4,6-diamine | | |
| 377 | N,N,2-trimethyl-N′-(cis-4-{[(2-{[4-(methyloxy)phenyl]oxy}-5- | 507 (M + H) | 3 |
| | nitrophenyl)methyl]amino}cyclohexyl)pyrimidine-4,6-diamine | | |
| 378 | N,N,2-trimethyl-N′-{cis-4-[(naphthalen-2- | 390 (M + H) | 3 |
| | ylmethyl)amino]cyclohexyl}pyrimidine-4,6-diamine | | |
| 379 | N,N,2-trimethyl-N′-(cis-4-{[(3- | 385 (M + H) | 3 |
| | nitrophenyl)methyl]amino}cyclohexyl)pyrimidine-4,6-diamine | | |
| 380 | N,N,2-trimethyl-N′-[cis-4-({[1-(4-nitrophenyl)-5- | 519 (M + H) | 3 |
| | (trifluoromethyl)-1H-pyrazol-4-yl]methyl}amino)cyclohexyl]- | | |
| | pyrimidine-4,6-diamine | | |
| 381 | N,N,2-trimethyl-N′-(cis-4-{[2- | 370 (M + H) | 3 |
| | (phenyloxy)ethyl]amino}cyclohexyl)pyrimidine-4,6-diamine | | |
| 382 | N,N,2-trimethyl-N′-{cis-4-[(2- | 354 (M + H) | 3 |
| | phenylethyl)amino]cyclohexyl}pyrimidine-4,6-diamine | | |
| 383 | N,N,2-trimethyl-N′-[cis-4-({[(2R)-2- | 380 (M + H) | 3 |
| | phenylcyclopropyl]methyl}amino)cyclohexyl]pyrimidine- | | |
| | 4,6-diamine | | |
| 384 | N,N,2-trimethyl-N′-[cis-4-({[1-phenyl-5-(trifluoromethyl)-1H- | 474 (M + H) | 3 |
| | pyrazol-4-yl]methyl}amino)cyclohexyl]pyrimidine-4,6-diamine | | |
| 385 | N,N,2-trimethyl-N′-[cis-4-({2-[(2- | 415 (M + H) | 3 |
| | nitrophenyl)oxy]ethyl}amino)cyclohexyl]pyrimidine-4,6-diamine | | |
| 386 | N,N,2-trimethyl-N′-[cis-4-({[3- | 408 (M + H) | 3 |
| | (trifluoromethyl)phenyl]methyl}amino)cyclohexyl]pyrimidine- | | |
| | 4,6-diamine | | |
| 387 | N,N,2-trimethyl-N′-(cis-4-{[(4- | 354 (M + H) | 3 |
| | methylphenyl)methyl]amino}cyclohexyl)pyrimidine-4,6-diamine | | |
| 388 | N,N,2-trimethyl-N′-{cis-4-[(2- | 346 (M + H) | 3 |
| | thienylmethyl)amino]cyclohexyl}pyrimidine-4,6-diamine | | |
| 389 | N,N,2-trimethyl-N′-[cis-4-({2- | 460 (M + H) | 3 |
| | [(pentafluorophenyl)oxy]ethyl}amino)cyclohexyl]pyrimidine- | | |
| | 4,6-diamine | | |
| 390 | N′-[cis-4-({2-[3,4-bis(methyloxy)phenyl]ethyl}amino)- | 414 (M + H) | 3 |
| | cyclohexyl]-N,N,2-trimethylpyrimidine-4,6-diamine | | |
| 391 | N,N,2-trimethyl-N′-(cis-4-{[2- | 386 (M + H) | 3 |
| | (phenylthio)ethyl]amino}cyclohexyl)pyrimidine-4,6-diamine | | |
| 392 | 4-{[(cis-4-{[6-(dimethylamino)-2-methylpyrimidin-4- | 442 (M + H) | 3 |
| | yl]amino}cyclohexyl)amino]methyl}-9H-fluoren-9-one | | |
| 393 | N,N,2-trimethyl-N′-{cis-4-[({4- | 424 (M + H) | 3 |
| | [(trifluoromethyl)oxy]phenyl}methyl)amino]cyclohexyl}- | | |
| | pyrimidine-4,6-diamine | | |
| 394 | N′-[cis-4-({[4-fluoro-2- | 426 (M + H) | 3 |
| | (trifluoromethyl)phenyl]methyl}amino)cyclohexyl]-N,N,2- | | |
| | trimethylpyrimidine-4,6-diamine | | |
| 395 | N′-(cis-4-{[2-(4-fluorophenyl)ethyl]amino}cyclohexyl)-N,N,2- | 372 (M + H) | 3 |
| | trimethylpyrimidine-4,6-diamine | | |
| 396 | N′-[cis-4-({[4-(heptyloxy)phenyl]methyl}amino)cyclohexyl]- | 454 (M + H) | 3 |
| | N,N,2-trimethylpyrimidine-4,6-diamine | | |
| 397 | N,N,2-trimethyl-N′-(cis-4-{[(4- | 410 (M + H) | 3 |
| | pentylphenyl)methyl]amino}cyclohexyl)pyrimidine-4,6-diamine | | |
| 398 | N′-{cis-4-[(cyclopentylmethyl)amino]cyclohexyl}-N,N,2- | 332 (M + H) | 3 |
| | trimethylpyrimidine-4,6-diamine | | |
| 399 | N,N,2-trimethyl-N′-(cis-4-{[(4- | 466 (M + H) | 3 |
| | nonylphenyl)methyl]amino}cyclohexyl)pyrimidine-4,6-diamine | | |
| 400 | N′-{cis-4-[(2-{[4-(1,1- | 426 (M + H) | 3 |
| | dimethylethyl)phenyl]oxy}ethyl)amino]cyclohexyl}-N,N,2- | | |
| | trimethylpyrimidine-4,6-diamine | | |
| 401 | N′-(cis-4-{[(3-chloro-4-fluorophenyl)methyl]amino}cyclohexyl)- | 392 (M + H) | 3 |
| | N,N,2-trimethylpyrimidine-4,6-diamine | | |
| 402 | N′-{cis-4-[(2-cyclopentylethyl)amino]cyclohexyl}-N,N,2- | 346 (M + H) | 3 |
| | trimethylpyrimidine-4,6-diamine | | |
| 403 | N,N,2-trimethyl-N′-{cis-4-[(3- | 368 (M + H) | 3 |
| | phenylpropyl)amino]cyclohexyl}pyrimidine-4,6-diamine | | |
| 404 | N-[(1S)-2-[(cis-4-{[6-(dimethylamino)-2-methylpyrimidine-4- | 573 (M + H) | 3 |
| | yl]amino}cyclohexyl)amino]-1-(phenylmethyl)ethyl]naphthalene- | | |
| | 1-sulfonamide | | |
| 405 | N,N,2-trimethyl-N′-{cis-4-[({4- | 440 (M + H) | 3 |
| | [(trifluoromethyl)thio]phenyl}methyl)amino]cyclohexyl}- | | |
| | pyrimidine-4,6-diamine | | |
| 406 | N′-(cis-4-{[2-(2-bromophenyl)ethyl]amino}cyclohexyl)-N,N,2- | 432 (M + H) | 3 |
| | trimethylpyrimidine-4,6-diamine | | |
| 407 | N′-[cis-4-({[3-(1,1-dimethylethyl)-1-(phenylmethyl)-1H- | 476 (M + H) | 3 |
| | pyrazol-5-yl]methyl}amino)cyclohexyl]-N,N,2- | | |
| | trimethylpyrimidine-4,6-diamine | | |
| 408 | N′-[cis-4-({2-[(4-chlorophenyl)oxy]-2-methylpropyl}- | 432 (M + H) | 3 |
| | amino)cyclohexyl]-N,N,2-trimethylpyrimidine-4,6-diamine | | |
| 409 | N′-[cis-4-({[1-[(2,4-dichlorophenyl)methyl]-3-(1,1-dimethylethyl)- | 544 (M + H) | 3 |
| | 1H-pyrazol-5-yl]methyl}amino)cyclohexyl]-N,N,2- | | |
| | trimethylpyrimidine-4,6-diamine | | |
| 410 | N′-(cis-4-{[(5-chloro-1-methyl-1H-pyrazol-4- | 378 (M + H) | 3 |
| | yl)methyl]amino}cyclohexyl)-N,N,2- | | |
| | trimethylpyrimidine-4,6-diamine | | |
| 411 | methyl N-{[(cis-4-{[6-(dimethylamino)-2-methylpyrimidin-4- | 455 (M + H) | 3 |
| | yl]amino}cyclohexyl)amino]carbonyl}phenylalaninate | | |
| 412 | N-[(2-chlorophenyl)methyl]-N′-(cis-4-{[6-(dimethylamino)-2- | 417 (M + H) | 3 |
| | methylpyrimidin-4-yl]amino}cyclohexyl)urea | | |
| 413 | N-(cis-4-{[6-(dimethylamino)-2-methylpyrimidin-4- | 401 (M + H) | 3 |
| | yl]amino}cyclohexyl)-N′-[(4-fluorophenyl)methyl]urea | | |
| 414 | N-(cis-4-{[6-(dimethylamino)-2-methylpyrimidin-4- | 459 (M + H) | 3 |
| | yl]amino}cyclohexyl)-N′-(diphenylmethyl)urea | | |
| 415 | N-(cis-4-{[6-(dimethylamino)-2-methylpyrimidin-4- | 447 (M + H) | 1 |
| | yl]amino}cyclohexyl)-N′-[1-(1-naphthyl)ethyl]urea | | |
| 416 | N-(4-bromo-2,6-dimethylphenyl)-N′-(cis-4-{[6-(dimethylamino)-2- | 475 (M + H) | 1 |
| | methylpyrimidin-4-yl]amino}cyclohexyl)urea | | |
| 417 | N-(cis-4-{[6-(dimethylamino)-2-methylpyrimidin-4- | 411 (M + H) | 3 |
| | yl]amino}cyclohexyl)-N′-(2,4,6-trimethylphenyl)urea | | |
| 418 | N-(4-chloro-2-methylphenyl)-N′-(cis-4-{[6-(dimethylaminio)-2- | 417 (M + H) | 3 |
| | methylpyrimidin-4-yl]amino}cyclohexyl)urea | | |
| 419 | N-(cis-4-{[6-(dimethylamino)-2-methylpyrimidin-4- | 439 (M + H) | 3 |
| | yl]amino}cyclohexyl)-N′-[2-ethyl-6-(1-methylethyl)phenyl]urea | | |
| 420 | N-(4-bromo-2-methylphenyl)-N′-(cis-4-{[6-(dimethylamino)-2- | 461 (M + H) | 3 |
| | methylpyrimidin-4-yl]amino}cyclohexyl)urea | | |
| 421 | N-(cis-4-{[6-(dimethylamino)-2-methylpyrimidin-4- | 411 (M + H) | 3 |
| | yl]amino}cyclohexyl)-N′-(2-ethyl-6-methylphenyl)urea | | |
| 422 | N-(2-tert-butyl-6-methylphenyl)-N′-(cis-4-{[6-(dimethylamino)-2- | 439 (M + H) | 2 |
| | methylpyrimidin-4-yl]amino}cyclohexyl)urea | | |
| 423 | N-[2,6-dibromo-4-(1-methylethyl)phenyl]-N′-(cis-4-{[6- | 567 (M + H) | 3 |
| | (dimethylamino)-2-methylpyrimidin-4-yl]amino}cyclohexyl)urea | | |
| 424 | N-(cis-4-{[6-(dimethylamino)-2-methylpyrimidin-4- | 453 (M + H) | 3 |
| | yl]amino}cyclohexyl)-N′-{2-[(trifluoromethyl)oxy]phenyl}urea | | |
| 425 | N-(cis-4-{[6-(dimethylamino)-2-methylpyrimidin-4- | 459 (M + H) | 1 |
| | yl]amino}cyclohexyl)-N′-(3,4,5-trimethoxyphenyl)urea | | |
| 426 | N-(5-chloro-2,4-dimethoxyphenyl)-N′-(cis-4-{[6-(dimethylamino)- | 463 (M + H) | 2 |
| | 2-methylpyrimidin-4-yl]amino}cyclohexyl)urea | | |
| 427 | N-[3-(cyclopentyloxy)-4-(methyloxy)phenyl]-N′-(cis-4-{[6- | 483 (M + H) | 3 |
| | (dimethylamino)-2-methylpyrimidin-4-yl]amino}cyclohexyl)urea | | |
| 428 | N-(cis-4-{[6-(dimethylamino)-2-methylpyrimidin-4- | 413 (M + H) | 3 |
| | yl]amino}cyclohexyl)-N′-[2-(ethyloxy)phenyl]urea | | |
| 429 | N-(cis-4-{[6-(dimethylamino)-2-methylpyrimidin-4- | 603 (M + H) | 1 |
| | yl]amino}cyclohexyl)-N′-(2,4,6-tribromophenyl)urea | | |
| 430 | N-(cis-4-{[6-(dimethylamino)-2-methylpyrimidin-4- | 471 (M + H) | 3 |
| | yl]amino}cyclohexyl)-N′-(2,4,6-trichlorophenyl)urea | | |
| 431 | N-(2,4-dibromo-6-fluorophenyl)-N′-(cis-4-{[6-(dimethylamino)-2- | 543 (M + H) | 3 |
| | methylpyrimidin-4-yl]amino}cyclohexyl)urea | | |
| 432 | N-(cis-4-{[6-(dimethylamino)-2-methylpyrimidin-4- | 419 (M + H) | 3 |
| | yl]amino}cyclohexyl)-N′-naphthalen-1-ylurea | | |
| 433 | N-(cis-4-{[6-(dimethylamino)-2-methylpyrimidin-4- | 450 (M + H) | 3 |
| | yl]amino}cyclohexyl)-N′-(3-methyl-5-phenylisoxazol-4-yl)urea | | |
| 434 | N-(cis-4-{[6-(dimethylamino)-2-methylpyrimidin-4- | 489 (M + H) | 3 |
| | yl]amino}cyclohexyl)-N′-(2,2-diphenylethyl)thiourea | | |
| 435 | N-[4-bromo-2-(trifluoromethyl)phenyl]-N′-(cis-4-{[6- | 532 (M + H) | 3 |
| | (dimethylamino)-2-methylpyrimidin-4-yl]amino}- | | |
| | cyclohexyl)thiourea | | |
| 436 | N-(4-bromo-2,6-dimethylphenyl)-N′-(cis-4-{[6-(dimethylamino)-2- | 492 (M + H) | 2 |
| | methylpyrimidin-4-yl]amino}cyclohexyl)thiourea | | |
| 437 | N-(cis-4-{[6-(dimethylamino)-2-methylpyrimidin-4- | 427 (M + H) | 2 |
| | yl]amino}cyclohexyl)-N′-mesitylthiourea | | |
| 438 | N-(2,6-diethylphenyl)-N′-(cis-4-{[6-(dimethylamino)-2- | 441 (M + H) | 2 |
| | methylpyrimidin-4-yl]amino}cyclohexyl)thiourea | | |
| 439 | N-(2,4-dichloro-6-methylphenyl)-N′-(cis-4-{[6-(dimethylamino)-2- | 468 (M + H) | 2 |
| | methylpyrimidin-4-yl]amino}cyclohexyl)thiourea | | |
| 440 | N-(cis-4-{[6-(dimethylamino)-2-methylpyrimidin-4- | 478 (M + H) | 3 |
| | yl]amino}cyclohexyl)-N′-[4-(dimethylamino)-1-naphthyl]thiourea | | |
| 441 | N-{4-bromo-2-[(trifluoromethyl)oxy]phenyl}-N′- | 548 (M + H) | 3 |
| | (cis-4-{[6-(dimethylamino)-2-methylpyrimidin-4- | | |
| | yl]amino}-cyclohexyl)thiourea | | |
| 442 | N-(cis-4-{[6-(dimethylamino)-2-methylpyrimidin-4- | 475 (M + H) | 1 |
| | yl]amino}cyclohexyl)-N′-(3,4,5-trimethoxyphenyl)thiourea | | |
| 443 | N-(5-chloro-2,4-dimethoxyphenyl)-N′-(cis-4-{[6- | 480 (M + H) | 2 |
| | (dimethylamino)-2-methylpyrimidin-4-yl]amino}- | | |
| | cyclohexyl)thiourea | | |
| 444 | N-[2,4-bis(methyloxy)phenyl]-N′-(cis-4-{[6-(dimethylamino)-2- | 445 (M + H) | 3 |
| | methylpyrimidin-4-yl]amino}cyclohexyl)thiourea | | |
| 445 | N-[3,4-bis(methyloxy)phenyl]-N′-(cis-4-{[6-(dimethylamino)-2- | 445 (M + H) | 3 |
| | methylpyrimidin-4-yl]amino}cyclohexyl)thiourea | | |
| 446 | N-(cis-4-{[6-(dimethylamino)-2-methylpyrimidin-4- | 429 (M + H) | 3 |
| | yl]amino}cyclohexyl)-N′-[2-(ethyloxy)phenyl]thiourea | | |
| 447 | N-(cis-4-{[6-(dimethylamino)-2-methylpyrimidin-4- | 621 (M + H) | 1 |
| | yl]amino}cyclohexyl)-N′-(2,4,6-tribromophenyl)thiourea | | |
| 448 | N-(2,4-dibromo-6-fluorophenyl)-N′-(cis-4-{[6-(dimethylamino)-2- | 559 (M + H) | 3 |
| | methylpyrimidin-4-yl]amino}cyclohexyl)thiourea | | |
| 449 | N-(cis-4-{[6-(dimethylamino)-2-methylpyrimidin-4- | 511 (M + H) | 3 |
| | yl]amino}cyclohexyl)-N′-(4-iodophenyl)thiourea | | |
| 450 | N-(4-cyanophenyl)-N′-(cis-4-{[6-(dimethylamino)-2- | 410 (M + H) | 3 |
| | methylpyrimidin-4-yl]amino}cyclohexyl)thiourea | | |
| 451 | methyl 3-({[(cis-4-{[6-(dimethylamino)-2-methylpyrimidin-4- | 463 (M + H) | 3 |
| | yl]amino}cyclohexyl)amino]carbonothioyl}amino)-4- | | |
| | methylthiophene-2-carboxylate | | |
| 452 | 2,2-dimethylpropyl (cis-4-{[6-(dimethylamino)-2-methylpyrimidin- | 364 (M + H) | 3 |
| | 4-yl]amino}cyclohexyl)carbamate | | |
| 453 | [4,5-bis(methyloxy)-2-nitrophenyl]methyl (cis-4-{[6- | 489 (M + H) | 3 |
| | (dimethylamino)-2-methylpyrimidin-4-yl]amino}- | | |
| | cyclohexyl)carbamate | | |
| 454 | 3-(trifluoromethyl)phenyl (cis-4-{[6-(dimethylamino)-2- | 438 (M + H) | 3 |
| | methylpyrimidin-4-yl]amino}cyclohexyl)carbamate | | |
| 455 | 4-bromophenyl (cis-4-{[6-(dimethylamino)-2-methylpyrimidin-4- | 448 (M + H) | 3 |
| | yl]amino}cyclohexyl)carbamate | | |
| 456 | 2-(methyloxy)phenyl (cis-4-{[6-(dimethylamino)-2- | 400 (M + H) | 3 |
| | methylpyrimidin-4-yl]amino}cyclohexyl)carbamate | | |
| 457 | 2-(methyloxy)ethyl (cis-4-{[6-(dimethylamino)-2-methylpyrimidin- | 352 (M + H) | 3 |
| | 4-yl]amino}cyclohexyl)carbamate | | |
| 458 | octyl (cis-4-{[6-(dimethylamino)-2-methylpyrimidin-4- | 406 (M + H) | 3 |
| | yl]amino}cyclohexyl)carbamate | | |
| 459 | ethyl (cis-4-{[6-(dimethylamino)-2-methylpyrimidin-4- | 322 (M + H) | 3 |
| | yl]amino}cyclohexyl)carbamate | | |
| 460 | (4-nitrophenyl)methyl (cis-4-{[6-(dimethylamino)-2- | 429 (M + H) | 3 |
| | methylpyrimidin-4-yl]amino}cyclohexyl)carbamate | | |
| 461 | naphthalen-2-yl (cis-4-{[6-(dimethylamino)-2-methylpyrimidin-4- | 420 (M + H) | 3 |
| | yl]amino}cyclohexyl)carbamate | | |
| 462 | prop-2-en-1-yl (cis-4-{[6-(dimethylamino)-2-methylpyrimidin-4- | 334 (M + H) | 3 |
| | yl]amino}cyclohexyl)carbamate | | |
| 463 | phenylmethyl (cis-4-{[6-(dimethylamino)-2-methylpyrimidin-4- | 384 (M + H) | 3 |
| | yl]amino}cyclohexyl)carbamate | | |
| 464 | phenyl (cis-4-{[6-(dimethylamino)-2-methylpyrimidin-4- | 370 (M + H) | 3 |
| | yl]amino}cyclohexyl)carbamate | | |
| 465 | (2S,5R)-5-methyl-2-(1-methylethyl)cyclohexyl (cis-4-{[6- | 432 (M + H) | 3 |
| | (dimethylamino)-2-methylpyrimidin-4-yl]amino}- | | |
| | cyclohexyl)carbamate | | |
| 466 | 4-methylphenyl (cis-4-{[6-(dimethylamino)-2-methylpyrimidin-4- | 384 (M + H) | 3 |
| | yl]amino}cyclohexyl)carbamate | | |
| 467 | methyl (cis-4-{[6-(dimethylamino)-2-methylpyrimidin-4- | 308 (M + H) | 3 |
| | yl]amino}cyclohexyl)carbamate | | |
| 468 | (2-chlorophenyl)methyl (cis-4-{[6-(dimethylamino)-2- | 418 (M + H) | 3 |
| | methylpyrimidin-4-yl]amino}cyclohexyl)carbamate | | |
| 469 | 9H-fluoren-9-ylmethyl (cis-4-{[6-(dimethylamino)-2- | 472 (M + H) | 3 |
| | methylpyrimidin-4-yl]amino}cyclohexyl)carbamate | | |
| 470 | 2,2,2-trichloroethyl (cis-4-{[6-(dimethylamino)-2-methylpyrimidin | 424 (M + H) | 3 |
| | 4-yl]amino}cyclohexyl)carbamate | | |
| 471 | (E)-N-(cis-4-{[6-(dimethylamino)-2-methylpyrimidin-4- | 416 (M + H) | 3 |
| | yl]amino}cyclohexyl)-2-phenylethenesulfonamide | | |
| 472 | N-(cis-4-{[6-(dimethylamino)-2-methylpyrimidin-4- | 472 (M + H) | 3 |
| | yl]amino}cyclohexyl)-1-[3-(trifluoromethyl)phenyl]- | | |
| | methanesulfonamide | | |
| 473 | 1-(3,4-dichlorophenyl)-N-(cis-4-{[6-(dimethylamino)-2- | 472 (M + H) | 3 |
| | methylpyrimidin-4-yl]amino}cyclohexyl)methanesulfonamide | | |
| 474 | N-(cis-4-{[6-(dimethylamino)-2-methylpyrimidim-4- | 422 (M + H) | 3 |
| | yl]amino}cyclohexyl)-1-(4-fluorophenyl)methanesulfonamide | | |
| 475 | N-(cis-4-{[6-(dimethylamino)-2-methylpyrimidin-4- | 449 (M + H) | 3 |
| | yl]amino}cyclohexyl)-1-(2-nitrophenyl)methanesulfonamide | | |
| 476 | N-(cis-4-{[6-(dimethylamino)-2-methylpyrimidin-4- | 404 (M + H) | 3 |
| | yl]amino}cyclohexyl)-1-phenylmethanesulfonamide | | |
| 477 | N-(cis-4-{[6-(dimethylamino)-2-methylpyrimidin-4- | 468 (M + H) | 3 |
| | yl]amino}cyclohexyl)-2-naphthalen-1-ylethanesulfonamide | | |
| 478 | N-(cis-4-{[6-(dimethylamino)-2-methylpyrimidin-4- | 328 (M + H) | 3 |
| | yl]amino}cyclohexyl)methanesulfonamide | | |
| 479 | N-(cis-4-{[6-(dimethylamino)-2-methylpyrimidin-4- | 356 (M + H) | 3 |
| | yl]amino}cyclohexyl)propane-2-sulfonamide | | |
| 480 | N-(cis-4-{[6-(dimethylamino)-2-methylpyrimidin-4- | 426 (M + H) | 3 |
| | yl]amino}cyclohexyl)octane-1-sulfonamide | | |
| 481 | methyl 2-{[(cis-4-{[6-(dimethylamino)-2-methylpyrimidin-4- | 448 (M + H) | 3 |
| | yl]amino}cyclohexyl)amino]sulfonyl}benzoate | | |
| 482 | N-(cis-4-{[6-(dimethylamino)-2-methylpyrimidin-4- | 416 (M + H) | 3 |
| | yl]amino}cyclohexyl)-4-ethenylbenzenesulfonamide | | |
| 483 | N-(cis-4-{[6-(dimethylamino)-2-methylpyrimidin-4- | 458 (M + H) | 3 |
| | yl]amino}cyclohexyl)-3-(trifluoromethyl)benzenesulfonamide | | |
| 484 | 4-acetyl-N-(cis-4-{[6-(dimethylamino)-2-methylpyrimidin-4- | 432 (M + H) | 3 |
| | yl]amino}cyclohexyl)benzenesulfonamide | | |
| 485 | 3-chloro-N-(cis-4-{[6-(dimethylamino)-2-methylpyrimidin-4- | 438 (M + H) | 3 |
| | yl]amino}cyclohexyl)-4-methylbenzenesulfonamide | | |
| 486 | N-(cis-4-{[6-(dimethylamino)-2-methylpyrimidin-4- | 432 (M + H) | 3 |
| | yl]amino}cyclohexyl)-2,4,6-trimethylbenzenesulfonamide | | |
| 487 | N-(cis-4-{[6-(dimethylamino)-2-methylpyrimidin-4- | 432 (M + H) | 3 |
| | yl]amino}cyclohexyl)-4-propylbenzenesulfonamide | | |
| 488 | N-(cis-4-{[6-(dimethylamino)-2-methylpyrimidin-4- | 460 (M + H) | 3 |
| | yl]amino}cyclohexyl)-4-(1,1-dimethylpropyl)benzenesulfonamide | | |
| 489 | N-(cis-4-{[6-(dimethylamino)-2-methylpyrimidin-4- | 466 (M + H) | 3 |
| | yl]amino}cyclohexyl)biphenyl-4-sulfonamide | | |
| 490 | 5-(dimethylamino)-N-(cis-4-{[6-(dimethylamino)-2- | 483 (M + H) | 3 |
| | methylpyrimidin-4-yl]amino}cyclohexyl)naphthalene- | | |
| | 1-sulfonamide | | |
| 491 | N-(cis-4-{[6-(dimethylamino)-2-methylpyrimidin-4- | 474 (M + H) | 3 |
| | yl]amino}cyclohexyl)-2-[(trifluoromethyl)oxy]- | | |
| | benzenesulfonamide | | |
| 492 | N-(cis-4-{[6-(dimethylamino)-2-methylpyrimidin-4- | 474 (M + H) | 3 |
| | yl]amino}cyclohexyl)-3-[(trifluoromethyl)oxy]- | | |
| | benzenesulfonamide | | |
| 493 | N-(cis-4-{[6-(dimethylamino)-2-methylpyrimidin-4- | 420 (M + H) | 3 |
| | yl]amino}cyclohexyl)-3-(methyloxy)benzenesulfonamide | | |
| 494 | 4-(butyloxy)-N-(cis-4-{[6-(dimethylamino)-2-methylpyrimidin-4- | 462 (M + H) | 3 |
| | yl]amino}cyclohexyl)benzenesulfonamide | | |
| 495 | 3,5-dichloro-4-[(2-chloro-4-nitrophenyl)oxy]-N-(cis-4-{[6- | 629 (M + H) | 3 |
| | (dimethylamino)-2-methylpyrimidin-4- | | |
| | yl]amino}cyclohexyl)benzenesulfonamide | | |
| 496 | N-(cis-4-{[6-(dimethylamino)-2-methylpyrimidin-4- | 482 (M + H) | 3 |
| | yl]amino}cyclohexyl)-4-(phenyloxy)benzenesulfonamide | | |
| 497 | 4-{[3-chloro-5-(trifluoromethyl)pyridin-2-yl]oxy}-N-(cis-4-{[6- | 585 (M + H) | 3 |
| | (dimethylamino)-2-methylpyrimidin-4- | | |
| | yl]amino}cyclohexyl)benzenesulfonamide | | |
| 498 | N-(cis-4-{[6-(dimethylamino)-2-methylpyrimidin-4- | 468 (M + H) | 3 |
| | yl]amino}cyclohexyl)-4-(methylsulfonyl)benzenesulfonamide | | |
| 499 | 3-cyano-N-(cis-4-{[6-(dimethylamino)-2-methylpyrimidin-4- | 415 (M + H) | 3 |
| | yl]amino}cyclohexyl)benzenesulfonamide | | |
| 500 | 3-bromo-N-(cis-4-{[6-(dimethylamino)-2-methylpyrimidin-4- | 468 (M + H) | 3 |
| | yl]amino}cyclohexyl)benzenesulfonamide | | |
| 501 | 4-bromo-N-(cis-4-{[6-(dimethylamino)-2-methylpyrimidin-4- | 552 (M + H) | 3 |
| | yl]amino}cyclohexyl)-2-[(trifluoromethyl)oxy]- | | |
| | benzenesulfonamide | | |
| 502 | 3,4-dichloro-N-(cis-4-{[6-(dimethylamino)-2-methylpyrimidin-4- | 458 (M + H) | 3 |
| | yl]amino}cyclohexyl)benzenesulfonamide | | |
| 503 | N-(cis-4-{[6-(dimethylamino)-2-methylpyrimidin-4- | 408 (M + H) | 3 |
| | yl]amino}cyclohexyl)-3-fluorobenzenesulfonamide | | |
| 504 | N-(cis-4-{[6-(dimethylamino)-2-methylpyrimidin-4- | 435 (M + H) | 3 |
| | yl]amino}cyclohexyl)-3-nitrobenzenesulfonamide | | |
| 505 | N-(cis-4-{[6-(dimethylamino)-2-methylpyrimidin-4- | 440 (M + H) | 3 |
| | yl]amino}cyclohexyl)naphthalene-1-sulfonamide | | |
| 506 | ethyl-4-{[(cis-4-{[6-(dimethylamino)-2-methylpyrimidin-4- | 617 (M + H) | 3 |
| | yl]amino}cyclohexyl)amino]sulfonyl}-2-methyl-1,5-diphenyl-1H- | | |
| | pyrrole-3-carboxylate | | |
| 507 | methyl 5-{[(cis-4-{[6-(dimethylamino)-2-methylpyrimidin-4- | 451 (M + H) | 3 |
| | yl]amino}cyclohexyl)amino]sulfonyl}-1-methyl-1H-pyrrole-2- | | |
| | carboxylate | | |
| 508 | methyl 5-{[(cis-4-{[6-(dimethylamino)-2-methylpyrimidin-4- | 452 (M + H) | 3 |
| | yl]amino}cyclohexyl)amino]sulfonyl}-2-methylfuran- | | |
| | 3-carboxylate | | |
| 509 | N-(cis-4-{[6-(dimethylamino)-2-methylpyrimidin-4- | 541 (M + H) | 3 |
| | yl]amino}cyclohexyl)-2-(trifluoroacetyl)-1,2,3,4- | | |
| | tetrahydroisoquinoline-7-sulfonamide | | |
| 510 | 5-{[3-chloro-5-(trifluoromethyl)pyridin-2-yl]methyl}-N-(cis-4-{[6- | 589 (M + H) | 3 |
| | (dimethylamino)-2-methylpyrimidin-4- | | |
| | yl]amino}cyclohexyl)thiophene-2-sulfonamide | | |
| 511 | 5-chloro-N-(cis-4-{[6-(dimethylamino)-2-methylpyrimidin-4- | 494 (M + H) | 3 |
| | yl]amino}cyclohexyl)-3-methyl-1-benzothiophene-2-sulfonamide | | |
| 512 | N-(cis-4-{[6-(dimethylamino)-2-methylpyrimidin-4- | 409 (M + H) | 3 |
| | yl]amino}cyclohexyl)-3,5-dimethylisoxazole-4-sulfonamide | | |
| 513 | N-(cis-4-{[6-(dimethylamino)-2-methylpyrimidin-4- | 422 (M + H) | 3 |
| | yl]amino}cyclohexyl)-1,3,5-trimethyl-1H-pyrazole-4-sulfonamide | | |
| 514 | ethyl 5-(4-chlorophenyl)-4-{[(cis-4-{[6-(dimethylamino)-2- | 651 (M + H) | 3 |
| | methylpyrimidin-4-yl]amino}cyclohexyl)amino]sulfonyl}-2- | | |
| | methyl-1-phenyl-1H-pyrrole-3-carboxylate | | |
| 515 | N-(cis-4-{[6-(dimethylamino)-2-methylpyrimidin-4- | 544 (M + H) | 3 |
| | yl]amino}cyclohexyl)-5-[1-methyl-3-(trifluoromethyl)-1H-pyrazole- | | |
| | 5-yl]thiophene-2-sulfonamide | | |
| 516 | 1-[3-chloro-5-(trifluoromethyl)pyridin-2-yl]-N-(cis-4-{[6- | 558 (M + H) | 3 |
| | (dimethylamino)-2-methylpyrimidin-4-yl]amino}cyclohexyl)-1H- | | |
| | pyrrole-2-sulfonamide | | |
| 517 | N-(cis-4-{[6-(dimethylamino)-2-methylpyrimidin-4- | 463 (M + H) | 3 |
| | yl]amino}cyclohexyl)-5-isoxazol-3-ylthiophene-2-sulfonamide | | |
| 518 | methyl 5-{[(cis-4-{[6-(dimethylamino)-2-methylpyrimidin-4- | 484 (M + H) | 3 |
| | yl]amino}cyclohexyl)amino]sulfonyl}-4-(methyloxy)thiophene-3- | | |
| | carboxylate | | |
| 519 | N-(cis-4-{[6-(dimethylamino)-2-methylpyrimidin-4- | 536 (M + H) | 3 |
| | yl]amino}cyclohexyl)-4-(phenylsulfonyl)thiophene- | | |
| | 2-sulfonamide | | |
| 520 | 5-bromo-N-(cis-4-{[6-(dimethylamino)-2-methylpyrimidin-4- | 474 (M + H) | 3 |
| | yl]amino}cyclohexyl)thiophene-2-sulfonamide | | |
| 521 | 7-chloro-N-(cis-4-{[6-(dimethylamino)-2-methylpyrimidin-4- | 466 (M + H) | 3 |
| | yl]amino}cyclohexyl)-2,1,3-benzoxadiazole-4-sulfonamide | | |
| 522 | N-(cis-4-{[6-(dimethylamino)-2-methylpyrimidin-4- | 441 (M + H) | 3 |
| | yl]amino}cyclohexyl)quinoline-8-sulfonamide | | |

### Assay Procedures

Assay for Determination of Constitutive Activity of Non-Endogenous GPCRs

### Example 523

### Intracellular IP₃ Accumulation Assay

On day 1, cells to be transfected can be plated onto 24 well plates, usually 1×10⁵ cells/well (although his umber can be optimized. On day 2 cells can be transfected by firstly mixing 0.25 μg DNA (e.g., pCMV vector or pCMV vector comprising polynucleotide encoding receptor) in 50 μl serum free DMEM/well and 2 μl lipofectamine in 50 μl serum-free DMEM/well. The solutions are gently mixed and incubated for 15-30 min at room temperature. Cells are washed with 0.5 mL PBS and 400 μl of serum free media is mixed with the transfection media and added to the cells. The cells are then incubated for 3-4 hrs at 37° C./5% CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 mL inositol-free/serum free media (GIBCO BRL) is added/well with 0.25 μCi of ³H-myo-inositol/well and the cells are incubated for 16-18 hrs o/n at 37° C./5% CO₂ On Day 4 the cells are washed with 0.5 ml PBS and 0.45 ml of assay medium is added containing inositol-free/serum free media 10 μM pargyline 10 mM lithium chloride or 0.4 mL of assay medium and 50 μl of 10× ketanserin (ket) to final concentration of 10 μM. The cells are then incubated for 30 min at 37° C. The cells are then washed with 0.5 mL PBS and 200 μl of fresh/ice cold stop solution (1M KOH; 18 mM Na-borate; 3.8 mM EDTA) is added/well. The solution is kept on ice for 5-10 min or until cells were lysed and then neutralized by 200 μl of fresh/ice cold neutralization sol. (7.5% HCL). The lysate is then transferred into 1.5 mL eppendorf tubes and 1 mL of chloroform/methanol (1:2) is added/tube. The solution is vortexed for 15 sec and the upper phase is applied to a Biorad AG1-X8™ anion exchange resin (100-200 mesh). Firstly, the resin is washed with water at 1:1.25 W/V and 0.9 mL of upper phase is loaded onto the column. The column is washed with 10 mls of 5 mM myo-inositol and 10 mL of 5 mM Na-borate/60 mM Na-formate. The inositol tris phosphates are eluted into scintillation vials containing 10 mL of scintillation cocktail with 2 mL of 0.1 M formic acid/1 M ammonium formate. The columns are regenerated by washing with 10 ml of 0.1 M formic acid/3M ammonium formate and rinsed twice with H₂O and stored at 4° C. in water.

### Example 524

### High Throughput Functional Screening

### FLIPR™

Subsequently, a functional based assay was used to confirm the lead hits, referred to as FLIPR™ (the Fluorometric Imaging Plate Reader) and FDSS6000™ (Functional Drug Screening System). This assay utilized a non-endogenous, constitutively active version of the MCH receptor.

The FLIPR and FDSS assays are able to detect intracellular Ca²⁺ concentration in cells, which can be utilized to assess receptor activation and determine whether a candidate compound is an, for example, antagonist, inverse agonist or agonist to a Gq-coupled receptor. The concentration of free Ca²⁺ in the cytosol of any cell is extremely low, whereas its concentration in the extracellular fluid and endoplasmic reticulum (ER) is very high. Thus, there is a large gradient tending to drive Ca²⁺ into the cytosol across both the plasma membrane and ER. The FLIPR™ and FDSS6000™ systems (Molecular Devices Corporation, HAMAMATSU Photonics K.K.) are designed to perform functional cell-based assays, such as the measurement of intracellular calcium for high-throughput screening. The measurement of fluorescent is associated with calcium release upon activation of the Gq-coupled receptors. Gi or Go coupled receptors are not as easily monitored through the FLIPR™ and FDSS6000™ systems because these G proteins do not couple with calcium signal pathways.

Fluorometric Imaging Plate Reader system was used to allow for rapid, kinetic measurements of intracellular fluorescence in 96 well microplates (or 384 well microplates). Simultaneous measurements of fluorescence in all wells can be made by FLIPR or FDSS6000™ every second with high sensitivity and precision. These systems are ideal for measuring cell-based functional assays such as monitoring the intracellular calcium fluxes that occur within seconds after activation of the Gq coupled receptor.

Briefly, the cells are seeded into 96 well at 5.5×10⁴ cells/well with complete culture media (Dulbecco's Modified Eagle Medium with 10% fetal bovine serum, 2 mM L-glutamine, 1 mM sodium pyruvate and 0.5 mg/mL G418, pH 7.4) for the assay next day. On the day of assay, the media is removed and the cells are incubated with 100 μl of loading buffer (4 μM Fluo-4-AM in complete culture media containing 2.5 mM Probenicid, 0.5 mg/ml and 0.2% bovine serum albumin) in 5% CO₂ incubator at 37° C. for 1 hr. The loading buffer is removed, and the cells are washed with wash buffer (Hank's Balanced Salt Solution containing 2.5 mM Probenicid, 20 mM HEPES, 0.5 mg/mL and 0.2% bovine serum albumin, pH 7.4). One hundred fifty μl of wash buffer containing various concentrations of test compound is added to the cells, and the cells are incubated in 5% CO₂ incubator at 37° C. for 30 min. Fifty μl of wash buffer containing various concentration of MCH are added to each well, and transient changes in [Ca²⁺]i evoked by MCH are monitored using the FLIPR or FDSS in 96 well plates at Ex. 488 nm and Em. 530 nm for 290 second. When antagonist activity of compound is tested, 50 nM of MCH is used.

Use of FLIPR™ and FDSS6000™ can be accomplished by following manufacturer's instruction (Molecular Device Corporation and HAMAMATSU Photonics K.K.).

Representative examples are shown below.

| Compound No. | IC₅₀ (nM) |
|---|---|
| Example 7 | 101 |
| Example 24 | 26 |

The results were shown on the tables in the Examples section and the table in the next page in accordance with the classification as defined below.

| Ex. No. | class |
|---|---|
| 1 | 3 |
| 2 | 2 |
| 3 | 3 |
| 4 | 3 |
| 5 | 1 |
| 6 | 1 |
| 7 | 2 |
| 8 | 1 |
| 9 | 2 |
| 10 | 1 |
| 11 | 1 |
| 12 | 2 |
| 13 | 2 |
| 14 | 1 |
| 15 | 3 |
| 16 | 2 |
| 17 | 1 |
| 18 | 1 |
| 19 | 3 |
| 20 | 3 |
| 21 | 1 |
| 22 | 3 |
| 23 | 2 |
| 24 | 1 |
| 25 | 2 |
| 26 | 2 |
| 27 | 2 |
| 28 | 3 |
| 29 | 3 |
| 30 | 3 |
| 31 | 2 |
| 32 | 1 |
| 33 | 2 |
| 34 | 3 |
| 35 | 3 |
| 36 | 3 |
| 37 | 3 |
| 38 | 3 |
| 39 | 2 |
| 40 | 1 |
| 41 | 3 |
| 42 | 3 |
| 43 | 2 |
| 44 | 1 |
| 45 | 2 |
| 46 | 3 |
| 47 | 3 |
| 168 | 3 |
| 169 | 2 |
| 170 | 1 |
| 171 | 3 |
| 172 | 3 |
| 173 | 3 |
| 174 | 3 |
| 175 | 3 |
| 176 | 3 |
| 177 | 1 |
| 178 | 3 |
| 179 | 1 |
| 180 | 3 |
| 181 | 3 |
| 182 | 3 |
| 183 | 3 |
| 184 | 3 |
| 185 | 3 |
| 186 | 3 |
| 187 | 2 |
| 188 | 2 |
| 189 | 3 |
| 190 | 3 |
| 191 | 1 |
| 192 | 2 |
| 193 | 1 |
| 194 | 3 |
| 195 | 3 |
| 196 | 3 |

Class 1: The value of percent of control at 10⁻⁷ M was less than 40% or the value of IC₅₀ was less than 50 nM.
Class 2: The value of percent of control at 10⁻⁷ M was from 40% to 60% or the value of IC₅₀ was from 50 nM to 200 nM.
Class 3: The value of percent of control at 10⁻⁷ M was more than 60% or the value of IC₅₀ was more than 200 nM.

### Example 525

### Receptor Binding Assay

In addition to the methods described herein, another means for evaluating a test compound is by determining binding affinities to the MCH receptor. This type of assay generally requires a radiolabelled ligand to the MCH receptor. Absent the use of known ligands for the MCH receptor and radiolabels thereof, compounds of Formula (I) can be labelled with a radioisotope and used in an assay for evaluating the affinity of a test compound to the MCH receptor.

A radiolabelled MCH compound of Formula (I) can be used in a screening assay to identify/evaluate compounds. In general terms, a newly synthesized or identified compound (i.e., test compound) can be evaluated for its ability to reduce binding of the “radiolabelled compound of Formula (I)” to the MCH receptor. Accordingly, the ability to compete with the “radio-labelled compound of Formula (I)” or Radiolabelled MCH Ligand for the binding to the MCH receptor directly correlates to its binding affinity of the test compound to the MCH receptor.

### Assay Protocol for Determining Receptor Binding for MCH:

A. MCH Receptor Preparation

293 cells (human kidney, ATCC), transiently transfected with 10 μg human MCH receptor and 60 μl Lipofectamine (per 15-cm dish), are grown in the dish for 24 hours (75% confluency) with a media change and removed with 10 mL/dish of Hepes-EDTA buffer (20 mM Hepes+10 mM EDTA, pH 7.4). The cells are then centrifuged in a Beckman Coulter centrifuge for 20 minutes, 17,000 rpm (JA-25.50 rotor). Subsequently, the pellet is resuspended in 20 mM Hepes+1 mM EDTA, pH 7.4 and homogenized with a 50-mL Dounce homogenizer and again centrifuged. After removing the supernatant, the pellets can be stored at −80° C., until used in binding assay. When used in the assay, membranes are thawed on ice for 20 minutes and then 10 mL of incubation buffer (20 mM Hepes, 1 mM MgCl₂, 100 mM NaCl, pH 7.4) added. The membranes are then vortexed to resuspend the crude membrane pellet and homogenized with a Brinkmann PT-3100 Polytron homogenizer for 15 seconds at setting 6. The concentration of membrane protein is determined using the BRL Bradford protein assay.

B. Binding Assay

For total binding, a total volume of 50 ul of appropriately diluted membranes (diluted in assay buffer containing 50 mM Tris HCl (pH 7.4), 1 mM MgCl₂, and 1 mM EDTA; 5-50 ug protein) is added to 96-well polyproylene microtiter plates followed by addition of 100 μl of assay buffer and 50 μl of Radiolabelled MCH Ligand. For nonspecific binding, 50 μl of assay buffer is added instead of 100 μl and an additional 50 μl of 10 uM cold MCH is added before 50 μl of Radiolabelled MCH Ligand is added. Plates are then incubated at room temperature for 60-120 minutes. The binding reaction is terminated by filtering assay plates through a Microplate Devices GF/C Unifilter filtration plate with a Brandell 96-well plate harvester followed by washing with cold 50 mM Tris HCl, pH 7.4 containing 0.9% NaCl. Then, the bottom of the filtration plate are sealed, 50 ul of Optiphase Supermix is added to each well, the top of the plates are sealed, and plates are counted in a Trilux MicroBeta scintillation counter. For compound competition studies, instead of adding 100 μl of assay buffer, 100 μl of appropriately diluted test compound is added to appropriate wells followed by addition of 50 μl of Radiolabelled MCH Ligand.

C. Calculations

The test compounds are initially assayed at 1 and 0.1 μM and then at a range of concentrations chosen such that the middle dose would cause about 50% inhibition of a Radiolabelled MCH Ligand binding (i.e., IC₅₀). Specific binding in the absence of test compound (B_{O}) is the difference of total binding (B_{T}) minus non-specific binding (NSB) and similarly specific binding (in the presence of test compound) (B) is the difference of displacement binding (B_{D}) minus non-specific binding (NSB). IC₅₀ is determined from an inhibition response curve, logit-log plot of % B/B_{O} vs. concentration of test compound.

Kᵢ is calculated by the Cheng and Prustoff transformation:

Kᵢ=IC₅₀/(1+[L]/K_{D})

wherein [L] is the concentration of a Radiolabelled MCH1 Ligand used in the assay and K_{D} is the dissociation constant of a Radiolabelled MCH Ligand determined independently under the same binding conditions.

It is intended that each of the patents, applications, printed publications, and other published documents mentioned or referred to in this specification be herein incorporated by reference in their entirety.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

1. A compound of Formula (I): 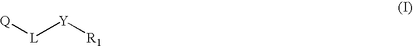
wherein Q is: 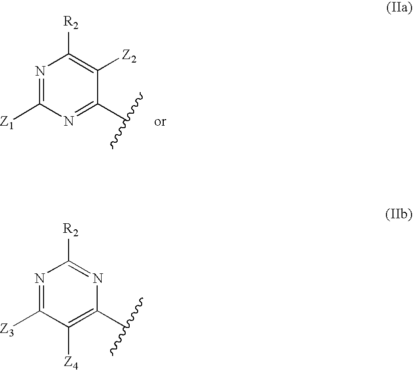
R₁ is selected from the group consisting of:
(i) C₁₋₁₆ alkyl, and
C₁₋₁₆ alkyl substituted by substituent(s) independently selected from the group consisting of:
halogen,
hydroxy,
oxo,
C₁₋₅ alkoxy,
C₁₋₅ alkoxy substituted by substituent(s) independently selected from the group consisting of:
carbocyclic aryl,
heterocyclyl, and
heterocyclyl substituted by C₁₋₅ alkyl,
C₁₋₅ alkylcarbonyloxy,
carbocyclyloxy,
carbocyclic aryloxy,
carbocyclic aryloxy substituted by substituent(s) independently selected from the group consisting of:
halogen,
hydroxy,
carboxy,
carbamoyl,
nitro,
cyano,
amino,
carbocyclic aryl,
carbocyclic aryl substituted by C₁₋₅ alkoxy,
C₁₋₅ alkoxy,
C₁₋₅ alkoxy substituted by halogen,
C₁₋₅ alkyl, and
C₁₋₅ alkyl substituted by substituent(s) independently selected from the group consisting of:
halogen,
hydroxy,
carboxy,
oxo,
mono-C₁₋₅ alkylamino,
di-C₁₋₅ alkylamino,
mono-C₁₋₅ alkylamino substituted by carbocyclic aryl,
di-C₁₋₅ alkylamino substituted by carbocyclic aryl,
mono-C₁₋₅ alkylamino substituted by halogenated carbocyclic aryl,
di-C₁₋₅ alkylamino substituted by halogenated carbocyclic aryl,
carbocyclic arylcarbonylamino, and
carbocyclic arylcarbonylamino substituted by halogen,
heterocyclyloxy,
heterocyclyloxy substituted by substituent(s) independently selected from the group consisting of:
halogen,
hydroxy,
carboxy,
carbamoyl,
nitro,
cyano,
amino,
carbocyclic aryl,
carbocyclic aryl substituted by C₁₋₅ alkoxy,
C₁₋₅ alkoxy,
C₁₋₅ alkoxy substituted by substituent(s) independently selected from the group consisting of:
halogen,
hydroxy, and
carboxy,
C₁₋₅ alkyl, and
C₁₋₅ alkyl substituted by substituent(s) independently selected from the group consisting of:
halogen,
hydroxy, and
carboxy,
substituted heterocyclyl-ethylideneaminooxy,
C₁₋₅ alkoxycarbonyl,
C₁₋₅ alkoxycarbonyl substituted by carbocyclic aryl,
mono-C₁₋₅ alkylaminocarbonyl,
di-C₁₋₅ alkylaminocarbonyl,
mono-C₁₋₅ alkylamino,
mono-C₁₋₅ alkylamino substituted by substituent(s) independently selected from the group consisting of:
cyano,
carbocyclic aryl, and
heterocyclyl,
di-C₁₋₅ alkylamino,
di-C₁₋₅ alkylamino substituted by substituent(s) independently selected from the group consisting of:
cyano,
carbocyclic aryl, and
heterocyclyl,
mono-carbocyclic arylamino,
mono-carbocyclic arylamino substituted by substituent(s) independently selected from the group consisting of:
halogen,
hydroxy,
carboxy,
carbamoyl,
nitro,
cyano,
amino,
carbocyclic aryl,
carbocyclic aryl substituted by C₁₋₅ alkoxy,
C₁₋₅ alkoxy,
C₁₋₅ alkoxy substituted by substituent(s) independently selected from the group consisting of:
halogen,
hydroxy, and
carboxy,
C₁₋₅ alkyl, and C₁₋₅ alkyl substituted by substituent(s) independently selected from the group consisting of:
halogen,
hydroxy, and
carboxy,
di-carbocyclic arylamino,
di-carbocyclic arylamino substituted by substituent(s) independently selected from the group consisting of:
halogen,
hydroxy,
carboxy,
carbamoyl,
nitro,
cyano,
amino,
carbocyclic aryl,
carbocyclic aryl substituted by C₁₋₅ alkoxy,
C₁₋₅ alkoxy,
C₁₋₅ alkoxy substituted by substituent(s) independently selected from the group consisting of:
halogen,
hydroxy, and
carboxy,
C₁₋₅ alkyl, and
C₁₋₅ alkyl substituted by substituent(s) independently selected from the group consisting of:
halogen,
hydroxy, and
carboxy,
mono-heterocyclylamino,
mono-heterocyclylamino substituted by substituent(s) independently selected from the group consisting of:
halogen,
hydroxy,
carboxy,
carbamoyl,
nitro,
cyano,
amino,
carbocyclic aryl,
carbocyclic aryl substituted by C₁₋₅ alkoxy,
C₁₋₅ alkoxy,
C₁₋₅ alkoxy substituted by substituent(s) independently selected from the group consisting of:
halogen,
hydroxy, and
carboxy,
C₁₋₅ alkyl, and
C₁₋₅ alkyl substituted by substituent(s) independently selected from the group consisting of:
halogen,
hydroxy, and
carboxy,
di-heterocyclylamino,
di-heterocyclylamino substituted by substituent(s) independently selected from the group consisting of:
halogen,
hydroxy,
carboxy,
carbamoyl,
nitro,
cyano,
amino,
carbocyclic aryl,
carbocyclic aryl substituted by C₁₋₅ alkoxy,
C₁₋₅ alkoxy,
C₁₋₅ alkoxy substituted by substituent(s) independently selected from the group consisting of:
halogen,
hydroxy, and
carboxy,
C₁₋₅ alkyl, and
C₁₋₅ alkyl substituted by substituent(s) independently selected from the group consisting of:
halogen,
hydroxy, and
carboxy,
C₁₋₅ alkylcarbonylamino,
—C₁₋₅ alkylcarbonylamino substituted by substituent(s) independently selected from the group consisting of:
C₁₋₅ alkylcarbonylamino,
carbocyclic arylcarbonylamino, and
heterocyclyl,
C₁₋₅ alkoxycarbonylamino,
carbocyclic arylcarbonylamino,
heterocyclyl carbonylamino,
carbocyclic arylsulfonylamino,
carbocyclic arylsulfonylamino substituted by substituent(s) independently selected from the group consisting of:
nitro,
C₁₋₅ alkyl,
mono-C₁₋₅ alkylamino, and
di-C₁₋₅ alkylamino,
C₁₋₅ alkylthio,
C₁₋₅ alkylthio substituted by substituent(s) independently selected from the group consisting of:
mono-carbocyclic arylaminocarbonyl,
mono-carbocyclic arylaminocarbonyl substituted by halogen,
di-carbocyclic arylaminocarbonyl,
di-carbocyclic arylaminocarbonyl substituted by halogen,
mono-carbocyclic arylamino,
mono-carbocyclic arylamino substituted by halogen,
di-carbocyclic arylamino,
di-carbocyclic arylamino substituted by halogen,
carbocyclic aryl, and
carbocyclic aryl substituted by substituent(s) independently selected from the group consisting of:
halogen, and
C₁₋₅ alkoxy,
carbocyclic arylthio,
carbocyclic arylthio substituted by substituent(s) independently selected from the group consisting of:
halogen,
C₁₋₅ alkyl, and
C₁₋₅ alkyl substituted by halogen,
carbocyclic arylsulfinyl,
carbocyclic arylsulfinyl substituted by substituent(s) independently selected from the group consisting of:
halogen,
C₁₋₅ alkyl, and
C₁₋₅ alkyl substituted by halogen,
carbocyclic arylsulfonyl,
carbocyclic arylsulfonyl substituted by substituent(s) independently selected from the group consisting of:
halogen,
C₁₋₅ alkyl, and
C₁₋₅ alkyl substituted by halogen,
heterocyclylthio,
heterocyclylthio substituted by substituent(s) independently selected from the group consisting of:
nitro, and
C₁₋₅ alkyl,
C₃₋₆ cycloalkyl,
C₃₋₆ cycloalkyl substituted by C₁₋₅ alkyl,
C₃₋₆ cycloalkyl substituted by carbocyclic aryl,
C₃₋₆ cycloalkenyl,
carbocyclyl,
carbocyclyl substituted by substituent(s) independently selected from the group consisting of:
halogen,
C₁₋₅ alkyl,
C₁₋₅ alkoxy,
C₂₋₅ alkenyl, and
C₂₋₅ alkenyl substituted by substituent(s) independently selected from the group consisting of:
carbocyclic aryl, and
carbocyclic aryl substituted by C₁₋₅ alkylsulfinyl,
carbocyclic aryl,
carbocyclic aryl substituted by substituent(s) independently selected from the group consisting of:
halogen,
hydroxy,
carboxy,
carbamoyl,
cyano,
nitro,
amino,
C₁₋₅ alkylcarbonylamino,
C₃₋₆ cycloalkylcarbonylamino,
C₁₋₅ alkyl,
C₁₋₅ alkyl substituted by substituent(s) independently selected from the group consisting of:
halogen,
hydroxy,
carboxy,
carbamoyl,
oxo,
carbocyclic aryl,
heterocyclyl,
mono-carbocyclic arylamino,
di-carbocyclic arylamino,
mono-carbocyclic arylamino substituted by substituent(s) independently selected from the group consisting of:
halogen,
nitro,
C₁₋₅ alkyl,
C₁₋₅ alkoxy, and
C₁₋₅ alkoxy substituted by halogen,
di-carbocyclic arylamino substituted by substituent(s) independently selected from the group consisting of:
halogen,
nitro,
C₁₋₅ alkyl,
C₁₋₅ alkoxy, and
C₁₋₅ alkoxy substituted by halogen,
C₂₋₅ alkenyl,
C₁₋₅ alkoxy,
C₁₋₅ alkoxy substituted by substituent(s) independently selected from the group consisting of:
halogen, and
carbocyclic aryl,
carbocyclic aryloxy,
C₁₋₅ alkoxycarbonyl,
C₁₋₅ alkylcarbonyloxy,
mono-C₁₋₅ alkylamino,
di-C₁₋₅ alkylamino,
mono-carbocyclic arylamino,
mono-carbocyclic arylamino substituted by halogen,
di-carbocyclic arylamino,
di-carbocyclic arylamino substituted by halogen,
mono-carbocyclic arylaminocarbonyl,
mono-carbocyclic arylaminocarbonyl substituted by substituent(s) selected from the group consisting of:
halogen,
nitro,
C₁₋₅ alkyl,
C₁₋₅ alkoxy, and
C₁₋₅ alkoxy substituted by halogen,
di-carbocyclic arylaminocarbonyl,
di-carbocyclic arylaminocarbonyl substituted by substituent(s) selected from the group consisting of:
halogen,
nitro,
C₁₋₅ alkyl,
C₁₋₅ alkoxy, and
C₁₋₅ alkoxy substituted by halogen,
mercapto,
C₁₋₅ alkylthio,
C₁₋₅ alkylthio substituted by halogen,
C₁₋₅ alkylsulfonyl,
C₃₋₆ cycloalkyl,
carbocyclic aryl, and
heterocyclyl,
heterocyclyl, and
heterocyclyl substituted by substituent(s) independently selected from the group consisting of:
halogen,
hydroxy,
carboxy,
carbamoyl,
cyano,
nitro,
amino,
C₁₋₅ alkyl,
C₁₋₅ alkyl substituted by substituent(s) independently selected from the group consisting of:
halogen,
hydroxy,
carboxy, and
carbamoyl,
C₁₋₅ alkyl substituted by carbocyclic aryl,
C₁₋₅ alkoxy,
C₁₋₅ alkoxy substituted by halogen,
C₁₋₅ alkoxy substituted by carbocyclic aryl,
carbocyclic aryl, and
carbocyclic aryl substituted by halogen,
(ii) C₂₋₈ alkenyl, and
C₂₋₈ alkenyl substituted by substituent(s) independently selected from the group consisting of:
halogen,
oxo,
C₁₋₅ alkoxy,
C₁₋₅ alkoxy substituted by carbocyclic aryl,
carbocyclic aryl,
carbocyclic aryl substituted by substituent(s) independently selected from the group consisting of:
halogen,
hydroxy,
nitro,
C₁₋₅ alkyl,
C₁₋₅ alkyl substituted by halogen,
C₁₋₅ alkoxy, and
C₁₋₅ alkoxy substituted by halogen,
heterocyclyl, and
heterocyclyl substituted by substituent(s) independently selected from the group consisting of:
hydroxy,
nitro,
C₁₋₅ alkyl, and
C₁₋₅ alkoxy,
(iii) C₂₋₅ alkynyl, and
C₂₋₅ alkynyl substituted by carbocyclic aryl,
(iv) C₃₋₁₂ cycloalkyl, and
C₃₋₁₂ cycloalkyl substituted by substituent(s) independently selected from the group consisting of:
C₁₋₅ alkyl,
C₁₋₅ alkyl substituted by substituent(s) independently selected from the group consisting of:
hydroxy,
oxo, and
carbocyclic aryl,
mono-C₁₋₅ alkylamino,
mono-C₁₋₅ alkylamino substituted by carbocyclic aryl,
di-C₁₋₅ alkylamino,
di-C₁₋₅ alkylamino substituted by carbocyclic aryl,
carbocyclic arylcarbonylamino,
carbocyclic aryl, and
carbocyclic aryl substituted by halogen,
(v) C₃₋₆ cycloalkenyl, and
C₃₋₆ cycloalkenyl substituted by C₁₋₅ alkyl,
(vi) carbocyclyl, and
carbocyclyl substituted by substitutent(s) independently selected from the group consisting of:
hydroxy, and
nitro,
(vii) carbocyclic aryl, and
carbocyclic aryl substituted by substituent(s) independently selected from the group consisting of
halogen,
hydroxy,
cyano,
nitro,
C₁₋₁₀ alkyl,
C₁₋₁₀ alkyl substituted by substituent(s) independently selected from the group consisting of:
halogen,
hydroxy,
carboxy,
carbamoyl,
oxo,
C₁₋₅ alkoxy,
carbocyclic aryloxy,
mono-C₁₋₅ alkylamino-N-oxy,
di-C₁₋₅ alkylamino-N-oxy,
mono-C₁₋₅ alkylamino,
di-C₁₋₅ alkylamino,
mono-C₁₋₅ alkylamino substituted by carbocyclic aryl,
di-C₁₋₅ alkylamino substituted by carbocyclic aryl,
mono-carbocyclic arylamino,
di-carbocyclic arylamino,
carbocyclylimino,
carbocyclylimino substituted by carbocyclic aryl,
mono-carbocyclic arylamino,
di-carbocyclic arylamino,
mono-carbocyclic arylamino substituted by C₁₋₅ alkoxy,
di-carbocyclic arylamino substituted by C₁₋₅ alkoxy,
mono-carbocyclic arylaminocarbonyl,
di-carbocyclic arylaminocarbonyl,
mono-carbocyclic arylaminocarbonyl substituted by C₁₋₅ alkoxy,
di-carbocyclic arylaminocarbonyl substituted by C₁₋₅ alkoxy,
carbocyclic aryl,
carbocyclic aryl substituted by substituent(s) independently selected from the group consisting of:
halogen,
C₁₋₅ alkyl, and
C₁₋₅ alkyl substituted by halogen,
heterocyclyl, and
heterocyclyl substituted by C₁₋₅ alkyl,
C₂₋₅ alkenyl,
C₂₋₅ alkenyl substituted by carbocyclic aryl,
C₁₋₉ alkoxy,
C₁₋₉ alkoxy substituted by substituent(s) independently selected from the group consisting of:
hydroxy,
halogen,
carboxy,
mono-C₁₋₅ alkylamino,
di-C₁₋₅ alkylamino,
carbocyclic aryl,
halogenated carbocyclic aryl,
heterocyclyl,
heterocyclyl substituted by substituent(s) independently selected from the group consisting of:
halogen,
heterocyclyl, and
heterocyclyl substituted by substituent(s) independently selected from the group consisting of:
halogen,
C₁₋₅ alkyl, and
C₁₋₅ alkyl substituted by halogen,
C₂₋₅ alkenyloxy,
C₃₋₆ cycloalkoxy,
C₁₋₅ alkylcarbonyloxy,
carbocyclic aryloxy,
carbocyclic aryloxy substituted by substituent(s) independently selected from the group consisting of:
halogen,
hydroxy,
carboxy,
carbamoyl,
cyano,
nitro,
amino,
C₁₋₅ alkyl,
C₁₋₅ alkyl substituted by substituent(s) independently selected from the group consisting of:
halogen,
hydroxy,
carboxy, and
carbamoyl,
C₁₋₅ alkoxy, and
C₁₋₅ alkoxy substituted by halogen,
heterocyclyloxy,
heterocyclyloxy substituted by substituent(s) independently selected from the group consisting of:
halogen,
hydroxy,
carboxy,
carbamoyl,
cyano,
nitro,
amino,
C₁₋₅ alkyl,
C₁₋₅ alkyl substituted by substituent(s) independently selected from the group consisting of:
halogen,
hydroxy,
carboxy, and
carbamoyl,
C₁₋₅ alkoxy, and
C₁₋₅ alkoxy substituted by halogen,
(carbocyclic aryl)S(O)₂O,
carboxy,
carbamoyl,
C₁₋₅ alkoxycarbonyl,
mono-C₁₋₅ alkylaminocarbonyl,
di-C₁₋₅ alkylaminocarbonyl,
mono-C₁₋₅ alkylaminocarbonyl substituted by carbocyclic aryl,
di-C₁₋₅ alkylaminocarbonyl substituted by carbocyclic aryl,
mono-carbocyclic arylaminocarbonyl,
di-carbocyclic arylaminocarbonyl,
mono-carbocyclic arylaminocarbonyl substituted by C₁₋₅ alkyl,
di-carbocyclic arylaminocarbonyl substituted by C₁₋₅ alkyl,
amino,
mono-C₁₋₅ alkylamino,
di-C₁₋₅ alkylamino,
mono-C₁₋₅ alkylamino substituted by cyano,
di-C₁₋₅ alkylamino substituted by cyano,
mono-carbocyclic arylamino,
di-carbocyclic arylamino,
C₁₋₅ alkylcarbonylamino,
C₃₋₆ cycloalkylcarbonylamino,
C₂₋₅ alkynylcarbonylamino,
C₂₋₅ alkynylcarbonylamino substituted by carbocyclic aryl,
C₁₋₅ alkoxycarbonylamino,
carbocyclic arylsulfonylamino,
carbocyclic arylsulfonylamino substituted by C₁₋₅ alkyl,
(carbocyclic aryl)NHC(O)NH,
(carbocyclic aryl)NHC(O)NH substituted by C₁₋₅ alkoxy,
(carbocyclic aryl)NHC(O)NH substituted by halogenated C₁₋₅ alkoxy,
carbocyclic aryl azo,
carbocyclic aryl azo substituted by mono-C₁₋₅ alkylamino,
carbocyclic aryl azo substituted by di-C₁₋₅ alkylamino,
C₁₋₅ alkylthio,
C₁₋₅ alkylthio substituted by halogen,
carbocyclic arylthio,
carbocyclic arylthio substituted by substituent(s) independently selected from the group consisting of:
halogen,
nitro,
cyano, and
C₁₋₅ alkyl,
aminosulfonyl,
heterocyclylthio,
C₁₋₅ alkylsulfonyl,
mono-C₁₋₅ alkylaminosulfonyl,
di-C₁₋₅ alkylaminosulfonyl,
heterocyclylsulfonyl,
C₃₋₆ cycloalkyl,
C₃₋₆ cycloalkyl substituted by C₁₋₅ alkyl,
carbocyclic aryl,
carbocyclic aryl substituted by substituent(s) independently selected from the group consisting of:
C₁₋₇ alkyl, and
C₁₋₇ alkyl substituted by halogen,
heterocyclyl, and
heterocyclyl substituted by substituent(s) independently selected from the group consisting of:
C₁₋₅ alkyl,
carbocyclic aryl, and
halogenated carbocyclic aryl,
C₁₋₅ alkoxycarbonyl substituted by carbocyclic aryl, and
(viii) heterocyclyl, and
heterocyclyl substituted by substituent(s) independently selected from the group consisting of:
halogen,
hydroxy,
carboxy,
carbamoyl,
cyano,
nitro,
amino,
C₁₋₅ alkyl,
C₁₋₅ alkyl substituted by substituent(s) independently selected from the group consisting of:
halogen,
hydroxy,
carboxy,
carbamoyl,
oxo,
C₁₋₅ alkylcarbonyloxy,
carbocyclic arylcarbonylamino,
carbocyclic arylcarbonylamino substituted by halogen,
C₁₋₅ alkoxycarbonyl,
C₁₋₅ alkylthio,
C₁₋₅ alkylthio substituted by carbocyclic aryl,
C₁₋₅ alkylthio substituted by halogenated carbocyclic aryl,
carbocyclic aryl,
carbocyclic aryl substituted by substituent(s) independently selected from the group consisting of:
halogen, and
nitro,
heterocyclyl, and
heterocyclyl substituted by substituent(s) independently selected from the group consisting of:
halogen,
C₁₋₅ alkyl, and
C₁₋₅ alkyl substituted by halogen,
C₁₋₅ alkoxy,
C₁₋₅ alkoxy substituted by halogen,
C₁₋₅ alkoxy substituted by carbocyclic aryl,
carbocyclic aryloxy,
carbocyclic aryloxy substituted by substituent(s) independently selected from the group consisting of:
halogen,
nitro,
cyano,
hydroxy,
carboxy,
carbamoyl,
amino,
C₁₋₅ alkyl,
C₁₋₅ alkyl substituted by substituent(s) independently selected from the group consisting of:
halogen,
hydroxy,
carboxy, and
carbamoyl,
mono-C₁₋₅ alkylamino,
di-C₁₋₅ alkylamino,
C₁₋₅ alkylcarbonylamino,
C₃₋₆ cycloalkycarbonylamino,
C₁₋₅ alkoxy,
C₁₋₅ alkoxy substituted by halogen,
C₃₋₆ cycloalkyl,
C₂₋₅ alkenyl,
C₂₋₅ alkynyl,
carboxy,
C₁₋₅ alkoxycarbonyl,
mono-C₁₋₅ alkylaminocarbonyl,
di-C₁₋₅ alkylaminocarbonyl,
mono-C₃₋₆ cycloalkylaminocarbonyl,
di-C₃₋₆ cycloalkylaminocarbonyl,
mono-C₁₋₅ alkylaminocarbonylamino,
di-C₁₋₅ alkylaminocarbonylamino,
mono-C₃₋₆ cycloalkylaminocarbonylamino,
di-C₃₋₆ cycloalkylaminocarbonylamino,
C₁₋₅ alkylthio,
C₁₋₅ alkylthio substituted by halogen,
C₁₋₅ alkylsulfinyl,
C₁₋₅ alkylsulfinyl substituted by halogen,
C₁₋₅ alkylsulfonyl, and
C₁₋₅ alkylsulfonyl substituted by halogen,
heterocyclyloxy,
heterocyclyloxy substituted by substituent(s) independently selected from the group consisting of:
halogen,
nitro,
hydroxy,
carboxy,
carbamoyl,
cyano,
amino,
C₁₋₅ alkyl,
C₁₋₅ alkyl substituted by substituent(s) independently selected from the group consisting of:
halogen,
hydroxy,
carboxy, and
carbamoyl,
C₁₋₅ alkoxy, and
C₁₋₅ alkoxy substituted by halogen,
mono-C₁₋₅ alkylamino,
di-C₁₋₅ alkylamino,
C₁₋₅ alkylcarbonylamino,
C₁₋₅ alkylthio,
C₂₋₅ alkenylthio,
carbocyclic arylthio,
carbocyclic arylthio substituted by halogen,
carbocyclic arylthio substituted by C₁₋₅ alkoxycarbonyl,
heterocyclylthio,
heterocyclylthio substituted by C₁₋₅ alkyl,
C₁₋₅ alkylsulfinyl,
C₁₋₅ alkylsulfonyl,
carbocyclic arylsulfinyl,
carbocyclic arylsulfinyl substituted by halogen,
carbocyclic arylsulfonyl,
carbocyclic arylsulfonyl substituted by halogen,
carbocyclic arylsulfonyl substituted by C₁₋₅ alkyl,
C₁₋₅ alkoxycarbonyl,
C₁₋₅ alkoxycarbonyl substituted by carbocyclic aryl,
carbocyclic aryl,
carbocyclic aryl substituted by substituent(s) independently selected from the group consisting of:
halogen,
nitro,
C₁₋₅ alkyl,
C₁₋₅ alkyl substituted by halogen,
C₁₋₅ alkoxy, and
C₁₋₅ alkoxy substituted by halogen,
heterocyclyl, and
heterocyclyl substituted by substituent(s) independently selected from the group consisting of:
halogen,
C₁₋₅ alkyl,
C₁₋₅ alkyl substituted by halogen,
C₁₋₅ alkoxy, and
C₁₋₅ alkoxycarbonyl;
R₂ is halogen, C₁₋₅ alkyl, C₁₋₅ alkyl substituted by halogen, C₁₋₅ alkyl substituted by hydroxy, C₁₋₅ alkyl substituted by carbocyclic aryl, C₁₋₅ alkyl substituted by halogenated carbocyclic aryl, C₁₋₅ alkyl substituted by heterocyclyl, C₁₋₅ alkyl substituted by halogenated heterocyclyl, C₂₋₅ alkenyl, C₂₋₅ alkynyl, C₁₋₅ alkoxy, C₁₋₅ alkoxy substituted by halogen, C₁₋₅ alkylthio, —N(R₂ₐ)(R_{2b}); wherein R₂ₐ and R_{2b} are each independently hydrogen, C₁₋₅ alkyl, or C₁₋₅ alkyl substituted by substituent(s) independently selected from the group consisting of:
halogen,
hydroxy,
carboxy,
carbamoyl,
C₁₋₅ alkoxy,
amino,
C₃₋₆ cycloalkyl,
carbocyclic aryl,
carbocyclic aryl substituted by substituent(s) independently selected from the group consisting of:
halogen,
C₁₋₅ alkyl,
C₁₋₅ alkoxy,
C₁₋₅ alkyl substituted by halogen,
C₁₋₅ alkoxy substituted by halogen, and
—SO₂NH₂,
heterocyclyl, and
heterocyclyl substituted by substituent(s) independently selected from the group consisting of:
halogen,
C₁₋₅ alkyl,
C₁₋₅ alkoxy,
C₁₋₅ alkyl substituted by halogen, and
C₁₋₅ alkoxy substituted by halogen,
C₃₋₆ cycloalkyl, carbocyclic aryl, carbocyclic aryl substituted by substituent(s) independently selected from the group consisting of:
halogen,
C₁₋₅ alkyl,
C₁₋₅ alkoxy,
C₁₋₅ alkyl substituted by halogen, and
C₁₋₅ alkoxy substituted by halogen,
heterocyclyl, or heterocyclyl substituted by substituent(s) independently selected from the group consisting of:
halogen,
C₁₋₅ alkyl,
C₁₋₅ alkoxy,
C₁₋₅ alkyl substituted by halogen, and
C₁₋₅ alkoxy substituted by halogen;
L is selected from the group consisting of Formulae (III), (IIIa), (IIIb), (IV), (IVa), and (IVb); 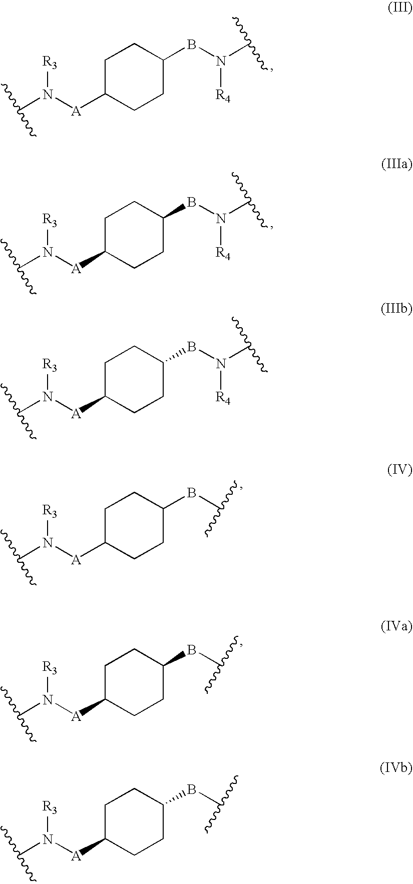
wherein R₃ and R₄ are each independently hydrogen or C₁₋₅ alkyl; and A and B are each independently a single bond, —CH₂—, or —(CH₂)₂—;
Z₁, Z₂, Z₃, and Z₄ are each independently hydrogen, halogen, C₁₋₅ alkyl, C₁₋₅ alkyl substituted by halogen, C₁₋₅ alkyl substituted by hydroxy, C₁₋₅ alkyl substituted by carbocyclic aryl, C₁₋₅ alkyl substituted by halogenated carbocyclic aryl, C₁₋₅ alkyl substituted by heterocyclyl, C₁₋₅ alkyl substituted by halogenated heterocyclyl, C₂₋₅ alkenyl, C₂₋₅ alkynyl, C₃₋₆ cycloalkyl, C₁₋₅ alkoxy, C₁₋₅ alkoxy substituted by halogen, mono-C₁₋₅ alkyl amino, di-C₁₋₅ alkyl amino, C₁₋₅ alkylthio, carbocyclic aryl, substituted carbocyclic aryl, heterocyclyl, or substituted heterocyclyl; or
R₂ and Z₂ are bonded to each other to form a ring and —R₂-Z₂- is —(CH₂)n- or —(CH₂)o-CH═CH—(CH₂)p—; wherein one —CH₂— group of —R₂-Z₂- can optionally be replaced by C(O), NR₆, O, S, S(O), or S(O)₂; wherein n is 2, 3, 4, 5, or 6; o and p are each independently 0, 1, 2, 3, or 4 provided that o+p=0, 1, 2, 3, or 4; and R₆ is hydrogen, C₁₋₅ alkyl, or substituted C₁₋₅ alkyl;
and
Y represents:
(i) —C(O)NR₅—, —C(S)NR₅—, —C(O)O—, —S(O)₂—, —C(O)—, —C(S)—, or —(CH₂)ₘ— when L is selected from the group consisting of Formulae (III), (IIIa), and (IIIb); or
(ii) —C(O)NR₅—, —C(S)NR₅—, —C(O)O—, or —OC(O)— when L is selected from the group consisting of Formulae (IV), (IVa), and (IVb);
wherein R₅ is hydrogen or C₁₋₅ alkyl; and m is 0, 1, 2, 3, 4, or 5;
wherein carbocyclic aryl is phenyl, naphthyl, anthranyl, phenanthryl, or biphenyl;
carbocyclyl is 10,11-dihydro-5-oxo-dibenzo[a,d]cycloheptyl, 1-oxo-indanyl, 7,7-dimethyl-2-oxo-bicyclo[2.2.1]heptyl, 9H-fluorenyl, 9-oxo-fluorenyl, acenaphthyl, anthraquinonyl, C-fluoren-9-ylidene, indanyl, indenyl, menthyl, 1,2,3,4-tetrahydro-naphthyl, or bicyclo[2.2.1]heptenyl;
heterocyclyl is 1,2,3,4-tetrahydro-isoquinolyl, 1,2,3-thiadiazolyl, 1,2,3-triazolyl, 1,2-dihydro-3-oxo-pyrazolyl, 1,3,4-thiadiazolyl, 1,3-dioxo-isoindolyl, 1,3-dioxolanyl, 1H-indolyl, 1H-pyrrolo[2,3-c]pyridyl, 1H-pyrrolyl, 1-oxo-3H-isobenzofuranyl, 2,2′,5′,2″-terthiophenyl, 2,2′-bithiophenyl, 2,3-dihydro-1-oxo-isoindolyl, 2,3-dihydro-benzo[1,4]dioxinyl, 2,3-dihydro-benzofuryl, 2,4-dihydro-3-oxo-pyrazolyl, 2H-benzopyranyl, 2-oxo-benzopyranyl, 2-oxo-pyrrolidinyl, 3,4-dihydro-2H-benzo[1,4]oxazinyl, 3,4-dihydro-2H-benzo[b][1,4]dioxepinyl, 4H-benzo[1,3]dioxinyl, 4H-benzopyranyl, 4-oxo-1,5,6,7-tetrahydro-indolyl, 4-oxo-3,4-dihydro-phthalazinyl, 4-oxo-benzopyranyl, 9,10,10-trioxo-thioxanthenyl, 9H-carbazolyl, 9H-xanthenyl, azetidinyl, benzimidazolyl, benzo[1,3]dioxolyl, benzo[2,1,3]oxadiazolyl, benzo[1,2,5]oxadiazolyl, benzo[b]thienyl, benzofuryl, benzothiazolyl, cinnolyl, furyl, imidazo[2,1-b]thiazolyl, imidazolyl, isoxazolyl, morpholino, morpholinyl, oxazolyl, oxolanyl, piperazyl, piperidyl, piridyl, pyrazolo[5,1-b]thiazolyl, pyrazolyl, pyrazinyl, pyridyl, pyrimidyl, pyrrolidyl, quinolyl, quinoxalyl, thiazolidyl, thiazolyl, thienyl, thiolanyl, 2,3-dihydro-benzofuryl, tetrahydro-thienyl, or benzofuranyl;
halogen is fluoro, chloro, bromo, or iodo;
or a pharmaceutically acceptable salt, hydrate, or solvate thereof.

2. The compound according to claim 1 wherein Q is Formula (IIa);
Z₁ is hydrogen, halogen, C₁₋₅ alkyl, C₁₋₅ alkyl substituted by halogen, C₃₋₆ cycloalkyl, C₁₋₅ alkoxy, C₁₋₅ alkoxy substituted by halogen, or C₁₋₅ alkylthio; or a pharmaceutically acceptable salt, hydrate, or solvate thereof.

3. The compound according to claim 2 wherein R₁ is selected from the group consisting of:
(i) C₁₋₁₀ alkyl, and
C₁₋₁₀ alkyl substituted by substituent(s) independently selected from the group consisting of:
halogen,
oxo,
C₁₋₅ alkoxy,
C₁₋₅ alkoxy substituted by carbocyclic aryl,
C₁₋₅ alkylcarbonyloxy,
C₁₋₅ alkoxycarbonyl,
C₁₋₅ alkoxycarbonyl substituted by carbocyclic aryl,
carbocyclic aryloxy, and
carbocyclic aryloxy substituted by substituent(s) independently selected from the group consisting of:
halogen,
nitro,
C₁₋₅ alkyl, and
C₁₋₅ alkyl substituted by oxo,
heterocyclyloxy,
heterocyclyloxy substituted by C₁₋₅ alkyl,
mono-carbocyclic arylamino,
di-carbocyclic arylamino,
carbocyclic arylsulfonylamino,
carbocyclic arylsulfonylamino substituted by C₁₋₅ alkyl,
C₁₋₅ alkylthio,
C₁₋₅ alkylthio substituted by carbocyclic aryl,
carbocyclic arylthio,
carbocyclic arylthio substituted by halogen,
carbocyclic arylthio substituted by C₁₋₅ alkyl,
carbocyclic arylsulfonyl,
carbocyclic arylsulfonyl substituted by halogen,
heterocyclylthio,
heterocyclylthio substituted by C₁₋₅ alkyl,
C₃₋₆ cycloalkyl,
C₃₋₆ cycloalkenyl,
carbocyclyl,
carbocyclyl substituted by C₁₋₅ alkoxy,
carbocyclic aryl, and
carbocyclic aryl substituted by substituent(s) independently selected from the group consisting of:
halogen,
nitro,
C₁₋₅ alkyl, and
C₁₋₅ alkyl substituted by substituent(s) independently selected from the group consisting of:
halogen,
carbocyclic aryl, and
heterocyclyl,
C₁₋₅ alkoxy,
C₁₋₅ alkoxy substituted by halogen,
C₁₋₅ alkoxy substituted by carbocyclic aryl,
carbocyclic aryloxy,
mono-carbocyclic arylaminocarbonyl, and
mono-carbocyclic arylaminocarbonyl substituted by substituent(s) selected from the group consisting of:
halogen,
C₁₋₅ alkyl,
C₁₋₅ alkoxy, and
C₁₋₅ alkoxy substituted by halogen,
di-carbocyclic arylaminocarbonyl, and
di-carbocyclic arylaminocarbonyl substituted by substituent(s) selected from the group consisting of:
halogen,
C₁₋₅ alkyl,
C₁₋₅ alkoxy, and
C₁₋₅ alkoxy substituted by halogen,
C₁₋₅ alkylthio,
C₁₋₅ alkylthio substituted by halogen,
C₁₋₅ alkylsulfonyl,
carbocyclic aryl, and
heterocyclyl,
heterocyclyl, and
heterocyclyl substituted by substituent(s) independently selected from the group consisting of:
C₁₋₅ alkyl,
C₁₋₅ alkoxy,
C₁₋₅ alkoxy substituted by carbocyclic aryl,
carbocyclic aryl, and
carbocyclic aryl substituted by halogen,
(ii) C₂₋₅ alkenyl, and
C₂₋₅ alkenyl substituted by substituent(s) independently selected from the group consisting of:
carbocyclic aryl, and
carbocyclic aryl substituted by substituent(s) independently selected from the group consisting of:
nitro,
halogen,
C₁₋₅ alkyl,
C₁₋₅ alkyl substituted by halogen,
C₁₋₅ alkoxy, and
C₁₋₅ alkoxy substituted by halogen,
(iii) C₃₋₆ cycloalkyl, and
C₃₋₆ cycloalkyl substituted by substituent(s) independently selected from the group consisting of:
C₁₋₅ alkyl,
C₁₋₅ alkyl substituted by carbocyclic aryl,
carbocyclic arylcarbonylamino, and
carbocyclic aryl,
(iv) carbocyclyl, and
carbocyclyl substituted by nitro,
(v) carbocyclic aryl, and
carbocyclic aryl substituted by substituent(s) independently selected from the group consisting of:
halogen,
cyano,
nitro,
C₁₋₉ alkyl, and
C₁₋₉ alkyl substituted by substituent(s) independently selected from the group consisting of:
halogen,
oxo,
mono-carbocyclic arylaminocarbonyl,
di-carbocyclic arylaminocarbonyl,
mono-carbocyclic arylaminocarbonyl substituted by C₁₋₅ alkoxy,
di-carbocyclic arylaminocarbonyl substituted by C₁₋₅ alkoxy,
carbocyclic aryloxy,
carbocyclic aryl, and
carbocyclic aryl substituted by substituent(s) independently selected from the group consisting of:
halogen,
C₁₋₅ alkyl, and
C₁₋₅ alkyl substituted by halogen,
heterocyclyl, and
heterocyclyl substituted by C₁₋₅ alkyl,
C₂₋₅ alkenyl,
C₁₋₇ alkoxy,
C₁₋₇ alkoxy substituted by halogen,
C₁₋₇ alkoxy substituted by carbocyclic aryl,
C₃₋₆ cycloalkoxy,
carbocyclic aryloxy, and
carbocyclic aryloxy substituted by substituent(s) independently selected from the group consisting of:
halogen,
nitro, and
C₁₋₅ alkoxy
heterocyclyloxy, and
heterocyclyloxy substituted by substituent(s) independently selected from the group consisting of:
halogen,
C₁₋₅ alkyl, and
C₁₋₅ alkyl substituted by halogen,
C₁₋₅ alkoxycarbonyl,
mono-C₁₋₅ alkylaminocarbonyl,
di-C₁₋₅ alkylaminocarbonyl,
mono-C₁₋₅ alkylaminocarbonyl substituted by carbocyclic aryl,
di-C₁₋₅ alkylaminocarbonyl substituted by carbocyclic aryl,
mono-carbocyclic arylaminocarbonyl,
di-carbocyclic arylaminocarbonyl,
mono-carbocyclic arylaminocarbonyl substituted by C₁₋₅ alkyl,
di-carbocyclic arylaminocarbonyl substituted by C₁₋₅ alkyl,
mono-C₁₋₅ alkylamino,
di-C₁₋₅ alkylamino,
C₁₋₅ alkylthio,
C₁₋₅ alkylthio substituted by halogen,
C₁₋₅ alkylsulfonyl,
carbocyclic aryl, and
carbocyclic aryl substituted by substituent(s) independently selected from the group consisting of:
C₁₋₇ alkyl, and
C₁₋₇ alkyl substituted by halogen,
(vi) heterocyclyl, and
heterocyclyl substituted by substituent(s) independently selected from the group consisting of:
halogen,
C₁₋₅ alkyl, and
C₁₋₅ alkyl substituted by substituent(s) independently selected from the group consisting of
halogen,
oxo,
carbocyclic aryl,
carbocyclic aryl substituted by halogen,
heterocyclyl, and
heterocyclyl substituted by substituent(s) independently selected from the group consisting of:
halogen,
C₁₋₅ alkyl, and
C₁₋₅ alkyl substituted by halogen,
C₁₋₅ alkoxy,
C₁₋₅ alkylthio,
carbocyclic arylthio,
C₁₋₅ alkylsulfonyl,
carbocyclic arylsulfonyl,
carbocyclic arylsulfonyl substituted by halogen,
carbocyclic arylsulfonyl substituted by C₁₋₅ alkyl,
C₁₋₅ alkoxycarbonyl,
carbocyclic aryl, and
carbocyclic aryl substituted by substituent(s) independently selected from the group consisting of:
halogen,
nitro, and
C₁₋₅ alkyl,
heterocyclyl, and
heterocyclyl substituted by substituent(s) independently selected from the group consisting of:
halogen,
C₁₋₅ alkyl, and
C₁₋₅ alkyl substituted by halogen;
wherein carbocyclic aryl is phenyl, naphthyl, or anthranyl;
carbocyclyl is 1-oxo-indanyl, 9H-fluorenyl, 9-oxo-fluorenyl, anthraquinonyl, C-fluoren-9-ylidene, indanyl, or menthyl;
heterocyclyl is 1,2,3,4-tetrahydro-isoquinolyl, 1,2,3-thiadiazolyl, 1,2,3-triazolyl, 1,3-dioxo-isoindolyl, 1H-indolyl, 1H-pyrrolyl, 2,3-dihydro-1-oxo-isoindolyl, 2,3-dihydro-benzo[1,4]dioxinyl, 2H-benzopyranyl, 2-oxo-benzopyranyl, 2-oxo-pyrrolidinyl, 4-oxo-benzopyranyl, 9H-xanthenyl, benzo[1,3]dioxolyl, benzo[2,1,3]oxadiazolyl, benzo[1,2,5]oxadiazolyl, benzo[b]thienyl, furyl, isoxazolyl, morpholinyl, oxazolyl, pyrazolyl, pyridyl, pyrimidyl, pyrrolidyl, quinolyl, quinoxalyl, thiazolyl, thienyl, imidazolyl, or piperazyl;
halogen is fluoro, chloro, bromo, or iodo,
or a pharmaceutically acceptable salt, hydrate, or solvate thereof.

4. The compound according to claim 3 wherein:
R₂ is halogen, C₁₋₅ alkyl, C₁₋₅ alkoxy, —N(R₂ₐ)(R_{2b}), or heterocyclyl; wherein R₂ₐ and R_{2b} are each independently hydrogen, C₁₋₅ alkyl, C₁₋₅ alkyl substituted by hydroxy, C₁₋₅ alkyl substituted by carbocyclic aryl, C₁₋₅ alkyl substituted by heterocyclyl, C₃₋₆ cycloalkyl, or carbocyclic aryl;
L is selected from the group consisting of Formulae (IIIa) and (IVa);
wherein R₃ and R₄ are each independently hydrogen or C₁₋₅ alkyl; and A and B are each independently a single bond, —CH₂—, or —(CH₂)₂—;
Z₁ is hydrogen, halogen, C₁₋₅ alkyl, C₁₋₅ alkyl substituted by halogen, C₁₋₅ alkoxy, or C₁₋₅ alkylthio; Z₂ is hydrogen, halogen, or C₁₋₅ alkyl; or
R₂ and Z₂ are bonded to each other to form a ring and —R₂-Z₂- is —NR₆—CH═CH—; wherein
R₆ is hydrogen or C₁₋₅ alkyl; and
Y represents:
(i) —C(O)NR₅—, —C(S)NR₅—, —C(O)O—, —S(O)₂—, —C(O)—, or —(CH₂)ₘ— when L is selected from the group consisting of Formula (IIIa); or
(ii) —C(O)NR₅— or —C(O)O— when L is selected from the group consisting of Formula (IVa);
wherein R₅ is hydrogen or C₁₋₅ alkyl; and m is 0, 1, or 2;
or a pharmaceutically acceptable salt, hydrate, or solvate thereof.

5. The compound according to claim 4 wherein R₁ is selected from the group consisting of:
(i) C₁₋₅ alkyl substituted by substituent(s) independently selected from the group consisting of:
hydroxy,
carbocyclic aryl,
carbocyclic aryl substituted by halogen, and
C₁₋₅ alkylthio,
(ii) C₃₋₆ cycloalkyl, and
(iii) carbocyclic aryl, and
carbocyclic aryl substituted by substituent(s) independently selected from the group consisting of:
halogen,
nitro,
cyano,
C₁₋₅ alkyl,
C₁₋₅ alkyl substituted by halogen,
C₁₋₅ alkoxy,
C₁₋₅ alkoxy substituted by halogen,
C₁₋₅ alkoxy substituted by carbocyclic aryl,
carbocyclic aryloxy, and
carbocyclic aryloxy substituted by C₁₋₅ alkoxy,
(iv) heterocyclyl, and
heterocyclyl substituted by substituent(s) independently selected from the group consisting of:
halogen,
C₁₋₅ alkyl,
carbocyclic aryl, and
carbocyclic aryl substituted by halogen;
R₂ is —N(R₂ₐ)(R_{2b}) or heterocyclyl; wherein R₂ₐ and R_{2b} are each independently hydrogen or C₁₋₅ alkyl;
Z₁ is hydrogen, C₁₋₅ alkyl, or C₁₋₅ alkylthio; Z₂ is hydrogen or C₁₋₅ alkyl or
R₂ and Z₂ are bonded to each other to form a ring and —R₂-Z₂- is —NR₆—CH═CH—; wherein
R₆ is hydrogen or C₁₋₅ alkyl;
L is Formula (IIIa) or (IVa), wherein R₃ and R₄ are hydrogen, A is a single bond and B is a single bond or —CH₂—;
and
Y represents:
(i) —C(O)NH—, —C(S)NH, —C(O)—, or —CH₂— when L is selected from the group consisting of Formula (IIIa); or
(ii) —C(O)NH— when L is selected from the group consisting of Formula (IVa);
wherein carbocyclic aryl is phenyl or naphthyl;
heterocyclyl is furyl, 1H-indolyl, morpholinyl, oxazolyl, piperidyl, pyridyl, pyrrolidyl, or 9H-xanthenyl;
halogen is fluoro, chloro, or bromo;
or a pharmaceutically acceptable salt, hydrate, or solvate thereof.

6. The compound according to claim 5 wherein R₁ is selected from the group consisting of:
(i) carbocyclic aryl, and
carbocyclic aryl substituted by substituent(s) independently selected from the group consisting of:
halogen,
C₁₋₅ alkyl,
C₁₋₅ alkyl substituted by halogen,
C₁₋₅ alkoxy, and
C₁₋₅ alkoxy substituted by halogen,
(ii) heterocyclyl, and
heterocyclyl substituted by halogen;
and
Z₁ is hydrogen, C₁₋₅ alkyl, or C₁₋₅ alkylthio; Z₂ is hydrogen or C₁₋₅ alkyl;
wherein carbocyclic aryl is phenyl;
heterocyclyl is furyl, pyridyl, or pyrrolidyl;
halogen is fluoro, chloro, or bromo;
or a pharmaceutically acceptable salt, hydrate, or solvate thereof.

7. The compound according to claim 1 selected from the group consisting of:
N-(cis-4-{[6-(dimethylamino)pyrimidin-4-yl]amino}cyclohexyl)-3,4-difluorobenzamide;
N-(cis-4-{[6-(dimethylamino)-2-methylpyrimidin-4-yl]amino}cyclohexyl)-4-fluorobenzamide;
4-chloro-N-(cis-4-{[6-(dimethylamino)-2-methylpyrimidin-4-yl]amino}cyclohexyl)-3-fluorobenzamide;
N-(cis-4-{[6-(dimethylamino)-2-methylpyrimidin-4-yl]amino}cyclohexyl)-3,5-difluorobenzamide;
3-chloro-N-(cis-4-{[6-(dimethylamino)-2-methylpyrimidin-4-yl]amino}cyclohexyl)-4-(trifluoromethoxy)benzamide;
3-chloro-4-fluoro-N-(cis-4-{[2-methyl-6-(methylamino)pyrimidin-4-yl]amino}cyclohexyl)benzamide;
N-(cis-4-{[6-(dimethylamino)-2-methylpyrimidin-4-yl]amino}cyclohexyl)-3-fluorobenzamide;
4-chloro-N-(cis-4-{[6-(dimethylamino)-2-methylpyrimidin-4-yl]amino}cyclohexyl)benzamide;
N-(cis-4-{[6-(dimethylamino)-2-methylpyrimidin-4-yl]amino}cyclohexyl)-3-fluoro-5-(trifluoromethyl)benzamide;
N-(cis-4-{[6-(dimethylamino)-2-methylpyrimidin-4-yl]amino}cyclohexyl)-3,5-bis(trifluoromethyl)benzamide;
3-chloro-4-fluoro-N-{cis-4-[(2-methyl-6-piperidin-1-ylpyrimidin-4-yl)amino]cyclohexyl}benzamide;
3-chloro-4-fluoro-N-{cis-4-[(2-methyl-6-morpholin-4-ylpyrimidin-4-yl)amino]cyclohexyl}benzamide;
3-chloro-4-fluoro-N-{cis-4-[(7-methyl-7H-pyrrolo[2,3-d]pyrimidin-4-yl)amino]cyclohexyl}benzamide;
3,4,5-trifluoro-N-{cis-4-[(7-methyl-7H-pyrrolo[2,3-d]pyrimidin-4-yl)amino]cyclohexyl}benzamide;
3,4,5-trifluoro-N-(cis-4-{[2-methyl-6-(methylamino)pyrimidin-4-yl]amino}cyclohexyl)benzamide;
cis-N-(3,4-difluorophenyl)-4-{[6-(dimethylamino)-2-methylpyrimidin-4-yl]amino}cyclohexanecarboxamide;
1-(4-chlorophenyl)-N-(cis-4-{[6-(dimethylamino)-2-methylpyrimidin-4-yl]amino}cyclohexyl)cyclopentanecarboxamide;
3-(2-chloro-6-fluorophenyl)-N-(cis-4-{[6-(dimethylamino)-2-methylpyrimidin-4-yl]amino}cyclohexyl)-5-methylisoxazole-4-carboxamide;
N-(cis-4-{[6-(dimethylamino)-2-methylpyrimidin-4-yl]amino}cyclohexyl)-2-(4-methoxyphenoxy)-5-nitrobenzamide;
N-(cis-4-{[6-(dimethylamino)-2-methylpyrimidin-4-yl]amino}cyclohexyl)-5-iodo-2-furamide;
N-(cis-4-{[6-(dimethylamino)-2-methylpyrimidin-4-yl]amino}cyclohexyl)-2-(ethylthio)-2,2-diphenylacetamide;
N-(cis-4-{[6-(dimethylamino)-2-methylpyrimidin-4-yl]amino}cyclohexyl)-9H-xanthene-9-carboxamide;
N-(cis-4-{[6-(dimethylamino)-2-methylpyrimidin-4-yl]amino}cyclohexyl)-N′-[1-(1-naphthyl)ethyl]urea;
N-(cis-4-{[6-(dimethylamino)-2-methylpyrimidin-4-yl]amino}cyclohexyl)-N′-(3,4,5-trimethoxyphenyl)urea;
N-(5-chloro-2,4-dimethoxyphenyl)-N′-(cis-4-{[6-(dimethylamino)-2-methylpyrimidin-4-yl]amino}cyclohexyl)urea;
N-(cis-4-{[6-(dimethylamino)-2-methylpyrimidin-4-yl]amino}cyclohexyl)-N′-(2,4,6-tribromophenyl)urea;
N-(cis-4-{[6-(dimethylamino)-2-methylpyrimidin-4-yl]amino}cyclohexyl)-N′-mesitylthiourea;
N-(2,6-diethylphenyl)-N′-(cis-4-{[6-(dimethylamino)-2-methylpyrimidin-4-yl]amino}cyclohexyl)thiourea;
N-(2,4-dichloro-6-methylphenyl)-N′-(cis-4-{[6-(dimethylamino)-2-methylpyrimidin-4-yl]amino}cyclohexyl)thiourea;
N-(5-chloro-2,4-dimethoxyphenyl)-N′-(cis-4-{[6-(dimethylamino)-2-methylpyrimidin-4-yl]amino}cyclohexyl)thiourea;
N-[4-bromo-2-(trifluoromethyl)phenyl]-N′-(cis-4-{[6-(dimethylamino)-2-methylpyrimidin-4-yl]amino}cyclohexyl)thiourea;
N-(cis-4-{[6-(dimethylamino)-2-methylpyrimidin-4-yl]amino}cyclohexyl)-3-nitrobenzamide;
N-[cis-4-(6-dimethylamino-2-methyl-pyrimidin-4-ylamino)-cyclohexyl]-3,4-diethoxy-benzamide;
N-[cis-4-(6-dimethylamino-2-methyl-pyrimidin-4-ylamino)-cyclohexyl]-3-ethoxy-benzamide;
N-[cis-4-(6-dimethylamino-2-methyl-pyrimidin-4-ylamino)-cyclohexyl]-3,5-diethoxy-benzamide;
N-[cis-4-(6-dimethylamino-2-methyl-pyrimidin-4-ylamino)-cyclohexyl]-3-isopropoxy-benzamide;
3-bromo-N-[cis-4-(6-dimethylamino-2-methyl-pyrimidin-4-ylamino)-cyclohexyl]-4-fluoro-benzamide;
4-difluoromethoxy-N-[cis-4-(6-dimethylamino-2-methyl-pyrimidin-4-ylamino)-cyclohexyl]-benzamide;
4-chloro-N-[cis-4-(6-dimethylamino-2-methyl-pyrimidin-4-ylamino)-cyclohexyl]-3-methyl-benzamide;
3-difluoromethoxy-N-[cis-4-(6-dimethylamino-2-methyl-pyrimidin-4-ylamino)-cyclohexyl]-benzamide;
3-chloro-N-[cis-4-(6-dimethylamino-2-methyl-pyrimidin-4-ylamino)-cyclohexyl]-4-methyl-benzamide;
4-bromo-N-[cis-4-(6-dimethylamino-2-methyl-pyrimidin-4-ylamino)-cyclohexyl]-benzamide;
N-[cis-4-(6-dimethylamino-2-methyl-pyrimidin-4-ylamino)-cyclohexyl]-3,5-dimethoxy-benzamide;
4-cyano-N-[cis-4-(6-dimethylamino-2-methyl-pyrimidin-4-ylamino)-cyclohexyl]-benzamide;
N-[cis-4-(6-dimethylamino-2-methyl-pyrimidin-4-ylamino)-cyclohexyl]-4-methoxy-benzamide;
3-cyano-N-[cis-4-(6-dimethylamino-2-methyl-pyrimidin-4-ylamino)-cyclohexyl]-benzamide;
N-[cis-4-(6-dimethylamino-2-methyl-pyrimidin-4-ylamino)-cyclohexyl]-3-methoxy-benzamide;
N-[cis-4-(6-dimethylamino-2-methyl-pyrimidin-4-ylamino)-cyclohexyl]-4-fluoro-3-methyl-benzamide;
4-bromo-N-[cis-4-(6-dimethylamino-2-methyl-pyrimidin-4-ylamino)-cyclohexyl]-3-methyl-benzamide;
N-[cis-4-(6-dimethylamino-2-methyl-pyrimidin-4-ylamino)-cyclohexyl]-3-fluoro-4-methyl-benzamide;
N-[cis-4-(6-dimethylamino-2-methyl-pyrimidin-4-ylamino)-cyclohexyl]-3-ethyl-benzamide;
3-bromo-N-[cis-4-(6-dimethylamino-2-methyl-pyrimidin-4-ylamino)-cyclohexyl]-benzamide;
N-[cis-4-(6-dimethylamino-2-methyl-pyrimidin-4-ylamino)-cyclohexyl]-3-fluoro-4-trifluoromethyl-benzamide;
N-[cis-4-(6-dimethylamino-2-methyl-pyrimidin-4-ylamino)-cyclohexyl]-4-trifluoromethoxy-benzamide;
N-[cis-4-(6-dimethylamino-2-methyl-pyrimidin-4-ylamino)-cyclohexyl]-4-methyl-benzamide;
N-[cis-4-(6-dimethylamino-2-methyl-pyrimidin-4-ylamino)-cyclohexyl]-3-methyl-benzamide;
N-[cis-4-(6-dimethylamino-2-methyl-pyrimidin-4-ylamino)-cyclohexyl]-4-trifluoromethyl-benzamide;
2,2-difluoro-benzo[1,3]dioxole-5-carboxylic acid[cis-4-(6-dimethylamino-2-methyl-pyrimidin-4-ylamino)-cyclohexyl]-amide;
N-{cis-4-[(1H-indol-2-ylmethyl)-amino]-cyclohexyl}-2,N′,N′-trimethyl-pyrimidine-4,6-diamine;
2,N,N-trimethyl-N′-[cis-4-(3-trifluoromethoxy-benzylamino)-cyclohexyl]-pyrimidine-4,6-diamine;
N-[cis-4-(3,4-difluoro-benzylamino)-cyclohexyl]-2,N′,N′-trimethyl-pyrimidine-4,6-diamine;
1-(3,4-dimethoxy-phenyl)-3-[cis-4-(6-dimethylamino-2-methyl-pyrimidin-4-ylamino)-cyclohexyl]-urea;
1-[cis-4-(6-dimethylamino-2-methyl-pyrimidin-4-ylamino)-cyclohexyl]-3-(2-ethoxy-phenyl)-urea;
1-(4-benzyloxy-phenyl)-3-[cis-4-(6-dimethylamino-2-methyl-pyrimidin-4-ylamino)-cyclohexyl]-urea;
3,5-dibromo-N-[cis-4-(6-dimethylamino-2-methyl-pyrimidin-4-ylamino)-cyclohexyl]-benzamide;
3-bromo-4-chloro-N-[cis-4-(6-dimethylamino-2-methyl-pyrimidin-4-ylamino)-cyclohexyl]-benzamide;
4-chloro-N-[cis-4-(6-dimethylamino-2-methyl-pyrimidin-4-ylamino)-cyclohexyl]-3-trifluoromethyl-benzamide;
2-(3,5-bis-trifluoromethyl-phenyl)-N-[cis-4-(6-dimethylamino-2-methyl-pyrimidin-4-ylamino)-cyclohexyl]-2-hydroxy-acetamide;
N-[cis-4-(6-dimethylamino-2-methyl-pyrimidin-4-ylamino)-cyclohexylmethyl]-3-fluoro-4-trifluoromethyl-benzamide,
N-[cis-4-(6-dimethylamino-2-methyl-pyrimidin-4-ylamino)-cyclohexylmethyl]-3-trifluoromethoxy-benzamide;
N-[cis-4-(6-dimethylamino-2-methyl-pyrimidin-4-ylamino)-cyclohexylmethyl]-3-methoxy-benzamide;
4-chloro-N-[cis-4-(6-dimethylamino-2-methyl-pyrimidin-4-ylamino)-cyclohexylmethyl]-benzamide;
N-[cis-4-(6-dimethylamino-2-methyl-pyrimidin-4-ylamino)-cyclohexylmethyl]-3-trifluoromethyl-benzamide;
N-[cis-4-(6-dimethylamino-2-methyl-pyrimidin-4-ylamino)-cyclohexylmethyl]-4-trifluoromethyl-benzamide;
N-[cis-4-(6-dimethylamino-2-methyl-pyrimidin-4-ylamino)-cyclohexylmethyl]-3-methyl-benzamide;
N-[cis-4-(6-dimethylamino-2-methyl-pyrimidin-4-ylamino)-cyclohexylmethyl]-3,5-difluoro-benzamide;
N-[cis-4-(6-dimethylamino-2-methyl-pyrimidin-4-ylamino)-cyclohexylmethyl]-3-ethyl-benzamide;
2,2-difluoro-benzo[1,3]dioxole-5-carboxylic acid [cis-4-(6-dimethylamino-2-methyl-pyrimidin-4-ylamino)-cyclohexylmethyl]-amide;
N-[cis-4-(6-dimethylamino-2-methyl-pyrimidin-4-ylamino)-cyclohexylmethyl]-3-fluoro-4-methyl-benzamide;
N-[cis-4-(6-dimethylamino-2-methyl-pyrimidin-4-ylamino)-cyclohexylmethyl]-4-fluoro-benzamide;
3,4-dichloro-N-[cis-4-(6-dimethylamino-2-methyl-pyrimidin-4-ylamino)-cyclohexylmethyl]-benzamide;
4-bromo-N-[cis-4-(6-dimethylamino-2-methyl-pyrimidin-4-ylamino)-cyclohexylmethyl]-benzamide;
N-[cis-4-(6-dimethylamino-2-methyl-pyrimidin-4-ylamino)-cyclohexylmethyl]-3,4-difluoro-benzamide;
3,5-dichloro-N-[cis-4-(6-dimethylamino-2-methyl-pyrimidin-4-ylamino)-cyclohexylmethyl]-benzamide;
3-chloro-N-[cis-4-(6-dimethylamino-2-methyl-pyrimidin-4-ylamino)-cyclohexylmethyl]-4-fluoro-benzamide;
N-[cis-4-(6-dimethylamino-2-methyl-pyrimidin-4-ylamino)-cyclohexylmethyl]-4-fluoro-3-methyl-benzamide; and
3-chloro-N-[cis-4-(6-dimethylamino-2-methyl-pyrimidin-4-ylamino)-cyclohexylmethyl]-benzamide;
or a pharmaceutically acceptable salt, hydrate, or solvate thereof.

8. The compound according to claim 1 selected from the group consisting of:
N-(cis-4-{[6-(dimethylamino)-2-methylpyrimidin-4-yl]amino}cyclohexyl)-3,4-difluorobenzamide;
N-(cis-4-{[6-(dimethylamino)-2-ethylpyrimidin-4-yl]amino}cyclohexyl)-3,4-difluorobenzamide;
3-chloro-N-(cis-4-{[6-(dimethylamino)-2-methylpyrimidin-4-yl]amino}cyclohexyl)-4-fluorobenzamide;
3,4-dichloro-N-(cis-4-{[6-(dimethylamino)-2-methylpyrimidin-4-yl]amino}cyclohexyl)benzamide;
3-chloro-N-(cis-4-{[6-(dimethylamino)-2-methylpyrimidin-4-yl]amino}cyclohexyl)-5-fluorobenzamide;
N-(cis-4-{[6-(dimethylamino)-2-methylpyrimidin-4-yl]amino}cyclohexyl)-3,4,5-trifluorobenzamide;
5-bromo-N-(cis-4-{[6-(dimethylamino)-2-methylpyrimidin-4-yl]amino}cyclohexyl)nicotinamide;
N-(cis-4-{[6-(dimethylamino)-2-methylpyrimidin-4-yl]amino}cyclohexyl)-4-fluoro-3-(trifluoromethyl)benzamide;
N-(cis-4-{[6-(dimethylamino)-2-methylpyrimidin-4-yl]amino}cyclohexyl)-3-(trifluoromethyl)benzamide;
N-(cis-4-{[6-(dimethylamino)-2-methylpyrimidin-4-yl]amino}cyclohexyl)-3-(trifluoromethoxy)benzamide;
3,5-dichloro-N-(cis-4-{[6-(dimethylamino)-2-methylpyrimidin-4-yl]amino}cyclohexyl)benzamide;
3-chloro-N-(cis-4-{[6-(dimethylamino)-2-methylpyrimidin-4-yl]amino}cyclohexyl)benzamide;
3-chloro-4-fluoro-N-{cis-4-[(2-methyl-6-pyrrolidin-1-ylpyrimidin-4-yl)amino]cyclohexyl}benzamide;
N-(cis-4-{[6-(dimethylamino)-2-ethylpyrimidin-4-yl]amino}cyclohexyl)-3,4,5-trifluorobenzamide;
cis-N-(3-chloro-4-fluorophenyl)-4-{[6-(dimethylamino)-2-methylpyrimidin-4-yl]amino}cyclohexanecarboxamide;
N-(cis-4-{[2-benzyl-6-(dimethylamino)pyrimidin-4-yl]amino}cyclohexyl)-3-chloro-4-fluorobenzamide;
cis-4-{[6-(dimethylamino)-2-methylpyrimidin-4-yl]amino}-N-(3,4,5-trifluorophenyl)cyclohexanecarboxamide;
N-(4-bromo-2,6-dimethylphenyl)-N′-(cis-4-{[6-(dimethylamino)-2-methylpyrimidin-4-yl]amino}cyclohexyl)urea;
N-(4-bromo-2,6-dimethylphenyl)-N′-(cis-4-{[6-(dimethylamino)-2-methylpyrimidin-4-yl]amino}cyclohexyl)thiourea;
N-(cis-4-{[6-(dimethylamino)-2-methylpyrimidin-4-yl]amino}cyclohexyl)-N′-(3,4,5-trimethoxyphenyl)thiourea;
N-(cis-4-{[6-(dimethylamino)-2-methylpyrimidin-4-yl]amino}cyclohexyl)-N′-(2,4,6-tribromophenyl)thiourea;
5-bromo-furan-2-carboxylic acid [cis-4-(6-dimethylamino-2-methyl-pyrimidin-4-ylamino)-cyclohexyl]-amide;
N-[cis-4-(3,5-dimethoxy-benzylamino)-cyclohexyl]-2,N′,N′-trimethyl-pyrimidine-4,6-diamine;
N-[cis-4-(3-bromo-benzylamino)-cyclohexyl]-2,N′,N′-trimethyl-pyrimidine-4,6-diamine;
1-[cis-4-(6-dimethylamino-2-methyl-pyrimidin-4-ylamino)-cyclohexyl]-3-(3-methoxy-phenyl)-urea;
1-(3,5-difluoro-phenyl)-3-[cis-4-(6-dimethylamino-2-methyl-pyrimidin-4-ylamino)-cyclohexyl]-urea;
N-[cis-4-(6-dimethylamino-2-methylsulfanyl-pyrimidin-4-ylamino)-cyclohexyl]-3,4-difluoro-benzamide;
N-[cis-4-(6-dimethylamino-5-methyl-pyrimidin-4-ylamino)-cyclohexyl]-3,4-difluoro-benzamide;
N-[cis-4-(6-dimethylamino-2-methyl-pyrimidin-4-ylamino)-cyclohexylmethyl]-3,5-bis-trifluoromethyl-benzamide; and
N-[cis-4-(6-dimethylamino-2-methyl-pyrimidin-4-ylamino)-cyclohexylmethyl]-4-trifluoromethoxy-benzamide;
or a pharmaceutically acceptable salt, hydrate, or solvate thereof.

9. The compound according to claim 2 wherein:
R₁ represents:
(i) hydrogen, —CO₂^{t}Bu, or —CO₂Bn (Bn is a benzyl group) when L is selected from the group consisting of Formulae (III), (IIIa), and (IIIb); or
(ii) hydrogen, C₁₋₅ alkyl, substituted C₁₋₅ alkyl, Bn, or substituted Bn when L is selected from the group consisting of Formulae (IV), (IVa), and (IVb);
wherein R₃ and R₄ are each independently hydrogen or C₁₋₅ alkyl; and A and B are each independently a single bond, —CH₂—, or —(CH₂)₂—;
R₂ is halogen, C₁₋₅ alkyl, C₁₋₅ alkoxy, —N(R₂ₐ)(R_{2b}), or heterocyclyl; wherein R₂ₐ and R_{2b} are each independently hydrogen, C₁₋₅ alkyl, C₁₋₅ alkyl substituted by hydroxy, C₁₋₅ alkyl substituted by carbocyclic aryl, C₁₋₅ alkyl substituted by heterocyclyl, C₃₋₆ cycloalkyl, or carbocyclic aryl;
Z₁ is hydrogen, halogen, C₁₋₅ alkyl, C₁₋₅ alkyl substituted by halogen, C₁₋₅ alkoxy, or C₁₋₅ alkylthio; Z₂ is hydrogen, halogen, or C₁₋₅ alkyl; or
R₂ and Z₂ are bonded to each other to form a ring and —R₂-Z₂- is —NR₆—CH═CH—; wherein
R₆ is hydrogen or C₁₋₅ alkyl;
and
Y represents:
(i) a single bond when L is selected from the group consisting of Formulae (III), (IIIa), and (IIIb); or
(ii) —C(O)O— when L is selected from the group consisting of Formulae (IV), (IVa), and (IVb);
or a pharmaceutically acceptable salt, hydrate, or solvate thereof.

10. The compound according to claim 9 wherein:
R₁ represents:
(i) hydrogen, —CO₂^{t}Bu, or —CO₂Bn (Bn is a benzyl group) when L is selected from the group consisting of Formula (IIIa); or
(ii) hydrogen, C₁₋₅ alkyl, substituted C₁₋₅ alkyl, Bn, or substituted Bn when L is selected from the group consisting of Formula (IVa);
wherein R₃ and R₄ are each hydrogen; and A and B are each independently a single bond or —CH₂—;
R₂ is —N(R₂ₐ)(R_{2b}) or heterocyclyl; wherein R₂ₐ and R_{2b} are each independently hydrogen or C₁₋₅ alkyl
Z₁ is hydrogen, C₁₋₅ alkyl, or C₁₋₅ alkylthio; Z₂ is hydrogen or C₁₋₅ alkyl; or
R₂ and Z₂ are bonded to each other to form a ring and —R₂-Z₂- is —NR₆—CH═CH—; wherein
R₆ is hydrogen or C₁₋₅ alkyl;
and
Y represents:
(i) a single bond when L is selected from the group consisting of Formula (IIIa); or
(ii) —C(O)O— when L is selected from the group consisting of Formula (IVa);
heterocyclyl is furyl, 1H-indolyl, morpholinyl, oxazolyl, piperidyl, pyridyl, pyrrolidyl, or 9H-xanthenyl;
or a pharmaceutically acceptable salt, hydrate, or solvate thereof.

11. The compound according to claim 1 wherein Q is Formula (IIb);
R₂ is C₁₋₅ alkyl substituted by hydroxy, C₁₋₅ alkyl substituted by carbocyclic aryl, C₁₋₅ alkyl substituted by halogenated carbocyclic aryl, C₁₋₅ alkyl substituted by heterocyclyl, C₁₋₅ alkyl substituted by halogenated heterocyclyl, C₂₋₅ alkenyl, C₂₋₅ alkynyl, or —N(R₂ₐ)(R_{2b}); wherein R₂ₐ and R_{2b} are each independently hydrogen,
C₁₋₅ alkyl, or C₁₋₅ alkyl substituted by substituent(s) independently selected from the group consisting of:
halogen,
hydroxy,
carboxy,
carbamoyl,
C₁₋₅ alkoxy,
amino,
C₃₋₆ cycloalkyl,
carbocyclic aryl,
carbocyclic aryl substituted by substituent(s) independently selected from the group consisting of:
halogen,
C₁₋₅ alkyl,
C₁₋₅ alkoxy,
C₁₋₅ alkyl substituted by halogen,
C₁₋₅ alkoxy substituted by halogen, and
SO₂NH₂,
heterocyclyl, and
heterocyclyl substituted by substituent(s) independently selected from the group consisting of:
halogen,
C₁₋₅ alkyl,
C₁₋₅ alkoxy,
C₁₋₅ alkyl substituted by halogen, and
C₁₋₅ alkoxy substituted by halogen,
carbocyclic aryl, carbocyclic aryl substituted by substituent(s) independently selected from the group consisting of:
halogen,
C₁₋₅ alkyl,
C₁₋₅ alkoxy,
C₁₋₅ alkyl substituted by halogen, and
C₁₋₅ alkoxy substituted by halogen,
heterocyclyl, or heterocyclyl substituted by substituent(s) independently selected from the group consisting of:
halogen,
C₁₋₅ alkyl,
C₁₋₅ alkoxy,
C₁₋₅ alkyl substituted by halogen, and
C₁₋₅ alkoxy substituted by halogen;
or a pharmaceutically acceptable salt, hydrate, or solvate thereof.

12. The compound according to claim 11 wherein R₁ is selected from the group consisting of:
(i) C₁₋₁₀ alkyl, and
C₁₋₁₀ alkyl substituted by substituent(s) independently selected from the group consisting of:
halogen,
hydroxy,
oxo,
C₁₋₅ alkoxy,
C₁₋₅ alkoxy substituted by carbocyclic aryl,
C₁₋₅ alkylcarbonyloxy,
C₁₋₅ alkoxycarbonyl,
C₁₋₅ alkoxycarbonyl substituted by carbocyclic aryl,
carbocyclic aryloxy, and
carbocyclic aryloxy substituted by substituent(s) independently selected from the group consisting of:
halogen,
nitro,
C₁₋₅ alkyl, and
C₁₋₅ alkyl substituted by oxo,
heterocyclyloxy,
heterocyclyloxy substituted by C₁₋₅ alkyl,
mono-carbocyclic arylamino,
di-carbocyclic arylamino,
carbocyclic arylsulfonylamino,
carbocyclic arylsulfonylamino substituted by C₁₋₅ alkyl,
C₁₋₅ alkylthio,
C₁₋₅ alkylthio substituted by carbocyclic aryl,
carbocyclic arylthio,
carbocyclic arylthio substituted by halogen,
carbocyclic arylthio substituted by C₁₋₅ alkyl,
carbocyclic arylsulfonyl,
carbocyclic arylsulfonyl substituted by halogen,
heterocyclylthio,
heterocyclylthio substituted by C₁₋₅ alkyl,
C₃₋₆ cycloalkyl,
C₃₋₆ cycloalkenyl,
carbocyclyl,
carbocyclyl substituted by C₁₋₅ alkoxy,
carbocyclic aryl, and
carbocyclic aryl substituted by substituent(s) independently selected from the group consisting of:
halogen,
nitro,
C₁₋₅ alkyl, and
C₁₋₅ alkyl substituted by substituent(s) independently selected from the group consisting of:
halogen,
carbocyclic aryl, and
heterocyclyl,
C₁₋₅ alkoxy,
C₁₋₅ alkoxy substituted by halogen,
C₁₋₅ alkoxy substituted by carbocyclic aryl,
carbocyclic aryloxy,
mono-carbocyclic arylaminocarbonyl, and
mono-carbocyclic arylaminocarbonyl substituted by substituent(s) selected from the group consisting of:
halogen,
C₁₋₅ alkyl,
C₁₋₅ alkoxy, and
C₁₋₅ alkoxy substituted by halogen,
di-carbocyclic arylaminocarbonyl, and
di-carbocyclic arylaminocarbonyl substituted by substituent(s) selected from the group consisting of:
halogen,
C₁₋₅ alkyl,
C₁₋₅ alkoxy, and
C₁₋₅ alkoxy substituted by halogen,
C₁₋₅ alkylthio,
C₁₋₅ alkylthio substituted by halogen,
C₁₋₅ alkylsulfonyl,
carbocyclic aryl, and
heterocyclyl,
heterocyclyl, and
heterocyclyl substituted by substituent(s) independently selected from the group consisting of:
C₁₋₅ alkyl,
C₁₋₅ alkoxy,
C₁₋₅ alkoxy substituted by carbocyclic aryl,
carbocyclic aryl, and
carbocyclic aryl substituted by halogen,
(ii) C₂₋₅ alkenyl, and
C₂₋₅ alkenyl substituted by substituent(s) independently selected from the group consisting of:
carbocyclic aryl, and
carbocyclic aryl substituted by substituent(s) independently selected from the group consisting of:
nitro,
halogen,
C₁₋₅ alkyl,
C₁₋₅ alkyl substituted by halogen,
C₁₋₅ alkoxy, and
C₁₋₅ alkoxy substituted by halogen,
(iii) C₃₋₆ cycloalkyl, and
C₃₋₆ cycloalkyl substituted by substituent(s) independently selected from the group consisting of:
C₁₋₅ alkyl,
C₁₋₅ alkyl substituted by carbocyclic aryl,
carbocyclic arylcarbonylamino, and
carbocyclic aryl,
(iv) carbocyclyl, and
carbocyclyl substituted by nitro,
(v) carbocyclic aryl, and
carbocyclic aryl substituted by substituent(s) independently selected from the group consisting of:
halogen,
cyano,
nitro,
C₁₋₉ alkyl, and
C₁₋₉ alkyl substituted by substituent(s) independently selected from the group consisting of:
halogen,
oxo,
mono-carbocyclic arylaminocarbonyl,
di-carbocyclic arylaminocarbonyl,
mono-carbocyclic arylaminocarbonyl substituted by C₁₋₅ alkoxy,
di-carbocyclic arylaminocarbonyl substituted by C₁₋₅ alkoxy,
carbocyclic aryloxy,
carbocyclic aryl, and
carbocyclic aryl substituted by substituent(s) independently selected from the group consisting of:
halogen,
C₁₋₅ alkyl, and
C₁₋₅ alkyl substituted by halogen,
heterocyclyl, and
heterocyclyl substituted by C₁₋₅ alkyl,
C₂₋₅ alkenyl,
C₁₋₇ alkoxy,
C₁₋₇ alkoxy substituted by halogen,
C₁₋₇ alkoxy substituted by carbocyclic aryl,
C₃₋₆ cycloalkoxy,
carbocyclic aryloxy, and
carbocyclic aryloxy substituted by substituent(s) independently selected from the group consisting of:
halogen,
nitro, and
C₁₋₅ alkoxy
heterocyclyloxy, and
heterocyclyloxy substituted by substituent(s) independently selected from the group consisting of:
halogen,
C₁₋₅ alkyl, and
C₁₋₅ alkyl substituted by halogen,
C₁₋₅ alkoxycarbonyl,
mono-C₁₋₅ alkylaminocarbonyl,
di-C₁₋₅ alkylaminocarbonyl,
mono-C₁₋₅ alkylaminocarbonyl substituted by carbocyclic aryl,
di-C₁₋₅ alkylaminocarbonyl substituted by carbocyclic aryl,
mono-carbocyclic arylaminocarbonyl,
di-carbocyclic arylaminocarbonyl,
mono-carbocyclic arylaminocarbonyl substituted by C₁₋₅ alkyl,
di-carbocyclic arylaminocarbonyl substituted by C₁₋₅ alkyl,
mono-C₁₋₅ alkylamino,
di-C₁₋₅ alkylamino,
C₁₋₅ alkylthio,
C₁₋₅ alkylthio substituted by halogen,
C₁₋₅ alkylsulfonyl,
carbocyclic aryl, and
carbocyclic aryl substituted by substituent(s) independently selected from the group consisting of:
C₁₋₇ alkyl, and
C₁₋₇ alkyl substituted by halogen,
(vi) heterocyclyl, and
heterocyclyl substituted by substituent(s) independently selected from the group consisting of:
halogen,
C₁₋₅ alkyl, and
C₁₋₅ alkyl substituted by substituent(s) independently selected from the group consisting of:
halogen,
oxo,
carbocyclic aryl,
carbocyclic aryl substituted by halogen,
heterocyclyl, and
heterocyclyl substituted by substituent(s) independently selected from the group consisting of:
halogen,
C₁₋₅ alkyl, and
C₁₋₅ alkyl substituted by halogen,
C₁₋₅ alkoxy,
C₁₋₅ alkylthio,
carbocyclic arylthio,
C₁₋₅ alkylsulfonyl,
carbocyclic arylsulfonyl,
carbocyclic arylsulfonyl substituted by halogen,
carbocyclic arylsulfonyl substituted by C₁₋₅ alkyl,
C₁₋₅ alkoxycarbonyl,
carbocyclic aryl, and
carbocyclic aryl substituted by substituent(s) independently selected from the group consisting of:
halogen,
nitro, and
C₁₋₅ alkyl,
heterocyclyl, and
heterocyclyl substituted by substituent(s) independently selected from the group consisting of:
halogen,
C₁₋₅ alkyl, and
C₁₋₅ alkyl substituted by halogen;
wherein carbocyclic aryl is phenyl, naphthyl, or anthranyl;
carbocyclyl is 1-oxo-indanyl, 9H-fluorenyl, 9-oxo-fluorenyl, anthraquinonyl, C-fluoren-9-ylidene, indanyl, or menthyl;
heterocyclyl is 1,2,3,4-tetrahydro-isoquinolyl, 1,2,3-thiadiazolyl, 1,2,3-triazolyl, 1,3-dioxo-isoindolyl, 1H-indolyl, 1H-pyrrolyl, 2,3-dihydro-1-oxo-isoindolyl, 2,3-dihydro-benzo[1,4]dioxinyl, 2H-benzopyranyl, 2-oxo-benzopyranyl, 2-oxo-pyrrolidinyl, 4-oxo-benzopyranyl, 9H-xanthenyl, benzo[1,3]dioxolyl, benzo[2,1,3]oxadiazolyl, benzo[1,2,5]oxadiazolyl, benzo[b]thienyl, furyl, isoxazolyl, morpholinyl, oxazolyl, pyrazolyl, pyridyl, pyrimidyl, pyrrolidyl, quinolyl, quinoxalyl, thiazolyl, or thienyl;
halogen is fluoro, chloro, bromo, or iodo;
or a pharmaceutically acceptable salt, hydrate, or solvate thereof.

13. The compound according to claim 12 wherein:
R₂ is C₁₋₅ alkyl substituted by carbocyclic aryl, C₁₋₅ alkyl substituted by halogenated carbocyclic aryl, C₁₋₅ alkyl substituted by heterocyclyl, C₁₋₅ alkyl substituted by halogenated heterocyclyl, carbocyclic aryl, carbocyclic aryl by halogen, heterocyclyl, heterocyclyl by halogen, or —N(R₂ₐ)(R_{2b}); wherein R₂ₐ and R_{2b} are each independently hydrogen, C₁₋₅ alkyl, C₁₋₅ alkyl substituted by hydroxy, or C₁₋₅ alkyl substituted by halogen;
L is Formula (IIIa); wherein R₃ and R₄ are each independently hydrogen or C₁₋₅ alkyl;
and A and B are each independently a single bond, —CH₂—, or —(CH₂)₂—;
Z₃ and Z₄ are each independently hydrogen, halogen, C₁₋₅ alkyl, C₁₋₅ alkyl substituted by halogen, mono-C₁₋₅ alkyl amino, or di-C₁₋₅ alkyl amino;
and
Y is —C(O)—, —C(O)NR₅—, —C(S)NR₅—, or —(CH₂)ₘ—; wherein R₅ is hydrogen or C₁₋₅ alkyl;
and m is 0, 1, or 2; Y is not —(CH₂)ₘ— provided that either R₂ₐ or R_{2b} is hydrogen;
or a pharmaceutically acceptable salt, hydrate, or solvate thereof.

14. The compound according to claim 13 wherein R₁ is selected from the group consisting of:
(i) C₁₋₅ alkyl substituted by substituent(s) independently selected from the group consisting of:
hydroxy,
carbocyclic aryl,
carbocyclic aryl substituted by halogen, and
carbocyclic aryl substituted by halogenated C₁₋₅ alkyl,
(ii) carbocyclic aryl, and
carbocyclic aryl substituted by substituent(s) independently selected from the group consisting of:
halogen,
cyano,
C₁₋₅ alkyl,
C₁₋₅ alkyl substituted by halogen,
C₁₋₅ alkoxy, and
C₁₋₅ alkoxy substituted by halogen,
(iii) heterocyclyl, and
heterocyclyl substituted by halogen;
R₂ is C₁₋₅ alkyl substituted by carbocyclic aryl or —N(R₂ₐ)(R_{2b}); wherein R₂ₐ and R_{2b} are each independently hydrogen or C₁₋₅ alkyl;
L is Formula (IIIa); wherein R₃ and R₄ are each hydrogen; and A and B are each a single bond;
Z₃ and Z₄ are each independently hydrogen, C₁₋₅ alkyl, mono-C₁₋₅ alkyl amino, or di-C₁₋₅ alkyl amino;
and
Y is —C(O)—;
wherein carbocyclic aryl is phenyl;
heterocyclyl is furyl or pyridyl;
halogen is fluoro, chloro, or bromo;
or a pharmaceutically acceptable salt, hydrate, or solvate thereof.

15. The compound according to claim 14 wherein R₁ is selected from the group consisting of:
carbocyclic aryl, and
carbocyclic aryl substituted by substituent(s) independently selected from the group consisting of:
halogen,
cyano, and
C₁₋₅ alkoxy;
Z₃ is hydrogen when Z₄ is C₁₋₅ alkyl; or Z₃ is C₁₋₅ alkyl, mono-C₁₋₅ alkyl amino, or di-C₁₋₅ alkyl amino when Z₄ is hydrogen;
or a pharmaceutically acceptable salt, hydrate, or solvate thereof.

16. The compound according to claim 1 selected from the group consisting of:
3-chloro-N-(cis-4-{[2-(dimethylamino)-6-methylpyrimidin-4-yl]amino}cyclohexyl)-4-fluorobenzamide;
N-(cis-4-{[2-(dimethylamino)-6-methylpyrimidin-4-yl]amino}cyclohexyl)-3,4-difluorobenzamide;
N-[cis-4-(2-dimethylamino-5-methyl-pyrimidin-4-ylamino)-cyclohexyl]-3-methoxy-benzamide;
N-[cis-4-(2-dimethylamino-5-methyl-pyrimidin-4-ylamino)-cyclohexyl]-3-trifluoromethyl-benzamide;
N-[cis-4-(2-dimethylamino-5-methyl-pyrimidin-4-ylamino)-cyclohexyl]-3,5-bis-trifluoromethyl-benzamide;
2,2-difluoro-benzo[1,3]dioxole-5-carboxylic acid [cis-4-(2-dimethylamino-5-methyl-pyrimidin-4-ylamino)-cyclohexyl]-amide;
4-cyano-N-[cis-4-(2-dimethylamino-5-methyl-pyrimidin-4-ylamino)-cyclohexyl]-benzamide;
4-chloro-N-[cis-4-(2-dimethylamino-5-methyl-pyrimidin-4-ylamino)-cyclohexyl]-benzamide;
N-[cis-4-(2-dimethylamino-5-methyl-pyrimidin-4-ylamino)-cyclohexyl]-3-ethyl-benzamide;
N-[cis-4-(2-dimethylamino-5-methyl-pyrimidin-4-ylamino-cyclohexyl]-3,4-difluoro-benzamide;
5-bromo-N-[cis-4-(2-dimethylamino-5-methyl-pyrimidin-4-ylamino)-cyclohexyl]-nicotinamide;
5-bromo-furan-2-carboxylic acid [cis-4-(2-dimethylamino-5-methyl-pyrimidin-4-ylamino)-cyclohexyl]-amide;
3,5-dibromo-N-[cis-4-(2-dimethylamino-5-methyl-pyrimidin-4-ylamino)-cyclohexyl]-benzamide;
N-[cis-4-(2-dimethylamino-5-methyl-pyrimidin-4-ylamino)-cyclohexyl]-3-ethoxy-benzamide;
2-(3,5-bis-trifluoromethyl-phenyl)-N-[cis-4-(2-dimethylamino-5-methyl-pyrimidin-4-ylamino)-cyclohexyl]-2-hydroxy-acetamide;
2-(4-bromo-phenyl)-N-[cis-4-(2-dimethylamino-5-methyl-pyrimidin-4-ylamino)-cyclohexyl]-2-hydroxy-acetamide;
N-[cis-4-(2-dimethylamino-5-methyl-pyrimidin-4-ylamino)-cyclohexyl]-3,5-diethoxy-benzamide;
3-bromo-N-[cis-4-(2-dimethylamino-5-methyl-pyrimidin-4-ylamino)-cyclohexyl]-4-fluoro-benzamide;
N-[cis-4-(2-dimethylamino-6-methyl-pyrimidin-4-ylamino)-cyclohexyl]-3-ethoxy-benzamide;
N-[cis-4-(2-dimethylamino-6-methyl-pyrimidin-4-ylamino)-cyclohexyl]-3-trifluoromethyl-benzamide;
N-[cis-4-(2-dimethylamino-6-methyl-pyrimidin-4-ylamino)-cyclohexyl]-3,5-bis-trifluoromethyl-benzamide;
2,2-difluoro-benzo[1,3]dioxole-5-carboxylic acid [cis-4-(2-dimethylamino-6-methyl-pyrimidin-4-ylamino)-cyclohexyl]-amide;
4-chloro-N-[cis-4-(2-dimethylamino-6-methyl-pyrimidin-4-ylamino)-cyclohexyl]-benzamide;
N-[cis-4-(2-dimethylamino-6-methyl-pyrimidin-4-ylamino)-cyclohexyl]-3-ethyl-benzamide;
N-[cis-4-(2-dimethylamino-6-methyl-pyrimidin-4-ylamino)-cyclohexyl]-4-methyl-benzamide;
5-bromo-N-[cis-4-(2-dimethylamino-6-methyl-pyrimidin-4-ylamino)-cyclohexyl]-nicotinamide;
5-bromo-furan-2-carboxylic acid [cis-4-(2-dimethylamino-6-methyl-pyrimidin-4-ylamino)-cyclohexyl]-amide;
3,5-dibromo-N-[cis-4-(2-dimethylamino-6-methyl-pyrimidin-4-ylamino)-cyclohexyl]-benzamide;
N-[cis-4-(2-dimethylamino-6-methyl-pyrimidin-4-ylamino)-cyclohexyl]-3-ethoxy-benzamide;
2-(3,5-bis-trifluoromethyl-phenyl)-N-[cis-4-(2-dimethylamino-6-methyl-pyrimidin-4-ylamino)-cyclohexyl]-2-hydroxy-acetamide;
2-(4-bromo-phenyl)-N-[cis-4-(2-dimethylamino-6-methyl-pyrimidin-4-ylamino)-cyclohexyl]-2-hydroxy-acetamide;
N-[cis-4-(2-dimethylamino-6-methyl-pyrimidin-4-ylamino)-cyclohexyl]-3,5-diethoxy-benzamide; and
3-bromo-N-[cis-4-(2-dimethylamino-6-methyl-pyrimidin-4-ylamino)-cyclohexyl]-4-fluoro-benzamide;
or a pharmaceutically acceptable salt, hydrate, or solvate thereof.

17. The compound according to claim 1 selected from the group consisting of:
3-chloro-N-(cis-4-{[2-(dimethylamino)pyrimidin-4-yl]amino}cyclohexyl)-4-fluorobenzamide;
N-(cis-4-{[2,6-bis(dimethylamino)pyrimidin-4-yl]amino}cyclohexyl)-3,4-difluorobenzamide;
N-(cis-4-{[2-benzyl-6-(dimethylamino)pyrimidin-4-yl]amino}cyclohexyl)-3-chloro-4-fluorobenzamide;
3,4-dichloro-N-[cis-4-(2-dimethylamino-6-methyl-pyrimidin-4-ylamino-cyclohexyl]-benzamide;
4-cyano-N-[cis-4-(2-dimethylamino-6-methyl-pyrimidin-4-ylamino)-cyclohexyl]-benzamide;
N-[cis-4-(2-dimethylamino-6-methyl-pyrimidin-4-ylamino-cyclohexyl]-3,4-diethoxy-benzamide;
3-chloro-N-[cis-4-(2-dimethylamino-6-methyl-pyrimidin-4-ylamino)-cyclohexyl]-5-fluoro-benzamide;
N-[cis-4-(2-dimethylamino-5-methyl-pyrimidin-4-ylamino-cyclohexyl]-3,5-dimethoxy-benzamide;
3,4-dichloro-N-[cis-4-(2-dimethylamino-5-methyl-pyrimidin-4-ylamino)-cyclohexyl]-benzamide;
N-[cis-4-(2-dimethylamino-5-methyl-pyrimidin-4-ylamino)-cyclohexyl]-3,4-diethoxy-benzamide; and
3-chloro-N-[cis-4-(2-dimethylamino-5-methyl-pyrimidin-4-ylamino)-cyclohexyl]-5-fluoro-benzamide;
or a pharmaceutically acceptable salt, hydrate, or solvate thereof.

18. The compound according to claim 11 wherein:
R₁ is selected from hydrogen, —CO₂^{t}Bu, or —CO₂Bn (Bn is a benzyl group);
R₂ is C₁₋₅ alkyl substituted by carbocyclic aryl, C₁₋₅ alkyl substituted by halogenated carbocyclic aryl, C₁₋₅ alkyl substituted by heterocyclyl, C₁₋₅ alkyl substituted by halogenated heterocyclyl, carbocyclic aryl, carbocyclic aryl by halogen, heterocyclyl, heterocyclyl by halogen, or —N(R₂ₐ)(R_{2b}); wherein R₂ₐ and R_{2b} are each independently hydrogen, C₁₋₅ alkyl, C₁₋₅ alkyl substituted by hydroxy, or C₁₋₅ alkyl substituted by halogen;
L is Formula (IIIa); wherein R₃ and R₄ are each independently hydrogen or C₁₋₅ alkyl; and A and B are each independently a single bond, —CH₂—, or —(CH₂)₂—;
Z₃ and Z₄ are each independently hydrogen, halogen, C₁₋₅ alkyl, C₁₋₅ alkyl substituted by halogen, mono-C₁₋₅ alkyl amino, or di-C₁₋₅ alkyl amino; and
Y is a single bond;
or a pharmaceutically acceptable salt, hydrate, or solvate thereof.

19. The compound according to claim 18 wherein:
R₂ is C₁₋₅ alkyl substituted by carbocyclic aryl or —N(R₂ₐ)(R_{2b}); wherein R₂ₐ and R_{2b} are each independently hydrogen or C₁₋₅ alkyl;
L is Formula (IIIa); wherein R₃ and R₄ are each hydrogen; and A and B are each a single bond; and
Z₃ and Z₄ are each independently hydrogen, C₁₋₅ alkyl, mono-C₁₋₅ alkyl amino, or di-C₁₋₅ alkyl amino;
wherein carbocyclic aryl is phenyl;
heterocyclyl is furyl or pyridyl;
halogen is fluoro, chloro, or bromo;
or a pharmaceutically acceptable salt, hydrate, or solvate thereof.

20. A pharmaceutical composition comprising a therapeutically effective amount of a compound according to any one of claims 1 to 19 in combination with a pharmaceutically acceptable carrier.

21. A method for the prophylaxis or treatment of improving memory function, sleeping and arousal, anxiety, depression, mood disorders, seizure, obesity, diabetes, appetite and eating disorders, cardiovascular disease, hypertension, dyslipidemia, myocardial infarction, binge eating disorders including bulimia, anorexia, mental disorders including manic depression, schizophrenia, delirium, dementia, stress, cognitive disorders, attention deficit disorder, substance abuse disorders and dyskinesias including Parkinson's disease, epilepsy, and addiction comprising administering to an individual suffering from said condition a therapeutically effective amount of a compound according to claim 1.

22. A method for the prophylaxis or treatment of an eating disorder, obesity or an obesity related disorder comprising administering to an individual suffering from said condition a therapeutically effective amount of a compound according to claim 1.

23. A method for the prophylaxis or treatment of anxiety, depression, schizophrenia, addiction, or epilepsy comprising administering to an individual suffering from said condition a therapeutically effective amount of a compound according to claim 1.

24. A compound according to claim 1 for use in a method of treatment of the human or animal body by therapy.

25. A compound according to claim 1 for use in a method of prophylaxis or treatment of an eating disorder, obesity or an obesity related disorder of the human or animal body by therapy.

26. A compound according to claim 1 for use in a method of prophylaxis or treatment of anxiety, depression, schizophrenia, addiction, or epilepsy of the human or animal body by therapy.

27. A compound according to claim 1 for the manufacture of a medicament for use in the prophylaxis or treatment of an eating disorder, obesity or obesity related disorders.

28. A compound according to claim 1 for the manufacture of a medicament for use in the prophylaxis or treatment of anxiety, depression, schizophrenia, addiction, or epilepsy.

29. A method of decreasing food intake of an individual comprising administering to said individual a therapeutically effective amount of a compound according to claim 1.

30. A method of inducing satiety in an individual comprising administering to said individual a therapeutically effective amount of a compound according to claim 1.

31. A method of controlling or reducing weight gain in an individual comprising administering to said individual a therapeutically effective amount of a compound according to claim 1.

32. A method of modulating a MCH receptor in an individual comprising contacting the receptor with a compound according to claim 1.

33. The method of modulating the MCH receptor according to claim 32 wherein the compound is an antagonist.

34. The method of modulating the MCH receptor according to claims 32 or 33 wherein the modulation of the MCH receptor is for the prophylaxis or treatment of an eating disorder, obesity or obesity related disorder.

35. The method of modulating the MCH receptor according to claims 32 or 33 wherein the modulation of the MCH receptor reduces food intake of the individual.

36. The method of modulating the MCH receptor according to claims 32 or 33 wherein the modulation of the MCH receptor induces satiety in the individual.

37. The method of modulating the MCH receptor according to claims 32 or 33 wherein the modulation of the MCH receptor controls or reduces weight gain of the individual.

38. The method of modulating the MCH receptor according to claims 32 or 33 wherein the modulation of the MCH receptor is for prophylaxis or treatment of anxiety, depression, schizophrenia, addiction, or epilepsy.

39. The method of modulating the MCH receptor according to any one of claims 32 or 33 wherein the individual is a mammal.

40. The method of modulating the MCH receptor according to claim 39 wherein the mammal is a human.

41. The method according to claim 40 wherein the human has a body mass index of about 18.5 to about 45.

42. The method according to claim 41 wherein the human has a body mass index of about 25 to about 45.

43. The method according to claim 42 wherein the human has a body mass index of about 30 to about 45.

44. The method according to claim 43 wherein the human has a body mass index of about 35 to about 45.

45. A method of producing a pharmaceutical composition comprising admixing a compound according to claim 1 and a pharmaceutically acceptable carrier.


SEQUENCE LISTING (3 sequences)

SEQ ID NO: 1 - protein, 422 aa, Homo sapiens
MSVGAMKKGVGRAVGLGGGSGCQATEEDPLPNCGACAPGQGGRRWRLPQPAWVEGSSARLWEQATGTGWMDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 2 - DNA, 2423 bp, Homo sapiens
gggaggcagcctgtgtgctttgtccgtttgcctttgcagagacctcgatcttcacgcaaggcaagcagcagcccctgtaagcacacgagacaatcccaagtgtcagtgggaaggagatccctttcctgatggggctgcctgtgtccagtccctcccagcttccccagggccctggggctctgcaggcattcagaagtggaagccagccacagcctgggactgaagaggttaatgtgcatctgcctccgaatgttaatgtgtctaggtgatgtcagtgggagccatgaagaagggagtggggagggcagttgggcttggaggcggcagcggctgccaggctacggaggaagacccccttcccaactgcggggcttgcgctccgggacaaggtggcaggcgctggaggctgccgcagcctgcgtgggtggaggggagctcagctcggttgtgggagcaggcgaccggcactggctggatggacctggaagcctcgctgctgcccactggtcccaacgccagcaacacctctgatggccccgataacctcacttcggcaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctacctggccactgtccaccccatctcttccacgaagttccggaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcatacgtgaggatcctgcagcgcatgacgtcctcagtggcccccgcctcccagcgcagcatccggctgcggacaaagagggtgacccgcacagccatcgccatctgtctggtcttctttgtgtgctgggcaccctactatgtgctacagctgacccagttgtccatcagccgcccgaccctcacctttgtctacttatacaatgcggccatcagcttgggctatgccaacagctgcctcaacccctttgtgtacatcgtgctctgtgagacgttccgcaaacgcttggtcctgtcggtgaagcctgcagcccaggggcagcttcgcgctgtcagcaacgctcagacggctgacgaggagaggacagaaagcaaaggcacctgatacttcccctgccaccctgcacacctccaagtcagggcaccacaacacgccaccgggagagatgctgagaaaaacccaagaccgctcgggaaatgcaggaaggccgggttgtgaggggttgttgcaatgaaataaatacattccatggggctcacacgttgctggggaggcctggagtcaggtttggggttttcagatatcagaaatccccttgggggagcaggatgagacctttggatagaacagaagctgagcaagagaacatgttggtttggataaccggttgcactatatctgtgagctctcaaatgtcttcttcccaaggcaagaggtggaagggtactgactgggtttgtttaaagtcaggcagggctggagtgagcagccagggccatgttgcacaaggcctgagagacgggaaagggcccgatcgctctttcccgcctctcactggtgcgatggaaggtggcctttctcccaagctggtggataatgaaaaataaagcatcccatctctcggcgttccagcatcctgtcaatttcccttttgctctagaggatgcatgtttatttgaggggatgtggcactgagcccacaggagtaaaagcccagtttgctaggaggtctgcttactgaaaacaaggagacctggggtgggtgtggttgggggtcttaaaactaataaaagctggggtcggggggcttttgcagctctggtgacattctctccacggggcacatttgctcagtcactaatccagcttgagtgtccgtgtgttctgcatgtgcaggggtcattctagtgcccggtgtgttggcatcatctttttgctctagcccttcctctccaaaataaaatcaaataaaggaaaatctccacccaca

SEQ ID NO: 3 - protein, 19 aa, Homo sapiens
DFDMLRCMLGRVYRPCWQV